(19) United States
(12) Patent Application Publication
(10) Pub. No.: US 2003/0143708 A1
(43) Pub. Date: Jul. 31, 2003

(54) Method and reagent for the treatment of alzheimer's disease

(76) Inventors: La Wrence Blatt, Boulder, CO (US); James A. McSwiggen, Boulder, CO (US)

Correspondence Address: Anita J. Terpstra, McDonnell Boehnen Hulbert & Berghoff, 32nd Floor, 300 S. Wacker Drive, Chicago, IL, 60606, US

(21) Appl. No.: 09/745,237

(22) Filed: Dec. 20, 2000

Related U.S. Application Data
(60) Provisional application No. 60/173,612, filed on Dec. 29, 1999.

(51) Int. Cl. A61K 48/00; C07H 21/04; C12N 9/99; C12P 21/02; C12N 5/06

(52) U.S. Cl. 435/184; 435/69.2; 435/320.1; 435/325; 514/44; 536/23.2

(57) ABSTRACT

Nucleic acid molecules, including antisense and enzymatic nucleic acid molecules, such as hammerhead ribozymes, DNAzymes, and antisense, which modulate the expression of molecular targets impacting the development and progression of Alzheimer's disease, in particular, the expression of BACE and ps-2 gene.

### BACKGROUND OF THE INVENTION

The present invention concerns compounds, compositions, and methods for the study, diagnosis, and treatment of Alzheimer's disease (AD).

The following is a brief description of the current understanding of Alzheimer's disease. The discussion is not meant to be complete and is provided only to assist understanding the invention that follows. The summary is not an admission that any of the work described below is prior art to the claimed invention.

Alzheimer's disease (AD) is a progressive, degenerative disease of the brain which affects approximately 4 million people in the United States alone. An estimated 14 million Americans will have Alzheimer's disease by the middle of the next century if no cure or definitive prevention of the disease is found. Nearly one out of ten people over age 65 and nearly half of those over 85 have Alzheimer's disease. Alzheimer's disease is not confined to the elderly, a small percentage of people in their 30's and 40's are afflicted with early onset AD. Alzheimer's disease is the most common form of dementia, and amounts to the third most expensive disease in the US following heart disease and cancer. An estimated 100 billion dollars are spent annually on Alzheimer's disease (National Alzheimer's Association, 1999).

Alzheimer's disease is characterized by the progressive formation of insoluble plaques and vascular deposits in the brain consisting of the 4 kD amyloid β peptide (Aβ). These plaques are characterized by dystrophic neurites that show profound synaptic loss, neurofibrillary tangle formation, and gliosis. Aβ arises from the proteolytic cleavage of the large type I transmembrane protein, β-amyloid precursor protein (APP) (Kang et al., 1987, Nature, 325, 733). Processing of APP to generate AP requires two sites of cleavage by a β-secretase and a γ-secretase. β-secretase cleavage of APP results in the cytoplasmic release of a 100 kD soluble amino-terminal fragment, APPsβ, leaving behind a 12 kD transmembrane carboxy-terminal fragment, C99. Alternately, APP can be cleaved by a α-secretase to generate cytoplasmic APPsα and transmembrane C83 fragments. Both remaining transmembrane fragments, C99 and C83, can be further cleaved by a γ-secretase, leading to the release and secretion of Alzheimer's related Aβ and a non-pathogenic peptide, p3, respectively (Vassar et al., 1999, Science, 286, 735-741). Early onset familial Alzheimer's disease is characterized by mutant APP protein with a Met to Leu substitution at position P1, characterized as the “Swedish” familial mutation (Mullan et al., 1992, Nature Genet., 1, 345). This APP mutation is characterized by a dramatic enhancement in β-secretase cleavage (Citron et al., 1992, Nature, 360, 672).

The identification of β-secretase, and γ-secretase constituents involved in the release of β-amyloid protein is of primary importance in the development of treatment strategies for Alzheimer's disease. Characterization of α-secretase is also important in this regard since α-secretase cleavage may compete with β-secretase cleavage resulting in non-pathogenic vs. pathogenic protein production. Involvement of the two metalloproteases, ADAM 10, and TACE has been demonstrated in α-cleavage of AAP (Buxbaum et al., 1999, J. Biol. Chem., 273, 27765. and Lammich et al., 1999, Proc. Natl. Acad. Sci. U.S.A., 96, 3922). Studies of 7-secretase activity have demonstrated presenilin dependence (De Stooper et al. 1998, Nature, 391, 387, and De Stooper et al., 1999, Nature, 398, 518), and as such, presenilins have been proposed as γ-secretase even though presenilin does not present proteolytic activity (Wolfe et al., 1999, Nature, 398, 513).

Recently, Vassar et al., 1999, supra reported β-secretase cleavage of AAP by the transmembrane aspartic protease beta site APP cleaving enzyme, BACE. While other potential candidates for β-secretase have been proposed (for review see Evin et al., 1999, Proc. Natl. Acad. Sci. U.S.A., 96, 3922), none have demonstrated the full range of characteristics expected from this enzyme. Vassar et al., supra, demonstrate that BACE expression and localization are as expected for β-secretase, that BACE overexpression in cells results in increased β-secretase cleavage of APP and Swedish APP, that isolated BACE demonstrates site specific proteolytic activity on APP derived peptide substrates, and that antisense mediated endogenous BACE inhibition results in dramatically reduced β-secretase activity.

Current treatment strategies for Alzheimer's disease rely on either the prevention or the alleviation of symptoms and/or the slowing down of disease progression. Two drugs approved in the treatment of Alzheimer's, donepezil (Aricept®) and tacrine (Cognex®), both cholinomimetics, attempt to slow the loss of cognitive ability by increasing the amount of acetylcholine available to the brain. Antioxidant therapy through the use of antioxidant compounds such as alpha-tocopherol (vitamin E), melatonin. and selegeline (Eldepryl®) attempt to slow disease progression by minimizing free radical damage. Estrogen replacement therapy is thought to incur a possible preventative benefit in the development of Alzheimer's disease based on limited data. The use of anti-inflammatory drugs may be associated with a reduced risk of Alzheimer's as well. Calcium channel blockers such as Nimodipine® are considered to have a potential benefit in treating Alzheimer's disease due to protection of nerve cells from calcium overload, thereby prolonging nerve cell survival. Nootropic compounds, such as acetyl-L-carnitine (Alcar®) and insulin, have been proposed to have some benefit in treating Alzheimer's due to enhancement of cognitive and memory function based on cellular metabolism.

Whereby the above treatment strategies may all improve quality of life in Alzheimer's patients, there exists an unmet need in the comprehensive treatment and prevention of this disease. As such, there exists the need for therapeutics effective in reversing the physiological changes associated with Alzheimer's disease, specifically, therapeutics that can eliminate and/or reverse the deposition of amyloid β peptide. The use of compounds to modulate the expression of proteases that are instrumental in the release of amyloid β peptide, namely β-secretase (BACE), and γ-secretase (presenilin), is of therapeutic significance.

Tsai et al., 1999, Book of Abstrasts, 218^{th} ACS National Meeting, New Orleans, August 22-26, describe substrate-based alpha-aminoisobutyric acid derivatives of difluoro ketone peptidomimetic inhibitors of amyloid β peptide through γ-secretase inhibition.

Czech et al., International PCT publication No. WO/9921886, describe peptides capable of inhibiting the interaction between presenilins and the β-amyloid peptide or its precursor for therapeutic use.

Fournier et al., International PCT publication No. WO/9916874, describe human brain proteins capable of interacting with presenilins and cDNAs encoding them toward therapeutic use.

St. George-Hyslop et al., International PCT publication No. WO/9727296, describe genes for proteins that interact with presenilins and their role in Alzheimer's disease toward therapeutic use.

Vassar et al., 1999, Science, 286, 735-741, describe specific antisense oligonucleotides targeting BACE, used for inhibition studies of endogenous BACE expression in 101 cells and APPsw cells via lipid mediated transfection.

### SUMMARY OF THE INVENTION

The invention features novel nucleic acid-based techniques (e.g., enzymatic nucleic acid molecules, such as ribozymes), and methods for their use to modulate the expression of molecular targets impacting the development and progression of Alzheimer's disease.

In a preferred embodiment, the invention features use of such novel nucleic acid-based techniques, independently or in combination, to modulate, down regulate, or inhibit the expression of beta secretase, such as beta-site APP-cleaving enzyme (BACE, also known as Asp-2) (GenBank accession AF190725), and gamma secretase, such as presenilin 1 (ps-1) (GenBank accession L76517), and presenilin 2 (ps-2) (GenBank accession L43964) involved in cleaving beta-amyloid precursor protein to yield amyloid β peptide.

In more preferred embodiments, the invention features the use of an enzymatic nucleic acid molecule, preferably in the hammerhead, NCH, G-cleaver, zinzyme, amberzyme and/or DNAzyme motif, to inhibit the expression of beta-site APP-cleaving enzyme (BACE) gene and/or the presenilin (ps-2) gene.

By “inhibit” it is meant that the activity of a particular product(s) on the level of particular RNA(s), e.g., of BACE and/or ps-2 or level of RNAs or equivalent RNAs encoding one or more protein subunits of BACE and/or ps-2, is reduced below that observed in the absence of the nucleic acid. In one embodiment, inhibition with enzymatic nucleic acid molecule preferably is below that level observed in the presence of an enzymatically inactive or attenuated molecule that is able to bind to the same site on the target RNA, but is unable to cleave that RNA.

In another embodiment, inhibition with antisense oligonucleotides is preferably below that level observed in the presence of, for example, an oligonucleotide with scrambled sequence or with mismatches. In another embodiment, inhibition of BACE genes and/or ps-2 with the nucleic acid molecule of the instant invention is greater than in the presence of the nucleic acid molecule than in its absence.

By “enzymatic nucleic acid molecule” it is meant a nucleic acid molecule that has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme. RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “enzymatic portion” or “catalytic domain” is meant that portion/region of the enzymatic nucleic acid molecule essential for cleavage of a nucleic acid substrate (for example see FIG. 1).

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired. Such arms are shown generally in FIG. 1. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides and of sufficient length to stably interact with the target RNA; a specific embodiment 12-100 nucleotides; more preferably 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “NCH” motif is meant, an enzymatic nucleic acid molecule comprising a motif as described in Ludwig et al., U.S. Ser. No. 60/156,236, filed Sep. 27, 1999, entitled “COMPOSITIONS HAVING RNA CLEAVING ACTIVITY”, incorporated by reference herein in its entirety including the drawings.

By “G-cleaver” motif is meant, an enzymatic nucleic acid molecule comprising a motif as described in Eckstein et al., U.S. Serial No unassigned, entitled “NUCLEIC ACID CATALYSTS WITH ENDONUCLEASE ACTIVITY,” which was filed on Nov. 19, 1999 and which is a continuation-in-part of U.S. Ser. No. 09/159,274, These applications are incorporated by reference in their entireties, including the drawings.

By “zinzyme” motif is meant, a class II enzymatic nucleic acid molecule !comprising a motif as described in Beigelman et al., U.S. Ser. No. 09/301,511 filed Apr. 28, 1999, entitled “NUCLEOTIDE TRIPHOSPHATES AND THEIR INCORPORATION INTO OLIGONUCLEOTIDES”, incorporated by reference herein in its entirety including the drawings.

By “amberzyme” motif is meant, a class I enzymatic nucleic acid molecule comprising a motif as described in Beigelman et al., U.S. Ser. No. 09/301,511 filed Apr. 28, 1999, entitled “NUCLEOTIDE TRIPHOSPHATES AND THEIR INCORPORATION INTO OLIGONUCLEOTIDES”, incorporated by reference herein in its entirety including the drawings.

By “DNAzyme” is meant, an enzymatic nucleic acid molecule lacking a 2′-OH group. In particular embodiments the enzymatic nucleic acid molecule may have an attached linker(s) or other attached or associated (groups, moieties, or chains containing one or more nucleotides with 2′-OH groups.

By “sufficient length” is meant an oligonucleotide of greater than or equal to 3 nucleotides. In connection with the binding arms of an enzymatic nucleic acid molecule, “sufficient length” means that the binding arms are long enough to provide a stable interaction with a target RNA under the expected conditions. Preferably the binding arms are not so long as to prevent a useful level of turnover.

By “stably interact” is meant, interaction of the oligonucleotides with target nucleic acid (e.g., by forming hydrogen bonds with complementary nucleotides in the target under physiological conditions).

By “equivalent” RNA to BACE is meant to include those naturally occurring RNA molecules having homology (partial or complete) to BACE proteins or encoding for proteins with similar function as BACE in various organisms, including human, rodent, primate, rabbit, pig, protozoans, fungi, plants, and other microorganisms and parasites. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical. Preferably, the sequences are at least 70%, 80%, 90%, or 95% identical over an analysis window of at least 50 or 100 contiguous nucleotides.

By “antisense nucleic acid” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review see Stein and Cheng, 1993 Science 261, 1004). Typically, antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “2-5A antisense chimera” it is meant. an antisense oligonucleotide containing a 5′ phosphorylated 2′-5′-linked adenylate residues. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300).

By “triplex DNA” it is meant an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504).

By “gene” it is meant a nucleic acid that encodes an RNA.

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule Keith its target or complementary sequence is sufficient to allow the relevant function of the nucleic acid to proceed, e.g., ribozyme cleavage, antisense or triple helix inhibition. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785.) A percent complementarity indicates the percentage of contiguous residues in a nucleic acid molecule which can form hydrogen bonds (e.g., Watson-Crick base pairing) with a second nucleic acid sequence (e.g., 5. 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

At least seven basic varieties of naturally-occurring enzymatic RNAs are known presently. Each can catalyze the hydrolysis of RNA phosphodiester bonds in trans (and thus can cleave other RNA molecules) under physiological conditions. Table I summarizes some of the characteristics of these ribozymes. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target. it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

The enzymatic nucleic acid molecules that cleave the specified sites in BACE-specific RNAs and/or ps-2-specific RNAs represent a novel therapeutic approach to treat a variety of pathologic indications, including Alzheimer's disease and dementia.

In one of the preferred embodiments of the inventions described herein, the enzymatic nucleic acid molecule is formed in a hammerhead or hairpin motif, but may also be formed in the motif of a hepatitis delta virus, group I intron, group II intron or RNase P RNA (in association with an RNA guide sequence), Neurospora VS RNA, DNAzymes, NCH cleaving motifs, or G-cleavers. Examples of such hammerhead motifs are described by Dreyfus, supra, Rossi et al., 1992, AIDS Research and Human Retroviruses 8, 183. Examples of hairpin motifs by Hampel et al., EP0360257; Hampel and Tritz, 1989 Biochemistry 28, 4929; Feldstein et al., 1989. Gene 82, 53; Haseloff and Gerlach, 1989, Gene, 82, 43; Hampel et al., 1990 Nucleic Acids Res. 18, 299; Chowrira & McSwiggen, U.S. Pat. No. 5,631,359. Examples of the hepatitis delta virus motif is described by Perrotta and Been, 1992 Biochemistry 31, 16. The RNase P motif is described by Guenrier-Takada et al., 1983 Cell 35, 849; Forster and Altman, 1990, Science 249, 783; Li and Altman, 1996, Nucleic Acids Res. 24, 835. Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J. 14, 363). Group II introns are described by Griffin et al., 1995, Chem. Biol. 2, 761; Michels and Pyle, 1995, Biochemistry 34, 2965; Pyle et al., International PCT Publication No. WO 96/22689. The Group I intron motif is described by Cech et al., U.S. Pat. No. 4,987,071 and of DNAzymes by Usman et al., International PCT Publication No. WO 95/11304; Chartrand et al. 1995, NAR 23, 4092; Breaker et al., 1995, Chem. Bio. 2, 655; Santoro et al., 1997, PNAS 94, 4262. NCH cleaving motifs are described in Ludwig & Sproat, International PCT Publication No. WO 98/58058, and G-cleavers are described in Kore et al., 1998, Nucleic Acids Research 26. 4116-4120 and Eckstein et al., International PCT Publication No. WO 99/16871. Additional motifs such as the Aptazyme (Breaker et al. WO 98/43993). Amberzyme (Class I motif, FIG. 3; Beigelman et al. U.S. Ser. No. 09/301,511) and Zinzyme (Beigelman et al., U.S. Ser. No. 09/301,511). All these references are incorporated by reference herein, including drawings. Any of these motifs can be used in the present invention. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

In preferred embodiments of the present invention, a nucleic acid molecule, e.g. an antisense molecule, a triplex DNA, or a ribozyme, is 13 to 100 nucleotides in length, e.g., in specific embodiments 35, 36, 37, or 38 nucleotides in length (e.g., for particular ribozymes or antisense). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 21 nucleotides in length.

In a preferred embodiment, the invention provides a method for producing a class of nucleic acid-based gene-inhibiting agents which exhibit a high degree of specificity for the RNA of a desired target. For example, the enzymatic nucleic acid molecule is preferably targeted to a highly conserved sequence region of target RNAs encoding BACE proteins (specifically BACE gene) such that specific treatment of a disease or condition can be provided with either one or several nucleic acid molecules of the invention. Such nucleic acid molecules can be delivered exogenously to specific tissue or cellular targets as required. Alternatively, the nucleic acid molecules (e.g., ribozymes and antisense) can be expressed from DNA and, or RNA vectors that are delivered to specific cells.

By “BACE proteins” is meant, a protein or a mutant protein derivative thereof, comprising β-secretase associated proteolytic cleavage activity of APP. In particular embodiments, the BACE protein can be referred to by other names used to describe a secretase, such as Asp2 (Gurney, 1999, Nature, 402 533-537).

By “highly conserved sequence region” is meant, a nucleotide sequence of one or more regions in a target gene does not vary significantly from one generation to the other or from one biological system to the other as understood by those skilled in the art.

The nucleic acid-based inhibitors of BACE expression are useful for the prevention of the diseases and conditions Alzheimer's disease, dementia, and any other diseases or conditions that are related to the levels of BACE in a cell or tissue.

By “related” is meant that the reduction of expression on activity from a particular gene, e.g., BACE expression (specifically BACE gene) RNA and/or ps-2 expression (specifically ps-2 gene) RNA levels and thus reduction in the level of the respective protein will relieve, to some extent, the symptoms of the disease or condition.

The nucleic acid-based inhibitors of the invention are added directly, or can be complexed with cationic lipids, packaged within liposomes, or otherwise delivered. to target cells or tissues. The nucleic acid or nucleic acid complexes can be locally administered to relevant tissues ex vivo, or in vivo through injection, infusion pump or stent, with or without their incorporation in biopolymers. In preferred embodiments, the enzymatic nucleic acid inhibitors comprise sequences, which are complementary to the substrate sequences in Tables III to VIII. Examples of such enzymatic nucleic acid molecules also are shown in Tables III to VIII. Examples of such enzymatic nucleic acid molecules consist essentially of sequences defined in these Tables.

In yet another embodiment, the invention features antisense nucleic acid molecules and 2-5A chimera including sequences complementary to the substrate sequences shown in Tables III to VIII. Such nucleic acid molecules can include sequences as shown for the binding arms of the enzymatic nucleic acid molecules in Tables III to VIII. Similarly, triplex molecules can be provided targeted to the corresponding DNA target regions, and containing the DNA equivalent of a target sequence or a sequence complementary to the specified target (substrate) sequence. Typically. antisense molecules will be complementary to a target sequence along a single contiguous sequence of the antisense molecule. However, in certain embodiments, an antisense molecule may bind to substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

By “consists essentially of” is meant that the active ribozyme contains an enzymatic center or core equivalent to those in the examples. and binding arms able to bind mRNA such that cleavage at the target site occurs. Other sequences may be present which do not interfere with such cleavage. Thus, a core region may, for example, include one or more loop or stem-loop structures, which do not prevent enzymatic activity. “X” in the sequences in Tables III and IV can be such a loop. A core sequence for a hammerhead ribozyme can be CUGAUGAG X CGOA where X=GCCGUUAGGC or other stem II region known in the art.

In another aspect of the invention, ribozymes or antisense molecules that cleave target RNA molecules or inhibit the Alzheimer's disease related genes identified above are expressed from transcription units inserted into DNA or RNA vectors. Preferably, ribozymes or antisense molecules that cleave BACE (preferably BACE gene) activity are expressed from transcription units inserted into DNA or RNA vectors. The recombinant vectors are preferably DNA plasmids or viral vectors. Ribozyme or antisense expressing viral vectors can be constructed based on, but not limited to, adeno-associated virus, retrovirus, adenovirus, or alphavirus. Preferably, the recombinant vectors capable of expressing the ribozymes or antisense are delivered as described above, and persist in target cells. Alternatively, viral vectors may be used that provide for transient expression of ribozymes or antisense. Such vectors can be repeatedly administered as necessary. Once expressed, the ribozymes or antisense bind to the target RNA and inhibit its function or expression. Delivery of ribozyme or antisense expressing vectors can be systemic, such as by intravenous or intramuscular administration, by administration to target cells ex-planted from the patient followed by reintroduction into the patient. or by any other means that would allow for introduction into the desired target cell.

By “vectors” is meant any nucleic acid- and/or viral-based technique used to deliver a desired nucleic acid.

By “patient” is meant an organism, which is a donor or recipient of explanted cells or the cells themselves. “Patient” also refers to an organism to which the nucleic acid molecules of the invention can be administered. Preferably, a patient is a mammal or mammalian cells. More preferably, a patient is a human or human cells.

The nucleic acid molecules of the instant invention, individually, or in combination or in conjunction with other drugs. can be used to treat diseases or conditions discussed above. For example, to treat a disease or condition associated with the levels of BACE, the patient may be treated, or other appropriate cells may be treated, as is evident to those skilled in the art, individually or in combination with one or more drugs under conditions suitable for the treatment.

In a further embodiment, the described molecules, such as antisense or ribozymes, can be used in combination with other known treatments to treat conditions or diseases discussed above. For example, the described molecules could be used in combination with one or more known therapeutic agents to treat Alzheimer's disease and dementia.

In another preferred embodiment, the invention features nucleic acid-based inhibitors (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of genes (e.g., BACE) capable of progression and/or maintenance of Alzheimer's disease.

In another preferred embodiment, the invention features nucleic acid-based techniques (e.g., enzymatic nucleic acid molecules (ribozymes), antisense nucleic acids, 2-5A antisense chimeras, triplex DNA, antisense nucleic acids containing RNA cleaving chemical groups) and methods for their use to down regulate or inhibit the expression of BACE gene expression.

By “comprising” is meant including, but not limited to, whatever follows the word “comprising”. Thus, use of the term “comprising” indicates that the listed elements are required or mandatory, but that other elements are optional and may or may not be present. By “consisting of” is meant including. and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

First the drawings will be described briefly.

### DRAWINGS

FIG. 1 shows the secondary structure model for seven different classes of enzymatic nucleic acid molecules. Arrow indicates the site of cleavage.—indicate the target sequence. Lines interspersed with dots are meant to indicate tertiary interactions.—is meant to indicate base-paired interaction. Group I Intron: P1-P9.0 represent various stem-loop structures (Cech et al., 1994, Nature Struc. Bio., 1, 273). RNase P (mRNA): EGS represents external guide sequence (Forster et al., 1990, Science, 249, 783; Pace et al., 1990, J. Biol. Chem., 265, 3587). Group II Intron: 5′SS means 5′ splice site; 3′SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme:: I-IV are meant to indicate four stem-loop structures (Been er al., U.S. Pat. No. 5,625,047). Hammerhead Ribozyme:: I-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (i.e., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is ≧1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q”≧is 2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

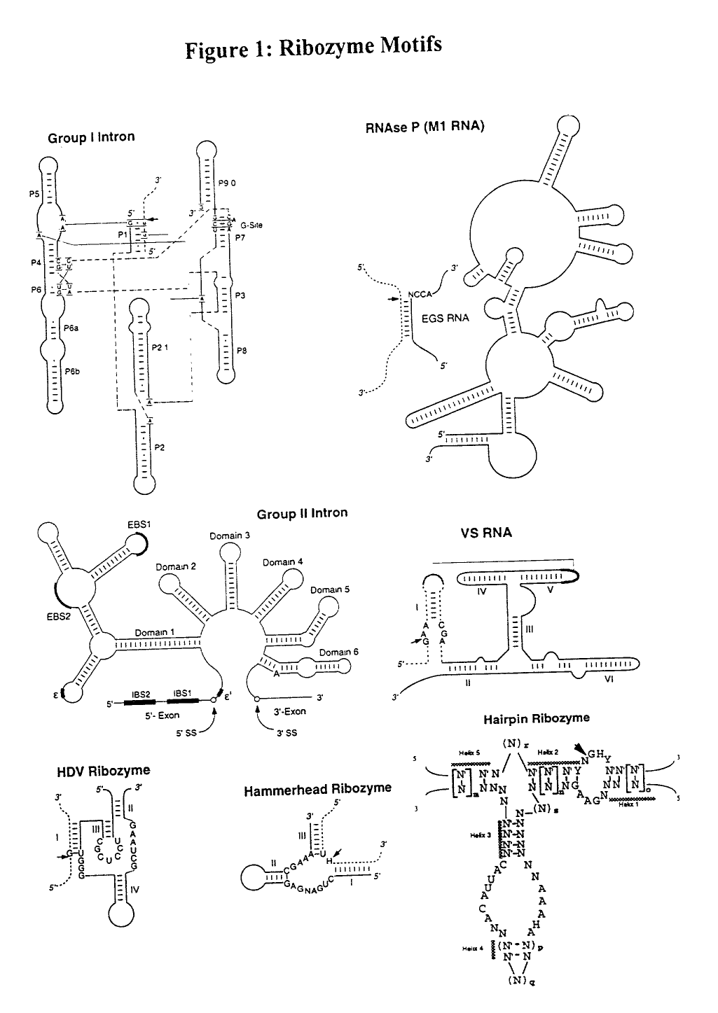

FIG. 2 shows examples of chemically stabilized ribozyme motifs. HH Rz, represents hammerhead ribozyme motif (Usman et al., 1996, Curr. Op. Struct. Bio., 1, 527); NCH Rz represents the NCH ribozyme motif (Ludwig & Sproat, International PCT Publication No. WO 98/58058); G-Cleaver, represents G-cleaver ribozyme motif (Kore et al., 1998, Nucleic Acids Research 26, 4116-4120). N or n, represent independently a nucleotide which may be same or different and have complementarity to each other; rI, represents ribo-Inosine nucleotide; arrow indicates the site of cleavage within the target. Position 4 of the HH Rz and the NCH Rz is shown as having 2′-C-allyl modification, but those skilled in the art will recognize that this position can be modified with other modifications well known in the art, so long as such modifications do not significantly inhibit the activity of the ribozyme.

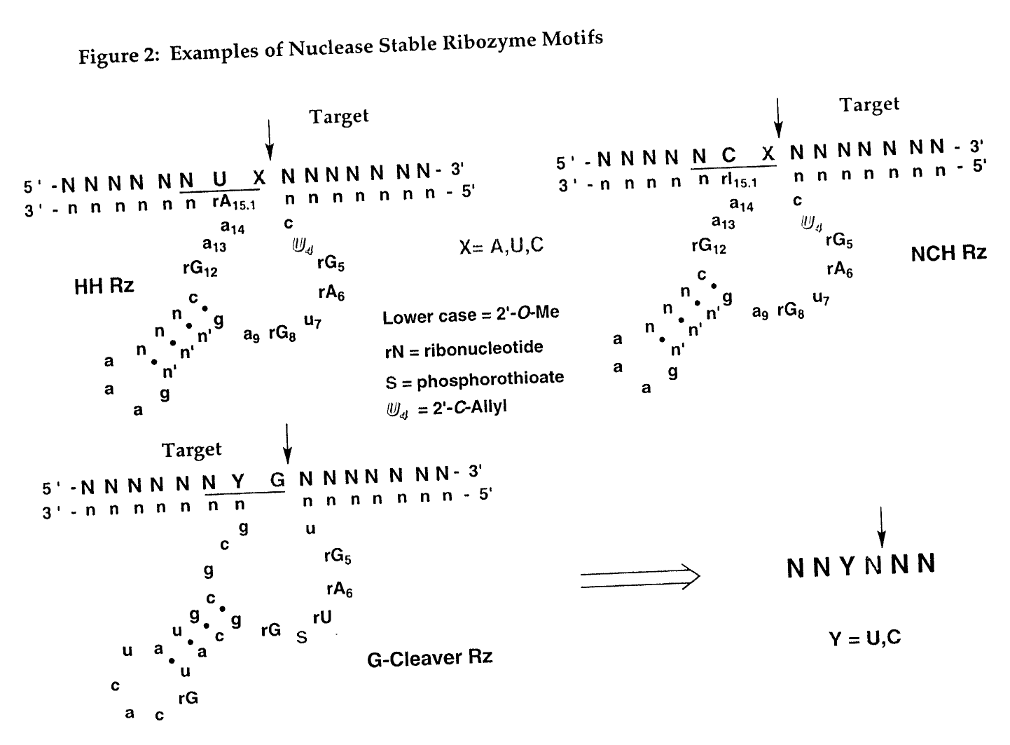

FIG. 3 shows an example of the Amberzyme ribozyme motif that is chemically stabilized (see for example Beigelman et al., U.S. Ser. No. 09/301,511, incorporated by reference herein; also referred to as Class I Motif).

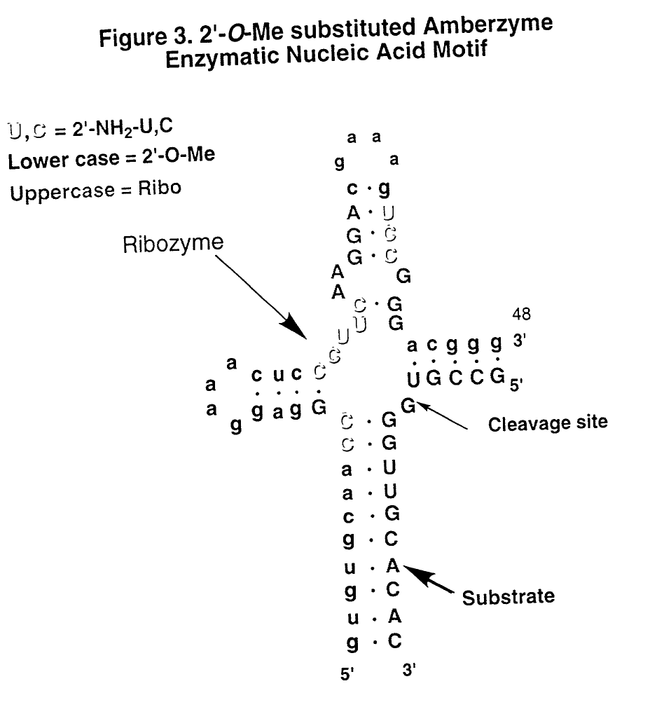

FIG. 4 shows an example of the Zinzyme A ribozyme motif that is chemically stabilized (see for example Beigelman et al., U.S. Ser. No. 09/301,511, incorporated by reference herein; also referred to as Class A or Class II Motif).

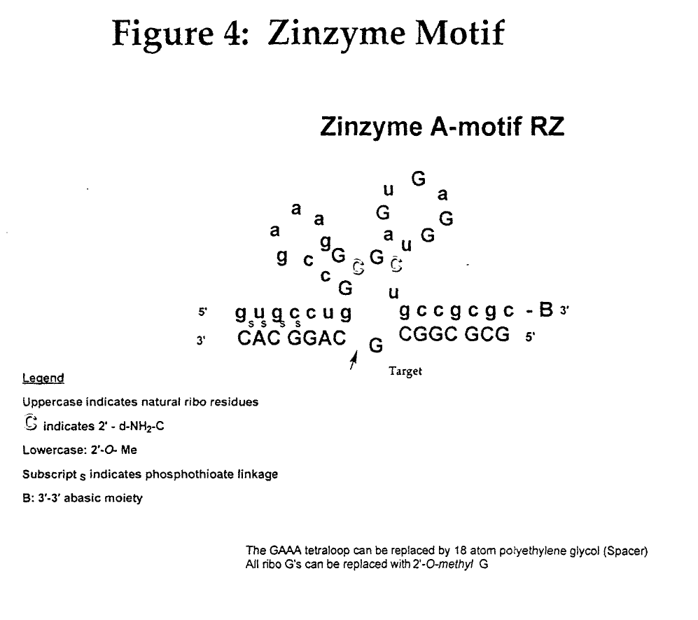

FIG. 5 shows an example of a DNAzyme motif described by Santoro et al., 1997, PNAS, 94, 4262.

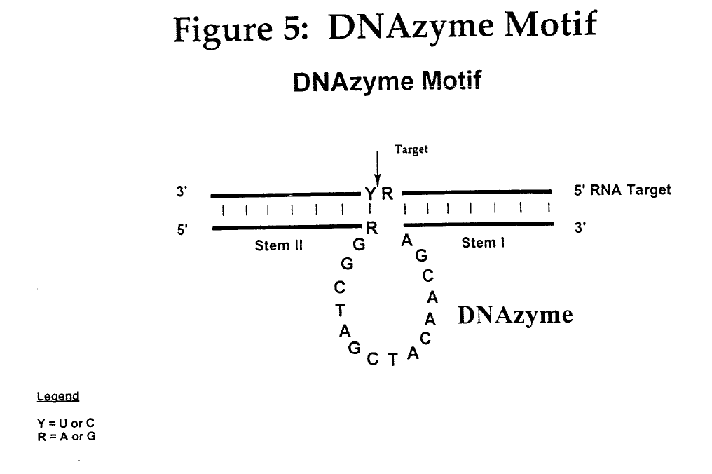

### MECHANISM OF ACTION OF NUCLEIC ACID MOLECULES OF THE INVENTION

Antisense: Antisense molecules may be modified or unmodified RNA. DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA may result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., U.S. Ser. No. 60/082,404 which was filed on Apr. 20, 1998; Hartmann et al., U.S. Ser. No. 60/101,174 which was filed on Sep. 21, 1998) all of these are incorporated by reference herein in their entirety.

Triplex Forming Oligonucleotides (TFO): Single stranded DNA may be designed to bind to genomic DNA in a sequence specific manner. TFOs are comprised of pyrimidine-rich oligonucleotides which bind DNA helices through Hoogsteen Base-pairing (Wu-Pong, supra). The resulting triple helix composed of the DNA sense, DNA antisense, and TFO disrupts RNA synthesis by RNA polymerase. The TFO mechanism may result in gene expression or cell death since binding may be irreversible (Mukhopadhyay & Roth. supra)

2-5A Antisense Chimera: The 2-.A system is an interferon-mediated mechanism for RNA degradation found in higher vertebrates (Mitra et al., 1996, Proc. Natl. Acad. Sci. U.S.A. 93, 6780-6785). Two types of enzymes. 2-5A synthetase and RNase L, are required for RNA cleavage. The 2-5A snthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures may be covalently linked to antisense molecules to form chimeric oligonucleotides capable of RNA cleavage (Torrence, supra). These molecules putatively bind and activate a 2-5A dependent RNase, the oligonucleotide/enzyme complex then binds to a target RNA molecule which can then be cleaved by the RNase enzyme.

Enzymatic Nucleic Acid: Seven basic varieties of naturally-occurring enzymatic RNAs are presently known. In addition. several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994. supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions.

Nucleic acid molecules of this invention will block to some extent BACE protein expression and can be used to treat disease or diagnose disease associated with the levels of BACE.

The enzymatic nature of a ribozyme has significant advantages, such as the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and achieve efficient cleavage in vitro (Zaug et al., 324, Nature 429 1986 Uhlenbeck, 1987 Nature 328, 596; Kim et al., 84 Proc. Natl. Acad. Sci. USA 8788, 1987; Dreyfus, 1988, Einstein Quart. J. Bio. Med. 6. 92; Haseloff and Gerlach, 334 Nature 585, 1988; Cech, 260 JAMA 3030, 1988; and Jefferies et al., 17 Nucleic Acids Research 1371, 1989; Santoro et al., 1997 supra).

Because of their sequence specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner synthesis of a protein associated with a disease state can be selectively inhibited (Warashina et al., 1999, Chemistry and Biology, 6, 237-250.

Target Sites

Targets for useful ribozymes and antisense nucleic acids can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468, all are hereby incorporated by reference herein in their totality. Other examples include the following PCT applications, which concern inactivation of expression of disease-related genes: WO 95/23225, WO 95/13380, WO 94/02595, all incorporated by reference herein. Rather than repeat the guidance provided in those documents here, specific examples of such methods are provided below, not limiting to those in the art. Ribozymes and antisense to such targets are designed as described in those applications and synthesized, to be tested in vitro and in vivo, as also described. The sequences of human BACE RNAs were screened for optimal enzymatic nucleic acid and antisense target sites using a computer-folding algorithm, Antisense, hammerhead, DNAzyme, NCH, amberzyme, zinzyme, or G-Cleaver ribozyme binding/cleavage sites were identified. These sites are shown in Tables III to VIII (all sequences are 5′ to 3 in the tables; X can be any base-paired sequence, the actual sequence is not relevant here). The nucleotide base position is noted in the Tables as that site to be cleaved by the designated type of enzymatic nucleic acid molecule. Thus, the position that is cleaved is following the substrate nucleotide that is written separated from the sequences on either side. For example, in Table III, for Seq. ID No. 1, nucleotide position 9 is the central “C”, and cleavage occurs at or following that nucleotide. While human sequences can be screened and enzymatic nucleic acid molecule and/or antisense thereafter designed, as discussed in Stinchcomb et al., WO 95/23225, mouse targeted ribozymes may be useful to test efficacy of action of the enzymatic nucleic acid molecule and/or antisense prior to testing in humans.

Antisense, hammerhead, DNAzyme, NCH, amberzyme, zinzyme or G-Cleaver ribozyme binding/cleavage sites were identified. The nucleic acid molecules were individually analyzed by computer folding (Jaeger et al., 1989 Proc. Natl. Acad. Sci. USA, 86, 7706) to assess whether the sequences fold into the appropriate secondary structure. Those nucleic acid molecules with unfavorable intramolecular interactions such as between the binding arms and the catalytic core were eliminated from consideration. Varying binding arm lengths can be chosen to optimize activity.

Antisense, hammerhead, DNAzyme, NCH, amberzyme, zinzyme or G-Cleaver ribozyme binding/cleavage sites were identified and were designed to anneal to various sites in the RNA target. The binding arms are complementary to the target site sequences described above. The nucleic acid molecules were chemically synthesized. The method of synthesis used follows the procedure for normal DNA/RNA synthesis as described below and in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684; Caruthers et al., 1992, Methods in Enzymology 211,3-19.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the hairpin ribozymes) are preferably used for exogenous delivery. The simple structure of these molecules increases the ability of the nucleic acid to invade targeted regions of RNA structure. Exemplary molecules of the instant invention were chemically synthesized, and others can similarly be synthesized. Oligodeoxyribonucleotides were synthesized using standard protocols as described in Caruthers et al., 1992, Methods in Enzymology 211. 3-19, and is incorporated herein by reference.

The method of synthesis used for normal RNA, including certain enzymatic nucleic acid molecules, follows the procedure as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses were conducted on a 394 Applied Biosystems, Inc. synthesizer using a 0.2 4 mol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M =6.6 4 mol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, were 97.5-99%. Other oligonucleotide synthesis reagents for the 394 Applied Biosystems. Inc. synthesizer; detritylation solution was 3% TCA in methylene chloride (ABI). capping was performed with 16% N-methyl imidazole in THF (ABI) and 10% acetic anhydride, 10% 2,6-lutidine in THF (ABI); oxidation solution was 16.9 mM I₂, 49 mM pyridine, 9% water in THF (PERSEPTIVE™). Burdick & Jackson Synthesis Grade acetonitrile was used directly from the reagent bottle. S-Ethyltetrazole solution (0.25 M in acetonitrile) was made up from the solid obtained from American International Chemical, Inc.

Deprotection of the RNA was performed using either a two-pot or one-pot protocol. For the two-pot protocol, the polymer-bound trityl-on oligoribonucleotide was transferred to a 4 mL glass screw top vial and suspended in a solution of 40% aq. methylamine (1 mL) at 65° C. for 10 min. After cooling to −20° C., the supernatant was removed from the polymer support. The support was washed three times with 1.0 mL of EtOH:MeCN:H20/3:1:1, vortexed and the supernatant was then added to the first supernatant. The combined supernatants, containing the oligoribonucleotide, were dried to a white powder. The base deprotected oligoribonucleotide was resuspended in anhydrous TEA/HF/NMP solution (300 μL of a solution of 1.5 mL N-methylpyrrolidinone, 750 μL TEA and 1 mL TEA3 HF to provide a 1.4 M HF concentration) and heated to 65° C. After 1.5 h, the oligomer was quenched with 1.5 M NH₄HCO₃.

Alternatively, for the one-pot protocol, the polymer-bound trityl-on oligoribonucleotide was transferred to a 4 mL glass screw top vial and suspended in a solution of 33% ethanolic methylamine/DMSO: 1/1 (0.8 mL) at 65° C. for 15 min. The vial was brought to r.t. TEA3 HF (0.1 mL) was added and the vial was heated at 65° C. for 15 min. The sample was cooled at −20° C. and then quenched with 1.5 M NH₄HCO₃.

For purification of the trityl-on oligomers, the quenched NH₄HCO; solution was loaded onto a C-18 containing cartridge that had been prewashed with acetonitrile followed by 50 mM TEAA. After washing the loaded cartridge with water, the RNA was detritylated with 0.5% TFA for 13 min. The cartridge was then washed again with water, salt exchanged with 1 M NaCI and washed with water again. The oligonucleotide was then eluted with 30% acetonitrile.

Inactive hammerhead ribozymes or binding attenuated control (BAC) oligonucleotides) were synthesized by substituting a U for G₅ and a U for A₁₄ (numbering from Hertel. K. J., et al., 1992, Nucleic Acids Res., 20, 3252). Similarly, one or more nucleotide substitutions can be introduced in other enzsmatic nucleic acid molecules to inactivate the molecule and such molecules can serve as a negative control.

The average stepwvise coupling yields were >98% (Wincott et al., 1995 Nucleic Acids Res. 23, 2677-2684). Those of ordinary skill in the art will recognize that the scale of synthesis can be adapted to be larger or smaller than the example described above, including but not limited to 96 well format. All that is important is the ratio of chemicals used in the reaction.

Alternatively, the nucleic acid molecules of the present invention can be synthesized separately and joined together post-synthetically, for example by ligation (Moore et al., 1992, Science 256, 9923; Draper et ill., International PCT publication No. WO 93/23569; Shabarova et al., 1991, Nucleic Acids Research 19, 4247; Bellon et al., 1997, Nucteosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Ribozymes are purified by gel electrophoresis using general methods or are purified by high pressure liquid chromatography (HPLC; See Wincott et al., szipra, the totality of which is hereby incorporated herein by reference) and are re-suspended in water.

The sequences of the ribozymes and antisense constructs that are chemically synthesized, useful in this study, are shown in Tables III to VIII. Those in the art will recognize that these sequences are representative only of many more such sequences where the enzymatic portion of the ribozyme (all but the binding arms) is altered to affect activity. The ribozyme and antisense construct sequences listed in Tables III to VIII may be formed of ribonucleotides or other nucleotides or non-nucleotides. Such ribozymes with enzymatic activity are equivalent to the ribozymes described specifically in the Tables.

Optimizing Activity of the Nucleic Acid Molecule of the Invention.

Chemically synthesizing nucleic acid molecules with modifications (base, sugar and/or phosphate) that prevent their degradatoio by serum ribonucleases may increase their potency (see e.g., Eckstein et al., International Publication No. WO 92/07065, Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). All these publications are hereby incorporated by reference herein. Modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuciease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Simp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci. , 1992, 17, 334-339; Usman et al. International Publication PCT No. WO 93/15187; Sproat, U.S. Pat. No. 5,334,711 and Beigelman et al., 1995. J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98113526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorodithioate, and/or 5′-methylphosphonate linkages improves stability. too many of these modifications may cause some toxicity. Therefore, when designing nucleic acid molecules, the amount of these internucleotide linkages should be minimized, but can be balanced to provide acceptable stability while reducing potential toxicity. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in a cell and/or in vivo the activity may not be significantly lowered. Therapeutic nucleic acid molecules delivered exogenously must optimally be stable within cells until translation of the target RNA has been inhibited long enough to reduce the levels of the undesirable protein. This period of time varies between hours to days depending upon the disease state. Clearly, exogenously delivered nucleic acid molecules should be resistant to nucleases in order to function as effective intracellular therapeutic agents. Improvements in the chemical synthesis of RNA and DNA (see, e.g., Wincott et al., 1995 Nucleic Acids Res. 23, 2677; Caruthers et al., 1992, Methods in Enzymology 21 1,3-19 (all incorporated by reference herein) have expanded the ability to modify nucleic acid molecules by introducing nucleotide modifications to enhance their nuclease stability as described above.

Use of the nucleic acid-based molecules of the invention will lead to better treatment of disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules may also include combinations of different types of nucleic acid molecules.

By “enhanced enzymatic activity” is meant to include activity measured in cells and/or in vivo where the activity is a reflection of both catalytic activity and ribozyme stability. In this invention, the product of these properties is increased or not significantly (less than 10-fold) decreased in vivo compared to an all RNA ribozyme or all DNA enzyme.

In yet another preferred embodiment, nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein. such ribozymes are useful in a cell and/or in vivo, even if activity over all is reduced 10-fold (Burgin et al., 1996, Biochemistry, 35, 14090). Such ribozymes herein are said to “maintain” the enzymatic activity on all RNA ribozyme.

In another aspect, the nucleic acid molecules comprise a 5′ and/or a 3′- cap structure.

By “cap structure” is meant chemical modifications, which have been incorporated at the terminus of the oligonucleotide (see for example Wincott et al., WO 97/26270. incorporated by reference herein). These terminal modifications protect the nucleic acid molecule from exonuclease degradation, and may help in delivery and/or localization within a cell. The cap may be present at the 5′-terminus (5′-cap) or at the 3′-terminus (3′-cap) or may be present on both ternini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Beigelman et al., International PCT publication No. WO 97/26270, incorporated by reference herein). In yet another preferred embodiment, the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; thtreo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, still more preferably 1 to 4 carbons. The alkyl group may be substituted or unsubstituted. When substituted the substituted group(s) is preferably. hydroxyl. cyano, alkoxy, ═O, ═S, NO₂ or N(CH₃)2, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, still more preferably 1 to 4 carbons. The alkenyl group may be substituted or unsubstituted. When substituted the substituted group(s) is preferably, hydroxyl, cyano, alkoxy, ═O, ═S, NO₂, halogen, N(CH₃)₂, amino, or SH. The termr “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group may be substituted or unsubstituted. When substituted the substituted group(s) is preferably, hydroxyl, cyano, alkoxy, ═O, ═S, NO₂ or N(CH₃)₂, amino or SH.

Such alkyl groups may also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which may be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen. sulfur and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety. (also referred to interchangeably as nucleotide analogs. modified nucleotides, non-natural nucleotides, non-standard nucleotides and other; see for example, Usman and McSwiggen, sitpra; Eckstein et al., International PCT Publication No. WO 92/07065; Usman et al., International PCT Publication No. WO 93 15187; Uhlman & Peyman, slipra; all are hereby incorporated by reference herein). There are several examples of modified nucleic acid bases known in the art. These have been recently summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene. 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne. and others (Burgin et al. 1996, Biochemnistry, 35, 14090; Uhlman & Peyman, supra). By “modified bases” in this aspect is meant nucleotide bases other than adenine, guanine, cytosine and uracil at 1′ position or their equivalents; such bases may be used at any position, for example, within the catalytic core of an enzymatic nucleic acid molecule and/or in the substrate-binding regions of the nucleic acid molecule.

By “abasic” is meant sugar moieties lacking a base or having other chemical groups in place of a base at the 1′ position.

By “ribonucleotide” is meant a nucleotide with a hydroxyl group at the 2′ position of a β-D-ribo-furanose moiety.

By “unmodified nucleoside” is meant one of the bases adenine. cytosine, guanine, uracil joined to the 1′ carbon of β-D-ribo-furanose and without substitutions on either moiety.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′—NH₂ or 2′-O—NH₂, which may be modified or unmodified.

Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and ribozyme) structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Use of these molecules will lead to better treatment of disease progression by affording the possibility of combination therapies (e.g., multiple ribozytmes targeted to different genes, ribozymes coupled with known small molecule inhibitors, or intermittent treatment with combinations of ribozymes (including different ribozyme motifs) and/or other chemical or biological molecules). The treatment of patients with nucleic acid molecules may also include combinations of different types of nucleic acid molecules. Therapies may be devised which include a mixture of ribozymes (including different ribozyme motifs), antisense and/or 2-5A chimera molecules to one or more targets to alleviate symptoms of a disease.

Administration of Nucleic Acid Molecules

Methods for the delivery of nucleic acid molecules are described in Akhtar et al. 1992, Trends Cell Bio., 2, 139; and Delivery, Strategies for Antisense Oligonticleotide Tlierapeutics, ed. Akhtar, 1995, which are both incorporated herein by reference. Sullivan et al., PCT WO 94102595, further describes the general methods for delivery of enzymatic RNA molecules. These protocols may be utilized for the delivery of virtually any nucleic acid molecule. Nucleic acid molecules may be administered to cells by a variety of methods known to those familiar to the art, including, but not restricted to, encapsulation in liposomes, by iontophoresis, or by incorporation into other vehicles, such as hydrogels. cyclodextrins, biodegradable nanocapsules, and bioadhesive microspheres. For some indications, nucleic acid molecules may be directly delivered ex vito to cells or tissues with or without the aforementioned vehicles. Alternatively, the nucleic acid/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of nucleic acid delivery and administration are provided in Sullivan et al., supra, Draper et al., PCT WO93/23569, Beigelman et al., PCT WO99/05094, and Klimuk et al., PCT WO99/04S19 all of which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a liposome delivery mechanism, standard protocols for formation of liposomes can be followed. The compositions of the present invention may also be formulated and used as tablets, capsules or elixirs for oral administration; suppositories for rectal administration; sterile solutions; suspensions for injectable administration; and the like.

The present invention also includes pharmnaceutically acceptable formulations of the compounds described. These formulations include salts of the above compounds, e.g., acid addition salts, for example, salts of hydrochloric. hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By pharmaceutically acceptable formulation is meant, a composition or formulation that allows for the effective distribution of the nucleic acid molecules of the instant invention in the physical location most suitable for their desired activity. Nonlimiting examples of agents suitable for formulation with the nucleic acid molecules of the instant invention include: P-glycoprotein inhibitors (such as Pluronic P85) which can enhance entry of drugs into the CNS (Jolliet-Riant and Tillement, 1999, Fundam. Clin. Pliarmacol., 13, 16-26); biodegradable polymers, such as poly (DL-lactide-coglycolide) microspheres for sustained release delivery after intracerebral implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include materials described in Boado et al., 1998. J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al. 1999, PNAS USA., 96, 7053-7058.

The invention also features the use compositions comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophiys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42. 24864-24870; Choi et al., International PCT Publication No. WO 96/10391; Ansell et al. International PCT Publication No. WO 96/10390; Holland et al., International PCT Publication No. WO 96/10392; all of these are incorporated by reference herein). Long-circulating liposomes are also likely to protect drugs from nuclease degradation to a greater extent compared to cationic liposomes, based on their ability to avoid accumulation in metabolically aggressive MPS tissues such as the liver and spleen. All of these references are incorporated by reference herein.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A.R.

Gennaro edit. 1985) hereby incorporated by reference herein. For example, preservatives, stabilizers, dyes and flavoring agents may be provided. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention may also be administered to a patient in combination with other therapeutic compounds to increase the overall therapeutic effect. The use of multiple compounds to treat an indication may increase the beneficial effects while reducing the presence of side effects.

Alternatively, certain of the nucleic acid molecules of the instant invention can be expressed within cells from eukaryoi:, promoters (e.g., Izant and Weintraub, 1985, Science, 229, 345; McGarry and Lindquist, 1986, Proc. Natl. Acad. Sci., USA 83, 399; Scanlon et al., 1991, Proc. Natl. Acad. Sci. USA, 88, 10591-5; Kashani-Sabet et al., 1992, Antisense Res. Dev., 2, 3-15; Dropulic et al., 1992, J. Virol., 66, 1432-41; Weerasinghe et al., 1991, J. Virol., 65. 5531-4; Ojwang et al., 1992, Proc. Natl. Acad. Sci. USA, 89, 10802-6; Chen et al., 1992, Nucleic Acids Res., 20, 4581-9; Sarver et al., 1990 Science, 247, 1222-1225; Tnompson et al., 1995, Nucleic Acids Res., 23, 2259; Good et al., 1997, Gene Therapy, 4. 45; all which are hereby incorporated by reference herein in their totalities). Those skilled in the art realize that any nucleic acid can be expressed in eukaryotic cells from the appropriate DNA/RNA vector. The activity of such nucleic acids can be augmented by their release from the primary transcript by a ribozyme (Draper et ai PCT WO 93/23569, and Sullivan et al., PCT WO 94/02595; Ohkawa et al., 1992, Nucleic Acids Symp. Ser., 27, 15-6; Taira et al., 1991, Nucleic Acids Res., 19, 5125-31-:. Ventura et al., 1993, Nucleic Acids Res., 21, 3249-55; Chowrira et al., 1994, J. Biol. Chem., 269, 25856; all which are hereby incorporated by reference herein in their totalities).

In another aspect of the invention, RNA molecules of the present invention are preferably expressed from transcription units (see, for example, Couture et al., 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors may be used that provide for transient expression of nucleic acid molecules. Such vectors might be repeatedly administered as necessary. Once expressed, the nucleic acid molecule binds to the target mRNA. Delivery of nucleic acid molecule expressing vectors could be systemic, such as by intravenous or intra-muscular administration, by administration to target cells ex-planted from the patient followed by reintroduction into the paient. or by any other means that would allow for introduction into the desired target cell (for a review see Couture et al., 1996, TIG., 12, 510).

In one aspect the invention features an expression vector comprising nucleic acid sequence encoding at least one of the nucleic acid molecules of the instant invention. The nucleic acid sequence encoding the nucleic acid molecule of the instant invention is operable linked in a manner which allows expression of that nucleic acid molecule.

In another aspect the invention features. an expression vector comprising: a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector may optionally include an open reading frame (ORF) for a protein operably linked on the 5′ side or the 3′-side of the gene encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pot HII promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. USA, 87, 6743-7; Gao and Huang 1993, Nucleic Acids Res., 21, 2867-72; Lieber et al., 1993, Methods Enzymol., 217, 47-66; Zhou et al., 1990, Mol. Cell. Biol., 10, 4529-37). Several investigators have demonstrated that nucleic acid molecules, such as ribozymes expressed from such promoters can function in mammalian cells (e.g., Kashani-Sabet et al., 1992, Antisense Res. Dev., 2, 3-15; Ojwang et al., 1992, Proc. Natl. Acad. Sci. USA, 89, 10802-6; Chen et al., 1992, Nucleic Acids Res., 20, 4581-9; Yu et al., 1993, Proc. Natl. Acad. Sci. U S A, 90, 6340-4; L'Huillier et al., 1992, EMBO J., 11, 4411-8; Lisziewicz et al., 1993, Proc. Natl. Acad. Sci. U.S.A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 5 45; Beigelman et al., International PCT Publication No. WVO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect, the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another preferred embodiment, the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment, the expression vector comprises: a) a transcription initiation region; b) a transcription termination region; c) an intron; d) a gene encoding at least one said nucleic acid molecule; and wherein said gene is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

### EXAMPLES

The following are non-limiting examples showing the selection, isolation, synthesis and activity of nucleic acids of the instant invention.

The following examples demonstrate the selection and design of antisense, hammerhead, DNAzyme, NCH, or G-Cleaver ribozvvme molecules and binding/cleavage sites within BACE RNA.

### Example 1

### Identification of Potential Target Sites in Human BACE RNA

The sequence of human BACE was screened for accessible sites using a computer-folding algorithm. Regions of the RNA that did not form secondary folding structures and contained potential ribozyme and/or antisense binding/cleavage sites were identified. The sequences of these cleavage sites are shown in Tables III-VIII.

### Example 2

### Selection of Enzymatic Nucleic Acid Cleavage Sites in Human BACE RNA

Ribozyme target sites were chosen by analyzing sequences of Human BACE (Genbank sequence accession number: AF190725) and prioritizing the sites on the basis of folding. Ribozymes were designed that could bind each target and were individually analyzed by computer folding (Christoffersen et al., 1994 J. Mol. Struc. Tl7eochem, 311, 273; Jaeger et al., 1989, Proc. Nactl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

### Example 3

### Chemical Synthesis and Purification of Ribozymes and Antisense for Efficient Cleavage and/or blocking of BACE RNA

Ribozymes and antisense constructs were designed to anneal to various sites in the RNA message. The binding arms of the ribozymes are complementary to the target site sequences described above, while the antisense constructs are fully complimentary to the target site sequences described above. The ribozymes and antisense constructs w%ere chemically synthesized. The method of synthesis used followed the procedure for normal RNA synthesis as described above and in Usman et al., (1987J. Am. Chem. Soc., 109, 7845), Scaringe et al., (1990 Nucleic. Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were >98%.

Ribozymes and antisense constructs were also synthesized from DNA templates using bacteriophage T7 RNA polymerase (Milligan and Uhlenbeck, 1989, Methods Enzymol. 180, 51). Ribozymes and antisense constructs were purified by gel electrophoresis using general methods or were purified by high pressure liquid chromatography (HPLC; See Wincott et al., supra; the totality of which is hereby incorporated herein by reference) and were resuspended in water. The sequences of the chemically synthesized ribozymes and antisense constructs used in this study are shown below in Table III-VIII.

### Example 4

### Ribozyme Cleavage of BACE RNA Target in vitro

Ribozymes targeted to the human BACE RNA are designed and synthesized as described above. These ribozymes can be tested for cleavage activity in vitro, for example, using the following procedure. The target sequences and the nucleotide location within the BACE RNA are given in Tables III-VIII.

Cleavage Reactions:

Full-length or partially full-length, internally-labeled target RNA for ribozyme cleavage assay is prepared by in vitro transcription in the presence of [a-³²p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2× concentration of purified ribozyme in ribozyme cleavage buffer (50 mM Tris-HCl, pH 7.5 at 37° C., 10 mM MgCl₂) and the cleavage reaction was initiated by adding the 2× ribozyme mix to an equal volume of substrate RNA (maximum of 1-5 nM) that was also pre-warmed in cleavage buffer. As an initial screen, assays are carried out for 1 hour at 37° C. using a final concentration of either 40 nNM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imagerg quantitation of bands representing the intact substrate and the cleavage products.

Cell Culture Models

Vassar et al., 1999, Science, 286, 735-741, describe a cell culture model for studying BACE inhibition. Specific antisense nucleic acid molecules targeting BACE mONA were used for inhibition studies of endogenous BACE expression in 101 cells and APPsw (Swedish type amyloid precursor protein expressing) cells via lipid mediated transfection. Antisense treatment resulted in dramatic reduction of both BACE mRNA by Northern blot analysis, and APPspsw (“Swedish” type β-secretase cleavage product) by ELISA, with maximum inhibition of both parameters at 75-80%. This model wvas also used to study the effect of BACE inhibition on amyloid P-peptide production in APPsw cells.

Animal Models

Games et al., 1995, Nature, 373, 523-527, describe a transgenic mouse model in which mutant human familial type APP (Phe 717 instead of Val) is overexpressed. This model results in mice that progressively develop many of the pathological hallmarks of Alzheimer's disease, and as such, provides a model for testing therapeutic drugs.

Indications

Particular degenerative and disease states that can be associated with BACE expression modulation include but are not limited to Alzheimer's disease and dementia.

The present body of knowledge in BACE research indicates the need for methods to assay BACE activity and for compounds that can regulate BACE expression for research, diagnostic, and therapeutic use.

Donepezil, tacrine, selegeline, and acetyl-L-carnitine are non-limiting examples of pharmaceutical agents that can be combined with or used in conjunction with the nucleic acid molecules (e.g. ribozymes and antisense molecules) of the instant invention. Those skilled in the art will recognize that other drugs such as diuretic and antihypertensive compounds and therapies can be similarly be readily combined with the nucleic acid molecules of the instant invention (e.g. ribozymes and antisense molecules) are hence within the scope of the instant invention.

Diagnostic Uses

The nucleic acid molecules of this invention (e.g., ribozymes) may be used as diagnostic tools to examine genetic drift and mutations within diseased cells or to detect the presence of BACE RNA in a cell. The close relationship between ribozyme activity and the structure of the target RNA allows the detection of mutations in any region of the molecule which alters the base-pairing and three-dimensional structure of the target RNA. By using multiple ribozymes described in this invention, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These experiments will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with BACE-related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA will be cleaved by both ribozymes to demonstrate the relative ribozyme efficiencies in the reactions and the absence of cleavage of the “non-targeted” RNA species. The cleavage products from the synthetic substrates will also serve to generate size markers for the analysis of wild-type and mutant RNAs in the sample population. Thus, each analysis will involve two ribozymes, two substrates and one unknown sample, which will be combined into six reactions. The presence of cleavage products will be determined using an RNAse protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (e.g., BACE) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate cleavage products with
3′-OH and 5′-guanosine.
Additional protein cofactors required in some cases to help folding and maintainance of the
active structure.
Over 300 known members of this class. Found as an intervening sequence in Tetrahymena
thermophila rRNA, fungal mitochondria, chloroplasts, phage T4, blue-green algae, and
others.
Major structural features largely established through phylogenetic comparisons,
mutagenesis, and biochemical studies [I, II].
Complete kinetic framework established for one ribozyme [III, IV, V, VI].
Studies of ribozyme folding and substrate docking underway [VII, VIII, IX].
Chemical modification investigation of important residues well established [X, XI].
The small (4-6 nt) binding site may make this ribozyme too non-specific for targeted RNA
cleavage, however, the Tetrahymena group I intron has been used to repair a “defective”-
galactosidase message by the ligation of new-galactosidase sequences onto the defective
message [XII].
RNAse P RNA (M1 RNA)
Size: ˜290 to 400 nucleotides.
RNA portion of a ubiquitous ribonucleoprotein enzyme.
Cleaves tRNA precursors to form mature tRNA [XIII].
Reaction mechanism: possible attack by M²⁺-OH to generate cleavage products with 3′-
OH and 5′-phosphate.
RNAse P is found throughout the prokaryotes and eukaryotes. The RNA subunit has been
sequenced from bacteria, yeast, rodents, and primates.
Recruitment of endogenous RNAse P for therapeutic applications is possible through
hybridization of an External Guide Sequence (EGS) to the target RNA [XIV, XV]
Important phosphate and 2′ OH contacts recently identified [XVI, XVII]
Group II Introns
Size: >1000 nucleotides.
Trans cleavage of target RNAs recently demonstrated [XVIII, XIX].
Sequence requirements not fully determined.
Reaction mechanism: 2′-OH of an internal adenosine generates cleavage products with 3′-
OH and a “lariat” RNA containing a 3′-5′ and a 2′-5′ branch point.
Only natural ribozyme with demonstrated participation in DNA cleavage [XX, XXI] in addition
to RNA cleavage and ligation.
Major structural features largely established through phylogenetic comparisons [XXII].
Important 2′ OH contacts beginning to be identified [XXIII]
Kinetic framework under development [XXIV]
Neurospora VS RNA
Size: ˜144 nucleotides.
Trans cleavage of hairpin target RNAs recently demonstrated [XXV].
Sequence requirements not fully determined.
Reaction mechanism: attack by 2′-OH 5′ to the scissile bond to generate cleavage products
with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate cleavage products
with 2′,3′-cyclic phosphate and 5′-OH ends.
14 known members of this class. Found in a number of plant pathogens (virusoids) that
use RNA as the infectious agent.
Essential structural features largely defined, including 2 crystal structures [XXVI, XXVII]
Minimal ligation activity demonstrated (for engineering through in vitro selection) [XXVII]
Complete kinetic framework established for two or more ribozymes [XXIX].
Chemical modification investigation of important residues well established [XXX].
Hairpin Ribozyme
Size: ˜50 nucleotides.
Requires the target sequence GUC immediately 3′ of the cleavage site.
Binds 4-6 nucleotides at the 5′-side of the cleavage site and a variable number to the 3′-side
of the cleavage site.
Reaction mechanism: attack by 2′-OH 5′ to the scissile bond to generate cleavage products
with 2′,3′-cyclic phosphate and 5′-OH ends.
3 known members of this class. Found in three plant pathogen (satellite RNAs of the
tobacco ringspot virus, arabis mosaic virus and chicory yellow mottle virus) which uses
RNA as the infectious agent.
Essential structural features largely defined [XXXI, XXXII, XXXIII, XXXIV]
Ligation activity (in addition to cleavage activity) makes ribozyme amenable to
engineering through in vitro selection [XXXV]
Complete kinetic framework established for one ribozyme [XXXVI].
Chemical modification investigation of important residues begun [XXXVII, XXXVIII].
Hepatitis Delta Virus (HDV) Ribozyme
Size: ˜60 nucleotides.
Trans cleavage of target RNAs demonstrated [XXXIX].
Binding sites and structural requirements not fully determined, although no sequences 5′ of
cleavage site are required. Folded ribozyme contains a pseudoknot structure [XL].
Reaction mechanism: attack by 2′-OH 5′ to the scissile bond to generate cleavage products
with 2′,3′-cyclic phosphate and 5′-OH ends.
Only 2 known members of this class. Found in human HDV.
Circular form of HDV is active and shows increased nuclease stability [XLI]
I MICHEL. FRANCOIS, WESTHOF, ERIC SLIPPERY SUBSTRATES NAT STRUCT BIOL (1994), 1(1), 5-7
II LISACEK. FREDERIQUE, DIAZ, YOLANDE; MICHEL, FRANCOIS AUTOMATIC DENTIFICATION OF GROUP 1 INTRON CORES IN GENOMIC DNA SEQUENCES J MOL BIOL. (1994), 235(4), 1206-17
III HERSCHLAG, DANIEL, CECH, THOMAS R CATALYSIS OF RNA CLEAVAGE BY THE TETRAHYMENA THERMOPHILA RIBOZYME. 1 KINETIC DESCRIPTION OF THE REACTION OF AN RNA SUBSTRATE COMPLEMENTARY TO THE ACTIVE SITE BIOCHEMISTRY (1990), 29(44), 10159-71
IV HERSCHLAG, DANIEL, CECH, THOMAS R CATALYSIS OF RNA CLEAVAGE BY THE TETRAHYMENA THERMOPHILA RIBOZYME 2 KINETIC DESCRIPTION OF THE REACTION OF AN RNA SUBSTRATE THAT FORMS A MISMATCH AT THE ACTIVE SITE. BIOCHEMISTRY (1990), 29(44), 10172-80
V KNITT, DEBORAH S, HERSCHLAG, DANIEL PH DEPENDENCIES OF THE TETRAHYMENA RIBOZYME REVEAL AN UNCONVENTIONAL ORIGIN OF AN APPARENT PKA BIOCHEMISTRY (1996), 35(5) 1560-70
VI BEVILACQUA, PHILIP C., SUGIMOTO, NAOKI, TURNER, DOUGLAS H A MECHANISTIC FRAMEWORK FOR THE SECOND STEP OF SPLICING CATALYZED BY THE TETRAHYMENA RIBOZYME BIOCHEMISTRY (1996), 35(2), 648-58
VII LI, YI, BEVILACQUA, PHILIP C , MATHEWS, DAVID, TURNER, DOUGLAS H THERMODYNAMIC AND ACTIVATION PARAMETERS FOR BINDING OF A PYRENE-LABELED SUBSTRATE BY THE TETRAHYMENA RIBOZYME: DOCKING IS NOT DIFFUSION-CONTROLLED AND IS DRIVEN BY A FAVORABLE ENTROPY CHANGE BIOCHEMISTRY (1995), 34(44), 14394-9
VIII BANERJEE, ALOKE RAJ; TURNER, DOUGLAS H THE TIME DEPENDENCE OF CHEMICAL MODIFICATION REVEALS SLOW STEPS IN THE FOLDING OF A GROUP 1 RIBOZYME BIOCHEMISTRY (1995), 34(19), 6504-12
IX ZARRINKAR, PATRICK P.; WILLIAMSON, JAMES R THE P9 1-P9 2 PERIPHERAL EXTENSION HELPS GUIDE FOLDING OF THE TETRAHYMENA RIBOZYME NUCLEIC ACIDS RES (1996), 24(5 854-8
X STROBEL, SCOTT A , CECH, THOMAS R MINOR GROOVE RECOGNITION OF THE CONSERVED G CNTDOT U PAIR AT THE TETRAHYMENA RIBOZYME REACTION SITE SCIENCE (WASHINGTON, DC) (1995), 267(5198), 675-9.
XI STROBEL, SCOTT A , CECH, THOMAS R., EXOCYCLIC AMINE OF THE CONSERVED G CNTDOT U PAIR AT THE CLEAVAGE SITE OF THE TETRAHYMENA RIBOZYME CONTRIBUTES TO 5′-SPLICE SITE SELECTION AND TRANSITION STATE STABILIZATION BIOCHEMISTRY (1996), 35(4), 1201-11
XII SULLENGER, BRUCE A.; CECH, THOMAS R RIBOZYME-MEDIATED REPA R OF DEFECTIVE MRNA BY TARGETED TRANS-SPLICING NATURE (LONDON) (1994), 371(6498), 619-22
XIII ROBERTSON. H D , ALTMAN, S., SMITH, J D J BIOL CHEM , 247, 5243-5251 (1972)
XIV FORSTER, ANTHONY C , ALTMAN, SIDNEY. EXTERNAL GUIDE SEQUENCES FOR AN RNA ENZYME SCIENCE (WASHINGTON. DC., 1883-) (1990), 249(4970), 783-6
XV YUAN, Y , HWANG, E S.; ALTMAN, S TARGETED CLEAVAGE OF MRNA BY HUMAN RNASE P PROC NATL ACAD SCI. USA (1992) 89, 8006-10
XVI HARRIS, MICHAEL E , PACE, NORMAN R IDENTIFICATION OF PHOSPHATES INVOLVED IN CATALYSIS BY THE RIBOZYME RNASE P RNA RNA (1995), 1(2), 210-18
XVII PAN, TAO, LORIA, ANDREW, ZHONG, KUN PROBING OF TERTIARY INTERACTIONS IN RNA 2′-HYDROXYL-BASE CONTACTS BETWEEN THE RNASE P RNA AND PRE-TRNA PROC NATL ACAD. SC USA (1995), 92(26), 12510-14
XVIII PYLE. ANNA MARIE, GREEN JUSTIN B BUILDING A KINETIC FRAMEWORK FOR GROUP II INTRON RIBOZYME ACTIVITY QUANTITATION OF INTERDOMAIN BINDING AND REACTION RATE BIOCHEMISTRY (1994), 33(9), 2716-25
XIX MICHELS, WILLIAM J JR , PYLE. ANNA MARIE CONVERSION OF A GROUP II INTRON INTO A NEW MULTIPLE-TURNOVER RIBOZYME THAT SELECTIVELY CLEAVES OLIGONUCLEOTIDES ELUCIDATION OF REACTION MECHANISM AND STRUCTURE/FUNCTION RELATIONSHIPS BIOCHEMISTRY (1995), 34(9), 2965-77
XX ZIMMERLY, STEVEN, GUO, HUATAO ESKES, ROBERT, YANG, JIAN, PERLMAN, PHILIP S . LAMBOWITZ, ALAN M., A GROUP II INTRON RNA IS A CATALYTIC COMPONENT OF A DNA ENDONUCLEASE INVOLVED IN INTRON MOBILITY CELL (CAMBRIDGE, MASS) (1995), 83(4), 529-38
XXI GRIFFIN, EDMUND A , JR . QIN, ZHIFENG, MICHELS, WILLIAMS J , JR , PYLE, ANNA MARIE GROUP II INTRON RIBOZYMES THAT CLEAVE DNA AND RNA LINKAGES WITH SIMILAR EFFICIENCY, AND LACK CONTACTS WITH SUBSTRATE 2′-HYDROXYL GROUPS CHEM BIOL (1995), 2(11), 761-70
XXII MICHEL, FRANCOIS, FERAT, JEAN LUC, STRUCTURE AND ACTIVITIES OF GROUP II INTRONS ANNU REV BIOCHEM (1995), 64, 435-61
XXIII ABRAMOVITZ, DANA L , FRIEDMAN, RICHARD A , PYLE, ANNA MARIE CATALYTIC ROLE OF 2′-HYDROXYL GROUPS WITHIN A GROUP II INTRON ACTIVE SITE SCIENCE (WASHINGTON, DC) (1996), 271(5254), 1410-13
XXIV DANIELS, DANETTE L , MICHELS, WILLIAM J . JR , PYLE, ANNA MARIE. TWO COMPETING PATHWAYS FOR SELF-SPLICING BY GROUP II INTRONS A QUANTITATIVE ANALYSIS OF IN VITRO REACTION RATES AND PRODUCTS J MOL. BIOL (1996), 256(1), 31-49
XXV GUO, HANS C T , COLLINS. RICHARD A EFFICIENT TRANS-CLEAVAGE OF A STEM-LOOP RNA SUBSTRATE BY A RIBOZYME DERIVED FROM NEUROSPORA VS RNA EMBO J (1995), 14(2), 368-76
XXVI SCOTT, W G , FINCH, J T . AARON. K THE CRYSTAL STRUCTURE OF AN ALL RNA HAMMERHEAD RIBOZYME.APROPOSED MECHANISM FOR RNA CATALYTIC CLEAVAGE CELL, (1995), 81, 991-1002
XXVII MCKAY, STRUCTURE AND FUNCTION OF THE HAMMERHEAD RIBOZYME AN UNFINISHED STORY RNA, (1996), 2, 395-403
XXVIII LONG, D , UHLENBECK, O , HERTEL, K LIGATION WITH HAMMERHEAD RIBOZYMES U.S. PAT. NO. 5,633,133
XXIX HERTEL, K J , HERSCHLAG, D , UHLENBECK, O A KINETIC AND THERMODYNAMIC FRAMEWORK FOR THE HAMMERHEAD RIBOZYME REACTION BIOCHEMISTRY, (1994) 33, 3374-3385 BEIGELMAN, L ET AL , CHEMICAL MODIFICATIONS OF HAMMERHEAD RIBOZYMES J BIOL CHEM , (1995) 270, 25702-25708
XXX BEIGELMAN, L., ET AL., CHEMICAL MODIFICATIONS OF HAMMERHEAD RIBOZYMES J BIOL CHEM , (1995) 270, 25702-25708
XXXI HAMPEL, ARNOLD, TRITZ, RICHARD, HICKS, MARGARET, CRUZ, PHILLIP ‘HAIRPIN’ CATALYTIC RNA MODEL EVIDENCE FOR HELIXES AND SEQUENCE REQUIREMENT FOR SUBSTRATE RNA NUCLEIC ACIDS RES (1990), 18(2), 299-304
XXXII CHOWRIRA, BHARAT M., BERZAL-HERRANZ, ALFREDO, BURKE, JOHN M NOVEL GUANOSINE REQUIREMENT FOR CATALYSIS BY THE HAIRPIN RIBOZYME NATURE (LONDON) (1991), 354(6351), 320-2
XXXIII BERZAL-HERRANZ, ALFREDO, JOSEPH, SIMPSON, CHOWRIRA, BHARAT M., BUTCHER, SAMUEL E . BURKE, JOHN M ESSENTIAL NUCLEOTIDE SEQUENCES AND SECONDARY STRUCTURE ELEMENTS OF THE HAIRPIN RIBOZYME EMBO J. (1993), 12(6), 2567-73
XXXIV JOSEPH, SIMPSON, BERZAL-HERRANZ, ALFREDO, CHOWRIRA, BHARAT M , BUTCHER. SAMUEL E SUBSTRATE SELECTION RULES FOR THE HAIRPIN RIBOZYME DETERMINED BY IN VITRO SELECTION, MUTATION, AND ANALYSIS OF MISMATCHED SUBSTRATES. GENES DEV (1993), 7(1), 130-8.
XXXV BERZAL-HERRANZ, ALFREDO. JOSEPH, SIMPSON, BURKE, JOHN M (N VITRO SELECTION OF ACTIVE HAIRPIN RIBOZYMES BY SEQUENTIAL RNA-CATALYZED CLEAVAGE AND LIGATION REACTIONS. GENES DEV (1992), 6(1), 129-34.
XXXVI HEGG, LISA A , FEDOR, MARTHA J KINETICS AND THERMODYNAMICS OF INTERMOLECULAR CATALYSIS BY HAIRPIN RIBOZYMES. BIOCHEMISTRY (1995), 34(48), 15813-28
XXXVII GRASBY, JANE A , MERSMANN, KARIN; SINGH, MOHINDER, GAIT, MICHAEL J., PURINE FUNCTIONAL GROUPS IN ESSENTIAL RESIDUES OF THE HAIRPIN RIBOZYME REQUIRED FOR CATALYTIC CLEAVAGE OF RNA BIOCHEMISTRY (1995), 34(12), 4068-76.
XXXVIII SCHMIDT, SABINE, BEIGELMAN, LEONID; KARPEISKY, ALEXANDER, USMAN, NASSIM; SORENSEN, ULRIK S , GAIT, MICHAEL J BASE AND SUGAR REQUIREMENTS FOR RNA CLEAVAGE OF ESSENTIAL NUCLEOSIDE RESIDUES IN INTERNAL LOOP B OF THE HAIRPIN RIBOZYME IMPLICATIONS FOR SECONDARY STRUCTURE NUCLEIC ACIDS RES (1996), 24(4), 573-81
XXXIX PERROTTA, ANNE T , BEEN, MICHAEL D . CLEAVAGE OF OLIGORIBONUCLEOTIDES BY A RIBOZYME DERIVED FROM THE HEPATITIS DELTA VIRUS RNA SEQUENCE BIOCHEMISTRY (1992), 31(1), 16-21
XL PERROTTA, ANNE T , BEEN, MICHAEL D A PSEUDOKNOT-LIKE STRUCTURE REQUIRED FOR EFFICIENT SELF-CLEAVAGE OF HEPATITIS DELTA VIRUS RNA NATURE (LONDON) (1991), 350(6317), 434-6
XLI PUTTARAJU, M , PERROTTA, ANNE T , BEEN, MICHAEL D A CIRCULAR TRANS-ACTING HEPATITIS DELTA VIRUS RIBOZYME NUCLEIC ACIDS RES. (1993), 21(18), 4253-8

TABLE II
A. 2.5 μmol Synthesis Cycle ABI 394 Instrument

| | | | | Wait |
|---|---|---|---|---|
| | | | Wait Time* | Time* |
| Reagent | Equivalents | Amount | 2′-O-methyl | RNA |
| Phosphoramidites | 6.5 | 163 μL | 2.5 min | 7.5 |
| S-Ethyl Tetrazole | 23.8 | 238 μL | 2.5 min | 7.5 |
| Acetic Anhydride | 100 | 233 μL | 5 sec | 5 sec |
| N-Methyl Imidazole | 186 | 233 μL | 5 sec | 5 sec |
| TCA | 110.1 | 2.3 mL | 21 sec | 21 sec |
| Iodine | 11.2 | 1.7 mL | 45 sec | 45 sec |
| Acetonitrile | NA | 6.67 mL | NA | NA |

B. 0.2 μmol Synthesis Cycle ABI 394 Instrument

| | | | | Wait |
|---|---|---|---|---|
| | | | Wait Time* | Time* |
| Reagent | Equivalents | Amount | 2′-O-methyl | RNA |
| Phosphoramidites | 15 | 31 μL | 233 sec | 465 sec |
| S-Ethyl Tetrazole | 38.7 | 31 μL | 233 min | 465 sec |
| Acetic Anhydride | 655 | 124 μL | 5 sec | 5 sec |
| N-Methyl Imidazole | 1245 | 124 μL | 5 sec | 5 sec |
| TCA | 700 | 732 μL | 10 sec | 10 sec |
| Iodine | 20.6 | 244 μL | 15 sec | 15 sec |
| Acetonitrile | NA | 2.64 mL | NA | NA |

C. 0.2 μmol Synthesis Cycle 96 well Instrument

| | Equivalents | Amount | | Wait |
|---|---|---|---|---|
| | 2′-O-methyl/ | 2′-O-methyl/ | Wait Time* | Time* |
| Reagent | Ribo | Ribo | 2′-O-methyl | Ribo |
| Phosphoramidites | 33/66 | 60/120 μL | 233 sec | 465 sec |
| S-Ethyl Tetrazole | 75/150 | 60/120 μL | 233 min | 465 sec |
| Acetic Anhydride | 50/50 | 50/50 μL | 10 sec | 10 sec |
| N-Methyl | 502/502 | 50/50 μL | 10 sec | 10 sec |
| Imidazole | | | | |
| TCA | 16,000/16,000 | 500/500 μL | 15 sec | 15 sec |
| Iodine | 6.8/6.8 | 80/80 μL | 30 sec | 30 sec |
| Acetonitrile | NA | 850/850 μL | NA | NA |

*Wait time does not include contact time during delivery.

TABLE III
Human BACE Hammerhead Ribozyme and Target Sequence

| | | | | Rz Seq |
|---|---|---|---|---|
| Pos | Substrate | Seq ID | Ribozyme | ID |
| 9 | CCACUCGU C CGCAGCCC | 1 | GGGCUGCU CUGAUGAG X CGAA ACGCGUGG | 1776 |
| 47 | AGCUGGAU U AUGGUGGC | 2 | GCCACCAU CUGAUGAG X CGAA AUCCAGCU | 1777 |
| 48 | GCUGGAUU A UGGUGGCC | 3 | GGCCACCA CUGAUGAG X CGAA AAUCCAGC | 1778 |
| 93 | GGAGCCCU U GCCCCUGC | 4 | GCAGGGGC CUGAUGAG X CGAA AGGGCUCC | 1779 |
| 163 | CCGCCCCU C CCAGCCCC | 5 | GGGGCUGG CUGAUGAG X CGAA AGGGGCGG | 1780 |
| 221 | GCCGAUGU A GCGGGCUC | 6 | GAGCCCGC CUGAUGAG X CGAA ACAUCGGC | 1781 |
| 229 | AGCGGGCU C CGGAUCCC | 7 | GGGAUCCG CUGAUGAG X CGAA AGCCCGCU | 1782 |
| 235 | CUCCGGAU C CCAGCCUC | 8 | GAGGCUGG CUGAGGAG X CGAA AUCCGGAG | 1783 |
| 243 | CCCAGCCU C UCCCCUGC | 9 | GCAGGGGA CUGAUGAG X CGAA AGGCUGGG | 1784 |
| 245 | CAGCCUCU C CCCUGCUC | 10 | GAGCAGGC CUGAUGAG X CGAA AGAGGCUG | 1785 |
| 253 | CCCCUGCU C CCGUGCUC | 11 | GAGCACGG CUGAUGAG X CGAA AGCAGGGG | 1786 |
| 261 | CCCGUGCU C UGCGGAUC | 12 | GAUCCGCA CUGAUGAG X CGAA AGCACGGG | 1787 |
| 269 | CUGCGGAU C UCCCCUGA | 13 | UCAGGGGA CUGAUGAG X CGAA AUCCGCAG | 1788 |
| 271 | GCGGAUCU C CCCUGACC | 14 | GGUCAGGG CUGAUGAG X CGAA AGAUCCGC | 1789 |
| 283 | UGACCGCU C UCCACAGC | 15 | GCUGUGGA CUGAUGAG X CGAA AGCGGUCA | 1790 |
| 285 | ACCGCUCU C CACAGCCC | 16 | GGGCUGUG CUGAUGAG X CGAA AGAGCGGU | 1791 |
| 334 | CCUGGCGU C CUGAUCGG | 17 | GGCAUCAG CUGAUGAG X CGAA ACGCCAGG | 1792 |
| 351 | CCCAAGCU C CCUCUCCU | 18 | AGGAGAGG CUGAUGAG X XGAA AGCUUGGG | 1793 |
| 355 | AGCUCCCU C UCCUGAGA | 19 | UCUCAGGA CUGAUGAG X CGAA AGGGAGCU | 1794 |
| 357 | CUCCCUCU C CUGAGAAG | 20 | CUUCUCAG CUGAUGAG X CGAA AGAGGGAG | 1795 |
| 386 | CCCAGACU U GGGGGCAG | 21 | CUGCCCCC CUGAUGAG X CGAA AGUCUGGG | 1796 |
| 477 | CCCUGGCU C CUGCUGUG | 22 | CACAGCAG CUGAUGAG X CGAA AGCCAGGG | 1797 |
| 531 | CACGGCAU C GCCCUGCC | 23 | GGCAGCCG CUGAUGAG X CGAA AUGCCGUG | 1798 |
| 632 | GGGCAGCU U UGUGGAGA | 24 | UCUCCACA CUGAUGAG X CGAA AGCUGCCC | 1799 |
| 633 | GGCAGCUU U GUGGAGAU | 25 | AUCUCCAC CUGAUGAG X CGAA AAGCUGCC | 1800 |
| 665 | GGGCAAGU C GGGGCAGG | 26 | CCUGCCCC CUGAUGAG X CGAA ACUUGCCC | 1801 |
| 667 | GCAGGGCU A CUACGUGG | 27 | CCACGUAG CUGAUGAG X CGAA AGCCCUGC | 1802 |
| 680 | GGGCUACU A CGUGGAGA | 28 | UCUCCACG CUGAUGAG X CGAA AGUACGGG | 1803 |
| 717 | CAGACGCU C AACAUCCU | 29 | AGGAUGUU CUGAUGAG X CGAA AGCGUCUG | 1804 |
| 723 | CUCAACAU C CUGGUGGA | 30 | UCCACCAG CUGAUGAG X CGAA AUGUUGAG | 1805 |
| 733 | UGGUGGAU A CAGGCAGC | 31 | GCUGCCUG CUGAUGAG X CGAA AUCCACCA | 1806 |
| 745 | GCAGCAGU A ACUUUGCA | 32 | UGCAAAGU CUGAUGAG X CGAA ACUGCUGC | 1807 |
| 749 | CAGUAACU U UGCAGUGG | 33 | CCACUGCA CUGAUGAG X CGAA AGUUACUG | 1808 |
| 750 | AGUAACUU U GCAGUGGG | 34 | CCCACUGC CUGAUGAG X CGAA AAGUUACU | 1809 |
| 776 | CCACCCCU U CCUGCAUC | 35 | GAUGCAGG CUGAUGAG X CGAA AGGGGUGG | 1810 |
| 777 | CACCCCUU C CUGCAUCG | 36 | CGAUGCAG CUGAUGAG X CGAA AAGGGGUG | 1811 |
| 784 | UCCUGCAU C GCUACUAC | 37 | GUAGUAGC CUGAUGAG X CGAA AUGCAGGA | 1812 |
| 788 | GCAUCGCU A CUACCAGA | 38 | UCUGGUAG CUGAUGAG X CGAA AGCGAUGC | 1813 |
| 791 | UCGCUACU A CCAGAGGC | 39 | GCCUCUGG CUGAUGAG X CGAA AGUAGCGA | 1814 |
| 806 | GCAGCUGU C CAGCACAU | 40 | AUGUGCUG CUGAUGAG X CGAA ACAGCUGC | 1815 |
| 815 | CAGCACAU A CCGGGACC | 41 | GGUCCCGG CUGAUGAG X CGAA AUGUGCUG | 1816 |
| 825 | CGGGACCU C CGGAAGGG | 42 | CCCUUCCG CUGAUGAG X CGAA AGGUCCCG | 1817 |
| 839 | GGGUGUGU A UGUGCCCU | 43 | AGGGCACA CUGAUGAG X CGAA ACACACCC | 1818 |
| 848 | UGUGCCCU A CACCCAGG | 44 | CCUGGGUG CUGAUGAG X CGAA AGGGCACA | 1819 |
| 891 | GACCUGGU A AGCAUCCC | 45 | GGGAUGCU CUGAUGAG X CGAA ACCAGGUC | 1820 |
| 897 | GUAAGCAU C CCCCAUGG | 46 | CCAUGGGG CUGAUGAG X CGAA AUGCUUAC | 1821 |
| 915 | CCCAACGU C ACUGUGCG | 47 | CGCACAGU CUGAUGAG X CGAA ACGUUGGG | 1822 |
| 933 | GCCAACAU U GCUGCCAU | 48 | AUGGCAGC CUGAUGAG X CGAA AUGUUGGC | 1823 |
| 942 | GCUGCCAU C ACUGAAUC | 49 | GAUUCAGU CUGAUGAG X CGAA AUGGCAGC | 1824 |
| 950 | CACUGAAU C AGACAAGU | 50 | ACUUGUCU CUGAUGAG X CGAA AUUCAGUG | 1825 |
| 959 | AGACAAGU U CUUCAUCA | 51 | UGAUGAAG CUGAUGAG X CGAA ACUUGUCU | 1826 |
| 960 | GACAAGUU C UUCAUCAA | 52 | UUGAUGAA CUGAUGAG X CGAA AACUUGUC | 1827 |
| 962 | CAAGUUCU U CAUCAACG | 53 | CGUUGAUG CUGAUGAG X CGAA AGAACUUG | 1828 |
| 963 | AAGUUCUU C AUCAACGG | 54 | CCGUUGAU CUGAUGAG X CGAA AAGAACUU | 1829 |
| 966 | UUCUUCAU C AACGGCUC | 55 | GAGCCGUU CUGAUGAG X CGAA AUGAAGAA | 1830 |
| 974 | CAACGGCU C CAACUGGG | 56 | CCCAGUUG CUGAUGAG X CGAA AGCCGUUG | 1831 |
| 990 | GAAGGCAU C CUGGGGCU | 57 | AGCCCCAG CUGAUGAG X CGAA AUGCCUUC | 1832 |
| 1004 | GCUGGCCU A UGCUGAGA | 58 | UCUCAGCA CUGAUGAG X CGAA AGGCCAGC | 1833 |
| 1014 | GCUGAGAU U GCCAGGCC | 59 | GGCCUGGC CUGAUGAG X CGAA AUCUCAGC | 1834 |
| 1031 | UGACGACU C CCUGGAGC | 60 | GCUCCAGG CUGAUGAG X CGAA AGUCGUCA | 1835 |
| 1042 | UGGAGCCU U UCUUUGAC | 61 | GUCAAAGA CUGAUGAG X CGAA AGGCUCCA | 1836 |
| 1043 | GGAGCCUU U CUUUGACU | 62 | AGUCAAAG CUGAUGAG X CGAA AAGGCUCC | 1837 |
| 1044 | GAGCCUUU C UUUGACUC | 63 | GAGUCAAA CUGAUGAG X CGAA AAAGGCUC | 1838 |
| 1046 | GCCUUUCU U UGACUCUC | 64 | GAGAGUCA CUGAUGAG X CGAA AGAAAGGC | 1839 |
| 1047 | CCUUUCUU U GACUCUCU | 65 | AGAGAGUC CUGAUGAG X CGAA AAGAAAGG | 1840 |
| 1052 | CUUUGACU C UCUGGUAA | 66 | UUACCAGA CUGAUGAG X CGAA AGUCAAAG | 1841 |
| 1054 | UUGACUCU C UGGUAAAG | 67 | CUUUACCA CUGAUGAG X CGAA AGAGUCAA | 1842 |
| 1059 | UCUCUGGU A AAGCAGAC | 68 | GUCUGCUU CUGAUGAG X CGAA ACCAGAGA | 1843 |
| 1074 | ACCCACGU U CCCAACCU | 69 | AGGUUGGG CUGAUGAG X CGAA ACGUGGGU | 1844 |
| 1075 | CCCACGUU C CCAACCUC | 70 | GAGGUUGG CUGAUGAG X CGAA AACGUGGG | 1845 |
| 1083 | CCCAACCU C UUCUCCCU | 71 | AGGGAGAA CUGAUGAG X CGAA AGGUUGGG | 1846 |
| 1085 | CAACCUCU U CUCCCUGC | 72 | GCAGGGAG CUGAUGAG X CGAA AGAGGUUG | 1847 |
| 1086 | AACCUCUU C UCCCUGCA | 73 | UGCAGGGA CUGAUGAG X CGAA AAGAGGUU | 1848 |
| 1088 | CCUCUUCU C CCUGCAGC | 74 | GCUGCAGG CUGAUGAG X CGAA AGAAGAGG | 1849 |
| 1098 | CUGGAGCU U UGUGGUGC | 75 | GCACCACA CUGAUGAG X CGAA AGCUGCAG | 1850 |
| 1099 | UGCAGCUU U GUGGUGCU | 76 | AGCACCAC CUGAUGAG X CGAA AAGCUGCA | 1851 |
| 1112 | UGCUGGCU U CCCCCUCA | 77 | UGAGGGGG CUGAUGAG X CGAA AGCCAGCA | 1852 |
| 1113 | GCUGGCUU C CCCCUCAA | 78 | UUGAGGGG CUGAUGAG X CGAA AAGCCAGC | 1853 |
| 1119 | UUCCCCCU C AACCAGUC | 79 | GACUGGUU CUGAUGAG X CGAA AGGGGGAA | 1854 |
| 1127 | CAACCAGU C UGAAGUGC | 80 | GCACUUCA CUGAUGAG X CGAA ACUGGUUG | 1855 |
| 1142 | GCUGGCCU C UGUCGGAG | 81 | CUCCGACA CUGAUGAG X CGAA AGGCCAGC | 1856 |
| 1146 | GCCUCUGU C GGAGGGAG | 82 | CCCCCUCC CUGAUGAG X CGAA ACAGAGGC | 1857 |
| 1161 | AGCAUGAU C AUUGGAGG | 83 | CCUCCAAU CUGAUGAG X CGAA AUCAUGCU | 1858 |
| 1164 | AUGAUCAU U GGAGGUAU | 84 | AUACCUCC CUGAUGAG X CGAA AUGAUCAU | 1859 |
| 1171 | UUGGAGGU A UCGACCAC | 85 | GUGGUCGA CUGAUGAG X CGAA ACCUCCAA | 1860 |
| 1173 | GGAGGUAU C GACCACUC | 86 | GAGUGGUC CUGAUGAG X CGAA AUACCUCC | 1861 |
| 1181 | CGACCACU C GCUCUACA | 87 | UGUACAGC CUGAUGAG X CGAA AGUGGUCG | 1862 |
| 1187 | CUCGCUGU A CACAGGCA | 88 | UGCCUGUG CUGAUGAG X CGAA ACAGCGAG | 1863 |
| 1198 | CAGGCAGU C UCUGGUAU | 89 | AUACCAGA CUGAUGAG X CGAA ACUGCCUG | 1864 |
| 1200 | GGCAGUCU C UGGUAUAC | 90 | GUAUACCA CUGAUGAG X CGAA AGACUGCC | 1865 |
| 1205 | UCUCUGGU A UACACCCA | 91 | UGGGUGUA CUGAUGAG X CGAA ACCAGAGA | 1866 |
| 1207 | UCUGGUAU A CACCCAUC | 92 | GAUGGGUG CUGAUGAG X CGAA AUACCAGA | 1867 |
| 1215 | ACACCCAU C CGGCGGGA | 93 | UCCCGCCG CUGAUGAG X CGAA AUGGGUGU | 1868 |
| 1229 | GGAGUGGU A UUAUGAGG | 94 | CUCCAUAA CUGAUGAG X CGAA ACCACUCC | 1869 |
| 1231 | AGUGGUAU U AUGAGGUG | 95 | CACCUCAU CUGAUGAG X CGAA AUACCACU | 1870 |
| 1232 | GUGGUAUU A UGAGGUGA | 96 | UCACCUCA CUGAUGAG X CGAA AAUACCAC | 1871 |
| 1242 | GAGGUGAU C AUUGUGCG | 97 | CGCACAAU CUGAUGAG X CGAA AUCACCUC | 1872 |
| 1245 | GUGAUCAU U GUGCGGGU | 98 | ACCCGCAC CUGAUGAG X CGAA AUGAUCAC | 1873 |
| 1260 | GUGGAGAU C AAUGGACA | 99 | UGUCCAUU CUGAUGAG X CGAA AUCUCCAC | 1874 |
| 1273 | GACAGGAU C UGAAAAUG | 100 | CAUUUUCA CUGAUGAG X CGAA AUCCUGUC | 1875 |
| 1295 | CAAGGAGU A CAACUAUG | 101 | CAUAGUUG CUGAUGAG X CGAA ACUCCUUG | 1876 |
| 1301 | GUACAACU A UGACAAGA | 102 | UCUUGUCA CUGAUGAG X CGAA AGUUGUAC | 1877 |
| 1314 | AAGAGCAU U GUGGACAG | 103 | CUGUCCAC CUGAUGAG X CGAA AUGCUCUU | 1878 |
| 1338 | ACCAACCU U CGUUUGCC | 104 | GGCAAACG CUGAUGAG X CGAA AGGUUGGU | 1879 |
| 1339 | CCAACCUU C GUUUGCCC | 105 | GGGCAAAC CUGAUGAG X CGAA AAGGUUGG | 1880 |
| 1342 | ACCUUCGU U UGCCCAAG | 106 | CUUGGGCA CUGAUGAG X CGAA ACGAAGGU | 1881 |
| 1343 | CCUUCGUU U GCCCAAGA | 107 | UCUUGGGC CUGAUGAG X CGAA AACGAAGG | 1882 |
| 1358 | GAAAGUGU U UGAAGCUG | 108 | CAGCUUCA CUGAUGAG X CGAA ACACUUUC | 1883 |
| 1359 | AAAGUGUU U GAAGCUGC | 109 | GCAGCUUC CUGAUGAG X CGAA AACACUUU | 1884 |
| 1371 | GCUGCAGU C AAAUCCAU | 110 | AUGGAUUU CUGAUGAG X CGAA ACUGCAGC | 1885 |
| 1376 | AGUCAAAU C CAUCAAGG | 111 | CCUUGAUG CUGAUGAG X CGAA AUUUGACU | 1886 |
| 1380 | AAAUCCAU C AAGGCAGC | 112 | GCUGCCUU CUGAUGAG X CGAA AUGGAUUU | 1887 |
| 1391 | GGCAGCCU C CUCCACGG | 113 | CCGUGGAG CUGAUGAG X CGAA AGGCUGCC | 1888 |
| 1394 | AGCCUCCU C CACGGAGA | 114 | UCUCCGUG CUGAUGAG X CGAA AAGAGGCU | 1889 |
| 1406 | GGAGAAGU U CCCUGAUG | 115 | CAUCAGGG CUGAUGAG X CGAA ACUUCUCC | 1890 |
| 1407 | GAGAAGUU C CCUGAUGG | 116 | CCAUCAGG CUGAUGAG X CGAA AACUUCUC | 1891 |
| 1417 | CUGAUGGU U UCUGGCUA | 117 | UAGCCAGA CUGAUGAG X CGAA ACCAUCAG | 1892 |
| 1418 | UGAUGGUU U CUGGCUAG | 118 | CUAGCCAG CUGAUGAG X CGAA AACCAUCA | 1893 |
| 1419 | GAUGGUUU C UGGCUAGG | 119 | CCUAGCCA CUGAUGAG X CGAA AAACCAUC | 1894 |
| 1425 | UUCUGGCU A GGAGAGCA | 120 | UGCUCUCC CUGAUGAG X CGAA AGCCAGAA | 1895 |
| 1465 | CCACCCCU U GGAACAUU | 121 | AAUGUUCC CUGAUGAG X CGAA AGGGGUGG | 1896 |
| 1473 | UGGAACAU U UUCCCAGU | 122 | ACUGGGAA CUGAUGAG X CGAA AUGUUCCA | 1897 |
| 1474 | GGAACAUU U UCCCAGUC | 123 | GACUGGGA CUGAUGAG X CGAA AAUGUUCC | 1898 |
| 1475 | GAACAUUU U CCCAGUCA | 124 | UGACUGGG CUGAUGAG X CGAA AAAUGUUC | 1899 |
| 1476 | AACAUUUU C CCAGUCAU | 125 | AUGACUGG CUGAUGAG X CGAA AAAAUGUU | 1900 |
| 1482 | UUCCCAGU C AUCUCACU | 126 | AGUGAGAU CUGAUGAG X CGAA ACUGGGAA | 1901 |
| 1485 | CCAGUCAU C UCACUCUA | 127 | UAGAGUGA CUGAUGAG X CGAA AUGACUGG | 1902 |
| 1487 | AGUCAUCU C ACUCUACC | 128 | GGUAGAGU CUGAUGAG X CGAA AGAUGACU | 1903 |
| 1491 | AUCUCACU C UACCUAAU | 129 | AUUAGGUA CUGAUGAG X CGAA AGUGAGAU | 1904 |
| 1493 | CUCACUCU A CCUAAUGG | 130 | CCAUUAGG CUGAUGAG X CGAA AGAGUGAG | 1905 |
| 1497 | CUCUACCU A AUGGGUGA | 131 | UCACCCAU CUGAUGAG X CGAA AGGUAGAG | 1906 |
| 1509 | GGUGAGGU U ACCAACCA | 132 | UGGUUGGU CUGAUGAG X CGAA ACCUCACC | 1907 |
| 1510 | GUGAGGUU A CCAACCAG | 133 | CUGGUUGG CUGAUGAG X CGAA AACCUCAC | 1908 |
| 1520 | CAACCAGU C CUUCCGCA | 134 | UGCGGAAG CUGAUGAG X CGAA ACUGGUUG | 1909 |
| 1523 | CCAGUCCU U CCGCAUCA | 135 | UGAUGCGG CUGAUGAG X CGAA AGGACUGG | 1910 |
| 1524 | CAGUCCUU C CGCAUCAC | 136 | GUGAUGCG CUGAUGAG X CGAA AAGGACUG | 1911 |
| 1530 | UUCCGCAU C ACCAUCCU | 137 | AGGAUGGU CUGAUGAG X CGAA AUGCGGAA | 1912 |
| 1536 | AUCACCAU C CUUCCGCA | 138 | UGCGGAAG CUGAUGAG X CGAA AUGGUGAU | 1913 |
| 1539 | ACCAUCCU U CCGCAGCA | 139 | UGCUGCCG CUGAUGAG X CGAA AGGAUGGU | 1914 |
| 1540 | CCAUCCUU C CGCAGCAA | 140 | UUGCUGCG CUGAUGAG X CGAA AAGGAUGG | 1915 |
| 1550 | GCAGCAAU A CCUGCGGC | 141 | GCCGCAGG CUGAUGAG X CGAA AUUGCUGC | 1916 |
| 1580 | GGCCACGU C CCAAGACG | 142 | CGUCUUGG CUGAUGAG X CGAA ACGUGGCC | 1917 |
| 1594 | ACGACUGU U ACAAGUUU | 143 | AAACUUGU CUGAUGAG X CGAA ACAGUCGU | 1918 |
| 1595 | CGACUGUU A CAAGUUUG | 144 | CAAACUUG CUGAUGAG X CGAA AACAGUCG | 1919 |
| 1601 | UUACAAGU U UGCCAUCU | 145 | AGAUGGCA CUGAUGAG X CGAA ACUUGUAA | 1920 |
| 1602 | UACAAGUU U GCCAUCUC | 146 | GAGAUGGC CUGAUGAG X CGAA AACUUGUA | 1921 |
| 1608 | UUUGCCAU C UCACAGUC | 147 | GACUGUGA GACUGUGA X CGAA AUGGCAAA | 1922 |
| 1610 | UGCCAUCU C ACAGUCAU | 148 | AUGACUGU CUGAUGAG X CGAA ACAGGGCA | 1923 |
| 1616 | CUCACAGU C AUCCACGG | 149 | CCGUGGAU CUGAUGAG X CGAA ACUGUGAG | 1924 |
| 1619 | ACAGUCAU C CACGGGCA | 150 | UGCCCGUG CUGAUGAG X CGAA AUGACUGU | 1925 |
| 1632 | GGCACUGU U AUGGGACG | 151 | GCUCCCAU CUGAUGAG X CGAA ACAGUGCC | 1926 |
| 1633 | GCACUGUU A UGGGAGCU | 152 | AGCUCCCA CUGAUGAG X CGAA AACAGUGC | 1927 |
| 1644 | GGAGCUGU U AUCAUGGA | 153 | UCCAUGAU CUGAUGAG X CGAA ACAGCUGG | 1928 |
| 1645 | GAGCUGUU A UCAUGGAG | 154 | CUCCAUGA CUGAUGAG X CGAA AACAGCUC | 1929 |
| 1647 | GCUGUUAU C AUGGAGGG | 155 | CCCUCCAU CUGAUGAG X CGAA AUAACAGC | 1930 |
| 1658 | GGAGGGCU U CUACCUUG | 156 | CAACGUAG CUGAUGAG X CGAA ACGGGUCC | 1931 |
| 1659 | GAGGGCUU C UACGUUGU | 157 | ACAACGUA CUGAUGAG X CGAA AAGCCCUC | 1932 |
| 1661 | GGGCUUCU A CGUUGUCU | 158 | AGACAACG CUGAUGAG X CGAA AGAAGCCC | 1933 |
| 1665 | UUCUACGU U GUCUUUGA | 159 | UCAAAGAC CUGAUGAG X CGAA ACGUAGAA | 1934 |
| 1668 | UACGUUGU C UUUGAUCG | 160 | CGAUCAAA CUGAUGAG X CGAA ACGGCGUA | 1935 |
| 1670 | CGUUGUCU U UGAUCGGG | 161 | CCCGAUCA CUGAUGAG X CGAA AGACAACG | 1936 |
| 1671 | GUUGUCUU U GAUCGGGC | 162 | GCCCGAUC CUGAUGAG X CGAA AAGACAAC | 1937 |
| 1675 | UCUUUGAU C GGGCCCGA | 163 | UCGGGCCC CUGAUGAG X CGAA AUCAAAGA | 1938 |
| 1692 | AAACGAAU U GGCUUUGC | 164 | CGAAAGCC CUGAUGAG X CGAA AUUCGUUU | 1939 |
| 1697 | AAUUGGCU U UGCUGUCA | 165 | UGACAGCA CUGAUGAG X CGAA ACGGAAUU | 1940 |
| 1698 | AUUGGCUU U GCUGUCAG | 166 | CUGACAGC CUGACAGC X CGAA AAGCCAAU | 1941 |
| 1704 | UUUGUCGU C AGCGCUUG | 167 | CAAGCGCU CUGAUGAG X CGAA ACAGCAAA | 1942 |
| 1711 | UCAGCGCU U CGGAUGUG | 168 | CACAUGGC CUGAUGAG X CGAA AGCGCUGA | 1943 |
| 1730 | CGAUGAGU U CAGGACGG | 169 | CCGUCCUG CUGAUGAG X CGAA ACUCAUCG | 1944 |
| 1731 | GAUGAGUU C AGGACGGC | 170 | GCCGUCCU CUGAUGAG X CGAA AACUCAUC | 1945 |
| 1756 | AAGGCCCU U UUGUCACC | 171 | GGUGACAA CUGAUGAG X CGAA AGGGCCUU | 1946 |
| 1757 | AGGCCCUU U UGUCACCU | 172 | AGGUGACA CUGAUGAG X CGAA AAGGGCCU | 1947 |
| 1758 | GGCCCUUU U GUCACCUU | 173 | AAGGUGAC CUGAUGAG X CGAA AAAGGGCC | 1948 |
| 1761 | CCUUUUGU U ACCUUGGA | 174 | UCCAAGGU CUGAUGAG X CGAA ACAAAAGG | 1949 |
| 1766 | UGUCACCU U GGACAUGG | 175 | CCAUGUCC CUGAUGAG X CGAA AGGUGACA | 1950 |
| 1787 | CUGUGGCU A CAACAUUC | 176 | GAAUGUUG CUGAUGAG X CGAA AGCCACAG | 1951 |
| 1794 | UACAACAU U CCACAGAC | 177 | GUCUGUGG CUGAUGAG X CGAA AUGUUGUA | 1952 |
| 1795 | ACAACAUU C CACAGACA | 178 | UGUCUGUG CUGAUGAG X CGAA AAUGUUGU | 1953 |
| 1811 | AGAUGAGU C AACCCUCA | 179 | UGAGGGUU CUGAUGAG X CGAA ACUCAUCU | 1954 |
| 1818 | UCAACCCU C AUGACCAU | 180 | AUGGUCAU CUGAUGAG X CGAA AGGGUUGA | 1955 |
| 1827 | AUGACCAU A GCCUAUGA | 181 | ACAUAGGC CUGAUGAG X CGAA AUGGUCAU | 1956 |
| 1832 | CAUAGCCU A UGUCAUGG | 182 | CCAUGACA CUGAUGAG X CGAA AGGCUAUG | 1957 |
| 1836 | GCCUAUGU C AUGGCUGC | 183 | GCAGCCAU CUGAUGAG X CGAA ACAUAGGC | 1958 |
| 1848 | GCUGCCAU C UGCGCCCU | 184 | AGGGCGCA CUGAUGAG X CGAA AUGGCAGC | 1959 |
| 1857 | UGCGCCCU C UUCAUGCU | 185 | AGCAUGAA CUGAUGAG X CGAA AGGGCGCA | 1960 |
| 1859 | CGCCCUCU U CAUGCUGC | 186 | GCAGCAUG CUGAUGAG X CGAA AGAGGGCG | 1961 |
| 1860 | GCCCUCUU U AUGCUGCC | 187 | GGCAGCAU CUGAUGAG X CGAA AAGAGGGC | 1962 |
| 1872 | CUGCCACU C UGCCUCAU | 188 | AUGAGGCA CUGAUGAG X CGAA AGUGGCAG | 1963 |
| 1878 | CUCUGCCU C AUGGUGUG | 189 | CACACCAU CUGAUGAG X CGAA AGGCAGAG | 1964 |
| 1888 | UGGUGUGU C AGUGGCGC | 190 | GCGCCACU CUGAUGAG X CGAA ACACACCA | 1965 |
| 1902 | CGCUGCCU C CGCUGCCU | 191 | AGGCAGCG CUGAUGAG X CGAA AGGCAGCG | 1966 |
| 1931 | UGAUGACU U UGCUGAUG | 192 | CAUCAGCA CUGAUGAG X CGAA AGUCAUCA | 1967 |
| 1932 | GAUGACUU U GCUGAUGA | 193 | UCAUCAGC CUGAUGAG X CGAA AAGUCAUC | 1968 |
| 1944 | GAUGACAU C UCCCUGCU | 194 | AGCAGGGA CUGAUGAG X CGAA AUGUCAUC | 1969 |
| 1946 | UGACAUCU C CCUGCUGA | 195 | UCAGCAGG CUGAUGAG X CGAA AGAUGUCA | 1970 |
| 1981 | CAGAAGAU A GAGAUUCC | 196 | GGAAUCUC CUGAUGAG X CGAA AUCUUCUG | 1971 |
| 1987 | AUAGAGAU U CCCCUGGA | 197 | UCCAGGGG CUGAUGAG X CGAA AUCUCUAU | 1972 |
| 1988 | UAGAGAUU C CCCUGGAC | 198 | GUCCAGGG CUGAUGAG X CGAA AAGCUCUA | 1973 |
| 2004 | CCACACCU C CGUGGUUC | 199 | GAACCACG CUGAUGAG X CGAA AGGUGUGG | 1974 |
| 2011 | UCCGUGGU U CACUUUGG | 200 | CCAAAGUG CUGAUGAG X CGAA ACCACGGA | 1975 |
| 2012 | CCGUCCUU C ACUUUGGU | 201 | ACCAAAGU CUGAUGAG X CGAA AACCACGG | 1976 |
| 2016 | GGUUCACU U UGGUCACA | 202 | UGUGACCA CUGAUGAG X CGAA AGUGAACC | 1977 |
| 2017 | GUUCACUU U GGUCACAA | 203 | UUGUGACC CUGAUGAG X CGAA AAGUGAAC | 1978 |
| 2021 | ACUUUGGU C ACAAGUAG | 204 | CUACUUGU CUGAUGAG X CGAA ACCAAAGU | 1979 |
| 2028 | UCACAAGU A GGAGACAC | 205 | GUGUCUCC CUGAUGAG X CGAA ACUUGUGA | 1980 |
| 2063 | GAGCACCU C AGGACCCU | 206 | AGGGUCCU CUGAUGAG X CGAA AGGUGCUC | 1981 |
| 2072 | AGAACCCU C CCCACCCA | 207 | UGGGUGGG CUGAUGAG X CGAA AGGGUCCU | 1982 |
| 2091 | AAAUGCCU C UGCCUUGA | 208 | UCAAGGCA CUGAUGAG X CGAA AGGCAUUU | 1983 |
| 2097 | CUCUGCCU U GAUGGAGA | 209 | UCUCCAUC CUGAUGAG X CGAA AGGCAGAG | 1984 |
| 2129 | AGGUGGGU U CCAGGGAC | 210 | GUCCCUGG CUGAUGAG X CGAA ACCCACCU | 1985 |
| 2130 | GGUGGGUU C CAGGGACU | 211 | AGUCCCUG CUGAGGAG X CGAA AACCCACC | 1986 |
| 2141 | GGGACUGU A CCUGUAGG | 212 | CCUACAGG CUGAUGAG X CGAA ACAGUCCC | 1987 |
| 2147 | GUACCUGU A GGAAACAG | 213 | CUGUUUCC CUGAUGAG X CGAA ACAGGUAC | 1988 |
| 2177 | GAAGCACU C UGCUGGCG | 214 | CGCCAGCA CUGAUGAG X CGAA AGUGCUUG | 1989 |
| 2191 | GCGGGAAU A CUCUUGGU | 215 | ACCAAGAG CUGAUGAG X CGAA AUUCCCGC | 1990 |
| 2194 | GGAAUACU C UUGGUCAC | 216 | GUGACCAA CUGAUGAG X CGAA AGUAUUCC | 1991 |
| 2196 | AAUACUCU U GGUCACCU | 217 | AGGUGACC CUGAUGAG X CGAA AGAGUAUU | 1992 |
| 2200 | CUCUUGGU C ACCUCAAA | 218 | UUUGAGGU CUGAUGAG X CGAA ACCAAGAG | 1993 |
| 2205 | GGUCACCU C AAAUUUAA | 219 | UUAAAUUU CUGAUGAG X CGAA AGGUGACC | 1994 |
| 2210 | CCUCAAAU U UAAGUCGG | 220 | CCGACUUA CUGAUGAG X CGAA AUUUGAGG | 1995 |
| 2211 | CUCAAAUU U AAGUCGGG | 221 | CCCGACUU CUGAUGAG X CGAA AAUUUGAG | 1996 |
| 2212 | UCAAAUUU A AGUCGGGA | 222 | UCCCGACU CUGAUGAG X CGAA AAAUUUGA | 1997 |
| 2216 | AUUUAAGU C GGGAAAUU | 223 | AAUUUCCC CUGAUGAG X CGAA ACUUAAAU | 1998 |
| 2224 | CGGGAAAU U CUGCUGCU | 224 | AGCAGCAG CUGAUGAG X CGAA AUUUCCCG | 1999 |
| 2225 | GGGAAAUU C UGCUGCUU | 225 | AAGCAGCA CUGAUGAG X CGAA AAUUUCCC | 2000 |
| 2233 | CUGCUGCU U GAAACUUC | 226 | GAAGUUUC CUGAUGAG X CGAA AGCAGCAG | 2001 |
| 2240 | UUGAAACU U CAGCCCUG | 227 | CAGGGCUG CUGAUGAG X CGAA AGUUUCAA | 2002 |
| 2241 | UGAAACUU C AGCCCUGA | 228 | UCAGGGCU CUGAUGAG X CGAA AAGUUUCA | 2003 |
| 2254 | CUGAACCU U UGUCCACC | 229 | GGUGGACA CUGAUGAG X CGAA AGGUUCAG | 2004 |
| 2255 | UGAACCUU U GUCCACCA | 230 | UGGUGGAC CUGAUGAG X CGAA AAGGUUCA | 2005 |
| 2258 | ACCUUUGU C CACCAUUC | 231 | GAAUGGUG CUGAUGAG X CGAA ACAAAGGU | 2006 |
| 2265 | UCCACCAU U CCUUUAAA | 232 | UUUAAAGG CUGAUGAG X CGAA AUGGUGGA | 2007 |
| 2266 | CCACCAUU C CUUUAAAU | 233 | AUUUAAAG CUGAUGAG X CGAA AAUGGUGG | 2008 |
| 2269 | CCAUUCCU U UAAAUUCU | 234 | AGAAUUUA CUGAUGAG X CGAA AGGAAUGG | 2009 |
| 2270 | CAUUCCUU U AAAUUCUC | 235 | GAGAAUUU CUGAUGAG X CGAA AAGGAAUG | 2010 |
| 2271 | AUUCCUUU A AAUUCUCC | 236 | GGAGAAUU CUGAUGAG X CGAA AAAGGAAU | 2011 |
| 2275 | CUUUAAAU U CUCCAACC | 237 | CCUUGGAG CUGAUGAG X CGAA AUUUAAAG | 2012 |
| 2276 | UUUAAAUU C UCCAACCC | 238 | GGGUUGGA CUGAUGAG X CGAA AAUUUAAA | 2013 |
| 2278 | UAAAUUCU C CAACCCAA | 239 | UUGGGUUG CUGAUGAG X CGAA AGAAUUUA | 2014 |
| 2290 | CCCAAAGU A UUCUUCUU | 240 | AAGAAGAA CUGAUGAG X CGAA ACUUUGGG | 2015 |
| 2292 | CAAAGUAU U CUUCUUUU | 241 | AAAAGAAG CUGAUGAG X CGAA AUACUUUG | 2016 |
| 2293 | AAAGUAUU C UUCUUUUC | 242 | GAAAAGAA CUGAUGAG X CGAA AAUACUUU | 2017 |
| 2295 | AGUAUUCU U CUUUUCUU | 243 | AAGAAAAG CUGAUGAG X CGAA AGAAUACU | 2018 |
| 2296 | GUAUUCUU C UUUUCUUA | 244 | UAAGAAAA CUGAUGAG X CGAA AAGAAUAC | 2019 |
| 2298 | AUUCUUCU U UUCUUAGU | 245 | ACUAAGAA CUGAUGAG X CGAA AGAAGAAU | 2020 |
| 2299 | UUCUUCUU U UCUUAGUU | 246 | AACUAAGA CUGAUGAG X CGAA AAGAAGAA | 2021 |
| 2300 | UCUUCUUU U CUUAGUUU | 247 | AAACUAAG CUGAUGAG X CGAA AAAGAAGA | 2022 |
| 2301 | CUUCUUUU U UUAGUUUC | 248 | GAAACUAA CUGAUGAG X CGAA AGAAAAGA | 2023 |
| 2303 | UCUUUUCU U AGUUUCAG | 249 | CUGAAACU CUGAUGAG X CGAA AGAAAAGA | 2024 |
| 2304 | CUUUUCUU A GUUUCAGA | 250 | UCUGAAAC CUGAUGAG X CGAA AAGAAAAG | 2025 |
| 2307 | UUCUUAGU U UCACAAGU | 251 | ACUUCUGA CUGAUGAG X CGAA ACUAAGAA | 2026 |
| 2308 | UCUUAGUU U CAGAAGUC | 252 | UACUUCUG CUGAUGAG X CGAA AACUAAGA | 2027 |
| 2309 | CUUAGUUU C AGAAGUAC | 253 | GUACUUCU CUGAUGAG X CGAA AAACUAAG | 2028 |
| 2316 | UCAGAAGU A CUGGCAUC | 254 | GAUGCCAG CUGAUGAG X CGAA ACUUCUGA | 2029 |
| 2324 | AUCGGCAU C ACACGCAG | 255 | CUGCGUGU CUGAUGAG X CGAA AUGCCAGU | 2030 |
| 2335 | ACGCAGGU U ACCUUGGC | 256 | GCCAAGGU CUGAUGAG X CGAA ACCUGCGU | 2031 |
| 2336 | CGCAGGUU A CCUUGGCU | 257 | CGCCAAGG CUGAUGAG X CGAA AACCUGCG | 2032 |
| 2340 | GGUUACCU U GGCGUGUG | 258 | CACACGCC CUGAUGAG X CGAA AGGUAACC | 2033 |
| 2350 | GCGUGUGU C CCUGUGGU | 259 | ACCACAGG CUGAUGAG X CGAA ACACACGC | 2034 |
| 2359 | CCUGUGGU A CCCUGGCA | 260 | UGCCAGGG CUGAUGAG X CGAA ACCACAGG | 2035 |
| 2384 | ACCAAGCU U GUUUCCCU | 261 | AGGGAAAC CUGAUGAG X CGAA AGCUUGGU | 2036 |
| 2387 | AAGCUUGU U UCCCUGCU | 262 | AGCAGGGA CUGAUGAG X CGAA ACAAGCUU | 2037 |
| 2388 | AGCUUGUU U CCCUGCUG | 263 | CAGCAGGG CUGAUGAG X CGAA AACAAGCU | 2038 |
| 2389 | GCUUCUUU C CCUGCUGG | 264 | CCAGCAGG CUGAUGAG X CGAA AAACAAGC | 2039 |
| 2405 | GCCAAAGU C AGUAGGAG | 265 | CUCCUACU CUGAUGAG X CGAA ACUUUGGC | 2040 |
| 2409 | AAGUCAGU A GGAGAGGA | 266 | UCCUCUCC CUGAUGAG X CGAA ACUGACUU | 2041 |
| 2426 | UGCACAGU U UGCUAUUU | 267 | AAAUAGCA CUGAUGAG X CGAA ACUGUGCA | 2042 |
| 2427 | GCACAGUU U GCUAUUUG | 268 | CAAAUAGC CUGAUGAG X CGAA AACUGUGC | 2043 |
| 2431 | AGUUUGCU A UUUGCUUU | 269 | AAAGCAAA CUGAUGAG X CGAA AGCAAACU | 2044 |
| 2433 | UUUGCUAU U UGCUUUAG | 270 | CUAAAGCA CUGAUGAG X CGAA AUACGAAA | 2045 |
| 2434 | UUGCUAUU U GCUUUAGA | 271 | UCUAAAGC CUGAUGAG X CGAA AAUAGCAA | 2046 |
| 2438 | UAUUUGCU U UAGAGACA | 272 | UGUCUCUA CUGAUGAG X CGAA AGCAAAUA | 2047 |
| 2439 | AUUUGCUU U AGAGACAG | 273 | CUGUCUCU CUGAUGAG X CGAA AAGCAAAU | 2048 |
| 2440 | UUUGCUUU A GAGACAGG | 274 | CCUGUCUC CUGAUGAG X CGAA AAAGCAAA | 2049 |
| 2455 | GGGACUGU A UAAACAAG | 275 | CUUGUUUA CUGAUGAG X CGAA ACAGUCCC | 2050 |
| 2457 | GACUGUAU A AACAAGCC | 276 | GGCUUGUU CUGAUGAG X CGAA AUACGAUC | 2051 |
| 2467 | ACAAGCCU A ACAUUGGU | 277 | ACCAAUGU CUGAUGAG X CGAA AGGCUUGU | 2052 |
| 2472 | CCUAACAU U GGUGCAAA | 278 | UUUGCACC CUGAUGAG X CGAA AUGUUAGG | 2053 |
| 2484 | GCAAAGAU U GCCUGUUG | 279 | CAAGAGGC CUGAUGAG X CGAA AUCUUUGC | 2054 |
| 2489 | GAGGUCCU C UUGAAUUA | 280 | UAAUUCAA CUGAUGAG X CGAA AGGCAAUC | 2055 |
| 2491 | UUGCCUCU U GAAUUAAA | 281 | UUUAAUUC CUGAUGAG X CGAA AGAGGCAA | 2056 |
| 2496 | UCUUGAAU U AAAAAAAA | 282 | UUUUUUUU CUGAUGAG X CGAA AUUCAAGA | 2057 |
| 2497 | CUUGAAUU A AAAAAAAA | 283 | UUUUUUUU CUGAUGAG X CGAA AAUUCAAG | 2058 |
| 2510 | AAAAAACU A GAAAAAAA | 284 | UUUUUUUC CUGAUGAG X CGAA AGUUUUUU | 2059 |

Input Sequence = AF190725. Cut Site = G/.
Stem Length = 8 . Core Sequence = CUGAUGAG X CGAA (X = GCCGUUAGGC or other stem II)
AF190725 (Homo sapiens beta-site APP cleaving enzyme (BACE) mRNA; 2526 bp)

TABLE IV
Human BACE NCH Ribozyme and Target Sequence

| | | | | Rz Seq |
|---|---|---|---|---|
| Pos | Substrate | Seq ID | Ribozyme | ID |
| 10 | CACGCGUC C GCAGCCCG | 285 | CGGGCUGC CUGAUGAG X CGAA IACGCGUG | 2060 |
| 13 | GCGUCCGC A GCCCGCCC | 286 | GGGCGGGC CUGAUGAG X CGAA ICGGACGC | 2061 |
| 16 | UCCGCAGC C CGCCCGGG | 287 | CCCGGGCG CUGAUGAG X CGAA ICUGCGGA | 2062 |
| 17 | CCGCAGCC C GCCCGGGA | 288 | UCCCGGGC CUGAUGAG X CGAA IGCUGCGG | 2063 |
| 20 | CAGCCCGC C CGGGAGCU | 289 | AGUCCCCG CUGAUGAG X CGAA ICGGGCUG | 2064 |
| 21 | AGCCCGCC C GGGAGCUG | 290 | CAGCUCCC CUGAUGAG X CGAA IGCGGGCU | 2065 |
| 28 | CCGGGAGC U GCGAGCCG | 291 | CGGCUCGC CUGAUGAG X CGAA ICUCCCGG | 2066 |
| 35 | CUGCGACG C GCGAGCUG | 292 | CAGCUCGC CUGAUGAG X CGAA ICUCGCAG | 2067 |
| 42 | CCGCGAGC U GGAUUAUG | 293 | CAUAAUCC CUGAUGAG X CGAA ICUCGCGG | 2068 |
| 56 | AUGGUGGC C UGAGCAGC | 294 | GCUGCUCA CUGAUGAG X CGAA ICCACCAU | 2069 |
| 57 | UGGUGGCC U GAGCAGCC | 295 | GGCUGCUC CUGAUGAG X CGAA IGCCACCA | 2070 |
| 62 | CGGUGAGC A GCCAACGC | 296 | GCGUUGGC CUGAUGAG X CGAA ICUCAGGC | 2071 |
| 65 | UGAGCAGC C AACGCAGC | 297 | GCUGCGUU CUGAUGAG X CGAA ICUGCUCA | 2072 |
| 66 | GAGCAGCC A ACGCAGCC | 298 | GGCUGCGU CUGAUGAG X CGAA IGCUGCUC | 2073 |
| 71 | GCCAACGC A GCCGCAGG | 299 | CCUGCGGC CUGAUGAG X CGAA ICGUUGGC | 2074 |
| 74 | AACGCAGC C GCAGGAGC | 300 | GCUCCUGC CUGAUGAG X CGAA ICUGCGUU | 2075 |
| 77 | GCAGCCGC A GGAGCCCG | 301 | CGGGCUCC CUGAUGAG X CGAA ICGGCUGC | 2076 |
| 83 | GCAGGAGC C CGGAGCCC | 302 | GGGCUCCG CUGAUGAG X CGAA ICUCCUGC | 2077 |
| 84 | CAGGAGCC C GGAGCCCU | 303 | AGGGCUCC CUGAUGAG X CGAA IGCUCCUG | 2078 |
| 90 | CCCGGAGC C CUUGCCCC | 304 | GGGGCAAG CUGAUGAG X CGAA ICUCCGGG | 2079 |
| 91 | CCGGAGCC C UUGCCCCU | 305 | AGGGGCAA CUGAUGAG X CGAA IGCUCCGG | 2080 |
| 92 | CGGAGCCC U UGCCCCUG | 306 | CAGGGGCA CUGAUGAG X CGAA IGGCUCCG | 2081 |
| 96 | GCCCUUGC C CCUGCCCG | 307 | CGGGCAGG CUGAUGAG X CGAA ICAAGGGC | 2082 |
| 97 | CCCUUGCC C CUGCCCGC | 308 | GCGGGCAG CUGAUGAG X CGAA IGCAAGGG | 2083 |
| 98 | CCUUGCCC C UGCCCGCG | 309 | CGCGGGCA CUGAUGAG X CGAA IGGCAAGG | 2084 |
| 99 | CUUGCCCC U GCCCGCGC | 310 | GCGCGGGC CUGAUGAG X CGAA IGGGCAAG | 2085 |
| 102 | GCCCCUGC C CGCGCCGC | 311 | GCGGCGCG CUGAUGAG X CGAA ICAGGGGC | 2086 |
| 103 | CCCCUGCC C GCGCCGCC | 312 | GGCGGCGC CUGAUGAG X CGAA IGCAGGGG | 2087 |
| 108 | GCCCGCGC C GCCGCCCG | 313 | CGGGCGGC CUGAUGAG X CGAA ICGCGGGC | 2088 |
| 111 | CGCGCCGC C GCCCGCCG | 314 | CGGCGGGC CUGAUGAG X CGAA ICGGCGCG | 2089 |
| 114 | GCCGCCGC C CGCCGGGG | 315 | CCCCGGCG CUGAUGAG X CGAA ICGGCGGC | 2090 |
| 115 | CCGCCGCC C GCCGGGGG | 316 | CCCCCGGC CUGAUGAG X CGAA IGCGGCGG | 2091 |
| 118 | CCGCCCGC C GGGGGGAC | 317 | GUCCCCCC CUGAUGAG X CGAA ICGGGCGG | 2092 |
| 127 | GGGGGGAC C AGGGAAGC | 318 | GCUUCCCU CUGAUGAG X CGAA IUCCCCCC | 2093 |
| 128 | GGGGGACC A GGGAAGCC | 319 | GGCUUCCC CUGAUGAG X CGAA IGUCCCCC | 2094 |
| 136 | AGGGAAGC C GCCACCGG | 320 | CCGGUGGC CUGAUGAG X CGAA ICUUCCCU | 2095 |
| 139 | GAAGCCGC C ACCGGCCC | 321 | GGGCCGGU CUGAUGAG X CGAA ICGGCUUC | 2096 |
| 140 | AAGCCGCC A CCGGCCCG | 322 | CGGGCCGG CUGAUGAG X CGAA IGCGGCUU | 2097 |
| 142 | CGGCCCAC C GGCCCGCC | 323 | GGCGGGCC CUGAUGAG X CGAA IUGGCGGC | 2098 |
| 146 | CCACCGGC C CGCCAUGC | 324 | GCAUGGCG CUGAUGAG X CGAA ICCGGUGG | 2099 |
| 147 | CACCGGCC C GCCAUGCC | 325 | GGCAUGGC CUGAUGAG X CGAA IGCCGGUG | 2100 |
| 150 | CGGCCCGC C AUGCCCGC | 326 | GCGGGCAU CUGAUGAG X CGAA ICGGGCCG | 2101 |
| 151 | GGCCCGCC A UGCCCGCC | 327 | GGCGGGCA CUGAUGAG X CGAA IGCGGGCC | 2102 |
| 155 | CGCCAUGC C CGCCCCUC | 328 | GAGGGGCG CUGAUGAG X CGAA ICAUGGCG | 2103 |
| 156 | GCCAUGCC C GCCCCUCC | 329 | GGAGGGGC CUGAUGAG X CGAA IGCAUGGC | 2104 |
| 159 | AUGCCCGC C CCUCCCAG | 330 | CUGGGAGG CUGAUGAG X CGAA ICGGGCAU | 2105 |
| 160 | UGCCCGCC C CUCCCAGC | 331 | GCUCCCAG CUGAUGAG X CGAA IGCGGGCA | 2106 |
| 161 | GCCCGCCC C UCCCAGCC | 332 | GGCUGGGA CUGAUGAG X CGAA IGGCGGGC | 2107 |
| 162 | CCCGCCCC U CCCAGCCC | 333 | GGGCUGGG CUGAUGAG X CGAA IGGGCGGG | 2108 |
| 164 | CGCCCCUC C CAGCCCCG | 334 | CGGGGCUG CUGAUGAG X CGAA IAGGGGCG | 2109 |
| 165 | GCCCCUCC C AGCCCCGC | 335 | GCGGGGCU CUGAUGAG X CGAA IGAGGGGC | 2110 |
| 166 | CCCCUCCC A GCCCCGCC | 336 | GGCGGGGC CUGAUGAG X CGAA IGGAGGGG | 2111 |
| 169 | CUCCCAGC C CCGCCGGG | 337 | CCCGGCGG CUGAUGAG X CGAA ICUGGGAG | 2112 |
| 170 | UCCCAGCC C CGCCGGGA | 338 | UCCCGGCG CUGAUGAG X CGAA ICGUGGGA | 2113 |
| 171 | CCCAGCCC C GCCGGGAG | 339 | CUCCCGGC CUGAUGAG X CGAA IGGCUGGG | 2114 |
| 174 | AGCCCCGC C GGGAGCCC | 340 | GGGCUCCC CUGAUGAG X CGAA ICGGGGCU | 2115 |
| 181 | CCGGGAGC C CGCGCCCG | 341 | CGGGCGCG CUGAUGAG X CGAA ICUCCCGG | 2116 |
| 182 | CGGGAGCC C GCGCCCGC | 342 | GCGGGCGC CUGAUGAG X CGAA IGCUCCCG | 2117 |
| 187 | GCCCGCGC C CGCUGCCC | 343 | GGGCAGCG CUGAUGAG X CGAA ICGCGGGC | 2118 |
| 188 | CCCGCGCC C GCUGCCCA | 344 | UGGGCAGC CUGAUGAG X CGAA IGCGCGGG | 2119 |
| 191 | GCGCCCGC U GCCCAGGC | 345 | GCCUGGGC CUGAUGAG X CGAA ICGGGCGC | 2120 |
| 194 | CCCGCUGC C CAGGCUGG | 346 | CCAGCCUG CUGAUGAG X CGAA ICAGCGGG | 2121 |
| 195 | CCGCUGCC C AGGCUGGC | 347 | GCCAGCCU CUGAUGAG X CGAA IGCAGCGG | 2122 |
| 196 | CGCUGCCC A GGCUGGCC | 348 | GGCCAGCC CUGAUGAG X CGAA IGGCAGCG | 2123 |
| 200 | GCCCAGGC U GGCCGCCG | 349 | CGGCGGCC CUGAUGAG X CGAA ICCUGGGC | 2124 |
| 204 | AGGCUGGC C GCCGCCGU | 350 | ACGGCGGC CUGAUGAG X CGAA ICCAGCCU | 2125 |
| 207 | CUGGCCGC C GCCGUGCC | 351 | GGCACGGC CUGAUGAG X CGAA ICGGCCAG | 2126 |
| 210 | GCCGCCGC C GUGCCGAU | 352 | AUCGGCAC CUGAUGAG X CGAA ICGGCGGC | 2127 |
| 215 | CGCCGUGC C GAUGUAGC | 353 | GCUACAUC CUGAUGAG X CGAA ICACGGCG | 2128 |
| 228 | UAGCGGGC U CCGGAUCC | 354 | GGAUCCGG CUGAUGAG X CGAA ICCCGCUA | 2129 |
| 230 | GCGGGCUC C GGAUCCCA | 355 | UGGGAUCC CUGAUGAG X CGAA IAGCCCGC | 2130 |
| 236 | UCCGGAUC C CAGCCUCU | 356 | AGAGGCUG CUGAUGAG X CGAA IAUCCGGA | 2131 |
| 237 | CCGGAUCC C AGCCUCUC | 357 | GAGAGGCU CUGAUGAG X CGAA IGAUCCGG | 2132 |
| 238 | CGGAUCCC A GCCUCUCC | 358 | GGAGAGGC CUGAUGAG X CGAA IGGAUCCG | 2133 |
| 241 | AUCCCAGC C UCUCCCCU | 359 | AGGGGAGA CUGAUGAG X CGAA ICUGGGAU | 2134 |
| 242 | UCCCAGCC U CUCCCCUG | 360 | CAGGGGAG CUGAUGAG X CGAA IGCUGGGA | 2135 |
| 244 | CCAGCCUC U CCCCUGCU | 361 | AGCAGGGG CUGAUGAG X CGAA IAGGCUGG | 2136 |
| 246 | AGCCUCUC C CCUGCUCC | 362 | GGAGCAGG CUGAUGAG X CGAA IAGAGGCU | 2137 |
| 247 | GCCUCUCC C CUGCUCCC | 363 | GGGAGCAG CUGAUGAG X CGAA IGAGAGGC | 2138 |
| 248 | CCUCUCCC C UGCUCCCG | 364 | CGGGAGCA CUGAUGAG X CGAA IGGAGAGG | 2139 |
| 249 | CUCUCCCC U GCUCCCGU | 365 | ACGGGAGC CUGAUGAG X CGAA IGGGAGAG | 2140 |
| 252 | UCCCCUGC U CCCGUGCU | 366 | AGCACGGG CUGAUGAG X CGAA ICAGGGGA | 2141 |
| 254 | CCCUGCUC C CGUGCUCU | 367 | AGAGCACG CUGAUGAG X CGAA IAGCAGGG | 2142 |
| 255 | CCUGCUCC C CUGCUCUG | 368 | CAGAGCAC CUGAUGAG X CGAA IGAGCAGG | 2143 |
| 260 | UCCCGUGC U CUGCGGAU | 369 | AUCCGCAG CUGAGGAG X CGAA ICACGGGA | 2144 |
| 262 | UCGUGCUC U CGCCAUCU | 370 | AGAUCCGC CUGAUGAG X CGAA IAGCACGG | 2145 |
| 270 | UGCGGACU U CCCCUGAC | 371 | GUCAGGGG CUGAUGAG X CGAA IAUCCGCA | 2146 |
| 272 | CGGAUCUC C CCUGACCG | 372 | CGGUCAGG CUGAUGAG X CGAA IAGAUCCG | 2147 |
| 273 | GGAUCUCC C CUGACCGC | 373 | GCGGUCAG CUGAUGAG X CGAA IGAGAUCC | 2148 |
| 274 | GAUCUCCC C UGACCGCU | 374 | AGCGGUCA CUGAUGAG X CGAA IGGAGAUC | 2149 |
| 275 | AUCUCCCC U GACCGCUC | 375 | GAGCGGUC CUGAUGAG X CGAA IGGGAGAU | 2150 |
| 279 | CCCCUGAC C GCUCUCCA | 376 | UGGAGAGC CUGAUGAG X CGAA IUCAGGGG | 2151 |
| 282 | CUGACCGC U CUCCACAG | 377 | CUGUGGAG CUGAUGAG X CGAA ICGGUCAG | 2152 |
| 284 | GACCGCUC U CCACAGCC | 378 | GGCUGUGG CUGAUGAG X CGAA IAGCGGUC | 2153 |
| 286 | CCGCUCUC C ACAGCCCG | 379 | CGGGCUGU CUGAUGAG X CGAA IAGAGCGG | 2154 |
| 287 | CGCUCUCC A CAGCCCGG | 380 | CCGGGCUG CUGAUGAG X CGAA IGAGAGCG | 2155 |
| 289 | CUCUCCAC A GCCCGGAC | 381 | GUCCGGGC CUGAUGAG X CGAA IUGGAGAG | 2156 |
| 292 | UCCACAGC C CGGACCCG | 382 | CGGGUCCG CUGAUGAG X CGAA ICUGUGGA | 2157 |
| 293 | CCACAGCC C GGACCCGG | 383 | CCGGGUCC CUGAUGAG X CGAA IGCUGUGG | 2158 |
| 298 | GCCCGGAC C CGGGGGCU | 384 | AGCCCCCG CUGAUGAG X CGAA IUCCGGGC | 2159 |
| 299 | CCCGGACC C GGGCCUUG | 385 | CAGCCCCC CUGAUGAG X CGAA IGUCCGGG | 2160 |
| 306 | CCGGGGGC U GGCCCAGG | 386 | CCUGGGCC CUGAUGAG X CGAA ICCCCCGG | 2161 |
| 310 | GGGCUGGC C CAGGGCCC | 387 | GGGCCCUG CUGAUGAG X CGAA ICCAGCCC | 2162 |
| 311 | GGCUGGCC C AGGGCCCU | 388 | AGGGCCCU CUGAUGAG X CGAA IGCCAGCC | 2163 |
| 312 | GCUGGUUU A GGGCCCUG | 389 | CAGGGCCC CUGAUGAG X CGAA IGGCCAGC | 2164 |
| 317 | CCCAGGGC C CUGCAGGC | 390 | GCCUGCAG CUGAUGAG X CGAA ICCCUGGG | 2165 |
| 318 | CCAGGGCC C UGCAGGCC | 391 | GGCCUGCA CUGAUGAG X CGAA IGCCCUGG | 2166 |
| 319 | CAGGGCCC U GCAGGCCC | 392 | GGGCCUGC CUGAUGAG X CGAA IGGCCCUG | 2167 |
| 322 | GGCCCUGC A GGCCCUGG | 393 | CCAGGGCC CUGAUGAG X CGAA ICAGGGCC | 2168 |
| 326 | CUGCAGGC C CUGGCGUC | 394 | CACGCCAG CUGAUGAG X CGAA ICCUGCAG | 2169 |
| 327 | UGCAGGCC C UGGCGUCC | 395 | GGACGCCA CUGAUGAG X CGAA IGCCUGCA | 2170 |
| 328 | GCAGGCCC U GGCGUCCU | 396 | AGGACGCC CUGAUGAG X CGAA IGGCCUGC | 2171 |
| 335 | CUGGCGUC C UGAUGCCC | 397 | GGGCAUCA CUGAUGAG X CGAA IACGCCAG | 2172 |
| 336 | UGGCGUCC U GAUGCCCC | 398 | GGGGCAUC CUGAUGAG X CGAA IGACGCCA | 2173 |
| 342 | CCUGAUGC C CCCAAGCU | 399 | AGCUUGGG CUGAUGAG X CGAA ICAUCAGG | 2174 |
| 343 | CUGAUGCC C CCAAGCUC | 400 | GAGCUUGG CUGAUGAG X CGAA IGCAUCAG | 2175 |
| 344 | UGAUGCCC C CAAGCUCC | 401 | GGAGCUUG CUGAUGAG X CGAA IGGCAUCA | 2176 |
| 345 | GAUGCCCC C AAGCUCCC | 402 | GGGAGCUU CUGAUGAG X CGAA IGGGCAUC | 2177 |
| 346 | AUGCCCCC A AGCUCCCU | 403 | AGGGAGCU CUGAUGAG X CGAA IGGGGCAU | 2178 |
| 350 | CCCCAAGC U CCCUCUCC | 404 | GGAGAGGG CUGAUGAG X CGAA ICUUGGGG | 2179 |
| 352 | CCAAGCUC C CUCUCCUG | 405 | CAGCAGAG CUGAUGAG X CGAA IAGCUUGG | 2180 |
| 353 | CAAGCUCC C UCUCCUGA | 406 | UCAGGAGA CUGAUGAG X CGAA IGAGCUUG | 2181 |
| 354 | AAGCUCCC U CUCCUGAG | 407 | CUCAGGAG CUGAUGAG X CGAA IGGAGCUU | 2182 |
| 356 | GCUCCCUC U CCUGAGAA | 408 | UUCUCAGG CUGAUGAG X CGAA IAGGGAGC | 2183 |
| 358 | UCCCUCUC C UGAGAAGC | 409 | GCUUCUCA CUGAUGAG X CGAA IAGAGGGA | 2184 |
| 359 | CCCUCUCC U GAGAAGCC | 410 | GGCUUCUC CUGAUGAG X CGAA IGAGAGGG | 2185 |
| 367 | UGAGAAGC C ACCAGCAG | 411 | GUGCUGGU CUGAUGAG X CGAA ICUUCUCA | 2186 |
| 368 | GAGAAGCC A CCAGCAGG | 412 | GGUGCUGG CUGAUGAG X CGAA IGCUUCUC | 2187 |
| 370 | GAAGCCAC C AGCACCAC | 413 | GUGGUGCU CUGAUGAG X CGAA IUGGCUUC | 2188 |
| 371 | AAGCCACC A GCACCACC | 414 | GGUGGUGC CUGAUGAG X CGAA IGUGGCUU | 2189 |
| 374 | CCACCAGC A CCACCCAG | 415 | CUGGGUGG CUGAUGAG X CGAA ICUGGUGG | 2190 |
| 376 | ACCAGCAC C ACCCAGAC | 416 | GUCUGGGU CUGAUGAG X CGAA IUGCUGGU | 2191 |
| 377 | CCAGCACC A CCCAGACU | 417 | AGUCUGGG CUGAUGAG X CGAA IGUGCUGG | 2192 |
| 379 | AGCACCAC C CAGACUUG | 418 | CAAGUCUG CUGAUGAG X CGAA IGUUUGCU | 2193 |
| 380 | GCACCACC C AGACUUGG | 419 | CCAAGUCU CUGAUGAG X CGAA IGUGGUGC | 2194 |
| 381 | CACCACCC A GACUUGGG | 420 | CCCAAGUC CUGAUGAG X CGAA IGGUGGUG | 2195 |
| 385 | ACCCAGAC U UGGGGGCA | 421 | UGCCCCCA CUGAUGAG X CGAA IUCUGGGU | 2196 |
| 393 | UUGGGGGC A GGCGCCAG | 422 | CUGGCGCC CUGAUGAG X CGAA ICCCCCAA | 2197 |
| 399 | GCAGGCGC C AGGGACGG | 423 | CCGUCCCU CUGAUGAG X CGAA ICGGGUGC | 2198 |
| 400 | CACGCGCC A GGGACGGA | 424 | UCCGUCCC CUGAUGAG X CGAA IGCGCCUG | 2199 |
| 416 | ACGUGGGC C AGUGCGAG | 425 | CUCGCACU CUGAUGAG X CGAA ICCCACGU | 2200 |
| 417 | CGUGGGCC A GUGCGAGC | 426 | GCUCGCAC CUGAUGAG X CGAA IGCCCACG | 2201 |
| 426 | GUGCGAGC C CAGAGGGC | 427 | GCCCUCUG CUGAUGAG X CGAA ICUCGCAC | 2202 |
| 427 | UGCGAGCC C AGAGGGCC | 428 | GGCCCUCU CUGAUGAG X CGAA IGCUCGCA | 2203 |
| 428 | GCGAGCCC A GAGGGCCC | 429 | GGGCCCUC CUGAUGAG X CGAA IGGCUCGC | 2204 |
| 435 | CAGAGGGC C CGAAGGCC | 430 | GGCCUUCG CUGAUGAG X CGAA IGGGUCUG | 2205 |
| 436 | AGAGGGCC C GAAGGCCG | 431 | CGGCCUUC CUGAUGAG X CGAA ICCCUCUG | 2206 |
| 443 | CCGAAGGC C GGGGCCCA | 432 | UGGGCCCC CUGAUGAG X CGAA ICCUUCGG | 2207 |
| 449 | GCCGGGGC C CACCAUGG | 433 | CCAUGGUG CUGAUGAG X CGAA ICCCCGGC | 2208 |
| 450 | CCGGGGCC C ACCAUGGC | 434 | GCCAUGGU CUGAUGAG X CGAA IGCCCCGG | 2209 |
| 451 | CGGGGCCC A CCAUGGCC | 435 | GGCCAUGG CUGAUGAG X CGAA IGGCCCCG | 2210 |
| 453 | GGGCCCAC C AUGGCCCA | 436 | UGGGCCAU CUGAUGAG X CAGG IUGGGCCC | 2211 |
| 454 | GGCCCACC A UGGCCCAA | 437 | UUGGGCCA CUGAUGAG X CGAA IGUGGGCC | 2212 |
| 459 | ACCAUGGC C CAAGCCCU | 438 | AGGGCUUG CUGAUGAG X CGAA ICCAUGGU | 2213 |
| 460 | CCAUGGCC C AAGCCCUG | 439 | CAGGGCUU CUGAUGAG X CGAA IGCCAUGG | 2214 |
| 461 | CAUGGCCC A AGCCCUGC | 440 | GCAGGGCU CUGAUGAG X CGAA IGGCCAUG | 2215 |
| 465 | GCCCAAGC C CUGCCCUG | 441 | CAGGGCAG CUGAUGAG X CGAA ICUUGGGC | 2216 |
| 466 | CCCAAGCC C UGCCCUGG | 442 | CCAGGGCA CUGAUGAG X CGAA IGCUUGGG | 2217 |
| 467 | CCAAGCCC U GCCCUGGC | 443 | GCCAGGGC CUGAUGAG X CGAA IGGCUUGG | 2218 |
| 470 | AGCCCUGC C CUGGCUCC | 444 | GGAGCCAG CUGAUGAG X CGAA ICAGGGCU | 2219 |
| 471 | GCCCUGCC C UGGCUCCU | 445 | AGGAGCCA CUGAUGAG X CGAA IGCAGGGC | 2220 |
| 472 | CCCUGCCC U GGCUCCUG | 446 | CAGGAGCC CUGAUGAG X CGAA IGGCAGGG | 2221 |
| 476 | GCCCUGGC U CCUGCUGU | 447 | ACAGCAGG CUGAUGAG X CGAA ICCAGGGC | 2222 |
| 478 | CCUGGCUC C UGCUGUGG | 448 | CCACAGCA CUGAUGAG X CGAA IAGCCAGG | 2223 |
| 479 | CUGGCUCC U GCUGUGGA | 449 | UCCACAGC CUGAUGAG X CGAA IGAGCCAG | 2224 |
| 482 | GCUCCUGC U GUCCAUGG | 450 | CCAUCCAC CUGAUGAG X CGAA ICAGGAGC | 2225 |
| 503 | GGGAGUGC U GCCUGCCC | 451 | GGGCAGGC CUGAUGAG X CGAA ICACUCCC | 2226 |
| 506 | AGUGCUGC C UGCCCACG | 452 | CGUGGGCA CUGAUGAG X CGAA ICAGCACU | 2227 |
| 507 | GUGCUGCC U GCCCACGG | 453 | CCGUGGGC CUGAUGAG X CGAA IGCAGCAC | 2228 |
| 510 | CUGCCUGC C CACGGCAC | 454 | GUGCCGUG CUGAUGAG X CGAA ICAGGCAG | 2229 |
| 511 | UGCCUGCC C ACGGCACC | 455 | GGUGCCGU CUGAUGAG X CGAA IGCAGGCA | 2230 |
| 512 | GCCUGCCC A CGGCACCC | 456 | GGGUGCCG CUGAUGAG X CGAA IGGCAGGC | 2231 |
| 517 | CCCACGGC A CCCAGCAC | 457 | GUGCUGGG CUGAUGAG X CGAA ICCGUGGG | 2232 |
| 519 | CACGGCAC C CAGCACGG | 458 | CCGUGCUG CUGAUGAG X CGAA IUGCCGUG | 2233 |
| 520 | ACGGCACC C AGCACGGC | 459 | GCCGUGCU CUGAUGAG X CGAA IGUGCCGU | 2234 |
| 521 | CCGCACCC A GCACGGCA | 460 | UGCCGUGC CUGAUGAG X CGAA IGGUGCCG | 2235 |
| 524 | CACCCAGC A CGGCAUCC | 461 | GGAUGCCG CUGAUGAG X CGAA ICUGGGUG | 2236 |
| 529 | AGCACGGC A UCCGGCUG | 462 | CAGCCGGA CUGAUGAG X CGAA ICCGUGCU | 2237 |
| 532 | ACGGCAUC C GGCUGCCC | 463 | GGGCAGCC CUGAUGAG X CGAA IAUGCCGU | 2238 |
| 536 | CAUCCGGC U CGGGGUGC | 464 | GCAGGGGC CUGAUGAG X CGAA ICCGGAUG | 2239 |
| 539 | CCGGCUGC C CCUGCGCA | 465 | UGCGCAGG CUGAUGAG X CGAA ICAGCCGG | 2240 |
| 540 | CGGCUGCC C CUGCGCAG | 466 | CUGCGCAG CUGAUGAG X CGAA IGCAGCCG | 2241 |
| 541 | GGCUGCCC C UGCGCAGC | 467 | GCUGCGCA CUGAUGAG X CGAA IGGCAGCC | 2242 |
| 542 | GCUGCCCC U GCGCAGCG | 468 | CGCUGCGC CUGAUGAG X CGAA IGGGCAGC | 2243 |
| 547 | CCCUGCGC A GCGGCCUG | 469 | CAGGCCGC CUGAUGAG X CGAA ICGCAGGG | 2244 |
| 553 | GCAGCGGC C UGGGGGGC | 470 | GCCCCCCA CUGAUGAG X CGAA ICCGCUGC | 2245 |
| 554 | CAGCGGCC U GGGGGGCG | 471 | CGCCCCCC CUGAUGAG X CGAA IGCCGCUG | 2246 |
| 564 | GGGGGCGC C CCCCUGGG | 472 | CCCAGGGG CUGAUGAG X CGAA ICGCCCCC | 2247 |
| 565 | GGGGCGCC C CCCUGGGG | 473 | CCCCAGGG CUGAUGAG X CGAA IGCGCCCC | 2248 |
| 566 | GGGCGCCC C CCUGGGGC | 474 | GCCCCAGG CUGAUGAG X CGAA IGGCGCCC | 2249 |
| 567 | GGCGCCCC C CUGGGGCU | 475 | AGCCCCAG CUGAUGAG X CGAA IGGGCGCC | 2250 |
| 568 | GCGCCCCC C UGGGGCUG | 476 | CAGCCCCA CUGAUGAG X CGAA IGGGGCGC | 2251 |
| 569 | CGCCCCCC U GGGGCUGC | 477 | GCAGCCCC CUGAUGAG X CGAA IGGGGGCG | 2252 |
| 575 | CCUGGGGC U GCGGCUGC | 478 | GCAGCCGC CUGAUGAG X CGAA ICCCCAGG | 2253 |
| 581 | GCUGCGGC U GCCCCGGG | 479 | CCCGGGGC CUGAUGAG X CGAA ICCGCAGC | 2254 |
| 584 | GCGGCUGC C CCGGGAGA | 480 | UCUCCCGG CUGAUGAG X CGAA ICAGCCGC | 2255 |
| 585 | CGGCUGCC C GCCCAGAC | 481 | GUCUCCCG CUGAUGAG X CGAA IGCAGCCG | 2256 |
| 586 | GGCUGCCC C GGGAGACC | 482 | GGUCUCCC CUGAUGAG X CGAA IGGCAGCC | 2257 |
| 594 | CGGGAGAC C GACGAAGA | 483 | UCUUCGUC CUGAUGAG X CGAA IUCUCCCG | 2258 |
| 605 | CGAAGACG C CGAGGAGC | 484 | GCUCCUCG CUGAUGAG X CGAA ICUCUUCG | 2259 |
| 606 | GAAGAGCC C GAGGAGCC | 485 | GGCUCCUC CUGAUGAG X CGAA IGCUCUUC | 2260 |
| 614 | CGAGGAGC C CGGCCGGA | 486 | UCCGGCCU CUGAUGAG X CGAA ICUCCUCG | 2261 |
| 615 | GAGGAGCC C GGCCGGAG | 487 | CUCCGGCC CUGAUGAG X CGAA IGCUCCUG | 2262 |
| 619 | AGCCCGGC C GGAGGGGC | 488 | GCCCCUCC CUGAUGAG X CGAA ICCGGGCU | 2263 |
| 628 | GGAGGGGC A GCUUUGUG | 489 | CACAAAGC CUGAUGAG X CGAA ICCCCUCC | 2264 |
| 631 | GGGGCAGC U UUGUGAAG | 490 | CUCCACAA CUGAUGAG X CGAA ICUGCCCC | 2265 |
| 649 | UGGUGGAC A ACCUGAGG | 491 | CCUCAGGU CUGAUGAG X CGAA IUCCACCA | 2266 |
| 652 | UGGACAAC C UGAGGGGC | 492 | GCCCCUCA CUGAUGAG X CGAA IUUGUCCA | 2267 |
| 653 | GGACAACC U GAGGGGCA | 493 | UGCCCCUC CUGAUGAG X CGAA IGUUGUCC | 2268 |
| 661 | UGAGGGGC A AGUCGGGG | 494 | CCCCGACU CUGAUGAG X CGAA ICCCCUCA | 2269 |
| 671 | GUCGGGGC A GGGCUACU | 495 | AGUAGCCC CUGAUGAG X CGAA ICCCCGAC | 2270 |
| 676 | GGCAGGGC U ACUACGUG | 496 | CACGUAGU CUGAUGAG X CGAA ICCCUGCC | 2271 |
| 679 | AGGGCUAC U ACGUGGAG | 497 | CUCCACGU CUGAUGAG X CGAA IUAGCCCU | 2272 |
| 693 | GAGAUGAC C GUGGGCAG | 498 | CUGCCCAC CUGAUGAG X CGAA IUCAUCUC | 2273 |
| 700 | CCGUGGGC A GCCCCCCG | 499 | CGGGGGGC CUGAUGAG X CGAA ICCCACGG | 2274 |
| 703 | GGGCAGCC C CCCGCAGA | 500 | CUGCGGGG CUGAUGAG X CGAA ICUGCCCA | 2275 |
| 704 | GGGCAGCC C CCCGCAGA | 501 | UCUGCGGG CUGAUGAG X CGAA IGCUGCCC | 2276 |
| 705 | GGCAGCCC C CCGCAGAC | 502 | GUCUGCGG CUGAUGAG X CGAA IGGCUGCC | 2277 |
| 706 | GCAGCCCC C CGCAGACG | 503 | CGUCUGCG CUGAUGAG X CGAA IGGGCUGC | 2278 |
| 707 | CAGCCCCC C GCAGACGA | 504 | GCGUCUGC CUGAUGAG X CGAA IGGGGCUG | 2279 |
| 710 | CCCCCCGC A GACGCUCA | 505 | UGAGCGUC CUGAUGAG X CGAA ICGGGGGG | 2280 |
| 716 | GCAGACGC U CAACAUCC | 506 | GGAUGUUG CUGAUGAG X CGAA ICGUCUGC | 2281 |
| 718 | AGACGCUC A ACAUGGUC | 507 | CAGGAUGU CUGAUGAG X CGAA IAGCUUCU | 2282 |
| 721 | CGCUCAAC A UCCUGGUG | 508 | CACCAGGA CUGAUGAG X CGAA IUUGAGCG | 2283 |
| 724 | UCAACAUC C UGGUGGAU | 509 | AUCCACCA CUGAUGAG X CGAA IAUGUUGA | 2284 |
| 725 | CAACAUCC U GGUGGAUA | 510 | UAUCCACC CUGAUGAG X CGAA IGAUGUUG | 2285 |
| 735 | GUGGAUAC A GGCAGGAG | 511 | CUGCUGCC CUGAUGAG X CGAA IUAUCCAC | 2286 |
| 739 | AUACAGGC A GCAGUAAC | 512 | GUUACUGC CUGAUGAG X CGAA ICCUGUAU | 2287 |
| 742 | CAGGCAGC A GUAACUUU | 513 | AAAGUUAC CUGAUGAG X CGAA ICUGCCUG | 2288 |
| 748 | GCAGUAAC U UUGCAGUG | 514 | CACUGCAA CUGAUGAG X CGAA IUUACUGC | 2289 |
| 753 | AACUUUGC A GUGGGUGC | 515 | GCACCCAC CUGAUGAG X CGAA ICAAAGUU | 2290 |
| 762 | GUGGGUGC U GCCCCCCA | 516 | UGGGGGGC CUGAUGAG X CGAA ICACCCAC | 2291 |
| 765 | GGUGCUGC C CCCCACCC | 517 | GGGUGGGG CUGAUGAG X CGAA ICAGCACC | 2292 |
| 766 | CUGCUGCC C CCCACCCC | 518 | GGGGUGGG CUGAUGGA X CGAA IGCAGCAC | 2293 |
| 767 | UGCUGCCC C CCACCCCU | 519 | AGGGGUGG CUGAIGAG X CGAA ICCGAGCA | 2294 |
| 768 | GCUGCCCC C CACCCCUU | 520 | AAGGGGUG CUGAUGAG X CGAA IGGGCAGC | 2295 |
| 769 | CUGCCCCC C ACCCCUUC | 521 | GAAGGGGU CUGAUGAG X CGAA IGGGGCAG | 2296 |
| 770 | UGCCCCCC A CCCCUUCC | 522 | GGAAGGGG CUGAUGAG X CGAA IGGGGGCA | 2297 |
| 772 | CCCCCCAC C CCUUCCUG | 523 | CAGGAAGG CUGAUGAG X CGAA IUGGGGGG | 2298 |
| 773 | CCCCCACC C CUUCCUGC | 524 | GCAGGAAC CUGAUGAG X CGAA IGUGGGGG | 2299 |
| 774 | CCCCACCC C UUCCUGCA | 525 | UGCAGGAA CUGAUGAG X CGAA IGGUGGGG | 2300 |
| 775 | CCCACCCC U UCCUGCAU | 526 | AUGCAGGA CUGAUGAG X CGAA IGGGUGGG | 2301 |
| 778 | ACCCCUUC C UGCAUCGC | 527 | GCGAUGCA CUGAUGAG C CGAA IAAGGGGU | 2302 |
| 779 | CCCCUUCC U GCAUCGCU | 528 | AGCGAUGC CUGAUGAG X CGAA IGAAGGGG | 2303 |
| 782 | CUUCCUGC A UCGCGACU | 529 | AGUAGCGA CUGAUGAG X CGAA ICAGGAAG | 2304 |
| 787 | UGCAUCGC U ACUACCAG | 530 | CUGGUAGU CUGAUGAG X CGAA ICGAUGCA | 2305 |
| 790 | AUGCCUAC U ACCAGAGG | 531 | CCUCUGGU CUGAUGAG X CGAA IUAGCGAU | 2306 |
| 793 | GCUACUAC C AGAGGCAG | 532 | CUGCCUCU CUGAUGAG X CGAA IUAGUAGC | 2307 |
| 794 | CUACUACC A GAGGCAGC | 533 | GCUGCCUG CUGAUGAG X CGAA IGUAGUAG | 2308 |
| 800 | CCAGAGGC A GCUGUCCA | 534 | UGGACAGC CUGAUGAG X CGAA ICCUCUGG | 2309 |
| 803 | GAGGCAGC U GUCCAGCA | 535 | UGCUGGAC CUGAUGAG X CGAA ICUGCCUC | 2310 |
| 807 | CAGCUGUC C AGCACAUA | 536 | UAUGUGCU CUGAUGAG X CGAA IACAGCUG | 2311 |
| 808 | AGCUGUCC A GACGAUAC | 537 | GUAUGUGC CUGAUGAG X CGAA IGACAGCU | 2312 |
| 811 | UGUCCAGC A CAUACCGG | 538 | CCGGUAUG CUGAUGAG X CGAA ICUGGACA | 2313 |
| 813 | UCCAGCAC A UACCGGGA | 539 | UCCCGGUA CUGAUGAG X CGAA IUGCUGGA | 2314 |
| 817 | GCACAUAC C GGGACCUC | 540 | CAGGUCCC CUGAUGAG X CGAA IUAUGUGC | 2315 |
| 823 | ACCGGGAC C UCCGGAAG | 541 | CUUCCGGA CUGAUGAG X CGAA IUCCCGGU | 2316 |
| 824 | CCGGGACC U CCGGAAGG | 542 | CCUUCCGG CUGAUGAG X CGAA IGUCCCGG | 2317 |
| 826 | GGGACCUC C GGAAGGGU | 543 | ACCCUUCC CUGAUGAG X CGAA IAGGUCCC | 2318 |
| 845 | GUAUGUGC C CUACACCC | 544 | GGGUGUAG CUGAUGAG X CGAA ICACAUAC | 2319 |
| 846 | UAUGUGCC C UACACCCA | 545 | UGGGUGUA CUGAUGAG X CGAA IGCACAUA | 2320 |
| 847 | AUGUGCCC U ACACCCAG | 546 | CUGGGUGU CUGAUGAG X CGAA IGGCACAU | 2321 |
| 850 | UGCCCUAC A CCCAGGGC | 547 | GCCCUGGG CUGAUGAG X CGAA IUAGGGCA | 2322 |
| 852 | CCCUACAC C CAGGGCAA | 548 | UUGCCCUG CUGAUGAG X CGAA IUGUAGGG | 2323 |
| 853 | CCUACACC C AGGGCAAG | 549 | CUUGCCCU CUGAUGAG X CGAA IGUGUAGG | 2324 |
| 854 | CUACACCC A GGGCAAGU | 550 | ACUUGCCC CUGAUGAG X CGAA IGGUGUAG | 2325 |
| 859 | CCCAGGGC A AGUGGGAA | 551 | UUCCCACU CUGAUGAG X CGAA ICCCUGGG | 2326 |
| 875 | AGGGGAGC U GGGCACCG | 552 | CGGUGCCC CUGAUGAG X CGAA ICUCCCCU | 2327 |
| 880 | AGCUGGGC A CCGACCUG | 553 | CAGGUCGG CUGAUGAG X CGAA ICCCAGCU | 2328 |
| 882 | CUGGGCAC C GACCUGGU | 554 | ACCAGGUC CUGAUGAG X CGAA IUGCCCAG | 2329 |
| 886 | GCACCGAC C UGGUAAGC | 555 | GCUUACCA CUGAUGAG X CGAA IUCGGUGC | 2330 |
| 887 | CACCGACC U GGUAAGCA | 556 | UGCUUACC CUGAUGAG X CGAA IGUCGGUG | 2331 |
| 895 | UGGUAAGC A UCCCCCAU | 557 | AUGGGGGA CUGAUGAG X CGAA ICUUACCA | 2332 |
| 898 | UAAGCAUC C CCCAUGGC | 558 | GCCAUGGG CUGAUGAG X CGAA IAUGCUUA | 2333 |
| 899 | AAGCAUCC C CCAUGGCC | 559 | GGCCAUGG CUGAUGAG X CGAA IGAUGCUU | 2334 |
| 900 | AGCAUCCC C CAUGGCCC | 560 | GGGCCAUG CUGAUGAG X CGAA IGGAUGCU | 2335 |
| 901 | GCAUCCCC C AUGGCCCC | 561 | GGGGCCAU CUGAUGAG X CGAA IGGGAUGC | 2336 |
| 902 | CAUCCCCC A UGGCCCCA | 562 | UGGGGCCA CUGAUGAG X CGAA IGGGGAUG | 2337 |
| 907 | CCCAUGGC C CCAACGUC | 563 | GACGUUGG CUGAUGAG X CGAA ICCAUGGG | 2338 |
| 908 | CCAUGGCC C CAACGUCA | 564 | UGACGUUG CUGAUGAG X CGAA IGCCAUGG | 2339 |
| 909 | CAUGGCCC C AACGUCAC | 565 | GUGACGUU CUGAUGAG X CGAA IGGCGAUG | 2340 |
| 910 | AUGGCCCC A ACGUCACU | 566 | AGUGAGCU CUGAUGAG X CGAA IGGGCCAU | 2341 |
| 916 | CCAACGUC A CUGUGCGU | 567 | ACGCACAG CUGAUGAG X CGAA IACGUUGG | 2342 |
| 918 | AACGUCAC U GUCGCUGC | 568 | GCACGCAC CUGAUGAG X CGAA IUGACGUU | 2343 |
| 927 | GUGCGUGC C AACAUUGC | 569 | GCAAUGUU CUGAUGAG X CGAA ICACGCAC | 2344 |
| 928 | UGCGUGCC A ACAUUGCU | 570 | AGCAAUGU CUGAUGAG X CGAA IGCACGCA | 2345 |
| 931 | GUGCCAAC A UUGCUGCC | 571 | GGCAGCAA CUGAUGAG X CGAA IUUGGCAC | 2346 |
| 936 | AACAUUGC U GCCAUCAC | 572 | GUGAUGGC CUGAUGAG X CGAA ICAAUGUU | 2347 |
| 939 | AUUGCUGC C AUCACUGA | 573 | UCAGUGAU CUGAUGAG X CGAA ICAGCAAU | 2348 |
| 940 | UUGCUGCC A UCACUGAA | 574 | UUCAGUGA CUGAUGAG X CGAA IGCAGCAA | 2349 |
| 943 | CUGCCAUC A CUGAAUCA | 575 | UGAUUCAG CUGAUGAG X CGAA IAUGGCAG | 2350 |
| 945 | GCCAUCAC U GAAUCAGA | 576 | UCUGAUUC CUGAUGAG X CGAA IUGAUGGC | 2351 |
| 951 | ACUGAAUC A GACAAGUU | 577 | AACUUGUC CUGAUGAG X CGAA IAUUCAGU | 2352 |
| 955 | AAUCAGAC A AGUUCUUC | 578 | GAAGAACU CUGAUGAG X CGAA IUCUGAUU | 2353 |
| 961 | ACAAGUUC U UCAUCAAC | 579 | GUUGAUGA CUGAUGAG X CGAA IAACUUGU | 2354 |
| 964 | AGUUCUUC A UCAACGGC | 580 | GCCGUUGA CUGAUGAG X CGAA IAAGAACU | 2355 |
| 967 | UCUUCAUC A ACGGCUCC | 581 | GGAGCCGU CUGAUGAG X CGAA IAUGAAGA | 2356 |
| 973 | UCAACGGC U CCAACUGG | 582 | CCAGUUGG CUGAUGAG X CGAA ICCGUUGA | 2357 |
| 975 | AACGGCUC C AACUGGGA | 583 | UCCCAGUU CUGAUGAG X CGAA IAGCCGUU | 2358 |
| 976 | ACGGCUCC A ACUGGGAA | 584 | UUCCCAGU CUGAUGAG X CGAA IGAGCCGU | 2359 |
| 979 | GCUCCAAC U GGGAAGGC | 585 | GCCUUCCC CUGAUGAG X CGAA IUUGGAGC | 2360 |
| 988 | GGGAAGGC A UCCUGGGG | 586 | CCCCAGGA CUGAUGAG X CGAA ICCUUCCC | 2361 |
| 991 | AAGGCAUC C UGGGGCUG | 587 | CAGCCCCA CUGAUGAG X CGAA IGAUGCCU | 2362 |
| 992 | AGGCAUCC U GGGGCUGG | 588 | CCAGCCCC CUGAUGAG X CGAA IGAUGCCU | 2363 |
| 998 | CCUGGGGC U GGCCUAUG | 589 | CAUGAGCC CUGAUGAG X CGAA ICCCCAGG | 2364 |
| 1002 | GGGCUGGC C UAUGCUGA | 590 | UCAGCAUA CUGAUGAG X CGAA ICCAGCCC | 2365 |
| 1003 | GGCUGGCC U AUGCUGAG | 591 | CUCAGCAU CUGAUGAG X CGAA ICGGAGCC | 2366 |
| 1008 | GCCUAUGC U GAGAUUGC | 592 | GCAAUCUC CUGAUGAG X CGAA ICAUAGGC | 2367 |
| 1017 | GAGAUUGC C AGGCCUGA | 593 | UCAGGCCU CUGAUGAG X CGAA ICAAUCUC | 2368 |
| 1018 | AGAUUGCC A GGCCUGAC | 594 | GUCAGGCC CUGAUGAG X CGAA IGCAAUCU | 2369 |
| 1022 | UGCCAGGC C UGACGACU | 595 | AGUCGUCA CUGAUGAG X CGAA ICCUGGCA | 2370 |
| 1023 | GCCAGGCC U GACGACUC | 596 | GAGUCGUC CUGAUGAG X CGAA IGCCUGGC | 2371 |
| 1030 | CUGACGAC U CCCUGGAG | 597 | CUGGAGGG CUGAUGAG X CGAA IUCGUCAG | 2372 |
| 1032 | GACGACUC U CUGGAGCC | 598 | GGCUCCAG CUGAUGAG X CGAA IAGUCGUC | 2373 |
| 1033 | ACGACUCC C UGGAGCCU | 599 | AGGCUCCA CUGAUGAG X CGAA IGAGUCGU | 2374 |
| 1034 | CGACUCCC U GGAGCCUU | 600 | AAGGCUCC CUGAUGAG X CGAA IGGAGUCG | 2375 |
| 1040 | CCUGGAGC C UUUCUUUG | 601 | CAAAGAAA CUGAUGAG X CGAA ICUCCAGG | 2376 |
| 1041 | CUGGACGG U UUCUUUGA | 602 | UCAAAGAA CUGAUGAG X CGAA IGCUCCAG | 2377 |
| 1045 | AGCCUUUC U UUGACUCU | 603 | AGAGUCAA CUGAUGAG X CGAA IAAAGGCU | 2378 |
| 1051 | UCUUUGAC U CUCUGGUA | 604 | UACCAGAG CUGAUGAG X CGAA IUCAAAGA | 2379 |
| 1053 | UUUGACUC U CUGGUAAA | 605 | UUUACCAG CUGAUGAG X CGAA IAGUCAAA | 2380 |
| 1055 | UGACUCUC U GGUAAAGC | 606 | GCUUUACC CUGAUGAG X CGAA IAGAGUCA | 2381 |
| 1064 | GGUAAAGC A GACCCACG | 607 | CGUGGGUC CUGAUGAG X CGAA ICUUUACC | 2382 |
| 1068 | AAGCAGAC C CACGUUCC | 608 | GGAACGUG CUGAUGAG X CGAA IUCUGCUU | 2383 |
| 1069 | AGCAGACC C ACGUUCCC | 609 | GGGAACGU CUGAUGAG X CGAA IGUCUGCU | 2384 |
| 1070 | GCAGACCC A CGUUCCCA | 610 | UGGGAACG CUGAUGAG X CGAA IGGUCUGC | 2385 |
| 1076 | CCACGUUC C CAACCUCU | 611 | AGAGGUUG CUGAUGAG X CGAA IAACGUGG | 2386 |
| 1077 | CACGUUCC C AACCUCUU | 612 | AAGAGGUU CUGAUGAG X CGAA IGAACGUG | 2387 |
| 1078 | ACGUUCCC A ACCUCUUC | 613 | GAAGAGGU CUGAUGAG X CGAA IGGAACGU | 2388 |
| 1081 | UUCCCAAC C UCUUCUCC | 614 | GGAGAAGA CUGAUGAG X CGAA IUUGGGAA | 2389 |
| 1082 | UCCCAACC U CUUCUCCC | 615 | GGGAGAAG CUGAUGAG X CGAA IGUUGGGA | 2390 |
| 1084 | CCAACCUC U UCUCCCUG | 616 | CAGGGAGA CUGAUGAG X CGAA IAGGUUGG | 2391 |
| 1087 | ACCUCUUC U CCCUGCAG | 617 | CUGCAGGG CUGAUGAG X CGAA IAAGAGGU | 2392 |
| 1089 | CUCUUCUC C CUGCAGCU | 618 | AGCUGCAG CUGAUGAG X CGAA IAGAAGAG | 2393 |
| 1090 | UCUUCUCC C UGCAGCUU | 619 | AAGCUGCA CUGAUGAG X CGAA IGAGAAGA | 2394 |
| 1091 | CUUCUCCC U GCAGCUUU | 620 | AAAGCUGC CUGAUGAG X CGAA IGGAGAAG | 2395 |
| 1094 | CUCCCUGC A GCUUUGUG | 621 | CACAAAGC CUGAUGAG X CGAA ICAGGGAG | 2396 |
| 1097 | CCUGCAGC U UUGUGGUG | 622 | CACCACAA CUGAUGAG X CGAA ICUGCAGG | 2397 |
| 1107 | UGUGGUGC U GGCUUCCC | 623 | GGGAAGCC CUGAUGAG X CGAA ICACCACA | 2398 |
| 1111 | GUGCUGGC U UCCCCCUC | 624 | GAGGGGGA CUGAUGAG X CGAA ICCAGCAC | 2399 |
| 1114 | CUGGCUUC C CCCUCAAC | 625 | GUUGAGGG CUGAGGAG X CGAA IAAGCCAG | 2400 |
| 1115 | UGGCUUCC C CCUCAACC | 626 | GGUUGAGG CUGAUGAG X CGAA IGAACGGA | 2401 |
| 1116 | GGCUUCCC C CUCAACCA | 627 | UGGUUGAG CUGAUGAG X CGAA IGGAAGCC | 2402 |
| 1117 | GCUUCCCC C UCAACCAG | 628 | CUGGUUGA CUGAUGAG X CGAA IGGGAAGC | 2403 |
| 1118 | CUUCCCCC U CAACCAGU | 629 | ACUGGUUG CUGAUGAG X CGAA IGGGGAAG | 2404 |
| 1120 | UCCCCCUC A ACCAGUCU | 630 | AGACUGGU CUGAUGAG X CGAA IAGGGGGA | 2405 |
| 1123 | CCCUCAAC C AGUCUGAA | 631 | UUCAGACU CUGAUGAG X CGAA IUUGAGGG | 2406 |
| 1124 | CCUCAACC A GUCUGAAG | 632 | CUUCAGAC CUGAUGAG X CGAA IGUUGAGG | 2407 |
| 1128 | AACCAGUC U GAAGUGCU | 633 | AGCACUUC CUGAUGAG X CGAA IACUGGUU | 2408 |
| 1136 | UGAAGUGC U GGCCUCUG | 634 | CAGAGGCC CUGAUGAG X CGAA ICACUUCA | 2409 |
| 1140 | GUGCUGGC C UCUGUCGG | 635 | CCGACAGA CUGAUGAG X CGAA ICCAGCAC | 2410 |
| 1141 | UGCUGGCC U CUGUCGGA | 636 | UCCGACAG CUGAUGAG X CGAA IGCCAGCA | 2411 |
| 1143 | GUGGCCUC U GUCGGAGG | 637 | CCUCCGAC CUGAUGAG X CGAA IAGGCCAG | 2412 |
| 1156 | GAGGGAGC A UGAUCAUU | 638 | AAUGAUCA CUGAUGAG X CGAA ICUCCCUC | 2413 |
| 1162 | GCAUGAUC A UUGGAGGU | 639 | ACCUCCAA CUGAUGAG X CGAA IAUGAUGC | 2414 |
| 1177 | GUAUCGAC C ACUCGCUG | 640 | CAGCGAGU CUGAGGAG X CGAA IUCGAUAC | 2415 |
| 1178 | UAUCGACC A CUCGCUGU | 641 | ACAGCGAG CUGAUGAG X CGAA IGUCGAUA | 2416 |
| 1180 | UCGACCAC U CGCUGUAC | 642 | GUACAGCG CUGAUGAG X CGAA IUGGUCGA | 2417 |
| 1184 | CCACUCGC U GUACACAG | 643 | CUGUGUAC CUGAUGAG X CGAA ICGAGUGG | 2418 |
| 1189 | CGCUGUAC A CAGGCAGU | 644 | ACUGCCUG CUGAUGAG X CGAA IUACAGCG | 2419 |
| 1191 | CUGUACAC A GGCAGUCU | 645 | AGACUGCC CUGAUGAG X CGAA IGUGACAG | 2420 |
| 1195 | ACACAGGC A GUCUCUGG | 646 | CCAGAGAC CUGAUGAG X CGAA ICCUGUGU | 2421 |
| 1199 | AGGCAGUC U CUGGUAUA | 647 | UAUACCAG CUGAUGAG X CGAA IACUGCCU | 2422 |
| 1201 | GCAGUCUC U GGUAUACA | 648 | UGUAUACC CUGAUGAG X CGAA IAGACUGC | 2423 |
| 1209 | UGGUAUAC A CCCAUCCG | 649 | CGGAUGGG CUGAUGAG X CGAA IUAUACCA | 2424 |
| 1211 | GUAUACAC C CAUCCGGC | 650 | GCCGGAUG CUGAUGAG X CGAA IUGUAUAC | 2425 |
| 1212 | UAUACACC C AUCCGGCG | 651 | CGCCGGAU CUGAUGAG X CGAA IGUGUAUA | 2426 |
| 1213 | AUACACCC A UCCGGCGG | 652 | CCGCCGGA CUGAUGAG X CGAA IGGUGUAU | 2427 |
| 1216 | CACCCAUC C GGCGGGAG | 653 | CUCCCGCC CUGAUGAG X CGAA IAUGGGUG | 2428 |
| 1243 | AGGUGAUC A UUGUGCGG | 654 | CCGCACAA CUGAUGAG X CGAA IAUCACCU | 2429 |
| 1261 | UGGAGAUC A AUGGACAG | 655 | CUGUCCAU CUGAUGAG X CGAA IAUCUCCA | 2430 |
| 1268 | CAAUGGAC A GGAUCUGA | 656 | UCAGAUCC CUGAUGAG X CGAA IUCCAUUG | 2431 |
| 1274 | ACAGGAUC U GAAAAUGG | 657 | CCAUUUUC CUGAUGAG X CGAA IAUCCUGU | 2432 |
| 1285 | AAAUGGAC U GCAAGGAG | 658 | CUCCUUGC CUGAUGAG X CGAA IUCCAUUU | 2433 |
| 1288 | UGGACUGC A AGGAGUAC | 659 | GUACUCCU CUGAUGAG X CGAA ICAGUCCA | 2434 |
| 1297 | AGGAGUAC A ACUAUGAC | 660 | GUCAUAGU CUGAUGAG X CGAA IUACUCCU | 2435 |
| 1300 | AGUACAAC U AUGACAAG | 661 | CUUGUCAU CUGAUGAG X CGAA IUUGUACU | 2436 |
| 1306 | ACUAUGAC A AGAGCAUU | 662 | AAUGCUCU CUGAUGAG X CGAA IUCAUAGU | 2437 |
| 1312 | ACAAGAGC A UUGUGGAC | 663 | GUCCACAA CUGAUGAG X CGAA ICUCUUGU | 2438 |
| 1321 | UUGUGGAC A GUGGCACC | 664 | GGUGCCAC CUGAGUGA X CGAA IUCCACAA | 2349 |
| 1327 | ACAGUGGC A CCACCAAC | 665 | GUUGGUGG CUGAUGAG X CGAA ICCACUGU | 2440 |
| 1329 | AGUGGCAC C ACCAACCU | 666 | AGGUUGGU CUGAUGAG X CGAA IUGCCACU | 2441 |
| 1330 | GUGGCACC A CCAACCUU | 667 | AAGGUUGG CUGAUGAG X CGAA IGUGCCAC | 2442 |
| 1332 | GGCACCAC C AACCUUCG | 668 | CGAAGGUU CUGAUGAG X CGAA IUGGUGCC | 2443 |
| 1333 | GCACCACC A ACCUUCGU | 669 | ACGAAGGU CUGAUGAG X CGAA IGUGGUGC | 2444 |
| 1336 | CCACCAAC C UUCGUUUG | 670 | CAAACGAA CUGAUGAG X CGAA IUUGGUGG | 2445 |
| 1337 | CACCAACC U UCGUUUGC | 671 | GCAAACGA CUGAUGAG X CGAA IGUUGGUG | 2446 |
| 1346 | UCGUUUGC C CAAGAAAG | 672 | CUUUCUUG CUGAUGAG X CGAA ICAAACGA | 2447 |
| 1347 | CGUUUGCC C AAGAAAGU | 673 | ACUUUCUU CUGAUGAG X CGAA IGCAAACG | 2448 |
| 1348 | GUUUGCCC A AGAAAGUG | 674 | CACUUUCU CUGAUGAG X CGAA IGGCAAAC | 2449 |
| 1365 | UUUGAAGC U CGAGUCCA | 675 | UUGACUGC CUGAUGAG X CGAA ICUUCAAA | 2450 |
| 1368 | GAAGCUGC A GUCAAAUC | 676 | GAUUUGAC CUGAUGAG X CGAA ICAGCUUC | 2451 |
| 1372 | CUGCAGUC A AAUCCAUC | 677 | GAGGGAUU CUGAUGAG X CGAA IACUGCAG | 2452 |
| 1377 | CUGAAAUC C AUCAAGGC | 678 | GCCUUGAU CUGAUGAG X CGAA IAUUUGAC | 2453 |
| 1378 | UCAAAUCC A UCAAGGCA | 679 | UGCCUUGA CUGAUGAG X CGAA IGAUUUGA | 2454 |
| 1381 | AAUCCAUC A AGGCAGCC | 680 | GGCUGCCU CUGAUGAG X CGAA IAUGGAUU | 2455 |
| 1386 | AUCAAGGC A GCCUGGUC | 681 | GAGGAGGC CUGAUGAG X CGAA ICCUUGAU | 2456 |
| 1389 | AAGGCAGC C UCUUCCAC | 682 | GUGGAGGA CUGAUGAG X CGAA ICUGCCUU | 2457 |
| 1390 | AGGCAGCC U CCUCCACG | 683 | CGUGGAGG CUGAUGAG X CGAA IGCUGCCU | 2458 |
| 1392 | GCAGCCUC C UCCACGGA | 684 | UCCGUGGA CUGAUGAG X CGAA IAGGCUGC | 2459 |
| 1393 | CAGCCUCC U CCACGGAG | 685 | CUCCGUGG CUGAUGAG X CGAA IGAGGCUG | 2460 |
| 1395 | GCCUCCUC C ACGGAGAA | 686 | UUCUCCGU CUGAUGAG X CGAA IAGGAGGC | 2461 |
| 1396 | CCUCCUCC A CGGAGAAG | 687 | CUUCUCCG CUGAUGAG X CGAA IGAGGAGG | 2462 |
| 1408 | AGAAGUUC C CUGAUGGU | 688 | ACCAUCAG CUGAUGAG X CGAA IAACUUCU | 2463 |
| 1409 | GAAGUUCC C UGAGGGUU | 689 | AACCAUCA CUGAUGAG X CGAA IGAACUUC | 2464 |
| 1410 | AAGUUCCC U GAUGGUUU | 690 | AAACCAUC CUGAUGAG X CGAA IGGAACUU | 2465 |
| 1420 | AUGGUUUC U GGCUAGGA | 691 | UCCUAGCC CUGAUGAG X CGAA IAAACCAU | 2466 |
| 1424 | UUUCUGGC U AGGAGAGC | 692 | GCUCUCCU CUGAUGAG X CGAA ICCAGAAA | 2467 |
| 1433 | AGGAGAGC A GCUGGUGU | 693 | ACACCAGC CUGAUGAG X CGAA ICUCUCCU | 2468 |
| 1436 | AGAGCAGC U GGUGUGCU | 694 | AGCACACC CUGAUGAG X CGAA ICUGCUCU | 2469 |
| 1444 | UGGUGUGC U GGCAAGCA | 695 | UGCUUGCC CUGAUGAG X CGAA ICACACCA | 2470 |
| 1448 | GUGCUGGC A AGCAGGCA | 696 | UGCCUGCU CUGAUGAG X CGAA ICCAGCAC | 2471 |
| 1452 | UGGCAAGC A GGCACCAC | 697 | GUGGUGCC CUGAUGAG X CGAA ICUUGCCA | 2472 |
| 1456 | AAGCAGGC A CCACCCCU | 698 | AGGGGUGG CUGAUGAG X CGAA ICCUGCUU | 2473 |
| 1458 | GCAGGCAC C ACCCCUUG | 699 | CAAGGGGU CUGAGUGA X CGAA IUGCCUGC | 2474 |
| 1459 | CAGGCACC A CCCCUUGG | 700 | CCAAGGGG CUGAUGAG X CGAA IGUGCCUG | 2475 |
| 1461 | GGCACCAC C CCUUGGAA | 701 | UUCCAAGG CUGAUGAG X CGAA IUGGUGCC | 2476 |
| 1462 | GCACCACC C CUUGGAAC | 702 | GUUCCAAG CUGAUGAG X CGAA IGUGGUGC | 2477 |
| 1463 | CACCACCC C UUGGAACA | 703 | UGUUCCAA CUGAUGAG X CGAA IGGUGGUG | 2478 |
| 1464 | ACCACCCC U UGGAACAU | 704 | AUGUUCCA CUGAUGAG X CGAA IGGGUGGU | 2479 |
| 1471 | CUUGGAAC A UUUUCCCA | 705 | UGGGAAAA CUGAUGAG X CGAA IUUCCAAG | 2480 |
| 1477 | ACAUUUUC C CAGUCAUC | 706 | GAGUACUG CUGAUGAG X CGAA IAAAAUGU | 2481 |
| 1478 | CAUUUUCC C AGUCAUCU | 707 | AGAUGACU CUGAUGAG X CGAA IGAAAAUG | 2482 |
| 1479 | AUUUUCCC A GUCAUCUC | 708 | GAGAUGAC CUGAUGAG X CGAA IGGAAAAU | 2483 |
| 1483 | UCCCAGUC A UCUCACUC | 709 | GAGUGAGA CUGAUGAG X CGAA IACUGGGA | 2484 |
| 1486 | CAGUCAUC U CACUCUAC | 710 | GUAGAGUG CUGAUGUG X CGAA IAUGACUG | 2485 |
| 1488 | GUCAUCUC A CUCUACCU | 711 | AGGUAGAG CUGAUGAG X CGAA IAGAUGAC | 2486 |
| 1490 | CAUCUCAC U CUACCUAA | 712 | UUAGGUAG CUGAUGAG X CGAA IUGAGUAG | 2487 |
| 1492 | UCUCACUC U ACCUAAUG | 713 | CAUUAGGU CUGAUGAG X CGAA IAGUGAGA | 2488 |
| 1495 | CACUCUAC C UAAUGGGU | 714 | ACCCAUUA CUGAUGAG X CGAA IUAGAGUG | 2489 |
| 1496 | ACUCUACC U AAUGGGUG | 715 | CACCCAUU CUGAUGAG X CGAA IGUAGAGU | 2490 |
| 1512 | GAGGUUAC C AACCAGUC | 716 | GACUGGUU CUGAUGAG X CGAA IUAACCUC | 2491 |
| 1513 | AGGUUACC A ACCAGUCC | 717 | GGACUGGU CUGAUGAG X CGAA IGUAACCU | 2492 |
| 1516 | UUACCAAC C AGUCCUUC | 718 | GAAGGACU CUGAUGAG X CGAA IUUGGUAA | 2493 |
| 1517 | UACCAACC A GUCCUUCC | 719 | GGAAGGAC CUGAUGAG X CGAA IGUUGGUA | 2494 |
| 1521 | AACCAGUC C UUCCGCAU | 720 | AUGCGGAA CUGAUGAG X CGAA IACUGGUU | 2495 |
| 1522 | ACCAGUCC U UCCGCAUC | 721 | GAUGCGGA CUGAUGAG X CGAA IGACUGGU | 2496 |
| 1525 | AGUCCUUC C GCAUCACC | 722 | GGUGAUGC CUGAUGAG X CGAA IAAGGACU | 2497 |
| 1528 | CCUUCCGC A UCACCACU | 723 | GAUGGUGA CUGAUGAG X CGAA ICGGAAGG | 2498 |
| 1531 | UCCGCAUC A CCAUCCUU | 724 | AAGGAUGG CUGAUGAG X CGAA IAUGCGGA | 2499 |
| 1533 | CGCAUCAC C AUCCUUCC | 725 | GGAAGGAU CUGAUGAG X CGAA IUGAUGCG | 2500 |
| 1534 | GCAUCACC A UCCUUCCG | 726 | CGGAAGGA CUGAUGAG X CGAA IGUGAUGC | 2501 |
| 1537 | UCACCAUC C UUCCGCAG | 727 | CUGCGGAA CUGAUGAG X CGAA IAUGGUGA | 2502 |
| 1538 | CACCAUCC U UCCGCAGC | 728 | CGUGCGGA CUGAUGAG X CGAA IGAUGGUG | 2503 |
| 1541 | CAUCCUUC C GCAGCAAU | 729 | AUUGCUGC CUGAUGAG X CGAA IAAGGAUG | 2504 |
| 1544 | CCUUCCGC A CGAAUACC | 730 | GGUAUUGC CUGAUGAG X CGAA ICGGAAGG | 2505 |
| 1547 | UCCGCAGC A AUACCUGC | 731 | GCAGGUAU CUGAUGAG X CGAA ICUGCGGA | 2506 |
| 1552 | AGCAAUAC C UGCGGCCA | 732 | UGGCCGCA CUGAUGAG X CGAA IUAUUGCU | 2507 |
| 1553 | GCAAUACC U GCGGCCAG | 733 | CUGGCCGC CUGAUGAG X CGAA IGUAUUGC | 2508 |
| 1559 | CCUGCGGC C AGUGGAAG | 734 | CUUCCACU CUGAUGAG X CGAA ICCGCAGG | 2509 |
| 1560 | CUGCGGCC A GUGGAAGA | 735 | UCUUCCAC CUGAUGAG X CGAA IGCCGCAG | 2510 |
| 1575 | GAUGUGGC C ACGUCCCA | 736 | UGGGACGU CUGAUGAG X CGAA ICCACAUC | 2511 |
| 1576 | AUGUGGCC A CGUCCCAA | 737 | UUGGGACG CUGAUGAG X CGAA IGCCACAU | 2512 |
| 1581 | GCCACGUC C CAAGACGA | 738 | UCGUCUUG CUGAUGAG X CGAA IACGUGGC | 2513 |
| 1582 | CCACGUCC C AAGACGAC | 739 | GUCGUCUU CUGAUGAG X CGAA IGACGUGG | 2514 |
| 1583 | CACGUCCC A AGACGACU | 740 | AGUCGUCU CUGAUGAG X CGAA IGGACGUG | 2515 |
| 1591 | AAGACGAC U GUUACAAG | 741 | CUUGUAAC CUGAUGAG X CGAA IUCGUCUU | 2516 |
| 1597 | ACUGUUAC A AGUUUGCC | 742 | GGCAAACU CUGAUGAG X CGAA IUAACAGU | 2517 |
| 1605 | AAGUUUGC C AUCUCACA | 743 | UGUGAGAU CUGAUGAG X CGAA ICAAACUU | 2518 |
| 1606 | AGUUUGCC A CUUCACAG | 744 | CUGUCAGA CUGAUGAG X CGAA IGCAAACU | 2519 |
| 1609 | UUGCCAUC U CACAGUCA | 745 | UGACUGUC CUGAUGAG X CGAA IAUGGCAA | 2520 |
| 1611 | GCCAUCUC A CAGUCAUC | 746 | GAUGACUG CUGAUGAG X CGAA IAGAUGGC | 2521 |
| 1613 | CAUCUCAC A GUCAUCCA | 747 | UGGAUGAC CUGAUGAG X CGAA IUGAGAUG | 2522 |
| 1617 | UCACAGUC A UCCACGGG | 748 | CCCGUGGA CUGAUGAG X CGAA IACUGUGA | 2523 |
| 1620 | CAGUCAUC C ACGGGCAC | 749 | GUGCCCGU CUGAUGAG X CGAA IAUGACUG | 2524 |
| 1621 | AGUCAUCC A CGGGCACU | 750 | AGUGCCCG CUGAUGAG X CGAA IGAUGACU | 2525 |
| 1627 | CCACGGGC A CUGUUAUG | 751 | CAUAACAG CUGAUGAG X CGAA ICCCGUGG | 2526 |
| 1629 | ACGGGCAC U GUUAUGGG | 752 | CCCAUAAC CUGAUGAG X CGAA IUGCCCGU | 2527 |
| 1641 | AUGGGAGC U GUUAUCAU | 753 | AUGAUAAC CUGAUGAG X CGAA ICUCCCAU | 2528 |
| 1648 | CUGUUAUC A UGGAGGGC | 754 | GCCCUCCA CUGAUGAG X CGAA IAUAACAG | 2529 |
| 1657 | UGGAGGGC U UCUACGUU | 755 | AACGUAGA CUGAUGAG X CGAA ICCCUCCA | 2530 |
| 1660 | AGGGCUUC U ACGUUGUC | 756 | GACAACGU CUGAUGAG X CGAA IAAGCCCU | 2531 |
| 1669 | ACGUUGUC U UUGAUCGG | 757 | CCGAUCAA CUGAUGAG X CGAA IACAACGU | 2532 |
| 1680 | GAGCGGGC C CGAAAACG | 758 | CGUUUUCG CUGAUGAG X CGAA ICCCGAUC | 2533 |
| 1681 | AUCGGGCC C GAAAACGA | 759 | UCGUUUUC CUGAUGAG X CGAA IGCCCGAU | 2534 |
| 1696 | GAAUUGGC U UUGCUGUC | 760 | GACAGCAA CUGAUGAG X CGAA ICCAAUUC | 2535 |
| 1701 | GGCUUUGC U GUCAGCGC | 761 | GCGCUGAC CUGAUGAG X CGAA ICAAAGCC | 2536 |
| 1705 | UUGCUGUC A GCGCUUGC | 762 | GCAAGCGC CUGAUGAG X CGAA IACAGCAA | 2537 |
| 1710 | GUCAGCGC U UGCCAUGU | 763 | ACAUGGCA CUGAUGAG X CGAA ICGCUGAC | 2538 |
| 1714 | GCGCUUGC C AUGUGCAC | 764 | GUGCACAU CUGAUGAG X CGAA ICAAGCGC | 2539 |
| 1715 | CGCUUGCC A UGUGCACG | 765 | CGUGCACA CUGAUGAG X CGAA IGCAAGCG | 2540 |
| 1721 | CCAUGUGC A CGAUGAGU | 766 | ACUCAUCG CUGAUGAG X CGAA ICACAUGG | 2541 |
| 1732 | AUGAGUUC A GGACGGCA | 767 | UGCCGUCC CUGAUGAG X CGAA IAACUCAU | 2542 |
| 1740 | AGGACGGC A GCGGUGGA | 768 | UCCACCGC CUGAUGAG X CGAA ICCGUCCU | 2543 |
| 1753 | UGGAAGGC C CUUUUGUC | 769 | GACAAAAG CUGAUGAG X CGAA ICCUUCCA | 2544 |
| 1754 | GGAAGGCC C UUUUGUCA | 770 | UGACAAAA CUGAUGAG X CGAA IGCCUUCC | 2545 |
| 1755 | GAAGGCCC U UUUGUCAC | 771 | GUGACAAA CUGAUGAG X CGAA IGGCCUUC | 2546 |
| 1762 | CUUUUGUC A CCUUGGAC | 772 | GUCCAAGG CUGAUGAG X CGAA IACAAAAG | 2547 |
| 1764 | UUUGUCAC C UUGGACAU | 773 | AUGUCCAA CUGAUGAG X CGAA IUGACAAA | 2548 |
| 1765 | UUGUCACC U UGGACAUG | 774 | CAUGUCCA CUGAUGAG X CGAA IGUGACAA | 2549 |
| 1771 | CCUUGGAC A UGGAAGAC | 775 | GUCUUCCA CUGAUGAG X CGAA IUCCAAGG | 2550 |
| 1780 | UGGAAGAC U CUGGCUAC | 776 | GUAGCCAC CUGAUGAG X CGAA IUCUUCCA | 2551 |
| 1786 | ACUGUGGC U ACAACAUU | 777 | AAUGUUGU CUGAUGAG X CGAA ICCACAGU | 2552 |
| 1789 | GUGGCUAC A ACAUUCCA | 778 | UGGAAUGU CUGAUGAG X CGAA IUGACCAC | 2553 |
| 1792 | GCUACAAC A UUCCACAG | 779 | CUGUGGAA CUGAUGAG X CGAA IUUGUAGC | 2554 |
| 1796 | CAACAUUC C ACAGACAG | 780 | CUGUCUGU CUGAUGAG X CGAA IAAUGUUG | 2555 |
| 1797 | AACAUUCC A CAGACAGA | 781 | UCUGUCUG CUGAUGAG X CGAA IGAAUGUU | 2556 |
| 1799 | CAUUCCAC A GACAGAUG | 782 | CAUCUGUC CUGAUGAG X CGAA IUGGAAUG | 2557 |
| 1803 | CCACAGAC A GAUGAGUC | 783 | GACUCAUC CUGAUGAG X CGAA IUCUGUGG | 2558 |
| 1812 | GAUGAUGC A ACCCUCAU | 784 | AUGAGGGU CUGAUGAG X CGAA IACUCAUC | 2559 |
| 1815 | GAGUCAAC C CUCAUGAC | 785 | GUCAUGAG CUGAUGAG X CGAA IUUGACUC | 2560 |
| 1816 | AGUCAACC C UCAUGACC | 786 | GGUCAUGA CUGAUGAG X CGAA IGUUGACU | 2561 |
| 1817 | GUCAACCC U CAUGACCA | 787 | UGGUCAUG CUGAUGAG X CGAA IGGUUGAC | 2562 |
| 1819 | CAACCCUC A UGACCAUA | 788 | UAUGGUCA CUGAUGAG X CGAA IAGGGUUG | 2563 |
| 1824 | CUCAUGAC C AUAGCCUA | 789 | UAGGCUAU CUGAUGAG X CGAA IUCAUGAG | 2564 |
| 1825 | UCAUGAXX A UAGCCUAU | 790 | AUAGGCUA CUGAUGAG X CGAA IGUCAUGA | 2565 |
| 1830 | ACCAUAGC C UAUGUCAU | 791 | AUGACAUA CUGAUGAG X CGAA ICUAUGGU | 2566 |
| 1831 | CCAUAGCC U AUGUCAUG | 792 | CAUGACAU CUGAUGAG X CGAA IGUCAUGG | 2567 |
| 1837 | CCUAUGUC A UGGCUGCC | 793 | GGCAGCCA CUGAUGAG X CGAA IACAUAGG | 2568 |
| 1842 | GUCAUGGC U GCCAUCUG | 794 | CAGAUGGC CUGAUGAG X CGAA ICCAUGAC | 2569 |
| 1845 | AUGGCUGC C AUCUGCCG | 795 | GCGCAGAU CUGAUGAG X CGAA ICAGCCAU | 2570 |
| 1846 | UGGCUGCC A UCUGCGCC | 796 | GGCGCAGA CUGAUGAG X CGAA IGCAGCCA | 2571 |
| 1849 | CUGCCAUC U GCGCCCUC | 797 | GAGGGCGC CUGAUGAG X CGAA IAUGGCAG | 2572 |
| 1854 | AUCUGCGC C CUCUUCAU | 798 | AUGAAGAG CUGAUGAG X CGAA ICGCAGAU | 2573 |
| 1855 | UCUGCGCC C UCUUCAUG | 799 | CAUGAAGA CUGAUGAG X CGAA IGCGCAGA | 2574 |
| 1856 | CUGCGCCC U CUUCAUGC | 800 | GCAUGAAG CUGAUGAG X CGAA IGGCGCAG | 2575 |
| 1858 | GCGCCCUC U UCAUGCUG | 801 | CAGCAUGA CUGAUGAG X CGAA IAGGGCGC | 2576 |
| 1861 | CCCUCUUC A UGCUGCCA | 802 | UGGCAGCA CUGAUGAG X CGAA IAAGAGGG | 2577 |
| 1865 | CUUCAUGC U CGGACUCU | 803 | AGAGUGGC CUGAUGAG X CGAA ICAUGAAG | 2578 |
| 1868 | CAUGCUGC C ACUCUGCC | 804 | GGCAGAGU CUGAUGAG X CGAA ICAGCAUG | 2579 |
| 1869 | AUGCUGCC A CUCUGCCU | 805 | AGGCAGAG CUGAUGAG X CGAA ICGAGCAU | 2580 |
| 1871 | GCUGCCAC U CUGCCUCA | 806 | UGAGGCAG CUGAUGAG X CGAA IUGGCAGC | 2581 |
| 1873 | UGCCACUC U GCCUCAUG | 807 | CAUGAGGC CUGAUGAG X CGAA IAGUGGCA | 2582 |
| 1876 | CACUCUGC C UCAUGGUG | 808 | CACCAUGA CUGAUGAG X CGAA ICAGAGUG | 2583 |
| 1877 | ACUCUGCC U CAUGGUGU | 809 | ACACCAUG CUGAUGAG X CGAA ICGAGAUG | 2584 |
| 1897 | UCUGCCUC A UGGUGUGU | 810 | ACACACCA CUGAUGAG X CGAA IAGGCAGA | 2585 |
| 1889 | GGUGUGUC A CUGGCGCU | 811 | AGCGCCAC CUGAUGAG X CGAA IACACACC | 2586 |
| 1897 | AGUGGCGC U GCCUCCGC | 812 | GCGGAGGC CUGAUGAG X CGAA ICGCCACU | 2587 |
| 1900 | GGCGCUGC C UCCGCUGC | 813 | GCAGCGGA CUGAUGAG X CGAA ICAGCGCC | 2588 |
| 1901 | GCGCUGCC U CCGCUGCC | 814 | GGCAGCGG CUGAUGAG X CGAA IGCAGCGC | 2589 |
| 1903 | GCUGCCUC C GCUGCCUG | 815 | CAGGCAGC CUGAUGAG X CGAA IAGGCAGC | 2590 |
| 1906 | GCCUCCGC U GCCUGCGC | 816 | GCGCAGGC CUGAUGAG X CGAA ICGGAGGC | 2591 |
| 1909 | UCCGCUGC C UGCGCCAG | 817 | CUGGCGCA CUGAUGAG X CGAA ICAGCGGA | 2592 |
| 1910 | CCGCUGCC U GCGCCAGC | 818 | GCUGGCGC CUGAUGAG X CGAA IGCAGCGG | 2593 |
| 1915 | GCCUGCGC C AGCAGCAU | 819 | AUGCUGCU CUGAUGAG X CGAA ICGCAGGC | 2594 |
| 1916 | CCUGCGCC A GCAGCAUG | 820 | CAUGCUGC CUGAUGAG X CGAA IGCGCAGG | 2595 |
| 1919 | GCGCCAGC A GCAUGAUG | 821 | CAUCAUGC CUGAUGAG X CGAA ICUGGCGC | 2596 |
| 1922 | CCAGCAGC A UGAUGACU | 822 | AGUCAUCA CUGAUGAG X CGAA ICUGCUGG | 2597 |
| 1930 | AUGAUGAC A UUGCUGAU | 823 | AUCAGCAA CUGAUGAG X CGAA IUCAUCAU | 2598 |
| 1935 | GACUUUGC U GAUGACUA | 824 | AUGUCAUC CUGAUGAG X CGAA ICAAAGUC | 2599 |
| 1942 | CUGAUGAC A UCUCCCUG | 825 | CAGGGAGA CUGAUGAG X CGAA IUCAUCAG | 2600 |
| 1945 | AUGACAUC U CCCUGCUG | 826 | CAGCAGGG CUGAUGAG X CGAA IAUGUCAU | 2601 |
| 1947 | GACAUCUC C CUGCUGAA | 827 | UUCAGCAG CUGAUGAG X CGAA IAGAUCUG | 2602 |
| 1948 | ACAUCUCC C UGCUGAAG | 828 | CUUCAGCA CUGAUGAG X CGAA IGAGAUGU | 2603 |
| 1949 | CAUCUCCC U GCUGAAGU | 829 | ACUUCAGC CUGAUGAG X CGAA IGGAGUAG | 2604 |
| 1952 | CUCCCUGC U GAAGUGAG | 830 | CUCACUUC CUGAUGAG X CGAA ICAGGGAG | 2605 |
| 1966 | GAGGAGGC C CAUGGGCA | 831 | UGCCCAUG CUGAUGAG X CGAA ICCUCCUC | 2606 |
| 1967 | AGGAGGCC C AUGGGCAG | 832 | CUGCCCAU CUGAUGAG X CGAA IGCCUCCU | 2607 |
| 1968 | GGAGGCCC A UGGGCAGA | 833 | UCUGCCCA CUGAUGAG X CGAA IGGCCUCC | 2608 |
| 1974 | CCAUGGGC A GAAGAUAG | 834 | CUAUCUUC CUGAUGAG X CGAA ICCCAUGG | 2609 |
| 1989 | AGAGAUUC C CCUGGACC | 835 | GGUCCAGG CUGAUGAG X CGAA IAAUCUCU | 2610 |
| 1990 | GAGAUUCC C CUGGACCA | 836 | UGGUCCAG CUGAUGAG X CGAA IGAAUCUC | 2611 |
| 1991 | AGAUUCCC C UGGACCAC | 837 | GUGGUCCA CUGAUGAG X CGAA IGGAAUCU | 2612 |
| 1992 | GAUUCCCC U GGACCACA | 838 | UGUGGUCC CUGAUGAG X CGAA IGGGAAUC | 2613 |
| 1997 | CCCUGGAC C ACACCUCC | 839 | GGAGGUGU CUGAUGAG X CGAA IUCCAGGG | 2614 |
| 1998 | CCUGGACC A CACCUCCG | 840 | CGGAGGUC CUGAUGAG X CGAA IGUCCAGG | 2615 |
| 2000 | UGGACCAC A CCUCCGUG | 841 | CACGGAGG CUGAUGAG X CGAA IUGGUCCA | 2616 |
| 2002 | GACCACAC C UCCGUGGU | 842 | AACCACGG CUGAUGAG X CGAA IUGUGGUC | 2617 |
| 2003 | ACCACACC U CCGUGGUU | 843 | AACCACGG CUGAUGAG X CGAA IGUGUGGU | 2618 |
| 2005 | CACACCUC C GUGGUUCA | 844 | UGAACCAC CUGAUGAG X CGAA IAGGUGUG | 2619 |
| 2013 | CGUGGUUC A CUUUGGUC | 845 | GACCAAAG CUGAUGAG X CGAA IAACCACG | 2620 |
| 2015 | UGGUUCAC U UUGGUCAC | 846 | GUGACCAA CUGAUGAG X CGAA IUGAACCA | 2621 |
| 2022 | CUUUGGUC A CAAGUAGG | 847 | CCUACUUG CUGAUGAG X CGAA IACCAAAG | 2622 |
| 2024 | UUGGUCAC A AGUAGGAG | 848 | CUCCAUCU CUGAUGAG X CGAA IUGACCAA | 2623 |
| 2035 | UAGGAGAC A CAGAUGGC | 849 | GCCAUCUG CUGAUGAG X CGAA IUCUCCUA | 2624 |
| 2037 | GGAGACAC A GAUGGCAC | 850 | GUGCCAUC CUGAUGAG X CGAA IUGUCUCC | 2625 |
| 2044 | CAGAUGGC A CCUGUGGC | 851 | GCCAGAGG CUGAUGAG X CGAA ICCAUCUG | 2626 |
| 2046 | GAUGGCAC C UGUGGCCA | 852 | UGGCCACA CUGAUGAG X CGAA IUGCCAUC | 2627 |
| 2047 | AUGGCACC U GUGGCCAG | 853 | CUGGCCAC CUGAUGAG X CGAA IGUGCCAU | 2628 |
| 2053 | CCUGUGGC C AGAGCACC | 854 | CCUGCUCU CUGAUGAG X CGAA ICCACAGG | 2629 |
| 2054 | CUGUGGCC A GAGCACCU | 855 | AGGUGCUG CUGAUGAG X CGAA IGCCACAG | 2630 |
| 2059 | GCCAGAGC A CCUCAGGA | 856 | UCCUGAGG CUGAUGAG X CGAA ICUCUGGC | 2631 |
| 2061 | CAGAGCAC C UCAGGACC | 857 | GGUCCUGA CUGAUGAG X CGAA IUGCUCUG | 2632 |
| 2062 | AGAGCACC U CAGGACCC | 858 | GGGUCCUG CUGAUGAG X CGAA IGUGCUCU | 2633 |
| 2064 | AGCACCUC A GGACCCUC | 859 | GAGGGUCC CUGAUGAG X CGAA IAGGUGCU | 2634 |
| 2069 | CUCAGGAC C CUCCCCAC | 860 | GUGGGGAG CUGAUGAG X CGAA IUCCUGAG | 2635 |
| 2070 | UCAGGACC C UCCCCACC | 861 | GGUGGGGA CUGAUGAG X CGAA IGUCCUGA | 2636 |
| 2071 | CAGGACCC U CCCCACCC | 862 | GGGUGGGG CUGAUGAG X CGAA IGGUCCUG | 2637 |
| 2073 | GGAGGGUC C CCACCCAC | 863 | GUGGGUGG CUGAUGAG X CGAA IAGGGUCC | 2638 |
| 2074 | GACCCUCC C CACCCACC | 864 | GGUGGGUC CUGAUGAG X CGAA IGAGGGUC | 2639 |
| 2075 | ACCCUCCC C ACCCACCA | 865 | UGGUGGGU CUGAUGAG X CGAA IGGAGGGU | 2640 |
| 2076 | CCCUCCCC A CCCACCAA | 866 | UUGGUGGG CUGAUGAG X CGAA IGGGAGGG | 2641 |
| 2078 | CUCCCCAC C CACCAAAU | 867 | AUUUGGUG CUGAUGAG X CGAA IUGGGGAG | 2642 |
| 2079 | UCCCCACC C ACCAAAUG | 868 | CAUUUGGU CUGAUGAG X CGAA IGUGGGGA | 2643 |
| 2080 | CCCCACCC A CCAAAUGC | 869 | GCAUUUGG CUGAUGAG X CGAA IGGUGGGG | 2644 |
| 2082 | CCACCCAC C AAAUGCCU | 870 | AGGCAUUU CUGAUGAG X CGAA IUGGGUGG | 2645 |
| 2083 | CACCCACC A AAUGCCUG | 871 | CAGGCAUU CUGAUGAG X CGAA IGUGGGUG | 2646 |
| 2089 | CCAAAUGC C UCUGCCUU | 872 | AAGGCAGA CUGAUGAG X CGAA ICAUUUGG | 2647 |
| 2090 | CAAAUGCC U CUGCCUUG | 873 | CAAGGCAG CUGAUGAG X CGAA IGCAUUUG | 2648 |
| 2092 | AAUGCCUG U GCCUUGAU | 874 | AUCAAGGC CUGAUGAG X CGAA IAGGCAUU | 2649 |
| 2095 | GCCUCUGC C UUGAUGGA | 875 | UCCAUCCA CUGAUGAG X CGAA ICAGAGGC | 2650 |
| 2096 | CCUCUGCC U UGAUGGAG | 876 | CUCCAUCA CUGAUGAG X CGAA IGCAGAGG | 2651 |
| 2116 | GAAAAGGC U GGCAAGGU | 877 | ACCUUGCC CUGAUGAG X CGAA ICCUUUUC | 2652 |
| 2120 | AGGCUGGC A AGGUGGGU | 878 | ACCCACCU CUGAUGAG X CGAA ICCAGCCU | 2653 |
| 2131 | GUGGGUUC C AGGGACUG | 879 | CAGUCCCU CUGAUGAG X CGAA IAACCCAC | 2654 |
| 2132 | UGGGUUCC A GGGACUGU | 880 | ACAGUCCC CUGAUGAG X CGAA IGAACCCA | 2655 |
| 2138 | CCAGGGAC U GUACCUGU | 881 | ACAGGUAC CUGAUGAG X CGAA IUCCCUGG | 2656 |
| 2143 | GACUGUAC C UGUAGGAA | 882 | UUCCUACA CUGAUGAG X CGAA IUACAGUC | 2657 |
| 2144 | ACUGUACC U GUAGGAAA | 883 | UUUCCUAC CUGAUGAG X CGAA IGUACAGU | 2658 |
| 2154 | UAGGAAAC A GAAAAGAG | 884 | CUCUUUUC CUGAUGAG X CGAA IUUUCCUA | 2659 |
| 2174 | AAAGAAGC A CUCUGCUG | 885 | CAGCAGAG CUGAUGAG X CGAA ICUUCUUU | 2660 |
| 2176 | AGAAGCAC U CUGCUGGC | 886 | GCCAGCAG CUGAUGAG X CGAA IUGCUUCU | 2661 |
| 2178 | AAGCACUC U GUCGGCGG | 887 | CCGCCAGC CUGAUGAG X CGAA IAGUGCUU | 2662 |
| 2181 | CACUCUGC U CUUGGUCA | 888 | UUCCCGCC CUGAUGAG X CGAA ICAGAGUG | 2663 |
| 2193 | GGGAAUAC U CUUGGUCA | 889 | UGACCAAG CUGAUGAG X CGAA IUAUUCCC | 2664 |
| 2195 | GAAUACUC U UGGUCACC | 890 | GGUGACCA CUGAUGAG X CGAA IAGUAUUC | 2665 |
| 2201 | UCUUGGUC A CCUGAAAU | 891 | AUUUGAGG CUGAUGAG X CGAA IACCAAGA | 2666 |
| 2203 | UUGGUCAC C UCAAAUUU | 892 | AAAUUUGA CUGAUGAG X CGAA IUGACCAA | 2667 |
| 2204 | UGGUCACC U CAAAUUUA | 893 | UAAAUUUG CUGAUGAG X CGAA IGUGACCA | 2668 |
| 2206 | GUCACCUC A AAUUUAAG | 894 | CUUAAAUU CUGAUGAG X CGAA IAGGUGAC | 2669 |
| 2226 | GGAAAUUC U GCUGCUUG | 895 | CAAGCAGC CUGAUGAG X CGAA IAAUUUCC | 2670 |
| 2229 | AAUUCUGC U GCUUGAAA | 896 | UUUCAAGC CUGAUGAG X CGAA ICAGAAUU | 2671 |
| 2232 | UCUGCUGC U UGAAACUU | 897 | AAGUUUCA CUGAUGAG X CGAA ICAGCAGA | 2672 |
| 2239 | CUUGAAAC U UCAGCCCU | 898 | AGGGCUGA CUGAUGAG X CGAA IUUUCAAG | 2673 |
| 2242 | GAAACUUC A GCCCUGAA | 899 | UUCAGGGC CUGAUGAG X CGAA IAAGUUUC | 2674 |
| 2245 | ACUUCAGC C CUGAACCU | 900 | AGGUUCAG CUGAUGAG X CGAA ICUGAAGU | 2675 |
| 2246 | CUUCAGCC C UGAACCUU | 901 | AAGGUUCA CUGAGUGA X CGAA IGCUGAAG | 2676 |
| 2247 | UUCAGCCC U GAACCUUU | 902 | AAAGGUUC CUGAUGAG X CGAA IGGCUGAA | 2677 |
| 2252 | CCCUGAAC C UUUGUCCA | 903 | UGGACAAA CUGAUGAG X CGAA IUUGAGGG | 2678 |
| 2253 | CCUGAACC U UUGUCCAC | 904 | GUGGACAA CUGAUGAG X CGAA IGUUCAGG | 2679 |
| 2259 | CCUUUGUC C ACCAUUCC | 905 | GGAAUGGU CUGAUGAG X CGAA IACAAAGG | 2680 |
| 2260 | CUUUGUCC A CCAUUCCU | 906 | AGGAAUGG CUGAUGAG X CGAA IGACAAAG | 2681 |
| 2262 | UUGUCCAC C AUUCCUUU | 907 | AAAGGAAU CUGAUGAG X CGAA IUGGACAA | 2682 |
| 2263 | UGUCCACC A UUCCUUUA | 908 | UAAAGGAA CUGAUGAG X CGAA IGUGGACA | 2683 |
| 2267 | CACCAUUC C UUUAAAUU | 909 | AAUUUAAA CUGAUGAG X CGAA IAAUGGUG | 2684 |
| 2268 | ACCAUUCC U UUAAAUUC | 910 | GAAUUUAA CUGAUGAG X CGAA IGAAUGGU | 2685 |
| 2277 | UUAAAUUC U CCAACCCA | 911 | UGGGUUGG CUGAUGAG X CGAA IAAUUUAA | 2686 |
| 2279 | AAAUUCUC C AACCCAAA | 912 | UUUGGGUU CUGAUGAG X CGAA IAGAAUUU | 2687 |
| 2280 | AAUUCUCC A ACCCAAAG | 913 | CUUUGGGU CUGAUGAG X CGAA IGAGAAUU | 2688 |
| 2283 | UCUCCAAC C CAAAGUAG | 914 | AUACUUUG CUGAUGAG X CGAA IUUGGAGA | 2689 |
| 2284 | CUCCAACC C AAAGUAUU | 915 | AAUACUUU CUGAUGAG X CGAA IGUUGGAG | 2690 |
| 2285 | UCCAACCC A AAGUAUUC | 916 | GAAUACUU CUGAUGAG X CGAA IGGUUGGA | 2691 |
| 2294 | AAGUAUUC U UCUUUUCU | 917 | AGAAAAGA CUGAUGAG X CGAA IAAUACUU | 2692 |
| 2297 | UAUUCUUC U UUUCUUAG | 918 | CUAAGAAA CUGAUGAG X CGAA IAAGAAUA | 2693 |
| 2302 | UUCUUUUC U UAGUUUCA | 919 | UGAAACUA CUGAUGAG X CGAA IAAAAGAA | 2694 |
| 2310 | UUAGUUUC A GAAGUACU | 920 | AGUACUUC CUGAGUGA X CGAA IAAACUAA | 2695 |
| 2318 | AGAAGUAC U GGCAUCAC | 921 | GUGAUGCC CUGAUGAG X CGAA IUACUUCU | 2696 |
| 2322 | GUACUGGC A UCACACGC | 922 | GCGUGUGA CUGAUGAG X CGAA ICCAGUAC | 2697 |
| 2325 | CUGGCAUC A CACGCAGG | 923 | CCUGCGUG CUGAUGAG X CGAA IAUGCCAG | 2698 |
| 2327 | GGCAUCAC A CGCAGGUU | 924 | AACCUGCG CUGAUGAG X CGAA IUGAUGCC | 2699 |
| 2331 | UCACACGC A GGUUACCU | 925 | AGGUAACC CUGAUGAG X CGAA ICGUGUGA | 2700 |
| 2338 | CAGGUUAC C UUGGCGUG | 926 | CACGCCAA CUGAUGAG X CGAA IUAACCUG | 2701 |
| 2339 | AGGUUACC U UGGCGUGU | 927 | ACACGCCA CUGAUGAG X CGAA IGUAACCU | 2702 |
| 2351 | CGUGCGUC C CUGUGGUA | 928 | UACCACAG CUGAUGAG X CGAA IACACACG | 2703 |
| 2352 | GUGUGUCC C UGUGGUAC | 929 | GUACCACA CUGAUGAG X CGAA IGACACAC | 2704 |
| 2353 | UGUGUCCC U GUGGUACC | 930 | GGUACCAC CUGAUGAG X CGAA IGGACACA | 2705 |
| 2361 | UGUGGUAC C CUGGCAGA | 931 | UCUGCCAG CUGAUGAG X CGAA IUACCACA | 2706 |
| 2362 | GUGGUACC C UGGCAGAG | 932 | CUCUGCCA CUGAUGAG X CGAA IGUACCAC | 2707 |
| 2363 | UGGUACCC U GGCAGAGA | 933 | UCUCUGCC CUGAUGAG X CGAA IGGUACCA | 2708 |
| 2367 | ACCCUGGC A GAGAAGAG | 934 | CUCUUCUC CUGAUGAG X CGAA ICCAGGGU | 2709 |
| 2378 | GAAGAGAC C AAGCUUGU | 935 | ACAAGCUU CUGAUGAG X CGAA IUCUCUUG | 2710 |
| 2379 | AAGAGACC A AGCUUGUU | 936 | AACAAGCU CUGAUGAG X CGAA IGUCUCUU | 2711 |
| 2383 | GACCAAGC U UGUUUCCC | 937 | GGGAAACA CUGAUGAG X CGAA ICUUGGUC | 2712 |
| 2390 | CUUGUUUC C CUGCUGGC | 938 | GCCAGCAG CUGAUGAG X CGAA IAAACAAG | 2713 |
| 2391 | UUGUUUCC C UGCUGGCC | 939 | GGCCAGCA CUGAUGAG X CGAA IGAAACCA | 2714 |
| 2392 | UGUUUCCC U GUCGGCCA | 940 | UGGCCAGC CUGAUGAG X CGAA IGGAAACA | 2715 |
| 2395 | UUCCCUGC U GGCCAAAG | 941 | CUUUGGCC CUGAUGAG X CGAA ICAGGGAA | 2716 |
| 2399 | CUGCUGGC C AAAGUCAG | 942 | CUGACUUU CUGAUGAG X CGAA ICCAGCAG | 2717 |
| 2400 | UGCUGGCC A AAGUCAGU | 943 | ACUGACUU CUGAUGAG X CGAA IGCCAGCA | 2718 |
| 2406 | CCAAAGUC A GUAGGAGA | 944 | UCUCCUAC CUGAUGAG X CGAA IACUUUGG | 2719 |
| 2421 | GAGGAUGC A CAGUUUGC | 945 | GCAAACUG CUGAUGAG X CGAA ICAUCCUC | 2720 |
| 2423 | GGAUGCAC A GUUUGCUA | 946 | UAGCAAAC CUGAUGAG X CGAA IUGCAUCC | 2721 |
| 2430 | CAGUUUGC U AUUUGCUU | 947 | AAGCAAAU CUGAUGAG X CGAA ICAAACUG | 2722 |
| 2437 | CUAUUUGC U UUAGAGAC | 948 | GUCUCUAA CUGAUGAG X CGAA ICAAAUAG | 2723 |
| 2446 | UUAGAGAC A GGGACUGU | 949 | ACAGUCCC CUGAUGAG X CGAA IUCUCUAA | 2724 |
| 2452 | ACAGGGAC U GUAUAAAC | 950 | GUUUAUAC CUGAUGAG X CGAA IUCCCUGU | 2725 |
| 2461 | GUAUAAAC A AGCCUAAC | 951 | GUUAGGCU CUGAUGAG X CGAA IUUUAUAC | 2726 |
| 2465 | AAACAAGC C UAACAUUG | 952 | CAAUGUUA CUGAUGAG X CGAA ICUUGUUU | 2727 |
| 2466 | AACAAGCC U AACAUUGG | 953 | CCAAUGUU CUGAUGAG X CGAA IGCUUGUU | 2728 |
| 2470 | AGCCUAAC A UUGGUGCA | 954 | UGCACCAA CUGAUGAG X CGAA IUUAGGCU | 2729 |
| 2478 | AUUGGUGC A AAGAUUGC | 955 | GCAAUCUU CUGAUGAG X CGAA ICACCAAU | 2730 |
| 2487 | AAGAUUGC C UCUUGAAU | 956 | AUUCAAGA CUGAUGAG X CGAA ICAAUCUU | 2731 |
| 2488 | AGAUUGCC U CUUGAAUU | 957 | AAUUCAAG CUGAUGAG X CGAA IGCAAUCU | 2732 |
| 2490 | AUUGCCUC U UGAAUUAA | 958 | UUAAUUCA CUGAUGAG X CGAA IAGGCAAU | 2733 |
| 2509 | AAAAAAAC U AGAAAAAA | 959 | UUUUUUCU CUGAUGAG X CGAA IUUUUUUU | 2734 |

Input Sequence = AF190725. Cut Site = 3/.
Stem Length = 8 . Core Sequence = CUGAUGAG X CGAA (X = GCCGUUAGGC or other stem II)
AF190725 (Homo sapiens beta-site APP cleaving enzyme (BACE) mRNA; 2526 bp)

TABLE V
Human BACE G-cleaver Ribozyme and Target Sequence

| | | | | Rz Seq |
|---|---|---|---|---|
| Pos | Substrate | Seq ID | Ribozyme | ID |
| 11 | ACGCUUCC G CAGCCCGC | 960 | GCGGGCUG UGAUG CGAUGCACUAUGC GCG GGACGCGU | 2735 |
| 18 | CGCAGCCC G CCCGGGAG | 961 | CUCCCGGG UGAUG GCAUGCACUAUGC GCG GGGCUGCG | 2736 |
| 29 | CGGGAGCU G CGAGCCGC | 962 | GCGGCUCG UGAUG CGAUGCACUAUGC GCG AGCUCCCG | 2737 |
| 31 | GGAGCUGC G AGCCGCGA | 963 | UCGCGGCU UGAUG GCAUGCACUAUGC GCG GCAGCUCC | 2738 |
| 36 | UGCGAGCC G CGAGCUGG | 964 | CCAGCUCG UGAUG GCAUGCACUAUGC GCG GGCUCGCA | 2739 |
| 38 | CGAGCCGC G AGCUGGAU | 965 | AUCCAGCU UGAUG GCAUGCACUAUGC GCG GCGGCUCG | 2740 |
| 58 | GGUGGCCU G AGCAGCCA | 966 | UGGCUGCU UGAUG GCAUGCACUAUGC GCG AGGCCACC | 2741 |
| 69 | CAGCCAAC G CAGCCGCA | 967 | UGCGGCUG UGAUG GCAUGCAGUAUGC GCG GUUGGCUG | 2472 |
| 75 | ACGCAGCC G CAGGAGCC | 968 | GGCUGGUC UGAUG GCAUGCACUAUGC GCG GGCUGCGU | 2743 |
| 94 | GAGCCCUU G CCCCUGCC | 969 | GGCAGGGG UGAUG GCAUGCACUAUGC GCG AAGGGCUG | 2744 |
| 100 | UUGCCCCU G CCCGCGCC | 970 | GGCGCGGG UGAUG GCAUGCACUAUGC GCG AGGGGCAA | 2745 |
| 104 | CCCUGCCC G CGCCGCCG | 971 | CGGCGGCG UGAUG GCAUGCACUAUGC GCG GGGCAGGG | 2746 |
| 106 | CUGCCCGC G CCGCCGCC | 972 | GGCGGCGG UGAUG GCAUGCACUAUGC GCG GCGGGCAG | 2747 |
| 109 | CCCGCGCC G CCGCCCGC | 973 | GCGGGCGG UGAUG GCAUGCACUAUGC GCG GGCGCGGG | 2748 |
| 112 | GCGCCGCC G CCCGCCGG | 974 | CCGGCGGG UGAUG GCAUGCACUAUGC GCG GGCGGCGC | 2749 |
| 116 | GGCCGCCC G CCGGGGGG | 975 | CCCCCCGG UGAUG GCAUGCACUAUGC GCG GGGCGGCG | 2750 |
| 137 | GGGAAGCC G CCACCGGC | 976 | GCCGGUGG UGAUG GCAUGCACUAUGC GCG GGCUUCCC | 2751 |
| 148 | ACCGGCCC G CCAUGCCC | 977 | GGGCAUGG UGAUG GCAUGCACUAUGC GCG GGGCCGGU | 2752 |
| 153 | CCCGCCAU G CCCGCCCC | 978 | GGGGCGGG UGAUG GCAUGCACUAUGC GCG AUGGCGGG | 2753 |
| 157 | CCAUGCCC G CCCCUCCC | 979 | GGGAGGGG UGAUG GCAUGCACUAUGC GCG GGGCAUGG | 2754 |
| 172 | CCAGCCCC G CCGGGAGC | 980 | GCUCCCGG UGAUG GCAUGCACUAUGC GCG GGGGCUGG | 2755 |
| 183 | GGGAGCCC G CGCCCGCU | 981 | AGCGGGCG UGAUG GCAUGCACUAUGC GCG GGGCUGGG | 2756 |
| 185 | GAGCCCGC G CCCGCUGC | 982 | GCAGCGGG UGAUG GCAUGCACUAUGC GCG GCGGGCUG | 2757 |
| 189 | CCGCGCCC G CUCCCGAG | 983 | CUGGGCAG UGAUG GCAUGCACUAUGC GCG GGGCGCGG | 2758 |
| 192 | CGCCCGCU G CCCAGGCU | 984 | AGCCUGGG UGAUG GCAUGCACUAUGC GCG AGCGGGCG | 2759 |
| 205 | GGCUGGCC G CCGCCGUG | 985 | CACGGCGG UGAUG GCAUGCACUAUGC GCG GGCCAGCC | 2760 |
| 208 | UGGCCGCC G CCGUGCCG | 986 | CGGCACGG UGAUG GCAUGCACUAUGC GCG GGCGGCCA | 2761 |
| 213 | GCCGCCGU G CCGAUGUA | 987 | UACAUCGG UGAUG GCAUGCACUAUGC GCG ACGGCGGC | 2762 |
| 216 | GCCGUGCC G AUGUAGCG | 988 | CGCUACAU UGAUG GCAUGCACUAUGC GCG GGCACGGC | 2763 |
| 250 | UCUCCCCU G CUCCCGUG | 989 | CACGGGAG UGAUG GCAUGCACUAUGC GCG AGGGGAGA | 2764 |
| 258 | GCUCCCGU G CUCUGCGG | 990 | CCGCAGAG UGAUG CGAUGCACUAUGC GCG ACGGGAGC | 2765 |
| 263 | CGUGCUCU G CGGAUCUC | 991 | GAGAUCCG UGAUG GCAUGCACUAUGC GCG AGAGCACG | 2766 |
| 276 | UCUCCCCU G ACCGCUCU | 992 | AGAGCGGU UGAUG GCAUGCACUAUGC CGC AGGGGAGA | 2767 |
| 280 | CCCUGACC G CUCUCCAC | 993 | GUGGAGAG UGAUG GCAUGCACUAUGC GCG GGUCAGGG | 2768 |
| 320 | AGGGCCCU G CAGGCCCU | 994 | AGGGCCUG UGAUG GCAUGCACUAUGC GCG AGGGCCCU | 2769 |
| 337 | GGCGUCCU G AUGCCCCC | 995 | GGGGGCAU UGAUG CGAUGCACUAUGC GCG AGGACGCC | 2770 |
| 340 | GUCCUGAU G CCCCCAAG | 996 | CUUGGGGG UGAUG GCAUGCACUAUGC GCG AUCAGGAC | 2771 |
| 360 | CCUCUCCU G AGAAGCCA | 997 | UGGCUUCU UGAUG GCAUGCACUAUGC GCG AGGAGAGG | 2772 |
| 397 | GGGCAGGC G CCAGGGAC | 998 | GUCCCUGG UGAUG GCAUGCACUAUGC GCG GCCUGCCC | 2773 |
| 420 | GGGCCAGU G CGAGCCCA | 999 | UGGGCUCG UGAUG GCAUGCACUAUGC GCG ACGUUCCC | 2774 |
| 422 | GCCAGUGC G AGCCCAGA | 1000 | UCUGGGCU UGAUG GCAUGCACUAUGC GCG GCACUGGC | 2775 |
| 437 | GAGGGCCC G AAGGCCGG | 1001 | CCGGCCUU UGAUG GCAUGCACUAUGC GCG GGGCCCUC | 2776 |
| 468 | CAAGCCCU G CCCUGGCU | 1002 | AGCCAGGG UGAUG GCAUGCACUAUGC GCG AGGGCUUG | 2777 |
| 480 | UGGCUCCU G CUGUGGAU | 1003 | AUCCACAG UGAUG GCAUGCACUAUGC GCG AGGAGCCA | 2778 |
| 493 | GGAUGGGC G CGGGAGUG | 1004 | CACUCCCG UGAUG GCAUGCACUAUGC CGC GCCCAUCC | 2779 |
| 501 | GCGGGAGU G CUGCCUGC | 1005 | GCAGGCAG UGAUG GCAUGCACUAUGC GCG ACUCCCGC | 2780 |
| 504 | GGAGUGCU G CCUGCCCA | 1006 | UGGGCAGG UGAUG GCAUGCACUAUGC GCG AGCACUCC | 2781 |
| 508 | UGCUGCCU G CCCACGCC | 1007 | GCCGUGGG UGAUG GCAUGCACUAUGC GCG AGGCAGCA | 2782 |
| 537 | AUCCGGCU G CCCCUGCG | 1008 | CGCAGGGG UGAUG GCAUGCACUAUGC GCG AGCCGGAU | 2783 |
| 543 | CUGCCCCU G CGCAGCGG | 1009 | CCGCUGCG UGAUG CGAUGCAGUAUGC GCG AGGGGCAG | 2784 |
| 545 | GCCCCUGC G CAGCGGCC | 1010 | GGCCGCUG UGAUG CGAUGCACUAUGC GCG GCAGGGGC | 2785 |
| 562 | UGGGGGGC G CCCCCCUG | 1011 | CAGGGGGG UGAUG GCAUGCACUAUGC GCG CGGGGGGA | 2786 |
| 576 | CUGGGGCU G CGGCUGCC | 1012 | GGCAGCCG UGAUG GCAUGCACUAUGC GCG AGCCCCAG | 2787 |
| 582 | CUGCGGCU G CCCCGGGA | 1013 | UCCCGGGG UGAUG CGAUGCACUAUGC GCG AGCCGCAG | 2788 |
| 595 | GGGAGACC G ACGAAGAG | 1014 | CUCUUCGU UGAUG GCAUGCACUAUGC GCG GGUCUGGG | 2789 |
| 598 | AGACCGAC G AAGAGCCC | 1015 | GGGCUCUU UGAUG GCAUGCACUAUGC GCG GUCGGUCU | 2790 |
| 607 | AAGAGCCC G AGGAGCCC | 1016 | GGGCUCCU UGAUG GCAUGCACUAUGC GCG GGGCUCUU | 2791 |
| 654 | GACAACCU G AGGGGCCA | 1017 | UUGCCCCU UGAUG GCAUGCACUAUGC GCG AGGUUGUC | 2792 |
| 690 | GUGGAGAU G ACCGUGGG | 1018 | CCCACGGU UGAUG GCAUGCACUAUGC GCG AUCUCCAC | 2793 |
| 708 | AGCCCCCC G CAGACGCU | 1019 | AGCGUCUG UGAUG GCAUGCACUAUGC GCG GGGGGGCU | 2794 |
| 714 | CCGCAGAC G CUCAACAU | 1020 | AUGUUGAG UGAUG GCAUGCACUAUGC GCG GUCUGCGG | 2795 |
| 751 | GUAACUUU G CAGUGGGU | 1021 | ACCCACUG UGAUG GCAUGCACUAUGC GCG AAAGUUAC | 2796 |
| 760 | CAGUGGGU G CUGCCCCC | 1022 | GGGGGCAG UGAUG GCAUGCACUAUGC GCG ACCCACUG | 2797 |
| 763 | UGGGUGCU G CCCCCCAC | 1023 | GUCCCCCC UGAUG GCAUGCACUAUGC GCG AGCACCCA | 2798 |
| 780 | CCCUUCCU G CAUCGCUA | 1024 | UAGCGAUG UGAUG GCAUGCACUAUGC GCG AGGAAGGG | 2799 |
| 785 | CCUGCAUC G CUACUACC | 1025 | GGUAGUAG UGAUG GCAUGCACUAUGC GCG GAUGCAGG | 2800 |
| 843 | GUGUAUGU G CCCUACAC | 1026 | GUGUAGGG UGAUG GCAUGCACUAUGC GCG ACAUACAC | 2801 |
| 883 | UGGGCACC G ACCUGGUA | 1027 | UACCAGGU UGAUG GCAUGCACUAUGC GCG GGUGCCCA | 2802 |
| 921 | GUCACUGU G CGUGCCAA | 1028 | UUGGCACG UGAUG GCAUGCACUAUGC GCG ACAGUGAC | 2803 |
| 925 | CUGUGCGU G CCAACAUU | 1029 | AAUGUUGG UGAUG GCAUGCACUAUGC GCG ACGCACAG | 2804 |
| 934 | CCAACAUU G CUGCCAUC | 1030 | GAUGGCAG UGAUG GCAUGCACUAUGC GCG AAUGUUGG | 2805 |
| 937 | ACAUUGCU G CCAUCACU | 1031 | AGUGAUGG UGAUG CGAUGCACUAUGC GCG AGCAAUGU | 2806 |
| 946 | CCAUCACU G AAUCAGAC | 1032 | GUCUGAUU UGAUG GCAUGCACUAUGC GCG AGUGAUGG | 2807 |
| 1006 | UGGCCUAU G CUGAGAUU | 1033 | AAUCUCAG UGAUG GCAUGCACUAUGC GCG AUAGGCCA | 2808 |
| 1009 | CCUAUGCG G AGAUUGCC | 1034 | GGCAAUCU UGAUG GCAUGCACUAUGC GCG AGCAUAGG | 2809 |
| 1015 | CUGAGAUU G CCAGGCCU | 1035 | AGGCCUGG UGAUG GCAUGCACUAUGC GCG AAUCUCAG | 2810 |
| 1024 | CCAGGCCU G ACGACUCC | 1036 | GGAGUCGU UGAUG GCAUGCACUAUGC GCG AGGCCUGG | 2811 |
| 1027 | GGCCUGAC G ACUCCCUG | 1037 | CAGGGAGU UGAUG GCAUGCACUAUGC GCG GUCAGGCC | 2812 |
| 1048 | CUUUCUUU G ACUCUCUG | 1038 | CAGAGAGU UGAUG GCAUGCACUAUGC GCG AAAGAAAG | 2813 |
| 1092 | UUCUCCCU G CAGCUUUG | 1039 | CAAAGCUG UGAUG GCAUGCACUAUGC GCG AGGGAGAA | 2814 |
| 1105 | UUUGUGGU G CUGGCUUC | 1040 | GAAGCCAG UGAUG GCAUGCACUAUGC GCG ACCACAAA | 2815 |
| 1129 | ACCAGUCU G AAGUGCUG | 1041 | CAGCACUU UGAUG GCAUGCACUAUGC GCG AGACUGGU | 2816 |
| 1134 | UCUGAAGU G CUGGCCUC | 1042 | GAGGCCAG UGAUG GCAUGCACUAUGC GCG ACGGCAGA | 2817 |
| 1158 | GGGAGCAU G AUCAUUGG | 1043 | CCAAUGAU UGAUG GCAUGCACUAUGC GCG AUGCUGGG | 2818 |
| 1174 | GAGGUAUC G ACCACUGG | 1044 | CGAGUGGU UGAUG GCAUGCACUAUGC GCG GAUACCUC | 2819 |
| 1182 | GACCACUC G CUGUACAC | 1045 | GUGUACAG UGAUG GCAUGCACUAUGC GCG GAGUGGUC | 2820 |
| 1234 | GGUAUUAU G AGGUGAUC | 1046 | GAUCACCU UGAUG CGAUGCACUAUGC GCG AUAAUACC | 2821 |
| 1239 | UAUGAGGU G AUCAUUGU | 1047 | ACAAUGAU UGAUG GCAUGCACUAUGC GCG ACCUCAUA | 2822 |
| 1248 | AUCAUUGU G CGGGUGGA | 1048 | UCCACCCG UGAUG GCAUGCACUAUGC GCG ACAAUGAU | 2823 |
| 1275 | CAGGAUCU G AAAAUGGA | 1049 | UCCAUUUU UGAUG GCAUGCACUAUGC GCG AGAUCCUG | 2824 |
| 1286 | AAUGGACU G CAAGGAUG | 1050 | ACUCCUUG UGAUG GCAUGCACUAUGC GCG AGUCCAUU | 2825 |
| 1303 | ACAACUAU G ACAAGAGC | 1051 | GCUCUUGU UGAUG GCAUGCACUAUGC GCG AUAGUUGU | 2826 |
| 1344 | CUUCGUUU G CCCAAGAA | 1052 | UUCUUGGG UGAUG GCAUGCACUAUGC GCG AAACGAAG | 2827 |
| 1360 | AAGUGUUU G AAGCUGCA | 1053 | UGCAGCUU UGAUG GCAUGCACUAUGC GCG AAACACUU | 2828 |
| 1366 | UUGAAGCU G CAGUCAAA | 1054 | UUUGACUG UGAUG GCAUGCACUAUGC GCG AGCUUCAA | 2829 |
| 1411 | AGUUCCCU G AUGGUUUC | 1055 | GAAACCAU UGAUG GCAUGCACUAUGC GCG AGGGAACG | 2830 |
| 1442 | GCUGGUGU G CUGGCAAG | 1056 | CUUGCCAG UGAUG GCAUGCACUAUGC GCG ACACCAGC | 2831 |
| 1504 | UAAUGGGU G AGGUUACC | 1057 | GGUAACCU UGAUG GCAUGCACUAUGC GCG ACCCAUUA | 2832 |
| 1526 | GUCCUUCC G CAUCACCA | 1058 | UGGUGAUG UGAUG GCAUGCACUAUGC GCG GGAAGGAC | 2833 |
| 1542 | AUCCUUCC G CAGCAAUA | 1059 | UAUUGCUG UGAUG GCAUGCACUAUGC GCG GGAAGGAU | 2834 |
| 1554 | CAAUACCU G CGGCCAGU | 1060 | ACUGGCCG UGAUG GCAUGCACUAUGC GCG AGGUAUUG | 2835 |
| 1588 | CCCAAGAC G ACUGUUAC | 1061 | GUAACAGU UGAUG GCAUGCACUAUGC GCG GUCUUGGG | 2836 |
| 1603 | ACAAGUUU G CCAUCUCA | 1062 | UGAGAUGG UGAUG GCAUGCACUAUGC GCG AAACUUGU | 2837 |
| 1672 | UUGUCUUU G AUCGGGCC | 1063 | GGCCCGAU UGAUG GCAUGCACUAUGC GCG AAAGACAA | 2838 |
| 1682 | UCGGGCCC G AAAACGAA | 1064 | UUCGUUUU UGAUG GCAUGCACUAUGC GCG GGGCCCGA | 2839 |
| 1688 | CCGAAAAC G AAUUGGCU | 1065 | AGCCAAUU UGAUG GCAUGCACUAUGC GCG GUUUUCGG | 2840 |
| 1699 | UUGGCUUU G CUGUCAGC | 1066 | GCUGACAG UGAUG GCAUGCACUAUGC GCG AAAGCCAA | 2841 |
| 1708 | CUGUCAGC G CUUGCCAU | 1067 | AUGGCAAG UGAUG GCAUGCACUAUGC GCG GCUGACAG | 2842 |
| 1712 | CAGCGCUU G CCAUGUGC | 1068 | GCACAUGG UGAUG GCAUGCACUAUGC GCG AAGCGCUG | 2843 |
| 1719 | UGCCAUGU G CACGAUGA | 1069 | UCAUCGUG UGAUG GCAUGCACUAUGC GCG ACAUGGCA | 2844 |
| 1723 | AUGUGCAC G AUGAGUUC | 1070 | GAACUCAU UGAUG GCAUGCACUAUGC GCG AUGCACAU | 2845 |
| 1726 | UGCACGAU G AGUUCAGG | 1071 | CCUGAACU UGAUG GCAUGCACUAUGC GCG AUCGUGCA | 2846 |
| 1807 | AGACAGAU G AGUCAACC | 1072 | GGUUGACU UGAUG GCAUGCACUAUGC GCG AUCUGUCU | 2847 |
| 1821 | ACCCUCAU G ACCAUAGC | 1073 | GCUAUGGU UGAUG GCAUGCACUAUGC GCG AUGAGGGU | 2848 |
| 1843 | UCAUGGCU G CCAUCUGC | 1074 | GCAGAUGG UGAUG GCAUGCACUAUGC GCG AGCCAUGA | 2849 |
| 1850 | UGCCAUCU G CGCCCUCU | 1075 | AGAGGGCG UGAUG GCAUGCACUAUGC GCG AGAUGGCA | 2850 |
| 1852 | CCAUCUGC G CCCUCUUC | 1076 | GAAGAGGG UGAUG GCAUGCACUAUGC GCG GCAGAUGG | 2851 |
| 1863 | CUCUUCAU G CUGCCACU | 1077 | AGUGGCAG UGAUG GCAUGCACUAUGC GCG AUGAAGAG | 2852 |
| 1866 | UUCAUGCU G CCACUCUG | 1078 | CAGAGUGG UGAUG GCAUGCACUAUGC GCG AGCAUGAA | 2853 |
| 1874 | GCCACUCU G CCUCAUGG | 1079 | CCAUGAGG UGAUG GCAUGCACUAUGC GCG AGAGUGGC | 2854 |
| 1895 | UCAGUGGC G CUGCCUGG | 1080 | GGAGGCAG UGAUG GCAUGCACUAUGC GCG GCCACUGA | 2855 |
| 1898 | GUGGCGCU C CCUCCGCU | 1081 | AGCGGAGG UGAUG GCAUGCACUAUGC GCG AGCGCCAG | 2856 |
| 1904 | CUGCCUGG G CUGCCUGC | 1082 | GCAGGCAG UGAUG GCAUGCACUAUGC GCG GGAGGCAG | 2857 |
| 1907 | CCUCCGCU G CCUGCGCC | 1083 | GGCGCAGG UGAUG GCAUGCACUAUGC GCG AGCGGAGG | 2858 |
| 1911 | CGCUGCCU G CGGGAGCA | 1084 | UGCUGGCG UGAUG GCAUGCACUAUGC GCG AGGCAGCG | 2859 |
| 1913 | CUGCCUGC G CCAGCAGC | 1085 | GCUGCUGG UGAUG GCAUGCACUAUGC GCG GCAGGCAG | 2860 |
| 1924 | AGCAGCAU G AUGACUUU | 1086 | AAAGUCAU UGAUG GCAUGCACUAUGC GCG AUGCUGCU | 2861 |
| 1927 | AGCAUGAU G ACUUUGCG | 1087 | AGCAAAGU UGAUG GCAUGCAGUAUGC GCG AUCAUGCU | 2862 |
| 1933 | AUGACUUU G CUGAUGAC | 1088 | GUCAUCAG UGAUG GCAUGCACUAUGC GCG AAAGUCAU | 2863 |
| 1936 | ACUUUGCU G AUGACAUC | 1089 | GAUGUCAU UGAUG GCAUGCACUAUGC GCG AGCAAAGU | 2864 |
| 1939 | UUGCUGAU G ACAUCUCC | 1090 | GGAGAUGU UGAUG GCAUGCACUAUGC GCG AUCAGCAA | 2865 |
| 1950 | AUCUCCCU G CUGAAGUG | 1091 | CACUUCAG UGAUG GCAUGCACUAUGC GCG AGGGAGAU | 2866 |
| 1953 | UCCCUGCU G AAGUGAGG | 1092 | CCUCACUU UGAUG GCAUGCACUAUGC GCG AGCAGGGA | 2867 |
| 1958 | GCGGAAGU G AGGAGGCC | 1093 | GGCCUCCU UGAUG GCAUGCACUAUGC GCG ACUUCAGC | 2868 |
| 2087 | CACCAAAU G CCUCUGCC | 1094 | GGCAGAGG UGAUG GCAUGCACUAUGC GCG AUUUGGUG | 2869 |
| 2093 | AUGCCUCU G CCUUGAUG | 1095 | CAUCAAGG UGAUG GCAUGCACUAUGC GCG AGAGGCAU | 2870 |
| 2098 | UCUGCCUU G AUGGAGAA | 1096 | UUCUCCAU UGAUG GCAUGCACUAUGC GCG AAGGCAGA | 2871 |
| 2179 | AGCACUCU G CUGGCGGG | 1097 | CCCUGGAG UGAUG GCAUGCACUAUGC GCG AGAGUGCU | 2872 |
| 2227 | GAAAUUCU G CUGCUUGA | 1098 | UCAAGCAG UGAUG GCAUGCACUAUGC GCG AGCCUUUC | 2873 |
| 2230 | AUUCUGCU G CUUGAAAC | 1099 | GUUUCAAG UGAUG GCAUGCACUAUGC GCG AGCAGAAU | 2874 |
| 2234 | UGCUGCUU G AAACUUCA | 1100 | UGAAGUUU UGAUG GCAUGCACUAUGC GCG AAGCAGCA | 2875 |
| 2248 | UCAGCCCU G AACCUUUG | 1101 | CAAAGGUU UGAUG GCAUGCACUAUGC GCG AGGGCUGA | 2876 |
| 2329 | CAUCACAC G CAGGUUAC | 1102 | GUAACCUG UGAUG GCAUGCACUAUGC GCG GUGUGAUG | 2877 |
| 2393 | GUUUCCCU G CUGGCCAA | 1103 | UUGGCCAG UGAUG GCAUGCACUAUGC GCG AGGGAAAC | 2878 |
| 2419 | GAGAGGAU G CACAGUUU | 1104 | AAACUGUG UGAUG GCAUGCACUAUGC GCG AUCCUCUC | 2879 |
| 2428 | CACACUUU G CUAUUUGC | 1105 | GCAAAUAG UGAUG GCAUGCACUAUGC GCG AAACUGUG | 2880 |
| 2435 | UGCUAUUU G CUUUAGAG | 1106 | CUCUAAAG UGAUG GCAUGCACUAUGC GCG AAAUAGCA | 2881 |
| 2476 | ACAUUGGU G CAAAGAUU | 1107 | AAUCUUUG UGAUG GCAUGCACUAUGC GCG ACCAAUGU | 2882 |
| 2485 | CAAAGAUU G CCUCUUGA | 1108 | UCAAGAGG UGAUG GCAUGCACUAUGC GCG AAUCUUUG | 2883 |
| 2492 | UGCCUCUU G AAUUAAAA | 1109 | UUUUAAUU UGAUG GCAUGCACUAUGC GCG AAGAGGCA | 2884 |
| 219 | GUGCCGAU G UAGCGGGC | 1110 | GCCCGCUA UGAUG GCAUGCACUAUGC GCG AUCGGCAC | 2885 |
| 483 | CUCCUGCU G UGGAUGGG | 1111 | CCCAUCCA UGAUG GCAUGCACUAUGC GCG AGCAGGAG | 2886 |
| 634 | GCAGCUUU G UGGAGAUG | 1112 | CAUCUCCA UGAUG GCAUGCACUAUGC GCG AAAGCUGC | 2887 |
| 804 | AGGCAGCU G UCCAGCAC | 1113 | GUGCUGGA UGAUG GCAUGCACUAUGC GCG AGUCGCCU | 2888 |
| 835 | GGAAGGGU G UGUAUGUG | 1114 | CACAUACA UGAUG GCAUGCACUAUGC GCG ACCCUUCC | 2889 |
| 837 | AAGGGUGU G UAUGUGCC | 1115 | GGCACAUA UGAUG GCAUGCACUAUGC GCG ACACCCUU | 2890 |
| 841 | GUGUGUAU G UGCCCUAC | 1116 | GUGGGGCA UGAUG GCAUGCACUAUGC GCG AUACACAC | 2891 |
| 919 | ACGUCACU G UGCGUGCC | 1117 | GGCACGCA UGAUG GCAUGCACUAUGC GCG AGUGACGU | 2892 |
| 1100 | GCAGCUUU G UGGUGCUG | 1118 | CAGCACCA UGAUG GCAUGCACUAUGC GCG AAAGCUGC | 2893 |
| 1144 | UGGCCUCU G UCGGAGGG | 1119 | CCCUCCGA UGAUG GCAUGCACUAUGC GCG AGAGGCCA | 2894 |
| 1185 | CACUCGCU G UACACAGG | 1120 | CCUGUGUA UGAUG GCAUGCACUAUGC GCG AGCCAGUG | 2895 |
| 1246 | UGAUCAUU G UGCGGGUG | 1121 | GACCCGCA UGAUG GCAUGCACUAUGC GCG AAUGAUCA | 2896 |
| 1315 | AGAGCAUU G UGGACAGU | 1122 | ACUGUCCA UGAUG GCAUGCACUAUGC GCG AAUGCUCU | 2897 |
| 1356 | AAGAAAGU G UUUGAAGC | 1123 | GCUUCAAA UGAUG GCAUGCACUAUGC GCG ACUUUCUU | 2898 |
| 1440 | CAGCUGGU G UGCUGGCA | 1124 | UGCCAGCA UGAUG GCAUGCACUAUGC GCG ACCAGCUG | 2899 |
| 1570 | UGGAAGAU G UGGCCACG | 1125 | CGUGGCCA UGAUG GCAUGCACUAUGC GCG ACUCUCCA | 2900 |
| 1592 | AGACGACU G UUACAAGU | 1126 | ACUUGUAA UGAUG GCAUGCACUAUGC GCG AUGCGUCU | 2901 |
| 1630 | CGGGCACU G UUAUGGGA | 1127 | UCCCAUCC UGAUG GCAUGCACUAUGC GCG AGUGCCCG | 2902 |
| 1642 | UGGGAGCU G UUAUCAUG | 1128 | CAUGAUAA UGAUG GCAUGCACUAUGC GCG AGCUCCCA | 2903 |
| 1666 | UCUACGUU G UCUUUGAU | 1129 | AUCAAAGA UGAUG GCAUGCACUAUGC GCG AACGUAGA | 2904 |
| 1702 | GCUUUGCU G UCAGCGCU | 1130 | AGCGCUGA UGAUG GCAUGCACUAUGC GCG AGCAAAGC | 2905 |
| 1717 | CUUGCCAU G UGCACGAU | 1131 | AUCGUGCA UGAUG GCAUGCACUAUGC GCG AUGGCAAG | 2906 |
| 1759 | GCCCUUUU G UCACCUUG | 1132 | CAAGGUGA UGAUG GCAUGCACUAUGC GCG AAAAGGGC | 2907 |
| 1781 | GGAAGACU G UGGCUACA | 1133 | UGUAGCCA UGAUG GCAUGCACUAUGC GCG AGUCUUCC | 2908 |
| 1834 | UAGCCUAU G UCAUGGCU | 1134 | AGCCAUGA UGAUG GCAUGCACUAUGC GCG AUAGGCUA | 2909 |
| 1884 | CUCAUGGU G UGUCAGUG | 1135 | CACUGACA UGAUG GCAUGCACUAUGC GCG ACCAUGAG | 2910 |
| 1886 | CAUGGUGU G UCAGUGGC | 1136 | GCCACUGA UGAUG GCAUGCACUAUGC GCG ACACCAUG | 2911 |
| 2048 | UGGCACCU G UGGCCAGA | 1137 | UCUGGCCA UGAUG GCAUGCACUAUGC GCG AGGUGCCA | 2912 |
| 2139 | CAGGGACU G UACCUGUA | 1138 | UACAGGUA UGAUG GCAUGCACUAUGC GCG AGUCCCUG | 2913 |
| 2145 | CUGUACCU G UAGGAAAC | 1139 | GUUUCCUA UGAUG GCAUGCACUAUGC GCG AGGUACAG | 2914 |
| 2256 | GAACCUUU G UCCACCAU | 1140 | AUGGUGGA UGAUG GCAUGCACUAUGC GCG AAAGGUUC | 2915 |
| 2346 | CUUGGCGU G UGUCCCUG | 1141 | CAGGGACA UGAUG GCAUGCACUAUGC GCG ACGCCAAG | 2916 |
| 2348 | UGGCGUGU G UCCCUGUG | 1142 | CACAGGGA UGAUG CGAUGCACUAUGC GCG ACACGCCA | 2917 |
| 2354 | GUGUCCCU G UGGUACCC | 1143 | GGGUACCA UGAUG GCAUGCACUAUGC GCG AGGGACAC | 2918 |
| 2385 | CCAAGCUU G UUUCCCUG | 1144 | CAGGGAAA UGAUG GCAUGCACUAUGC GCG AAGCUUGG | 2919 |
| 2453 | CAGGGACU G UAUAAACA | 1145 | UGUUUAUA UGAUG GCAUGCACUAUGC GCG AGUCCCUG | 2920 |

Input Sequence = AF190725. Cut Site = G/.
Stem Length = 8. Core Sequence = UGAUG GCAUGCACUAUGC GCG
AF190725 (Homo sapiens beta-site APP cleaving enzyme (BACE) mRNA; 2526 bp)

TABLE VI
Human BACE Zinzyme Ribozyme and Target Sequence

| | | | | Rz Seq |
|---|---|---|---|---|
| Pos | Substrate | Seq ID | Ribozyme | ID |
| 11 | ACGCGUCC G CAGCCCGC | 960 | GCGGGCUG GCCGAAAGGCGAGUCAAGGUCU GGACGCGU | 2921 |
| 18 | CGCAGCCC G CCCGGGAG | 961 | CUCCCGGG GCCGAAAGGCGAGUCAAGGUCU GGGCUGCG | 2922 |
| 29 | CGGGAGCU G CGAGCCGC | 962 | GCGGCUCG GCCGAAAGGCGAGUCAAGGUCU AGCUCCCG | 2923 |
| 36 | UGCGAGCC G CGAGUUGG | 964 | CCAGCUCG GCCGAAAGGCGAGUCAAGGUCU GGCUCGCA | 2924 |
| 69 | CAGCCAAC G CAGCCGCA | 967 | UGCGGCUG GCCGAAAGGCGAGUCAAGGUCU GUUGGCUG | 2925 |
| 75 | ACGCAGCC G GAGGAGCC | 968 | GGCUCCUG GCCGAAAGGCGAGUCAAGGUCU GGCUGCGU | 2926 |
| 94 | GAGCCCUU G CCCCUGCC | 969 | GGCAGGGG GCCGAAAGGCGAGUCAAGGUCU AAGGGCUC | 2927 |
| 100 | UUGCCCCU G CCCGCGCC | 970 | GGCGCGGG GCCGAAAGGCGAGUCAAGGUCU AGGGGCAA | 2928 |
| 104 | CCCUGCCC G CGCCGCCG | 971 | CGGCGGCG GCCGAAAGGCGAGUCAAGGUCU GGGCAGGG | 2929 |
| 106 | CUGCCCGC G CCGCCGCC | 972 | GGCGGCGG GCCGAAAGGCGAGUCAAGGUCU GCGGGCAG | 2930 |
| 109 | CCCGCGCC G CCGCCCGC | 973 | GCGGGCGG GCCGAAAGGCGAGUCAAGGUCU GGCGCGGG | 2931 |
| 112 | GCGCCGCC G CCCGCCGG | 974 | CCGGCGGG GCCGAAAGGCGAGUCAAGGUCU GGCGGCGC | 2932 |
| 116 | CGCCGCCC G CCGGGGGG | 975 | CCCCCCGG GCCGAAAGGCGAGUCAAGGCUC GGGCGGCG | 2933 |
| 137 | GGGAAGCC G CCACCGGC | 976 | GCCGGUGG GCCGAAAGGCGAGUCAAGGUCU GGCUUCCC | 2934 |
| 148 | ACCGGCCC G CCAUGCCC | 977 | GGGCAUGG GCCGAAAGGCGAGUCAAGGUCU GGGCCGGU | 2935 |
| 153 | CCCGCCAU G CCCGCCCC | 978 | GGGGCGGG GCCGAAAGGCGAGUCAAGGUCU AUGGCGGG | 2936 |
| 157 | CCAUGCCC G CCCCUCCC | 979 | GGGAGGGG GCCGAAAGGCGAGUCAAGGUCU GGGCAUGG | 2937 |
| 172 | CCAGCCCC G CCGGGAGC | 980 | GCUCCCGG GCCGAAAGGCGAGUCAAGGUCU GGGGCUGG | 2938 |
| 183 | GGGAGCCC G CGCCCGCU | 981 | AGCGGGCG GCCGAAAGGCGAGUCAAGGUCU GGGCUCCC | 2939 |
| 185 | GAGCCCGC G CCCGCUGC | 982 | GCAGCGGG GCCGAAAGGCGAGUCAAGGUCU GCGGGCUC | 2940 |
| 189 | CCGCGCCC G CUGCCCAG | 983 | CUGGGCAG GCCGAAAGGCGAGUCAAGGUCU GGGCGCGG | 2941 |
| 192 | CGCCCGCU G CCCAGGCU | 984 | AGCCUGGG GCCGAAAGGCGAGUCAAGGUCU AGCGGGCG | 2942 |
| 205 | GGCUGGCC G CCGCCGUG | 985 | CACGGCGG GCCGAAAGGCGAGUCAAGGUCU GGCCAGCC | 2943 |
| 208 | UGGCCGCC G CCGUGCCG | 986 | GCCCACGG GCCGAAAGGCGAGUCAAGGUCU GGCGGCCA | 2944 |
| 213 | GCCGCCGU G CCGAUGUA | 987 | UACAUCGG GCCGAAAGGCGAGUCAAGGUCU ACGGCGGC | 2945 |
| 250 | UCUCCCCU G CUCCCGUG | 989 | CACGGGAG GCCGAAAGGCGAGUCAAGGUCU AGGGGAGA | 2946 |
| 258 | GCUCCCGU G CUCUGCGG | 990 | CCGCAGAG GCCGAAAGGCGAGUCAAGGUCU ACGGGAGC | 2947 |
| 263 | CGUGCUCU G CGGAUCUC | 991 | GAGAUCCG GCCGAAAGGCGAGUCAAGGUCU AGAGCACG | 2948 |
| 280 | CCCUGACC G CUCUCCAC | 993 | GUGGAGAG GCCGAAAGGCGAGUCAAGGUCU GGUCAGGG | 2949 |
| 320 | AGGGCCCU G CAGGCCCU | 994 | AGGGCCUG GCCGAAAGGCGAGUCAAGGUCU AGGGCCCU | 2950 |
| 340 | GUCCUGAU G CCCCCAAG | 996 | CUUGGGGG GCCGAAAGGCGAGUCAAGGUCU AUCAGGAC | 2951 |
| 397 | GGGCAGGC G CCAGGGAC | 998 | GUCCCUGG GCCGAAAGGCGAGUCAAGGUCU GCCUGCCC | 2952 |
| 420 | GGGCCAGU G CGAGCCCA | 999 | UGGGCUCG GCCGAAAGGCGAGUCAAGGUCU ACUGGCCC | 2953 |
| 468 | CAAGCCCU G CCCUGGCU | 1002 | AGCCAGGG GCCGAAAGGCGAGUCAAGGUCU AGGGCUUG | 2954 |
| 480 | UGGCUCCU G CUGUGGAU | 1003 | AUCCACAG GCCGAAAGGCGAGUCAAGGUCU AGGAGCCA | 2955 |
| 493 | GGAUGGGC G CGGGAGUG | 1004 | CACUCCCG GCCGAAAGGCGAGUCAAGGUCU GCCCAUCC | 2956 |
| 501 | GCGGGAGU G CUGCCUGC | 1005 | GCAGGCAG GCCGAAAGGCGAGUCAAGGUCU ACUCCCGC | 2957 |
| 504 | GGAGUGCU G CCUGCCCA | 1006 | UGGGCAGG GCCGAAAGGCGAGUCAAGGUCU AGCACUCC | 2958 |
| 508 | UGCUGCCU G CCCACGGC | 1007 | GCCGUGGG GCCGAAAGGCGAGUCAAGGUCU AGGCAGCA | 2959 |
| 537 | AUCCGGCU G CCCCUGCG | 1008 | CGCAGGGG GCCGAAAGGCGAGUCAAGGUCU AGCCGGAU | 2960 |
| 543 | CUGCCCCU G CGCAGCGG | 1009 | CCGCUGCG GCCGAAAGGCGAGUCAAGGUCU AGGGGCAG | 2961 |
| 545 | GCCCCUGC G CAGCGGCC | 1010 | GGCCGCUG GCCGAAAGGCGAGUCAAGGUCU GCAGGGGC | 2962 |
| 562 | UGGGGGGC G CCCCCCUG | 1011 | CAGGGGGG GCCGAAAGGCGAGUCAAGGUCU GCCCCCCA | 2963 |
| 576 | CUGGGGCU G CGGCUGCC | 1012 | GGCAGCCG GCCGAAAGGCGAGUCAAGGUCU AGCCCCAG | 2964 |
| 582 | CUGCGGCU G CCCCGGGA | 1013 | UCCCGGGG GCCGAAAGGCGAGUCAAGGUCU AGCCGCAG | 2965 |
| 708 | AGCCCCCC G CAGACGCU | 1019 | AGCGUCUG GCCGAAAGGCGAGUCAAGGUCU GGGGGGCU | 2966 |
| 714 | CCGCAGAC G CUCAACAU | 1020 | AUGUUGAG GCCGAAAGGCGAGUCAAGGUCU GUCUGCGG | 2967 |
| 751 | GUAACUUU G CAGUGGGU | 1021 | ACCCACUG GCCGAAAGGCGAGUCAAGGUCU AAAGUUAC | 2968 |
| 760 | CAGUGGGU G CUGCCCCC | 1022 | GGGGGCAG GCCGAAAGGCGAGUCAAGGUCU ACCCACUG | 2969 |
| 763 | UGGGUGCU G CCCCCCAC | 1023 | GUGGGGGG GCCGAAAGGCGAGUCAAGGUCU AGCACCCA | 2970 |
| 780 | CCCUUCCU G CAUCGCUA | 1024 | UAGCGAUG GCCGAAAGGCGAGUCAAGGUCU AGGAAGGG | 2971 |
| 785 | CCUGCAUC G CUACUACC | 1025 | GGUAGUAG GCCGAAAGGCGAGUCAAGGUCU GAUGCAGG | 2972 |
| 843 | GUGUAUGU G CCCUACAC | 1026 | GUGUAGGG GCCGAAAGGCGAGUCAAGGUCU ACAUACAC | 2973 |
| 921 | GUCACUGU G CGUGCCAA | 1028 | UUGGCACG GCCGAAAGGCGAGUCAAGGUCU ACAGUGAC | 2974 |
| 925 | CUGUGCGU G CCAACAUU | 1029 | AAUGUUGG GCCGAAAGGCGAGUCAAGGUCU ACGCACAG | 2975 |
| 934 | CCAACAUU G CUGCCAUC | 1030 | GAUGGCAG GCCGAAAGGCGAGUCAAGGUCU AAUGUUGG | 2976 |
| 937 | ACAUUGCU G CCAUCACU | 1031 | AGUGAUGG GCCGAAAGGCGAGUCAAGGUCU AGCAAUGU | 2977 |
| 1006 | UGGCCUAU G CUGAGAUU | 1033 | AAUCUCAG GCCGAAAGGCGAGUCAAGGUCU AUAGGCCA | 2978 |
| 1015 | CUGAGAUU G CCAGGCCU | 1035 | AGGCCUGG GCCGAAAGGCGAGUCAAGGUCU AAUCUCAG | 2979 |
| 1092 | UUCUCCCU G CAGCUUUG | 1039 | GAAGCCAG GCCGAAAGGCGAGUCAAGGUCU AGGAGAAA | 2980 |
| 1105 | UUUGUGGU G CUGGCUUC | 1040 | GAAGCCAG GCCGAAAGGCGAGUCAAGGUCU ACCACAAA | 2981 |
| 1134 | UCUGAAGU G CUGGCCUC | 1042 | GAGGCCAG GCCGAAAGGCGAGUCAAGGUCU ACUUCAGA | 2982 |
| 1182 | GACCACUC G CUGUACAC | 1045 | GUGUACAG GCCGAAAGGCGAGUCAAGGUCU GAGUGGUC | 2983 |
| 1248 | AUCAUUGU G CGGGUGGA | 1048 | UCCACCCG GCCGAAAGGCGAGUCAAGGUCU ACAAUGAU | 2984 |
| 1286 | AAUGGACU G CAAGGAGU | 1050 | ACUCCUUG GCCGAAAGGCGAGUCAAGGUCU AGUCCAUU | 2985 |
| 1344 | CUUCGUUU G CCCAAGAA | 1052 | UUCUUGGG GCCGAAAGGCGAGUCAAGGUCU AAACGAAG | 2986 |
| 1366 | UUGAAGCU G CAGUCAAA | 1054 | UUUGACUG GCCGAAAGGCGAGUCAAGGUCU AGCUUCAA | 2987 |
| 1442 | GCUGGUGU G CUGGCAAG | 1056 | CUUGCCAG GCCGAAAGGCGAGUCAAGGUCU ACACCAGC | 2988 |
| 1526 | GUCCUUCC G CAUCACCA | 1058 | UGGUGAUG GCCGAAAGGCGAGUCAAGGUCU GGAAGGAC | 2989 |
| 1542 | AUCCUUCC G CAGCAAUA | 1059 | UAUUGCUG GCCGAAAGGCGAGUCAAGGUCU GGAAGGAU | 2990 |
| 1554 | CAAUACCU G CGGCCAGU | 1060 | ACUGGCCG GCCGAAAGGCGAGUCAAGGUCU AGGUAUUG | 2991 |
| 1603 | ACAAGUUU G CCAUCUCA | 1062 | UGAGAUGG CGGCAAAGGCGAGUCAAGGUCU AAACUUGU | 2992 |
| 1699 | UUGGCUUU G CUGUCAGC | 1066 | GCUGACAG GCCGAAAGGCGAGUCAAGGUCU AAAGCCAA | 2993 |
| 1708 | CUGUCAGC G CUUGCCAU | 1067 | AUGGCAAG GCCGAAAGGCGAGUCAAGGUCU GCUGACAG | 2994 |
| 1712 | CAGCGCUU G CCAUGUGC | 1068 | GCACAUGG GCCGAAAGGCGAGUCAAGGUCU AAGCGCUG | 2995 |
| 1719 | UGCCAUGU G CACGAUGA | 1069 | UCAUCGUG GCCGAAAGGCGAGUCAAGGUCU ACAUGGCA | 2996 |
| 1843 | UCAUGGCU G CCAUCUGC | 1074 | GCAGAUGG GCCGAAAGGCGAGUCAAGGUCU AGCCAUGA | 2997 |
| 1850 | UGCCAUCU G CGCCCUCU | 1075 | AGAGGGCG GCCGAAAGGCGAGUCAAGGUCU AGAUGGCA | 2998 |
| 1852 | CCAUCUGC G CCCUCUUC | 1076 | GAAGAGGG GCCGAAAGGCGAGUCAAGGUCU GCAGAUGG | 2999 |
| 1863 | CUCUUCAU G CUGCCACU | 1077 | AGUGGCAG GCCGAAAGGCGAGUCAAGGUCU AUGAAGAG | 3000 |
| 1866 | UUCAUGCU G CCACUCUG | 1078 | CAGAGUGG GCCGAAAGGCGAGUCAAGGUCU AGCAUGAA | 3001 |
| 1874 | GCCACUCU G CCUCAUGG | 1079 | CCAUGAGG GCCGAAAGGCGAGUCAAGGUCU AGAGUGGC | 3002 |
| 1895 | UCAGUGGC G CUGCCUCC | 1080 | GGAGGCAG GCCGAAAGGCGAGUCAAGGUCU GCCACUGA | 3003 |
| 1898 | GUGGCGCU G CCUCCGCU | 1081 | AGCGGAGG GCCGAAAGGCGAGUCAAGGUCU AGCGCCAC | 3004 |
| 1904 | CUGCCUCC G CUGCCUGC | 1082 | GCAGGCAG GCCGAAAGGCGAGUCAAGGUCU GGAGGCAG | 3005 |
| 1907 | CCUCCGCU G CCUGCGCC | 1083 | GGCGCAGG GCCGAAAGGCGAGUCAAGGUCU AGCGGAGG | 3006 |
| 1911 | CGCUGCCU G CGCCAGCA | 1084 | UGCUGGCG GCCGAAAGGCGAGUCAAGGUCU AGGCAGCG | 3007 |
| 1913 | CUGCCUGC G CCAGCAGC | 1085 | GCUGCUGG GCCGAAAGGCGAGUCAAGGUCU GCAGGCAG | 3008 |
| 1933 | AUGACUUU G CUGAUGAC | 1088 | GUCAUCAG GCCGAAAGGCGAGUCAAGGUCU AAAGUCAU | 3009 |
| 1950 | AUCUCCCU G CUGAAGUG | 1091 | CACUUCAG GCCGAAAGGCGAGUCAAGGUCU AGGGAGAU | 3010 |
| 2087 | CACCAAAU G CCUCUGCC | 1094 | GGCAGAGG GCCGAAAGGCGAGUCAAGGUCU AUUUGGUG | 3011 |
| 2093 | AUGCCUCU G CCUUGAUG | 1095 | CAUCAAGG GCCGAAAGGCGAGUCAAGGUCU AGAGGCAU | 3012 |
| 2179 | AGCAUCUU G CUGGCGGG | 1097 | CCCGCCAG GCCGAAAGGCGAGUCAAGGUCU AGAGUGCU | 3013 |
| 2227 | GAAAUUCU G CUGCUUGA | 1098 | UCAAGCAG GCCGAAAGGCGAGUCAAGGUCU AGAAUUUC | 3014 |
| 2230 | AUUCUGCU G CUUGAAAC | 1099 | GUUUCAAG GCCGAAAGGCGAGUCAAGGUCU AGCAGAAU | 3015 |
| 2329 | CAUCACAC G CAGGUUAC | 1102 | GUAACCUG GCCGAAAGGCGAGUCAAGGUCU GUGUGAUG | 3016 |
| 2393 | GUUUCCCU G CUGGCCAA | 1103 | UUGGCCAG CGGCAAAGGCGAGUCAAGGUCU AGGGAAAC | 3017 |
| 2419 | GAGAGGAU G CACAGUUU | 1104 | AAACUGUG GCCGAAAGGCGAGUCAAGGUCU AUCCUCUC | 3018 |
| 2428 | CACAGUUU G CUAUUUGC | 1105 | GCAAAUAG GCCGAAAGGCGAGUCAAGGUCU AAACUGUG | 3019 |
| 2435 | UGCUAUUU G CUUUAGAG | 1106 | CUCUAAAG CGGCAAAGGCGAGUCAAGGUCU AAAUAGCA | 3020 |
| 2476 | ACAUUGGU G CAAAGAUU | 1107 | AAUCUUUG GCCGAAAGGCGAGUCAAGGUCU ACCAAUGU | 3021 |
| 2485 | CAAAGAUU G CCUCUUGA | 1108 | UCAAGAGG GCCGAAAGGCGAGUCAAGGUCU AAUCUUUG | 3022 |
| 219 | GUGCCGAU G UAGCGGGC | 1110 | GCCCGCUA GCCGAAAGGCGAGUCAAGGUCU AUCGGCAC | 3023 |
| 483 | CUCCUGCU G UGGAUGGG | 1111 | CCCAUCCA GCCGAAAGGCGAGUCAAGGUCU AGCAGGAG | 3024 |
| 634 | GCAGCUUU G UGGAGAUG | 1112 | CAUCUCCA GCCGAAAGGCGAGUCAAGGUCU AAAGCUGC | 3025 |
| 804 | AGGCAGCU G UCCAGCAC | 1113 | GUGCUGGA GCCGAAAGGCGAGUCAAGGUCU AGCUGCCU | 3026 |
| 835 | GGAAGGGU G UGUAUGUG | 1114 | CACAUACA GCCGAAAGGCGAGUCAAGGUCU ACCCUUCC | 3027 |
| 837 | AAGGGUGU G UAUGUGCC | 1115 | GGCACAUA GCCGAAAGGCGAGUCAAGGUCU ACACCCUU | 3028 |
| 841 | GUGUGUAU G UGCCCUAC | 1116 | GUAGGGCA GCCGAAAGGCGAGUCAAGGUCU AUACACAC | 3029 |
| 919 | ACGUCACU G UGCGUGCC | 1117 | GGCACGCA GCCGAAAGGCGAGUCAAGGUCU AGUGACGU | 3030 |
| 1100 | GCAGCUUU G UGGUGCUG | 1118 | CAGCACCA GCCGAAAGGCGAGUCAAGGUCU AAAGCUGC | 3031 |
| 1144 | UGGCCUCU G UCGGAGGG | 1119 | CCCUCCGA GCCGAAAGGCGAGUCAAGGUCU AGAGGCCA | 3032 |
| 1185 | CACUCGCU G UACACAGG | 1120 | CCUGUGUA GCCGAAAGGCGAGUCAAGGUCU AGCGAGUG | 3033 |
| 1246 | UGAUCAUU G UGCGGGUG | 1121 | CACCCGCA GCCGAAAGGCGAGUCAAGGUCU AAUGAUCA | 3034 |
| 1315 | AGAGCAUU G UGGACAGU | 1122 | ACUGUCCA GCCGAAAGGCGAGUCAAGGUCU AAUGCUCU | 3035 |
| 1356 | AAGAAAGU G UUUGAAGC | 1123 | GCUUCAAA GCCGAAAGGCGAGUCAAGGUCU ACGGGCUU | 3036 |
| 1440 | CAGCUGGU G UGCUGGCA | 1124 | UGCCAGCA GCCGAAAGGCGAGUCAAGGUCU ACCAGCUG | 3037 |
| 1570 | UGGAAGAU G UGGCCACG | 1125 | CGUGGCCA GCCGAAAGGCGAGUCAAGGUCU AUCUUCCA | 3038 |
| 1592 | AGACGACU G UUACAAGU | 1126 | ACUUGUAA GCCGAAAGGCGAGUCAAGCUCU AGUCGUCU | 3039 |
| 1630 | CGGGCACU G UUAUGGGA | 1127 | UCCCAUAA GCCGAAAGGCGAGUCAAGGUCU AGUGCCCG | 3040 |
| 1642 | UGGGAGCU G UUAUCAUG | 1128 | CAUGAUGG GCCGAAAGGCGAGUCAAGGUCU AGCUCCCA | 3041 |
| 1666 | UCUACGUU G UCUUUGAU | 1129 | AUCAAAGA GCCGAAAGGCGAGUCAAGGUCU AACGUAGA | 3042 |
| 1702 | GUCUUGCU G UCAGCGCU | 1130 | AGCGCUGA GCCGAAAGGCGAGUCAAGGUCU AGCAAAGC | 3043 |
| 1717 | CUUGCCAU G UGCACGAU | 1131 | AUCGUGCA GCCGAAAGGCGAGUCAAGGUCU AUGGCAAG | 3044 |
| 1759 | GCCCUUUU G UCACCUUG | 1132 | CAAGGUGA GCCGAAAGGCGAGUCAAGGUCU AAAAGGGC | 3045 |
| 1781 | GGAAGACU G UGGCUACA | 1133 | UGUAGCCA GCCGAAAGGCGAGUCAAGGUCU AGUCUUCC | 3046 |
| 1834 | UAGCCUAU G UCAUGGCU | 1134 | AGCCAUGA GCCGAAAGGCGAGUCAAGGUCU AUAGGCUA | 3047 |
| 1884 | CUCAUGGU G UGUCAGUG | 1135 | CACUGACA GCCGAAAGGCGAGUCAAGGUCU ACCAUGAG | 3048 |
| 1886 | CAUGGUGU G UCAGUGGC | 1136 | GCCACUGA GCCGAAAGGCGAGUCAAGGUCU ACACCAUG | 3049 |
| 2048 | UGGCACCU G UGGCCAGA | 1137 | UCUGGCCA GCCGAAAGGCGAGUCAAGGUCU AGGUGCCA | 3050 |
| 2139 | CAGGGACU G UACCUGUA | 1138 | UACAGGUA GCCGAAAGGCGAGUCAAGGUCU AGUCCCUG | 3051 |
| 2145 | CUGUACCU G UAGGAAAC | 1139 | GUUUCCUA GCCGAAAGGCGAGCUAAGGUCU AGGUACAG | 3052 |
| 2256 | GAACCUUU G UCCACCAU | 1140 | AUGGUGGA GCCGAAAGGCGAGUCAAGGUCU AAAGGUUC | 3053 |
| 2346 | CUUGGCGU G UGUCCCUG | 1141 | CAGGGACA GCCGAAAGGCGAGUCAAGGUCU ACGCCAAG | 3054 |
| 2348 | UGGCGUGU G UCCCUGUG | 1142 | CACAGGGA GCCGAAAGGCGAGUCAAGGUCU ACACGCCA | 3055 |
| 2354 | GUGUCCCU G UGGUACCC | 1143 | GGGUACCA GCCGAAAGGCGAGUCAAGGUCU AGGGACAC | 3056 |
| 2385 | CCAAGCUU G UUUCCCUG | 1144 | CAGGGAAA GCCGAAAGGCGAGUCAAGGUCU AAGCUUGG | 3057 |
| 2453 | CAGGGACU G UAUAAACA | 1145 | UGUUUAUA GCCGAAAGGCGAGUCAAGGUCU AGUCCCUG | 3058 |
| 14 | CGUCCGCA G CCCGCCCG | 1146 | CGGGCGGG GCCGAAAGGCGAGUCAAGGUCU UGCGGACG | 3059 |
| 26 | GCCCGGGA G CUGCGACG | 1147 | GCUCGCAG GCCGAAAGGCGAGUCAAGGUCU UCCCGGGC | 3060 |
| 33 | AGCUGCGA G CCGCGAGC | 1148 | GCUGCGCC GCCGAAAGGCGAGUCAAGGUCU UCGCAGCU | 3061 |
| 40 | AGCCGCGA G CUGGAUUA | 1149 | UAAUCCAG GCCGAAAGGCGAGUCAAGGUCU UCGCGGCU | 3062 |
| 51 | GGAUUAUG G UGGCCUGA | 1150 | UCAGGCCA GCCGAAAGGCGAGUCAAGGUCU CAUAAUCC | 3063 |
| 54 | UUAUGGUG G CCUGAGCA | 1151 | UGCUCAGG GCCGAAAGGCGAGUCAAGGUCU CACCAUAA | 3064 |
| 60 | UGGCCUGA G CAGCCAAC | 1152 | GUUGGCUG GCCGAAAGGCGAGUCAAGGUCU UCAGGCCA | 3065 |
| 63 | CCUGAGCA G CCAACGCA | 1153 | UGCGUUGG GCCGAAAGGCGAGUCAAGGUCU UGCUCAGG | 3066 |
| 72 | CCAACGCA G CCGCAGGA | 1154 | UCCUGCGG GCCGAAAGGCGAGUCAAGGUCU UGCGUUGG | 3067 |
| 81 | CCGCAGGA G CCCGGAGC | 1155 | GCUCCGGG GCCGAAAGGCGAGUCAAGGUCU UCCUGCGG | 3068 |
| 88 | AGCCCGGA G CCCUUGCC | 1156 | GGCAAGGG GCCGAAAGGCGAGUCAAGGUCU UCCGGGCU | 3069 |
| 134 | CCAGGGAA G CCGCCACC | 1157 | GGUGGCGG GCCGAAAGGCGAGUCAAGGUCU UUCCCUGG | 3070 |
| 144 | CGCCACCG G CCCGCCAU | 1158 | AUGGCGGG GCCGAAAGGCGAGUCAAGGUCU CGGUGGCG | 3071 |
| 167 | CCCUCCCA G CCCCGCCG | 1159 | CGGCGGGG GCCGAAAGGCGAGUCAAGGUCU UGGGAGGG | 3072 |
| 179 | CGGGCCCA G CCCGCGCC | 1160 | GGCGCGGG GCCGAAAGGCGAGUCAAGGUCU UCCCGGCG | 3073 |
| 198 | CUGCCCAG G CUGGCCGC | 1161 | GCGGCCAG GCCGAAAGGCGAGUCAAGGUCU CUGGGCAG | 3074 |
| 202 | CCAGGCUG G CCGCCGCC | 1162 | GGCGGCGG GCCGAAAGGCGAGUCAAGGUCU CAGCCUGG | 3075 |
| 211 | CCGCCGCC G UGCCGAUG | 1163 | CAUCGGCA GCCGAAAGGCGAGUCAAGGUCU GGCGGCGG | 3076 |
| 222 | CCGAUGUA G CGGGCUCC | 1164 | GGAGCCCG GCCGAAAGGCGAGUCAAGGUCU UACAUCGG | 3077 |
| 226 | UGUAGCGG G CUCCGGAU | 1165 | AUCCGGAG GCCGAAAGGCGAGUCAAGGUCU CCGCUACA | 3078 |
| 239 | GGAUCCCA G CCUCUCCC | 1166 | GGGAGAGG GCCGAAAGGCGAGUCAAGGUCU UGGGAUCC | 3079 |
| 256 | CUGCUCCC G UGCUCUGC | 1167 | GCAGAGCA GCCGAAAGGCGAGUCAAGGUCU GGGAGCAG | 3080 |
| 290 | UCUCCACA G CCCGGACC | 1168 | GGUCCGGG GCCGAAAGGCGAGUCAAGGUCU UGUGGAGA | 3081 |
| 304 | ACCCGGGG G CUGGCCCA | 1169 | UGGGCCAG GCCGAAAGGCGAGUCAAGGUCU CCCCGGGU | 3082 |
| 308 | GGGGGCUG G CCCAGGGC | 1170 | GCCCUGGG GCCGAAAGGCGAGUCAAGGUCU CAGCCCCC | 3082 |
| 315 | GGCCCAGG G CCCUGCAG | 1171 | CUGCAGGG GCCGAAAGGCGAGUCAAGGUCU CCUGGGCC | 3084 |
| 324 | CCCUGCAG G CCCUGGCG | 1172 | CGCCAGGG GCCGAAAGGCGAGUCAAGGUCU CUGCAGGG | 3085 |
| 330 | AGGCCCUG G CGUCCUGA | 1173 | UCAGGACG GCCGAAAGGCGAGUCAAGGUCU CAGGGCCU | 3086 |
| 332 | GCCCUGGC G UCCUGAUG | 1174 | CAUCAGGA GCCGAAAGGCGAGUCAAGGUCU GCCAGGGC | 3087 |
| 348 | GCCCCCAA G CUCCCUCU | 1175 | AGAGGGAG GCCGAAAGGCGAGUCAAGGUCU UUGGGGGC | 3088 |
| 365 | CCUGAGAA G CCACCAGC | 1176 | GCUGGUGG GCCGAAAGGCGAGUCAAGGUCU UUCUCAGG | 3089 |
| 372 | AGCCACAA G CACCACCC | 1177 | GGGUGGUG GCCGAAAGGCGAGUCAAGGUCU UGGUGGCU | 3090 |
| 391 | ACUUGGGG G CAGGCGCC | 1178 | GGCGCCUG GCCGAAAGGCGAGUCAAGGUCU CCCCAAGU | 3091 |
| 395 | GGGGGCAG G CGCCAGGG | 1179 | CCCUGGCG GCCGAAAGGCGAGUCAAGGUCU CUGCCCCC | 3092 |
| 410 | GGACGGAC G UGGGCCAG | 1180 | CUGGCCCA GCCGAAAGGCGAGUCAAGGUCU GUCCGUCC | 3093 |
| 414 | GGACGUGG G CCAGUGCG | 1181 | CGCACUGG GCCGAAAGGCGAGUCAAGGUCU CCACGUCC | 3094 |
| 418 | GUCCGCCA G UGCGAGCC | 1182 | GGCUCGCA GCCGAAAGGCGAGUCAAGGUCU UGGCCCAC | 3095 |
| 424 | CAGUGCGA G CCCAGAGG | 1183 | CCUCUGGG GCCGAAAGGCGAGUCAAGGUCU UCGCACUG | 3096 |
| 433 | CCCAGAGG G CCCGAAGG | 1184 | CCUUCGGG GCCGAAAGGCGAGUCAAGGUCU CCUCUGGG | 3097 |
| 441 | GCCCGAAG G CCGGGGCC | 1185 | GGCCCCGG GCCGAAAGGCGAGUCAAGGUCU CUUCGGGC | 3098 |
| 447 | ACGCCGGG G CCCACCAU | 1186 | AUGGUGGG GCCGAAAGGCGAGUCAAGGUCU CCCGGCCU | 3099 |
| 457 | CCACCAUG G CCCAAGCC | 1187 | GGCUUGGG GCCGAAAGGCGAGUCAAGGUCU CAUGGUGG | 3100 |
| 463 | UGGCCCAA G CCCUGCCC | 1188 | GGGCAGGG GCCGAAAGGCGAGUCAAGGUCU UUGGGCCA | 3101 |
| 474 | CUGCCCUG G CUCCUGCU | 1189 | AGCAGGAG GCCGAAAGGCGAGUCAAGGUCU CAGGGCAG | 3102 |
| 491 | GUGGAUGG G CGCGGGAG | 1190 | CUCCCGCG GCCGAAAGGCGAGUCAAGGUCU CCAUCCAC | 3103 |
| 499 | GCGCGGGA G UGCUGCCU | 1191 | AGGCAGCA GCCGAAAGGCGAGUCAAGGUCU UCCCGCGC | 3104 |
| 515 | UGCCCACG G CACCCAGC | 1192 | GCUGGGUG GCCGAAAGGCGAGUCAAGGUCU CGUGGGCA | 3105 |
| 522 | GGCACCCA G CACGGCAU | 1193 | AUGCCGUG GCCCAAAGGCGAGUCAAGGUCU UGGGUGCC | 3106 |
| 527 | CCAGCACG G CAUCCGGC | 1194 | GCCGGAUG GCCGAAAGGCGAGUCAAGGUCU CGUGCUGG | 3107 |
| 534 | GGCAUCCG G CUGCCCCU | 1195 | AGGGGCAG GCCGAAAGGCGAGUCAAGGUCU CGGAUGCC | 4108 |
| 548 | CCUGCGCA G CGGCCUGG | 1196 | CCAGGCCG GCCGAAAGGCGAGUCAAGGUCU UGCGCAGG | 3109 |
| 551 | GCGCAGCG G CCUGGGGG | 1197 | CCCCCAGG GCCGAAAGGCGAGUCAAGGUCU CGCUGCGC | 3110 |
| 560 | CCUGGGGG G CGCCCCCC | 1198 | GGGGGGCG GCCGAAAGGCGAGUCAAGGUCU CCCCCAGG | 3111 |
| 573 | CCCCUGGG G CUGCGGCU | 1199 | AGCCGCAG GCCGAAAGGCGAGUCAAGGUCU CCCAGGGG | 3112 |
| 579 | GGGCUGCG G CUGCCCCG | 1200 | CGGGCGAG GCCGAAAGGCGAGUCAAGGUCU CGCAGCCC | 3113 |
| 603 | GACGAAGA G CCCGAGGA | 1201 | UCCUCGGG GCCGAAAGGCGAGUCAAGGUCU UCUUCGUC | 3114 |
| 612 | CCCGAGGA G CCCGGCCG | 1202 | CGGCCGGG GCCGAAAGGCGAGUCAAGGUCU UCCUCGGG | 3115 |
| 617 | GGAGCCCG G CCGGAGGG | 1203 | CCCUCCGG GCCGAAAGGCGAGUCAAGGUCU CGGGCUCC | 3116 |
| 626 | CCGGAGGG G CAGCUUUG | 1204 | CAAAGCUG GCCGAAAGGCGAGUCAAGGUCU CCCUCCGG | 3117 |
| 629 | GAGGGGCA G CUUUGUGG | 1205 | CCACAAAG GCCGAAAGGCGAGUCAAGGUCU UGCCCCUC | 3118 |
| 643 | UGGAGAUG G UGGACAAC | 1206 | GUUGUCCA GCCGAAAGGCGAGUCAAGGUCU CAUCUCCA | 3119 |
| 659 | CCUGAGGG G CAAGUCGG | 1207 | CCGACUUG GCCGAAAGGCGAGUCAAGGUCU CCCUCAGG | 3120 |
| 663 | AGGGGCAA G UCGGGGCA | 1208 | UGCCCCGA GCCGAAAGGCGAGUCAAGGUCU UUGCCCCU | 3121 |
| 669 | AAGUCGGG G CAGGGCUA | 1209 | UAGCCCUG GCCGAAAGGCGAGUCAAGGUCU CCCGACUU | 3122 |
| 674 | GGGGCAGG G CUACUACG | 1210 | CGUAGUAG GCCGAAAGGCGAGUCAAGGUCU CCUGCCCC | 3123 |
| 682 | GCUACUAC G UGGAGAUG | 1211 | CAUCUCCA GCCGAAAGGCGAGUCAAGGUCU GUAGUAGC | 3124 |
| 694 | AGAUGACC G UGGGCAGC | 1212 | GCUGCCCA GCCGAAAGGCGAGUCAAGGUCU GGUCAUCU | 3125 |
| 698 | GACCGUGG G CAGCCCCC | 1213 | GGGGGCUG GCCGAAAGGCGAGUCAAGGUCU CCACGGUC | 3126 |
| 701 | CGUGGGCA G CCCCCCGC | 1214 | GCGGGGGG GCCGAAAGGCGAGUCAAGGUCU UGCCCACG | 3127 |
| 727 | ACAUCCUG G UGGAUACA | 1215 | UGUAUCCA GCCGAAAGGCGAGUCAAGGUCU CAGGAUGU | 3128 |
| 737 | GGAUACAG G CAGCAGUA | 1216 | UACUGCUG GCCGAAAGGCGAGUCAAGGUCU CUGUAUCC | 3129 |
| 740 | UACAGGCA G CAGUAACU | 1217 | AGUUACUG GCCGAAAGGCGAGUCAAGGUCU UGCCUGUA | 3130 |
| 743 | AGGCAGCA G UAACUUUG | 1218 | CAAAGUUA GCCGAAAGGCGAGUCAAGGUCU UGCUGCCU | 3131 |
| 754 | ACUUUGCA G UGGGUGCU | 1219 | AGCACCCA GCCGAAAGGCGAGUCAAGGUCU UGCAAACU | 3132 |
| 758 | UGCAGUGG G UGCUGCCC | 1220 | GGGCAGCA GCCGAAAGGCGAGUCAAGGUCU CCACUGCA | 3133 |
| 798 | UACCAGAG G CAGCUGUC | 1221 | GACAGCUG GCCGAAAGGCGAGUCAAGGUCU CUCUGGUA | 3134 |
| 801 | CAGAGGCA G CUGUCCAG | 1222 | CUGGACAG GCCCAAAGGCGAGUCAAGGUCU UGCCUCUG | 3135 |
| 809 | GCUGUCCA G CACAUACC | 1223 | GGUAUGUG GCCGAAAGGCGAGUCAAGGUCU UGGACAGC | 3136 |
| 833 | CCGGAAGG G UGUGUAUG | 1224 | CAUACACA GCCGAAAGGCGAGUCAAGGUCU CCUUCCGG | 3137 |
| 857 | CACCCAGG G CAAGUGGG | 1225 | CCCACUUG GCCGAAAGGCGAGUCAAGGUCU CCUGGGUG | 3138 |
| 861 | CAGGGCAA G UGGGAAGG | 1226 | CCUUCCCA GCCGAAAGGCGAGUCAAGGUCU UUGCCCUG | 3139 |
| 873 | GAAGGGGA G CUGGGCAC | 1227 | GUGCCCAG GCCGAAAGGCGAGUCAAGGUCU UCCCCUUC | 3140 |
| 878 | GGAGCUGG G CACCGACC | 1228 | GGUCGGUG GCCGAAAGGCGAGUCAAGGUCU CCAGCUCC | 3141 |
| 889 | CCGACCUG G UAAGCAUC | 1229 | GAUGCUUA GCCGAAAGGCGAGUCAAGGUCU CAGGUCGG | 3142 |
| 893 | CCUGGUAA G CAUCCCCC | 1230 | GGGGGAUG GCCGAAAGGCGAGUCAAGGUCU UUACCAGG | 3143 |
| 905 | CCCCCAUG G CCCCAACG | 1231 | CGUUGGGG GCCGAAAGGCGAGUCAAGGUCU CAUGGGGG | 3144 |
| 913 | GCCCCAAC G UCACUGUG | 1232 | CACAGUGA GCCGAAAGGCGAGUCAAGGUCU GUUGGGGC | 3145 |
| 923 | CACUGUGC G UGCCAACA | 1233 | UGUUGGCA GCCGAAAGGCGAGUCAAGGUCU GCACAGUG | 3146 |
| 957 | UCAGACAA G UUCUUCAU | 1234 | AUGAAGAA GCCGAAAGGCGAGUCAAGGUCU UUGUCUGA | 3147 |
| 971 | CAUCAACG G CUCCAACU | 1235 | AGUUGGAG GCCGAAAGGCGAGUCAAGGUCU CGUUGAUG | 3148 |
| 986 | CUGGGAAG G CAUCCUGG | 1236 | CCAGGAUG GCCGAAAGGCGAGUCAAGGUCU CUUCCCAG | 3149 |
| 996 | AUCCUGGG G CUGGCCUA | 1237 | UAGGCCAG GCCGAAAGGCGAGUCAAGGUCU CCCAUUAU | 3150 |
| 1000 | UGGGGCUG G CCUAUGCU | 1238 | AGCAUAGG GCCGAAAGGCGAGUCAAGGUCU CAGCCCCA | 3151 |
| 1020 | AUUGCCAG G CCUGACGA | 1239 | UCGUCAGG GCCGAAAGGCGAGUCAAGGUCU CUGGCAAU | 3152 |
| 1038 | UCCCUGGA G CCUUUCUU | 1240 | AAGAAAGG GCCGAAAGGCGAGUCAAGGUCU UCCAGGGA | 3153 |
| 1057 | ACUCUCUG G UAAAGCAG | 1241 | CUGCUUUA GCCGAAAGGCGAGUCAAGGUCU CAGAGAGU | 3154 |
| 1062 | CUGGUAAA G CAGACCCA | 1242 | UGGGUCUG GCCGAAAGGCGAGUCAAGGUCU UUUACCAG | 3155 |
| 1072 | AGACCCAC G UUCCCAAC | 1243 | GUUGGGAA GCCGAAAGGCGAGUCAAGGUCU GUGGGUCU | 3156 |
| 1095 | UCCCUGCA G CUUUGUGG | 1244 | CCACAAAG GCCGAAAGGCGAGUCAAGGUCU UGCAGGGA | 3157 |
| 1103 | GCUUUGUG G UGCUGGCU | 1245 | AGCCAGCA GCCGAAAGGCGAGUCAAGGUCU CACAAAGC | 3158 |
| 1109 | UGGUGCUG G CUUCCCCC | 1246 | GGGGGAAG GCCGAAAGGCGAGUCAAGGUCU CAGCACCA | 3159 |
| 1125 | CUCAACCA G UCUGAAGU | 1247 | ACUUCAGA GCCGAAAGGCGAGUCAAGGUCU UGGUUGAG | 3160 |
| 1132 | AGUCUGAA G UGCUGGCC | 1248 | GGCCAGCA GCCGAAAGGCGAGUCAAGGUCU UUCAGACU | 3161 |
| 1138 | AAGUGCUG G CCUCUGUC | 1249 | GACAGAGG GCCGAAAGGCGAGUCAAGGUCU CAGCACUU | 3162 |
| 1154 | CGGAGGGA G CAUGAUCA | 1250 | UGAUCAUG GCCGAAAGGCGAGUCAAGGUCU UCCCUCCG | 3163 |
| 1169 | CAUUGGAG G UAUCGACC | 1251 | GGUCGAUA GCCGAAAGGCGAGUCAAGGUCU CUCCAAUG | 3164 |
| 1193 | GUACACAG G CAGUCUCU | 1252 | AGAGACUG GCCGAAAGGCGAGUCAAGGUCU CUGUGUAC | 3165 |
| 1196 | CACAGGCA G UCUCUGGU | 1253 | ACCAGAGA GCCGAAAGGCGAGUCAAGGUCU UGCCUGUG | 3166 |
| 1203 | AGUCUCUG G UAUACACC | 1254 | GGUGUAUA GCCGAAAGGCGAGUCAAGGUCU CAGAGACU | 3167 |
| 1218 | CCCAUCCG G CGGGAGUG | 1255 | CACUCCCG GCCGAAAGGCGAGUCAAGGUCU CGGAUGGG | 3168 |
| 1224 | CGGCGGGA G UGGUAUUA | 1256 | UAAUACCA GCCGAAAGGCGAGUCAAGGUCU UCCCGCCG | 3169 |
| 1227 | GCCCAGUG G UAUUAUGA | 1257 | UCAUAAUA GCCGAAAGGCGAGUCAAGGUCU CACUCCCG | 3170 |
| 1237 | AUUAUGAG G UAAUCAUU | 1258 | AAUGAUCA GCCGAAAGGCGAGUCAAGGUCU CUCAUAAU | 3171 |
| 1252 | UUGUGCGG G UGGAGAUC | 1259 | GAUCUCCA GCCGAAAGGCGAGUCAAGGUCU CCGCACAA | 3172 |
| 1293 | UGCAAGGA G UACAACUA | 1260 | UAGUUGUA GCCGAAAGGCGAGUCAAGGUCU UCCUUGCA | 3173 |
| 1310 | UGACAAGA G CAUUGUGG | 1261 | CCACAAUG GCCGAAAGGCGAGUCAAGGUCU UCUUGUCA | 3174 |
| 1322 | UGUGGACA G UGGCACCA | 1262 | UGGUGCCA GCCGAAAGGCGAGUCAAGGUCU UGUCCACA | 3175 |
| 1325 | GGACAGUG G CACCACCA | 1263 | UGGUGGUG GCCGAAAGGCGAGUCAAGGUCU CACUGUCC | 3176 |
| 1340 | CAACCUUC G UUUGCCCA | 1264 | UGGGCAAA GCCGAAAGGCGAGUCAAGGUCU GAAGGUUG | 3177 |
| 1354 | CCAAGAAA G UGUUUGAA | 1265 | UUCAAACA GCCGAAAGGCGACUCAAGGUCU UUUCUUGG | 3178 |
| 1363 | UGUUUGAA G CUGCAGUC | 1266 | GACUGCAG GCCGAAAGGCGAGUCAAGGUCU UUGAAACA | 3179 |
| 1369 | AAGCUGCA G UCAAAUCC | 1267 | GGAUUUGA GCCGAAAGGCGAGUCAAGGUCU UGCAGCUU | 3180 |
| 1384 | CCAUCAAG G CAGCCUCC | 1268 | GGAGGCUG GCCGAAAGGCGAGUCAAGGUCU CUUGAUGG | 3181 |
| 1387 | UCAAGGCA G CCUCCUCC | 1269 | GGAGGAGG GCCGAAAGGCGAGUCAAGGUCU UGCCUUGA | 3182 |
| 1404 | ACGGAGAA G UUCCCUGA | 1270 | UCAGGGAA GCCGAAAGGCGAGUCAAGGUCU UUCUCCGU | 3183 |
| 1415 | CCCUGAUG G UUUCUGGC | 1271 | CGGAGAAA GCCGAAAGGCGAGUCAAGGUCU CAUCAGGG | 3184 |
| 1422 | GGUUUCUG G CUAGGAGA | 1272 | UCUCCUAG GCCGAAAGGCGAGUCAAGGUCU GACAAACC | 3185 |
| 1431 | CUAGGAGA G CAGCUGGU | 1273 | ACCAGCUG GCCGAAAGGCGAGUCAAGGUCU UCUCCUAG | 3186 |
| 1434 | GGAGAGCA G CUGGUGUG | 1274 | CACACCAG GCCGAAAGGCGAGUCAAGGUCU UGCUCUCC | 3187 |
| 1438 | AGCAGCUG G UGUGCUGG | 1275 | CCAGCACA GCCGAAAGGCGAGUCAAGGUCU CAGCUGCU | 3188 |
| 1446 | GUGUGCUG G CAAGCAGG | 1276 | CCUGCUUG GCCGAAAGGCGAGUCAAGGUCU CAGCACAC | 3189 |
| 1450 | GCUGGCAA G CAGGCACC | 1277 | GGUGCCUG GCCGAAAGGCGAGUCAAGGUCU UUGCCAGC | 3190 |
| 1454 | GCAAGCAG G CACCACCC | 1278 | GGGUGGUG GCCGAAAGGCGAGUCAAGGUCU CUGCUUGC | 3191 |
| 1480 | UUUUCCCA G UCAUCUCA | 1279 | UGAGAUGA GCCGAAAGGCGAGUCAAGGUCU UGGAAAAA | 3192 |
| 1502 | CCUAAUGG G UGAGGUUA | 1280 | UAACCUCA GCCGAAAGGCGAGUCAAGGUCU CCAUUAGG | 3193 |
| 1507 | UGGGUGAG G UUACCAAC | 1281 | GUUGGUAA GCCGAAAGGCGAGUCAAGGUCU CUCACCCA | 3194 |
| 1518 | ACCAACCA G UCCUUCCG | 1282 | CGGAAGGA GCCGAAAGGCGAGUCAAGGUCU UGGUUGGU | 3195 |
| 1545 | CUUCCGCA G CAAUACCU | 1283 | AGGUAUUG GCCGAAAGGCGAGUCAAGGUCU UGCGGAAG | 3196 |
| 1557 | UACCUGCG G CCAGUGGA | 1284 | UCCACUGG GCCGAAAGGCGAGUCAAGGUCU CGCAGGUA | 3197 |
| 1561 | UGCGGCCA G UGGAAGAU | 1285 | AUCUUCCA GCCGAAAGGCGAGUCAAGGUCU UGGCCGCA | 3198 |
| 1573 | AAGAUGUG G CCACGUCC | 1286 | GGACGUGG GCCGAAAGGCGAGUCAAGGUCU CACAUCUU | 3199 |
| 1578 | GUGGCCAC G UCCCAAGA | 1287 | UCUUGGGA GCCGAAAGGCGAGUCAAGGUCU GUGGCCAC | 3200 |
| 1599 | UGUUACAA G UUUGCCAU | 1288 | AUGGCAAA GCCGAAAGGCGAGUCAAGGUCU UUGUAACA | 3201 |
| 1614 | AUCUCACA G UCAUCCAC | 1289 | GUGGAUGA GCCGAAAGGCGAGUCAAGGUCU UGUGAGAU | 3202 |
| 1625 | AUCCACGG G CACUGUUA | 1290 | UAACAGUG GCCGAAAGGCGAGUCAAGGUCU CCGUGGAU | 3203 |
| 1639 | UUAUGGGA G CUGUUAUC | 1291 | GAUAACAG GCCGAAAGGCGAGUCAAGGUCU UCCCAUAA | 3204 |
| 1655 | CAUGGAGG G CUUCUACG | 1292 | GGUAGAAG GCCGAAAGGCGAGUCAAGGUCU CCUCCAUG | 3205 |
| 1663 | GCUUCUAC G UUGUCUUU | 1293 | AAAGACAA GCCGAAAGGCGAGUCAAGGUCU GUAGAAGC | 3206 |
| 1678 | UUGAUCGG G CCGAAAAA | 1294 | UUUUCGGG GCCGAAAGGCGAGUCAAGGUCU CCGAUCAA | 3207 |
| 1694 | ACGAAUUG G CUUUGCUG | 1295 | CAGCAAAG GCCGAAAGGCGAGUCAAGGUCU CAAUUCGU | 3208 |
| 1706 | UGCUGUCA G CGCUUGCC | 1296 | GGCAAGCG GCCGAAAGGCGAGUCAAGGUCU UGACAGCA | 3209 |
| 1728 | CACGAUGA G UUCAGGAC | 1297 | GUCCUGAA GCCGAAAGGCGAGUCAAGGUCU UCAUCGUG | 3210 |
| 1738 | UCAGGACG G CAGCGGUG | 1298 | CACCGCUG GCCGAAAGGCGAGUCAAGGUCU GCUCCUGA | 3211 |
| 1741 | GGACGGCA G CGGUGGAA | 1299 | UUCCACCG GCCGAAAGGCGAGUCAAGGUCU UGCCGUCC | 3212 |
| 1744 | CGGCAGCG G UGGAAGGC | 1300 | GCCUUCCA GCCGAAAGGCGAGUCAAGGUCU CGCUGCCG | 3213 |
| 1751 | GGUGGAAG G CCCUUUUG | 1301 | CAAAAGGG GCCGAAAGGCGAGUCAAGGUCU CUUCCACC | 3214 |
| 1784 | AGACUGUG G CUACAACA | 1302 | UGUUGUAG GCCGAAAGGCGAGUCAAGGUCU CACAGUCU | 3215 |
| 1809 | ACAGAUGA G UCAACCCU | 1303 | AGGGUUGA GCCGAAAGGCGAGUCAAGGUCU AUCACUGU | 3216 |
| 1828 | UGACCAUA G CCUAUGUG | 1304 | GACAUAGG GCCGAAAGGCGAGUCAAGGUCU AUAGGUCA | 3217 |
| 1840 | AUGUCAUG G CUGCCAUC | 1305 | GAUGGCAG GCCGAAAGGCGAGUCAAGGUCU CAUGACAU | 3218 |
| 1882 | GCCUCAUG G UGUGUCAG | 1306 | CUGACACA GCCGAAAGGCGAGUCAAGGUCU CAUGAGGC | 3219 |
| 1890 | GUGUGUCA G UGGCGCUG | 1307 | CAGCGCCA GCCGAAAGGCGAGUCAAGGUCU UGACACAC | 3220 |
| 1893 | UGUCAGUG G CGCUGCCU | 1308 | AGGCAGCG GCCGAAAGGCGAGUCAAGGUCU CACUGACA | 3221 |
| 1917 | CUGCGCCA G CAGCAUGA | 1309 | UCAUGCUG GCCGAAAGGCGAGUCAAGGUCU UGGCGCAG | 3222 |
| 1920 | CGCCAGCA G CAUGAUGA | 1310 | UCAUCAUG GCCGAAAGGCGAGUCAAGGUCU UGCUGGCG | 3223 |
| 1956 | CUGCUGAA G UGAGGAGG | 1311 | CCUCCUCA GCCGAAAGGCGAGUCAAGGUCU UUCAGCAG | 3224 |
| 1964 | GUGAGGAG G CCCAUGGG | 1312 | CCCAUGGG GCCGAAAGGCGAGUCAAGGUCU CUCCUCAC | 3225 |
| 1972 | GCCCAUGG G CAGAAGAU | 1313 | AUCUUCUG GCCGAAAGGCGAGUCAAGGUCU CCAUGGGC | 3226 |
| 2006 | ACACCUCC G UGGUUCAC | 1314 | GUGAACCA GCCGAAAGGCGAGUCAAGGUCU GGAGGUGU | 3227 |
| 2009 | CCUCCGUG G UUCACUUU | 1315 | AAAGUGAA GCCGAAAGGCGAGUCAAGGUCU CACGGAGG | 3228 |
| 2019 | UCACUUUG G UCACAAGU | 1316 | ACUUGUGA GCCGAAAGGCGAGUCAAGGUCU UUGUGACC | 3229 |
| 2026 | GGUCACAA G UAGGAGAC | 1317 | GUCUCCUA GCCGAAAGGCGAGUCAAGGUCU UUGUGACC | 3230 |
| 2042 | CACAGAUG G CACCUGUG | 1318 | CACAGGUG GCCGAAAGGCGAGUCAAGGUCU CAUCUGUG | 3231 |
| 2051 | CACCUGUG G CCAGAGCA | 1319 | UGCUCUGG GCCGAAAGGCGAGUCAAGGUCU CACAGGUG | 3232 |
| 2057 | UGGCCAGA G CACCUCAG | 1320 | CUGAGGUG GCCGAAAGGCGAGUCAAGGUCU UCUGGCCA | 3233 |
| 2114 | AGGAAAAG G CUGGCAAG | 1321 | CUUGCCAG GCCGAAAGGCGAGUCAAGGUCU CUUUUCCU | 3234 |
| 2118 | AAAGGCUG G CAAGGUGG | 1322 | CCACCUUG GCCGAAAGGCGAGUCAAGGUCU CAGCCUUU | 3235 |
| 2123 | CUGGCAAG G UGGGUUCC | 1323 | GGAACCCA GCCGAAAGGCGAGUCAAGGUCU CUUGCCAG | 3236 |
| 2127 | CAAGGUGG G UUCCAGGG | 1324 | CCCUGGAA GCCGAAAGGCGAGUCAAGGUCU CCACCUUG | 3237 |
| 2172 | AGAAAGAA G CACUCUGC | 1325 | GCAGAGUG GCCGAAAGGCGAGUCAAGGUCU UUCUUUCU | 3238 |
| 2183 | CUCUGCUG G CGGGAAUA | 1326 | UAUUCCCG GCCGAAAGGCGAGUCAAGGUCU CAGCAGAG | 3239 |
| 2198 | UACUCUUG G UCACCUCA | 1327 | UGAGGUGA GCCGAAAGGCGAGUCAAGGUCU CAAGAGUA | 3240 |
| 2214 | AAAUUUAA G UCGGGAAA | 1328 | UUUCCCGA GCCGAAAGGCGAGUCAAGGUCU UUAAAUUU | 3241 |
| 2243 | AAACUUCA G CCCUGAAC | 1329 | GUUCAGGG GCCGAAAGGCGAGUCAAGGUCU UGAAGUUU | 3242 |
| 2288 | AACCCAAA G UAUUCUUC | 1330 | GAAGAAUA GCCGAAAGGCGAGUCAAGGUCU UUUGGGUU | 3243 |
| 2305 | UUUUCUUA G UUUCAGAA | 1331 | UUCUGAAA GCCGAAAGCGGAGUCAAGGUCU UAAGAAAA | 3244 |
| 2314 | UUUCAGAA G UACUGGCA | 1332 | UGCCAGUA GCCGAAAGGCGAGUCAAGGUCU UUCUGAAA | 3245 |
| 2320 | AAGUACUG G CAUCACAC | 1333 | GUGUGAUG GCCGAAAGGCGAGUCAAGGUCU CAGUACUU | 3246 |
| 2333 | ACACGCAG G UUACCUUG | 1334 | CAAGGUAA GCCGAAAGGCGAGUCAAGGUCU CUGCGUGU | 3247 |
| 2342 | UUACCUUG G CGUGUGUC | 1335 | GACACACG GCCGAAAGGCGAGUCAAGGUCU CAAGGUAA | 3248 |
| 2344 | ACCUUGGC G UGUGUCCC | 1336 | GGGACACA GCCGAAAGGCGAGUCAAGGUCU GCCAAGGU | 3249 |
| 2357 | UCCCUGUG G UACCCUGG | 1337 | CCAGGGUA GCCGAAAGGCGAGUCAAGGUCU CACAGGGA | 3250 |
| 2365 | GUACCCUG G CAGAGAAG | 1338 | CUUCUCUG GCCGAAAGGCGAGUCAAGGUCU CAGGGUAC | 3251 |
| 2381 | GAGACCAA G CUUGUUUC | 1339 | GAAACAAG GCCGAAAGGCGAGUCAAGGUCU UUGGUCUC | 3252 |
| 2397 | CCCUGCUG G CCAAAGUC | 1340 | GACUUUGG GCCGAAAGGCGAGUCAAGGUCU CAGCAGGG | 3253 |
| 2403 | UGGCCAAA G UCAGUAGG | 1341 | CCUACUGA GCCGAAAGGCGAGUCAAGGUCU UUUGGCCA | 3254 |
| 2407 | CAAAGUCA G UAGGAGAG | 1342 | CUCUCCUA GCCGAAAGGCGAGUCAAGGUCU UGACUUUG | 3255 |
| 2424 | GAUGCACA G UUUGCUAU | 1343 | AUAGCAAA GCCGAAAGGCGAGUCAAGGUCU UGUGCAUG | 3256 |
| 2463 | AUAAACAA G CCUAACAU | 1344 | AUGUUAGG GCCGAAAGGCGAGUCAAGGUCU UUGUUUAU | 3257 |
| 2474 | UAACAUGG G UGCAAAGA | 1345 | UCUUUGCA GCCGAAAGGCGAGUCAAGGUCU CAAUGUUA | 3258 |

Input Sequence = AF190725. Cut Site = g/.
Stem Length = 8 . Core Sequence = GCcgaaagGCGaGuCaaGGuCu
AF190725 (Homo sapiens beta-site APP cleaving enzyme (BACE) mRNA; 2526 bp)

TABLE VII

uz,11/36 Human BACE DNAzyme and Target Sequence

Rz Seq

| | | | | |
|---|---|---|---|---|
| Pos | Substrate | Seq ID | Ribozyme | ID |

| | | | | |
|---|---|---|---|---|
| 48 | GCUGGAUU A UGGUGGCC | 3 | GGCCACCA GGCTAGCTACAACGA AATCCAGC | 3259 |
| 677 | GCAGGGCU A CUACGUGG | 27 | CCACGTAG GGCTAGCTACAACGA AGCCCTGC | 3260 |
| 680 | GGGCUACU A CGUGGAGA | 28 | TCTCCACG GGCTAGCTACAACGA AGTAGCCC | 3261 |
| 733 | UGGUGGAU A CAGGCAGC | 31 | GCTGCCTG GGCTAGCTACAACGA ATCCACCA | 3262 |
| 788 | GCAUCGCU A CUACCAGA | 38 | TCTGGTAG GGCTAGCTACAACGA AGCGATGC | 3263 |
| 791 | UCGCUACU A CCAGAGGC | 39 | GCCTCTGG GGCTAGCTACAACGA AGTAGCGA | 3264 |
| 815 | CAGCACAU A CCGGGACC | 41 | GGTCCCGG GGCTAGCTACAACGA ATGTGCTG | 3265 |
| 839 | GGGUGUGU A UGUGCCCU | 43 | AGGGCACA GGCTAGCTACAACGA ACACACCC | 3266 |
| 848 | UGUGCCCU A CACCCAGG | 44 | CCTGGGTG GGCTAGCTACAACGA AGGGCACA | 3267 |
| 1004 | GCUGGCCU A UGCUGAGA | 58 | TCTCAGCA GGCTAGCTACAACGA AGGCCAGC | 3268 |
| 1171 | UUGGAGGU A UCGACCAC | 85 | GTGGTCGA GGCTAGCTACAACGA ACCTCCAA | 3269 |
| 1187 | CUCGCUGU A CACAGGCA | 88 | TGCCTGTG GGCTAGCTACAACGA ACAGCGAG | 3270 |
| 1205 | UCUCUGGU A UACACCCA | 91 | TGGGTGTA GGCTAGCTACAACGA ACCAGAGA | 3271 |
| 1207 | UCUGGUAU A CACCCAUC | 92 | GATGGGTG GGCTAGCTACAACGA ATACCAGA | 3272 |
| 1229 | GGAGUGGU A UUAUGAGG | 94 | CCTCATAA GGCTAGCTACAACGA ACCACTCC | 3273 |
| 1232 | GUGGUAUU A UGAGGUGA | 96 | TCACCTCA GGCTAGCTACAACGA AATACCAC | 3274 |
| 1295 | CAAGGAGU A CAACUAUG | 101 | CATAGTTG GGCTAGCTACAACGA ACTCCTTG | 3275 |
| 1301 | GUACAACU A UGACAAGA | 102 | TCTTGTCA GGCTAGCTACAACGA AGTTGTAC | 3276 |
| 1493 | CUCACUCU A CCUAAUGG | 130 | CCATTAGG GGCTAGCTACAACGA AGAGTGAG | 3277 |
| 1510 | GUGAGGUU A CCAACCAG | 133 | CTGGTTGG GGCTAGCTACAACGA AACCTCAC | 3278 |
| 1550 | GCAGCAAU A CCUGCGGC | 141 | GCCGCAGG GGCTAGCTACAACGA ATTGCTGC | 3279 |
| 1595 | CGACUGUU A CAAGUUUG | 144 | CAAACTTG GGCTAGCTACAACGA AACAGTCG | 3280 |
| 1633 | GCACUGUU A UGGGAGCU | 152 | AGCTCCCA GGCTAGCTACAACGA AACAGTGC | 3281 |
| 1645 | GAGCUGUU A UCAUGGAG | 154 | CTCCATGA GGCTAGCTACAACGA AACAGCTC | 3282 |
| 1661 | GGGCUUCU A CGUUGUCU | 158 | AGACAACG GGCTAGCTACAACGA AGAAGCCC | 3283 |
| 1787 | CUGUGGCU A CAACAUUC | 176 | GAATGTTG GGCTAGCTACAACGA AGCCACAG | 3284 |
| 1832 | CAUAGCCU A UGUCAUGG | 182 | CCATGACA GGCTAGCTACAACGA AGGCTATG | 3285 |
| 2141 | GGGACUGU A CCUGUAGG | 212 | CCTACAGG GGCTAGCTACAACGA ACAGTCCC | 3286 |
| 2191 | GCGGGAAU A CUCUUGGU | 215 | ACCAAGAG GGCTAGCTACAACGA ATTCCCGC | 3287 |
| 2290 | CCCAAAGU A UUCUUCUU | 240 | AAGAAGAA GGCTAGCTACAACGA ACTTTGGG | 3288 |
| 2316 | UCAGAAGU A CUGGCAUC | 254 | GATGCCAG GGCTAGCTACAACGA ACTTCTGA | 3289 |
| 2336 | CGCAGGUU A CCUUGGCG | 257 | CGCCAAGG GGCTAGCTACAACGA AACCTGCG | 3290 |
| 2359 | CCUGUGGU A CCCUGGCA | 260 | TGCCAGGG GGCTAGCTACAACGA ACCACAGG | 3291 |
| 2431 | AGUUUGCU A UUUGCUUU | 269 | AAAGCAAA GGCTAGCTACAACGA AGCAAACT | 3292 |
| 2455 | GGGACUGU A UAAACAAG | 275 | CTTGTTTA GGCTAGCTACAACGA ACAGTCCC | 3293 |
| 140 | AAGCCGCC A CCGGCCCG | 322 | CGGGCCGG GGCTAGCTACAACGA GGCGGCTT | 3294 |
| 151 | GGCCCGCC A UGCCCGCC | 327 | GGCGGGCA GGCTAGCTACAACGA GGCGGGCC | 3295 |
| 287 | CGCUCUCC A CAGCCCGG | 380 | CCGGGCTG GGCTAGCTACAACGA GGAGAGCG | 3296 |
| 368 | GAGAAGCC A CCAGCACC | 412 | GGTGCTGG GGCTAGCTACAACGA GGCTTCTC | 3297 |
| 374 | CCACCAGC A CCACCCAG | 415 | CTGGGTGG GGCTAGCTACAACGA GCTGGTGG | 3298 |
| 377 | CCAGCACC A CCCAGACU | 417 | AGTCTGGG GGCTAGCTACAACGA GGTGCTGG | 3299 |
| 451 | CGGGGCCC A CCAUGGCC | 435 | GGCCATGG GGCTAGCTACAACGA GGGCCCCG | 3300 |
| 454 | GGCCCACC A UGGCCCAA | 437 | TTGGGCCA GGCTAGCTACAACGA GGTGGGCC | 3301 |
| 512 | GCCUGCCC A CGGCACCC | 456 | GGGTGCCG GGCTAGCTACAACGA GGGCAGGC | 3302 |
| 517 | CCCACGGC A CCCAGCAC | 457 | GTGCTGGG GGCTAGCTACAACGA GCCGTGGG | 3303 |
| 524 | CACCCAGC A CGGCAUCC | 461 | GGATGCCG GGCTAGCTACAACGA GCTGGGTG | 3304 |
| 529 | AGCACGGC A UCCGGCUG | 462 | CAGCCGGA GGCTAGCTACAACGA GCCGTGCT | 3305 |
| 721 | CGCUCAAC A UCCUGGUG | 508 | CACCAGGA GGCTAGCTACAACGA GTTGAGCG | 3306 |
| 770 | UGCCCCCC A CCCCUUCC | 522 | GGAAGGGG GGCTAGCTACAACGA GGGGGGCA | 3307 |
| 782 | CUUCCUGC A UCGCUACU | 529 | AGTAGCGA GGCTAGCTACAACGA GCAGGAAG | 3308 |
| 811 | UGUCCAGC A CAUACCGG | 538 | CCGGTATG GGCTAGCTACAACGA GCTGGACA | 3309 |
| 813 | UCCAGCAC A UACCGGGA | 539 | TCCCGGTA GGCTAGCTACAACGA GTGCTGGA | 3310 |
| 852 | UGCCCUAC A CCCAGGGC | 547 | GCCCTGGG GGCTAGCTACAACGA GTAGGGCA | 3311 |
| 880 | AGCUGGGC A CCGACCUG | 553 | CAGGTCGG GGCTAGCTACAACGA GCCCAGCT | 3312 |
| 895 | UGGUAAGC A UCCCCCAU | 557 | ATGGGGGA GGCTAGCTACAACGA GCTTACCA | 3313 |
| 902 | CAUCCCCC A UGGCCCCA | 562 | TGGGGCCA GGCTAGCTACAACGA GGGGGATG | 3314 |
| 916 | CCAACGUC A CUGUGCGU | 567 | ACGCACAG GGCTAGCTACAACGA GACGTTGG | 3315 |
| 931 | GUGCCAAC A UUGCUGCC | 571 | GGCAGCAA GGCTAGCTACAACGA GTTGGCAC | 3316 |
| 940 | UUGCUGCC A UCACUGAA | 574 | TTCAGTGA GGCTAGCTACAACGA GGCAGCAA | 3317 |
| 943 | CUGCCAUC A CUGAAUCA | 575 | TGATTCAG GGCTAGCTACAACGA GATGGCAG | 3318 |
| 964 | AGUUCUUC A UCAACGGC | 580 | GCCGTTGA GGCTAGCTACAACGA GAAGAACT | 3319 |
| 988 | GGGAAGGC A UCCUGGGG | 586 | CCCCAGGA GGCTAGCTACAACGA GCCTTCCC | 3320 |
| 1070 | GCAGACCC A CGUUCCCA | 610 | TGGGAACG GGCTAGCTACAACGA GGGTCTGC | 3321 |
| 1156 | GAGGGAGC A UCAUCAUU | 638 | AATGATCA GGCTAGCTACAACGA GCTCCCTC | 3322 |
| 1162 | GCAUGAUC A UUGGAGGU | 639 | ACCTCCAA GGCTAGCTACAACGA GATCATGC | 3323 |
| 1178 | UAUCGACC A CUCGCUGU | 641 | ACAGCGAG GGCTAGCTACAACGA GGTCGATA | 3324 |
| 1189 | CGCUGUAC A CAGGCAGU | 644 | ACTGCCTG GGCTAGCTACAACGA GTACAGCG | 3325 |
| 1209 | UGGUAUAC A CCCAUCCG | 649 | CGGATGGG GGCTAGCTACAACGA GTATACCA | 3326 |
| 1213 | AUACACCC A UCCGGCGG | 652 | CCGCCGGA GGCTAGCTACAACGA GGGTGTAT | 3327 |
| 1243 | AGGUGAUC A UUGUGCGG | 654 | CCGCACAA GGCTAGCTACAACGA GATCACCT | 3328 |
| 1312 | ACAAGAGC A UUGUGGAC | 663 | GTCCACAA GGCTAGCTACAACGA GCTCTTGT | 3329 |
| 1327 | ACAGUGGC A CCACCAAC | 665 | GTTGGTGG GGCTAGCTACAACGA GCCACTGT | 3330 |
| 1330 | GUGGCACC A CCAACCUU | 667 | AAGGTTGG GGCTAGCTACAACGA GGTGCCAC | 3331 |
| 1378 | UCAAAUCC A UCAAGGCA | 679 | TGCCTTGA GGCTAGCTACAACGA GGATTTGA | 3332 |
| 1396 | CCUCCUCC A CGGAGAAG | 687 | CTTCTCCG GGCTAGCTACAACGA GGAGGAGG | 3333 |
| 1456 | AAGCAGGC A CCACCCCU | 698 | AGGGGTGG GGCTAGCTACAACGA GCCTGCTT | 3334 |
| 1459 | CAGGCACC A CCCCUUGG | 700 | CCAAGGGG GGCTAGCTACAACGA GGTGCCTG | 3335 |
| 1471 | CUUGGAAC A UUUUCCCA | 705 | TGGGAAAA GGCTAGCTACAACGA GTTCCAAG | 3336 |
| 1483 | UCCCAGUC A UCUCACUC | 709 | GAGTGAGA GGCTAGCTACAACGA GACTGGGA | 3337 |
| 1488 | GUCAUCUC A CUCUACCU | 711 | AGGTAGAG GGCTAGCTACAACGA GAGATGAC | 3338 |
| 1528 | CCUUCCGC A UCACCAUC | 723 | GATGGTGA GGCTAGCTACAACGA GCGGAAGG | 3339 |
| 1531 | UCCGCAUC A CCAUCCUU | 724 | AAGGATGG GGCTAGCTACAACGA GATGCGGA | 3340 |
| 1534 | GCAUCACC A UCCUUCCG | 726 | CGGAAGGA GGCTAGCTACAACGA GGTGATGC | 3341 |
| 1576 | AUGUGGCC A CGUCCCAA | 737 | TTGGGACG GGCTAGCTACAACGA GGCCACAT | 3342 |
| 1606 | AGUUUGCC A UCUCACAG | 744 | CTGTGAGA GGCTAGCTACAACGA GGCAAACT | 3343 |
| 1611 | GCCAUCUC A CAGUCAUC | 746 | GATGACTG GGCTAGCTACAACGA GAGATGGC | 3344 |
| 1617 | UCACAGUC A UCCACGGG | 748 | CCCGTGGA GGCTAGCTACAACGA GACTGTGA | 3345 |
| 1621 | AGUCAUCC A CGGGCACU | 750 | AGTGCCCG GGCTAGCTACAACGA GGATGACT | 3346 |
| 1627 | CCACGGGC A CUGUUAUG | 751 | CATAACAG GGCTAGCTACAACGA GCCCGTGG | 3347 |
| 1648 | CUGUUAUC A UGGAGGGC | 754 | GCCCTCCA GGCTAGCTACAACGA GATAACAG | 3348 |
| 1715 | CGCUUGCC A UGUGCACG | 765 | CGTGCACA GGCTAGCTACAACGA GGCAAGCG | 3349 |
| 1721 | CCAUGUGC A CGAUGAGU | 766 | ACTCATCG GGCTAGCTACAACGA GCACATGG | 3350 |
| 1762 | CUUUUGUC A CCIIGGAC | 772 | GTCCAAGG GGCTAGCTACAACGA GACAAAAG | 3351 |
| 1771 | CCUUGGAC A UGGAAGAC | 775 | GTCTTCCA GGCTAGCTACAACGA GTCCAAGG | 3352 |
| 1792 | GCUACAAC A UUCCACAG | 779 | CTGTGGAA GGCTAGCTACAACGA GTTGTAGC | 3353 |
| 1797 | AACAUUCC A CAGACAGA | 781 | TCTGTCTG GGCTAGCTACAACGA GGAATGTT | 3354 |
| 1819 | CAACCCUC A UGACCAUA | 788 | TATGGTCA GGCTAGCTACAACGA GAGGGTTG | 3355 |
| 1825 | UCAUGACC A UAGCCUAU | 790 | ATAGGCTA GGCTAGCTACAACGA GGTCATGA | 3356 |
| 1837 | CCUAUGUC A UGGCUGCC | 793 | GGCAGCCA GGCTAGCTACAACGA GACATAGG | 3357 |
| 1846 | UGGCUGCC A UCUGCGCC | 796 | GGCGCAGA GGCTAGCTACAACGA GGCAGCCA | 3358 |
| 1861 | CCCUCUUC A UGCUGCCA | 802 | TGGCAGCA GGCTAGCTACAACGA GAAGAGGG | 3359 |
| 1869 | AUGCUGCC A CUCUGCCU | 805 | AGGCAGAG GGCTAGCTACAACGA GGCAGCAT | 3360 |
| 1879 | UCUGCCUC A UGGUGUGU | 810 | ACACACCA GGCTAGCTACAACGA GAGGCAGA | 3361 |
| 1922 | CCAGCAGC A UGAUGACU | 822 | AGTCATCA GGCTAGCTACAACGA GCTGCTGG | 3362 |
| 1942 | CUGAUGAC A UCUCCCUG | 825 | CAGGGAGA GGCTAGCTACAACGA GTCATCAG | 3363 |
| 1968 | GGAGGCCC A UGGGCAGA | 833 | TCTGCCCA GGCTAGCTACAACGA GGGCCTCC | 3364 |
| 1998 | CCUGGACC A CACCUCCG | 840 | CGGAGGTG GGCTAGCTACAACGA GGTCCAGG | 3365 |
| 2000 | UGGACCAC A CCUCCGUG | 841 | CACGGAGG GGCTAGCTACAACGA GTGGTCCA | 3366 |
| 2013 | CGUGGUUC A CUUUGGUC | 845 | GACCAAAG GGCTAGCTACAACGA GAACCACG | 3367 |
| 2022 | CUUUGGUC A CAAGUAGG | 847 | CCTACTTG GGCTAGCTACAACGA GACCAAAG | 3368 |
| 2035 | UAGGAGAC A CAGAUGGC | 849 | GCCATCTG GGCTAGCTACAACGA GTCTCCTA | 3369 |
| 2044 | CAGAUGGC A CCUGUGGC | 851 | GCCACAGG GGCTAGCTACAACGA GCCATCTG | 3370 |
| 2059 | GCCAGAGC A CCUCAGGA | 856 | TCCTGAGG GGCTAGCTACAACGA GCTCTGGC | 3371 |
| 2076 | CCCUCCCC A CCCACCAA | 866 | TTGGTCGG GGCTAGCTACAACGA GGGGAGGG | 3372 |
| 2080 | CCCCACCC A CCAAAUGC | 869 | GCATTTGG GGCTAGCTACAACGA GGGTGGGG | 3373 |
| 2174 | AAAGAAGC A CUCUGCUG | 885 | CAGCAGAG GGCTAGCTACAACGA GCTTCTTT | 3374 |
| 2201 | UCUUGGUC A CCUCAAAU | 891 | ATTTGAGG GGCTAGCTACAACGA GACCAAGA | 3375 |
| 2260 | CUUUGUCC A CCAUUCCU | 906 | AGGAATGG GGCTAGCTACAACGA GGACAAAG | 3376 |
| 2263 | UGUCCACC A UUCCUUUA | 908 | TAAAGGAA GGCTAGCTACAACGA GGTGGACA | 3377 |
| 2322 | GUACUGGC A UCACACGC | 922 | GCGTGTGA GGCTAGCTACAACGA GCCAGTAC | 3378 |
| 2325 | CUGGCAUC A CACGCAGG | 923 | CCTGCGTG GGCTAGCTACAACGA GATGCCAG | 3379 |
| 2327 | GGCAUCAC A CGCAGGUU | 924 | AACCTGCG GGCTAGCTACAACGA GTGATGCC | 3380 |
| 2421 | GAGGAUGC A CAGUUUGC | 945 | GCAAACTG GGCTAGCTACAACGA GCATCCTC | 3381 |
| 2470 | AGCCUAAC A UUGGUGCA | 954 | TGCACCAA GGCTAGCTACAACGA GTTAGGCT | 3382 |
| 11 | ACGCGUCC G CAGCCCGC | 960 | GCGGGCTG GGCTAGCTACAACGA GGACGCGT | 3383 |
| 18 | CGCAGCCC G CCCGGGAG | 961 | CTCCCGGG GGCTAGCTACAACGA GGGCTGCG | 3384 |
| 29 | CGGGAGCU G CGAGCCGC | 962 | GCGGCTCG GGCTAGCTACAACGA AGCTCCCG | 3385 |
| 36 | UGCGAGCC G CGAGCUGG | 964 | CCAGCTCG GGCTAGCTACAACGA GGCTCGCA | 3386 |
| 69 | CAGCCAAC G CAGCCGCA | 967 | TGCGGCTG GGCTAGCTACAACGA GTTGGCTG | 3387 |
| 75 | ACGCAGCC G CAGGAGCC | 968 | GGCTCCTG GGCTAGCTACAACGA GGCTGCGT | 3388 |
| 94 | GAGCCCUU G CCCCUGCC | 969 | GGCAGGGG GGCTAGCTACAACGA AAGGGCTC | 3389 |
| 100 | UUGCCCCU G CCCGCGCC | 970 | GGCGCGGG GGCTAGCTACAACGA AGGGGCAA | 3390 |
| 104 | CCCUGCCC G CGCCGCCG | 971 | CGGCGGCG GGCTAGCTACAACGA GGGCAGGG | 3391 |
| 106 | CUGCCCGC G CCGCCGCC | 972 | GGCGGCGG GGCTAGCTACAACGA GCGGGCAG | 3392 |
| 109 | CCCGCGCC G CCGCCCGC | 973 | GCGGGCGG GGCTAGCTACAACGA GGCGCGGG | 3393 |
| 112 | GCGCCGCC G CCCGCCGG | 974 | CCGGCGGG GGCTAGCTACAACGA GGCGGCGC | 3394 |
| 116 | CGCCGCCC G CCGGGGGG | 975 | CCCCCCGG GGCTAGCTACAACGA GGGCGGCG | 3395 |
| 137 | GGGAAGCC G CCACCGGC | 976 | GCCGGTGG GGCTAGCTACAACGA GGCTTCCC | 3396 |
| 148 | ACCGGCCC G CCAUGCCC | 977 | GGGCATGG GGCTAGCTACAACGA GGGCCGGT | 3397 |
| 153 | CCCGCCAU G CCCGCCCC | 978 | GGGGCGGG GGCTAGCTACAACGA ATGGCGGG | 3398 |
| 157 | CCAUGCCC G CCCCUCCC | 979 | GGGAGGGG GGCTAGCTACAACGA GGGCATGG | 3399 |
| 172 | CCAGCCCC G CCGGGAGC | 980 | GCTCCCGG GGCTAGCTACAACGA GGGGCTGG | 3400 |
| 183 | GGGAGCCC G CGCCCGCU | 981 | AGCGGGCG GGCTAGCTACAACGA GGGCTCCC | 3401 |
| 185 | GAGCCCGC G CCCGCUGC | 982 | GCAGCGGG GGCTAGCTACAACGA GCGGGCTC | 3402 |
| 189 | CCGCGCCC G CUGCCCAG | 983 | CTGGGCAG GGCTAGCTACAACGA GGGCGCGG | 3403 |
| 192 | CGCCCGCU G CCCAGGCU | 984 | AGCCTGGG GGCTAGCTACAACGA AGCGGGCG | 3404 |
| 205 | GGCUGGCC G CCGCCGUG | 985 | CACGGCGG GGCTAGCTACAACGA GGCCAGCC | 3405 |
| 208 | UGGCCGCC G CCGUGCCG | 986 | CGGCACGG GGCTAGCTACAACGA GGCGGCCA | 3406 |
| 213 | GCCGCCGU G CCGAUGUA | 987 | TACATCGG GGCTAGCTACAACGA ACGGCGGC | 3407 |
| 250 | UCUCCCCU G CUCCCGUG | 989 | CACGGGAG GGCTAGCTACAACGA AGGGGAGA | 3408 |
| 258 | GCUCCCGU G CUCUGCGG | 990 | CCGCAGAG GGCTAGCTACAACGA ACGGGAGC | 3409 |
| 263 | CGUGCUCU G CGGAUCUC | 991 | GAGATCCG GGCTAGCTACAACGA AGAGCACG | 3410 |
| 280 | CCCUGACC G CUCUCCAC | 993 | GTGGAGAG GGCTAGCTACAACGA GGTCAGGG | 3411 |
| 320 | AGGGCCCU G CAGGCCCU | 994 | AGGGCCTG GGCTAGCTACAACGA AGGGCCCT | 3412 |
| 340 | GUCCUGAU G CCCCCAAG | 996 | CTTGGGGG GGCTAGCTACAACGA ATCAGGAC | 3413 |
| 397 | GGGCAGGC G CCAGGGAC | 998 | GTCCCTGG GGCTAGCTACAACGA GCCTGCCC | 3414 |
| 420 | GGGCCAGU G CGAGCCCA | 999 | TGGGCTCG GGCTAGCTACAACGA ACTGGCCC | 3415 |
| 468 | CAAGCCCU G CCCUGGCU | 1002 | AGCCAGGG GGCTAGCTACAACGA AGGGCTTG | 3416 |
| 480 | UGGCUCCU G CUGUGGAU | 1003 | ATCCACAG GGCTAGCTACAACGA AGGAGCCA | 3417 |
| 493 | GGAUGGGC G CGGGAGUG | 1004 | CACTCCCG GGCTAGCTACAACGA GCCCATCC | 3418 |
| 501 | GCGGGAGU G CUGCCUGC | 1005 | GCAGGCAG GGCTAGCTACAACGA ACTCCCGC | 3419 |
| 504 | GGAGUGCU G CCUGCCCA | 1006 | TGGGCAGG GGCTAGCTACAACGA AGCACTCC | 3420 |
| 508 | UGCUGCCU G CCCACGGC | 1007 | GCCGTGGG GGCTAGCTACAACGA AGGCAGCA | 3421 |
| 537 | AUCCGGCU G CCCCUGCG | 1008 | CGCAGGGG GGCTAGCTACAACGA AGCCGGAT | 3422 |
| 543 | CUGCCCCU G CGCAGCGG | 1009 | CCGCTGCG GGCTAGCTACAACGA AGGGGCAG | 3423 |
| 545 | GCCCCUGC G CAGCGGCC | 1010 | GGCCGCTG GGCTAGCTACAACGA GCAGGGGC | 3424 |
| 562 | UGGGGGGC G CCCCCCUG | 1011 | CAGGGGGG GGCTAGCTACAACGA GCCCCCCA | 3425 |
| 576 | CUGGGGCU G CGGCUGCC | 1012 | GGCAGCCG GGCTAGCTACAACGA AGCCCCAG | 3426 |
| 582 | CUGCGGCU G CCCCGGGA | 1013 | TCCCGGGG GGCTAGCTACAACGA AGCCGCAG | 3427 |
| 708 | AGCCCCCC G CAGACGCU | 1019 | AGCGTCTG GGCTAGCTACAACGA GGGGGGCT | 3428 |
| 714 | CCGCAGAC G CUCAACAU | 1020 | ATGTTGAG GGCTAGCTACAACGA GTCTGCGG | 3429 |
| 751 | GUAACUUU G CAGUGGGU | 1021 | ACCCACTG GGCTAGCTACAACGA AAAGTTAC | 3430 |
| 760 | CAGUGGGU G CUGCCCCC | 1022 | GGGGGCAG GGCTAGCTACAACGA ACCCACTG | 3431 |
| 763 | UGGGUGCU G CCCCCCAC | 1023 | GTGGGGGG GGCTAGCTACAACGA AGCACCCA | 3432 |
| 780 | CCCUUCCU G CAUCGCUA | 1024 | TAGCGATG GGCTAGCTACAACGA AGGAAGGG | 3433 |
| 785 | CCUGCAUC G CUACUACC | 1025 | GGTAGTAG GGCTAGCTACAACGA GATGCAGG | 3434 |
| 843 | GUGUAUGU G CCCUACAC | 1026 | GTGTAGGG GGCTAGCTACAACGA ACATACAC | 3435 |
| 921 | GUCACUGU G CGUGCCAA | 1028 | TTGGCACG GGCTAGCTACAACGA ACAGTGAC | 3436 |
| 925 | CUGUGCGU G CCAACAUU | 1029 | AATGTTGG GGCTAGCTACAACGA ACGCACAG | 3437 |
| 934 | CCAACAUU G CUGCCAUC | 1030 | GATGGCAG GGCTAGCTACAACGA AATGTTGG | 3438 |
| 937 | ACAUUGCU G CCAUCACU | 1031 | AGTGATGG GGCTAGCTACAACGA AGCAATGT | 3439 |
| 1006 | UGGCCUAU G CUGAGAUU | 1033 | AATCTCAG GGCTAGCTACAACGA ATAGGCCA | 3440 |
| 1015 | CUGAGAUU G CCAGGCCU | 1035 | AGGCCTGG GGCTAGCTACAACGA AATCTCAG | 3441 |
| 1092 | UUCUCCCU G CAGCUUUG | 1039 | CAAAGCTG GGCTAGCTACAACGA AGGGAGAA | 3442 |
| 1105 | UUUGUGGU G CUGGCUUC | 1040 | GAAGCCAG GGCTAGCTACAACGA ACCACAAA | 3443 |
| 1134 | UCUGAAGU G CUGGCCUC | 1042 | GAGGCCAG GGCTAGCTACAACGA ACTTCAGA | 3444 |
| 1182 | GACCACUC G CUGUACAC | 1045 | GTGTACAG GGCTAGCTACAACGA GAGTGGTC | 3445 |
| 1248 | AUCAUUGU G CGGGUGGA | 1048 | TCCACCCG GGCTAGCTACAACGA ACAATGAT | 3446 |
| 1286 | AAUGGACU G CAAGGAGU | 1050 | ACTCCTTG GGCTAGCTACAACGA AGTCCATT | 3447 |
| 1344 | CUUCGUUU G CCCAAGAA | 1052 | TTCTTGGG GGCTAGCTACAACGA AAACGAAG | 3448 |
| 1366 | UUGAAGCU G CAGUCAAA | 1054 | TTTGACTG GGCTAGCTACAACGA AGCTTCAA | 3449 |
| 1442 | GCUGGUGU G CUGGCAAG | 1056 | CTTGCCAG GGCTAGCTACAACGA ACACCAGC | 3450 |
| 1526 | GUCCUUCC G CAUCACCA | 1058 | TGGTGATG GGCTAGCTACAACGA GGAAGGAC | 3451 |
| 1542 | AUCCUUCC G CAGCAAUA | 1059 | TATTGCTG GGCTAGCTACAACGA GGAAGGAT | 3452 |
| 1554 | CAAUACCU G CGGCCAGU | 1060 | ACTGGCCG GGCTAGCTACAACGA AGGTATTG | 3453 |
| 1603 | ACAAGUUU G CCAUCUCA | 1062 | TGAGATGG GGCTAGCTACAACGA AAACTTGT | 3454 |
| 1699 | UUGGCUUU G CUGUCAGC | 1066 | GCTGACAG GGCTAGCTACAACGA AAAGCCAA | 3455 |
| 1708 | CUGUCAGC G CUUGCCAU | 1067 | ATGGCAAG GGCTAGCTACAACGA GCTGACAG | 3456 |
| 1712 | CAGCGCUU G CCAUGUGC | 1068 | GCACATGG GGCTAGCTACAACGA AAGCGCTG | 3457 |
| 1719 | UGCCAUGU G CACGAUGA | 1069 | TCATCGTG GGCTAGCTACAACGA ACATGGCA | 3458 |
| 1843 | UCAUGGCU G CCAUCUGC | 1074 | GCAGATGG GGCTAGCTACAACGA AGCCATGA | 3459 |
| 1850 | UGCCAUCU G CGCCCUCU | 1075 | AGAGGGCG GGCTAGCTACAACGA AGATGGCA | 3460 |
| 1852 | CCAUCUGC G CCCUCUUC | 1076 | GAAGAGGG GGCTAGCTACAACGA GCAGATGG | 3461 |
| 1863 | CUCUUCAU G CUGCCACU | 1077 | AGTGGCAG GGCTAGCTACAACGA ATGAAGAG | 3462 |
| 1866 | UUCAUGCU G CCACUCUG | 1078 | CAGAGTGG GGCTAGCTACAACGA AGCATGAA | 3463 |
| 1874 | GCCACUCU G CCUCAUGG | 1079 | CCATGAGG GGCTAGCTACAACGA AGAGTGGC | 3464 |
| 1895 | UCAGUGGC G CUGCCUCC | 1080 | GGAGGCAG GGCTAGCTACAACGA GCCACTGA | 3465 |
| 1898 | GUGGCGCU G CCUCCGCU | 1081 | AGCGGAGG GGCTAGCTACAACGA AGCGCCAC | 3466 |
| 1904 | CUGCCUCC G CUGCCUGC | 1082 | GCAGGCAG GGCTAGCTACAACGA GGAGGCAG | 3467 |
| 1907 | CCUCCGCU G CCUGCGCC | 1083 | GGCGCAGG GGCTAGCTACAACGA AGCGGAGG | 3468 |
| 1911 | CGCUGCCU G CGCCAGCA | 1084 | TGCTGGCG GGCTAGCTACAACGA AGGCAGCG | 3469 |
| 1913 | CUGCCUGC G CCAGCAGC | 1085 | GCTGCTGG GGCTAGCTACAACGA GCAGGCAG | 3470 |
| 1933 | AUGACUUU G CUGAUGAC | 1088 | GTCATCAG GGCTAGCTACAACGA AAAGTCAT | 3471 |
| 1950 | AUCUCCCU G CUGAAGUG | 1091 | CACTTCAG GGCTAGCTACAACGA AGGGAGAT | 3472 |
| 2087 | CACCAAAU G CCUCUGCC | 1094 | GGCAGAGG GGCTAGCTACAACGA ATTTGGTG | 3473 |
| 2093 | AUGCCUCU G CCUUGAUG | 1095 | CATCAAGG GGCTAGCTACAACGA AGAGGCAT | 3474 |
| 2179 | AGCACUCU G CUGGCGGG | 1097 | CCCGCCAG GGCTAGCTACAACGA AGAGTGCT | 3475 |
| 2227 | GAAAUUCU G CUGCUUGA | 1098 | TCAAGCAG GGCTAGCTACAACGA AGAATTTC | 3476 |
| 2230 | AUUCUGCU G CUUGAAAC | 1099 | GTTTCAAG GGCTAGCTACAACGA AGCAGAAT | 3477 |
| 2329 | CAUCACAC G CAGGUUAC | 1102 | GTAACCTG GGCTAGCTACAACGA GTGTGATG | 3478 |
| 2393 | GUUUCCCU G CUGGCCAA | 1103 | TTGGCCAG GGCTAGCTACAACGA AGGGAAAC | 3479 |
| 2419 | GAGAGGAU G CACAGUUU | 1104 | AAACTGTG GGCTAGCTACAACGA ATCCTCTC | 3480 |
| 2428 | CACAGUUU G CUAUUUGC | 1105 | GCAAATAG GGCTAGCTACAACGA AAACTGTG | 3481 |
| 2435 | UGCUAUUU G CUUUAGAG | 1106 | CTCTAAAG GGCTAGCTACAACGA AAATAGCA | 3482 |
| 2476 | ACAUUGGU G CAAAGAUU | 1107 | AATCTTTG GGCTAGCTACAACGA ACCAATGT | 3483 |
| 2485 | CAAAGAUU G CCUCUUGA | 1108 | TCAAGAGG GGCTAGCTACAACGA AATCTTTG | 3484 |
| 219 | GUGCCGAU G UAGCGGGC | 1110 | GCCCGCTA GGCTAGCTACAACGA ATCGGCAC | 3485 |
| 483 | CUCCUGCU G UGGAUGGG | 1111 | CCCATCCA GGCTAGCTACAACGA AGCAGGAG | 3486 |
| 634 | GCAGCUUU G UGGAGAUG | 1112 | CATCTCCA GGCTAGCTACAACGA AAAGCTGC | 3487 |
| 804 | AGGCAGCU G UCCAGCAC | 1113 | GTGCTGGA GGCTAGCTACAACGA AGCTGCCT | 3488 |
| 835 | GGAAGGGU G UGUAUGUG | 1114 | CACATACA GGCTAGCTACAACGA ACCCTTCC | 3489 |
| 837 | AAGGGUGU G UAUGUGCC | 1115 | GGCACATA GGCTAGCTACAACGA ACACCCTT | 3490 |
| 841 | GUGUGUAU G UGCCCUAC | 1116 | GTAGGGCA GGCTAGCTACAACGA ATACACAC | 3491 |
| 919 | ACGUCACU G UGCGUGCC | 1117 | GGCACGCA GGCTAGCTACAACGA AGTGACGT | 3492 |
| 1100 | GCAGCUUU G UGGUGCUG | 1118 | CAGCACCA GGCTAGCTACAACGA AAAGCTGC | 3493 |
| 1144 | UGGCCUCU G UCGGAGGG | 1119 | CCCTCCGA GGCTAGCTACAACGA AGAGGCCA | 3494 |
| 1185 | CACUCGCU G UACACAGG | 1120 | CCTGTGTA GGCTAGCTACAACGA AGCGAGTG | 3495 |
| 1246 | UGAUCAUU G UGCGGGUG | 1121 | CACCCGCA GGCTAGCTACAACGA AATGATCA | 3496 |
| 1315 | AGAGCAUU G UGGACAGU | 1122 | ACTGTCCA GGCTAGCTACAACGA AATGCTCT | 3497 |
| 1356 | AAGAAAGU G UUUGAAGC | 1123 | GCTTCAAA GGCTAGCTACAACGA ACTTTCTT | 3498 |
| 1440 | CAGCUGGU G UGCUGGCA | 1124 | TGCCAGCA GGCTAGCTACAACGA ACCAGCTG | 3499 |
| 1570 | UGGAAGAU G UGGCCACG | 1125 | CGTGGCCA GGCTAGCTACAACGA ATCTTCCA | 3500 |
| 1592 | AGACGACU G UUACAAGU | 1126 | ACTTGTAA GGCTAGCTACAACGA AGTCGTCT | 3501 |
| 1630 | CGGGCACU G UUAUGGGA | 1127 | TCCCATAA GGCTAGCTACAACGA AGTGCCCG | 3502 |
| 1642 | UGGGAGCU G UUAUCAUG | 1128 | CATGATAA GGCTAGCTACAACGA AGCTCCCA | 3503 |
| 1666 | UCUACGUU G UCUUUGAU | 1129 | ATCAAAGA GGCTAGCTACAACGA AACGTAGA | 3504 |
| 1702 | GCUUUGCU G UCAGCGCU | 1130 | AGCGCTGA GGCTAGCTACAACGA AGCAAAGC | 3505 |
| 1717 | CUUGCCAU G UGCACGAU | 1131 | ATCGTGCA GGCTAGCTACAACGA ATGGCAAG | 3506 |
| 1759 | GCCCUUUU G UCACCUUG | 1132 | CAAGGTGA GGCTAGCTACAACGA AAAAGGGC | 3507 |
| 1781 | GGAAGACU G UGGCUACA | 1133 | TGTAGCCA GGCTAGCTACAACGA AGTCTTCC | 3508 |
| 1834 | UAGCCUAU G UCAUGGCU | 1134 | AGCCATGA GGCTAGCTACAACGA ATAGGCTA | 3509 |
| 1884 | CUCAUGGU G UGUCAGUG | 1135 | CACTGACA GGCTAGCTACAACGA ACCATGAG | 3510 |
| 1886 | CAUGGUGU G UCAGUGGC | 1136 | GCCACTGA GGCTAGCTACAACGA ACACCATG | 3511 |
| 2048 | UGGCACCU G UGGCCAGA | 1137 | TCTGGCCA GGCTAGCTACAACGA AGGTGCCA | 3512 |
| 2139 | CAGGGACU G UACCUGUA | 1138 | TACAGGTA GGCTAGCTACAACGA AGTCCCTG | 3513 |
| 2145 | CUGUACCU G UAGGAAAC | 1139 | GTTTCCTA GGCTAGCTACAACGA AGGTACAG | 3514 |
| 2256 | GAACCUUU G UCCACCAU | 1140 | ATGGTGGA GGCTAGCTACAACGA AAAGGTTC | 3515 |
| 2346 | CUUGGCGU G UGUCCCUG | 1141 | CAGGGACA GGCTAGCTACAACGA ACGCCAAG | 3516 |
| 2348 | UGGCGUGU G UCCCUGUG | 1142 | CACAGGGA GGCTAGCTACAACGA ACACGCCA | 3517 |
| 2354 | GUGUCCCU G UGGUACCC | 1143 | GGGTACCA GGCTAGCTACAACGA AGGGACAC | 3518 |
| 2385 | CCAAGCUU G UUUCCCUG | 1144 | CAGGGAAA GGCTAGCTACAACGA AAGCTTGG | 3519 |
| 2453 | CAGGGACU G UAUAAACA | 1145 | TGTTTATA GGCTAGCTACAACGA AGTCCCTG | 3520 |
| 14 | CGUCCGCA G CCCGCCCG | 1146 | CGGGCGGG GGCTAGCTACAACGA TGCGGACG | 3521 |
| 26 | GCCCGGGA G CUGCGAGC | 1147 | GCTCGCAG GGCTAGCTACAACGA TCCCGGGC | 3522 |
| 33 | AGCUGCGA G CCGCGAGC | 1148 | GCTCGCGG GGCTAGCTACAACGA TCGCAGCT | 3523 |
| 40 | AGCCGCGA G CUGGAUUA | 1149 | TAATCCAG GGCTAGCTACAACGA TCGCGGCT | 3524 |
| 51 | GGAUUAUG G UGGCCUGA | 1150 | TCAGGCCA GGCTAGCTACAACGA CATAATCC | 3525 |
| 54 | UUAUGGUG G CCUGAGCA | 1151 | TGCTCAGG GGCTAGCTACAACGA CACCATAA | 3526 |
| 60 | UGGCCUGA G CAGCCAAC | 1152 | GTTGGCTG GGCTAGCTACAACGA TCAGGCCA | 3527 |
| 63 | CCUGAGCA G CCAACGCA | 1153 | TGCGTTGG GGCTAGCTACAACGA TGCTCAGG | 3528 |
| 72 | CCAACGCA G CCGCAGGA | 1154 | TCCTGCGG GGCTAGCTACAACGA TGCGTTGG | 3529 |
| 81 | CCGCAGGA G CCCGGAGC | 1155 | GCTCCGGG GGCTAGCTACAACGA TCCTGCGG | 3530 |
| 88 | AGCCCGGA G CCCUUGCC | 1156 | GGCAAGGG GGCTAGCTACAACGA TCCGGGCT | 3531 |
| 134 | CCAGGGAA G CCGCCACC | 1157 | GGTGGCGG GGCTAGCTACAACGA TTCCCTGG | 3532 |
| 144 | CGCCACCG G CCCGCCAU | 1158 | ATGGCGGG GGCTAGCTACAACGA CGGTGGCG | 3533 |
| 167 | CCCUCCCA G CCCCGCCG | 1159 | CGGCGGGG GGCTAGCTACAACGA TGGGAGGG | 3534 |
| 179 | CGCCGGGA G CCCGCGCC | 1160 | GGCGCGGG GGCTAGCTACAACGA TCCCGSCG | 3535 |
| 198 | CUGCCCAG G CUGGCCGC | 1161 | GCGGCCAG GGCTAGCTACAACGA CTGGGCAG | 3536 |
| 202 | CCAGGCUG G CCGCCGCC | 1162 | GGCGGCGG GGCTAGCTACAACGA CAGCCTGG | 3537 |
| 211 | CCGCCGCC G UGCCGAUG | 1163 | CATCGGCA GGCTAGCTACAACGA GGCGGCGG | 3538 |
| 222 | CCGAUGUA G CGGGCUCC | 1164 | GGAGCCCG GGCTAGCTACAACGA TACATCGG | 3539 |
| 226 | UGUAGCGG G CUCCGGAU | 1165 | ATCCGGAG GGCTAGCTACAACGA CCGCTACA | 3540 |
| 239 | GGAUCCCA G CCUCUCCC | 1166 | GGGAGAGG GGCTAGCTACAACGA TGGGATCC | 3541 |
| 256 | CUGCUCCC G UGCUCUGC | 1167 | GCAGAGCA GGCTAGCTACAACGA GGGAGCAG | 3542 |
| 290 | UCUCCACA G CCCGGACC | 1168 | GGTCCGGG GGCTAGCTACAACGA TGTGGAGA | 3543 |
| 304 | ACCCGGGG G CUGGCCCA | 1169 | TGGGCCAG GGCTAGCTACAACGA CCCCGGGT | 3544 |
| 308 | GGGGGCUG G CCCAGGGC | 1170 | GCCCTGGG GGCTAGCTACAACGA CAGCCCCC | 3545 |
| 315 | GGCCCAGG G CCCUGCAG | 1171 | CTGCAGGG GGCTAGCTACAACGA CCTGGGCC | 3546 |
| 324 | CCCUGCAG G CCCUGGCG | 1172 | CGCCAGGG GGCTAGCTACAACGA CTGCAGGG | 3547 |
| 330 | AGGCCCUG G CGUCCUGA | 1173 | TCAGGACG GGCTAGCTACAACGA CAGGGCCT | 3548 |
| 332 | GCCCUGGC G UCCUGAUG | 1174 | CATCAGGA GGCTAGCTACAACGA GCCAGGGC | 3549 |
| 348 | GCCCCCAA G CUCCCUCU | 1175 | AGAGGGAG GGCTAGCTACAACGA TTGGGGGC | 3550 |
| 365 | CCUGAGAA G CCACCAGC | 1176 | GCTGGTGG GGCTAGCTACAACGA TTCTCAGG | 3551 |
| 372 | AGCCACCA G CACCACCC | 1177 | GGGTGGTG GGCTAGCTACAACGA TGGTGGCT | 3552 |
| 391 | ACUUGGGG G CAGGCGCC | 1178 | GGCGCCTG GGCTAGCTACAACGA CCCCAAGT | 3553 |
| 395 | GGGGGCAG G CGCCAGGG | 1179 | CCCTGGCG GGCTAGCTACAACGA CTGCCCCC | 3554 |
| 410 | GGACGGAC G UGGGCCAG | 1180 | CTGGCCCA GGCTAGCTACAACGA GTCCGTCC | 3555 |
| 414 | GGACGUGG G CCAGUGCG | 1181 | CGCACTGG GGCTAGCTACAACGA CCACGTCC | 3556 |
| 418 | GUGGGCCA G UGCGAGCC | 1182 | GGCTCGCA GGCTAGCTACAACGA TGGCCCAC | 3557 |
| 424 | CAGUGCGA G CCCAGAGG | 1183 | CCTCTGGG GGCTAGCTACAACGA TCGCACTG | 3558 |
| 433 | CCCAGAGG G CCCGAAGG | 1184 | CCTTCGGG GGCTAGCTACAACGA CCTCTGGG | 3559 |
| 441 | GCCCGAAG G CCGGGGCC | 1185 | GGCCCCGG GGCTAGCTACAACGA CTTCGGGC | 3560 |
| 447 | AGGCCGGG G CCCACCAU | 1186 | ATGGTGGG GGCTAGCTACAACGA CCCGGCCT | 3561 |
| 457 | CCACCAUG G CCCAAGCC | 1187 | GGCTTGGG GGCTAGCTACAACGA CATGGTGG | 3562 |
| 463 | UGGCCCAA G CCCUGCCC | 1188 | GGGCAGGG GGCTAGCTACAACGA TTGGGCCA | 3563 |
| 474 | CUGCCCUG G CUCCUGCU | 1189 | AGCAGGAG GGCTAGCTACAACGA CAGGGCAG | 3564 |
| 491 | GUGGAUGG G CGCGGGAG | 1190 | CTCCCGCG GGCTAGCTACAACGA CCATCCAC | 3565 |
| 499 | GCGCGGGA G UGCUGCCU | 1191 | AGGCAGCA GGCTAGCTACAACGA TCCCGCGC | 3566 |
| 515 | UGCCCACG G CACCCAGC | 1192 | GCTGGGTG GGCTAGCTACAACGA CGTGGGCA | 3567 |
| 522 | GGCACCCA G CACGGCAU | 1193 | ATGCCGTG GGCTAGCTACAACGA TGGGTGCC | 3568 |
| 527 | CCAGCACG G CAUCCGGC | 1194 | GCCGGATG GGCTAGCTACAACGA CGTGCTGG | 3569 |
| 534 | GGCAUCCG G CUGCCCCU | 1195 | AGGGGCAG GGCTAGCTACAACGA CGGATGCC | 3570 |
| 548 | CCUGCGCA G CGGCCUGG | 1196 | CCAGGCCG GGCTAGCTACAACGA TGCGCAGG | 3571 |
| 551 | GCGCAGCG G CCUGGGGG | 1197 | CCCCCAGG GGCTAGCTACAACGA CGCTGCGC | 3572 |
| 560 | CCUGGGGG G CGCCCCCC | 1198 | GGGGGGCG GGCTAGCTACAACGA CCCCCAGG | 3573 |
| 573 | CCCCUGGG G CUGCGGCU | 1199 | AGCCGCAG GGCTAGCTACAACGA CCCAGGGG | 3574 |
| 579 | GGGCUGCG G CUGCCCCG | 1200 | CGGGGCAG GGCTAGCTACAACGA CGCAGCCC | 3575 |
| 603 | GACGAAGA G CCCGAGGA | 1201 | TCCTCGGG GGCTAGCTACAACGA TCTTCGTC | 3576 |
| 612 | CCCGAGGA G CCCGGCCG | 1202 | CGGCCGGG GGCTAGCTACAACGA TCCTCGGG | 3577 |
| 617 | GGAGCCCG G CCGGAGGG | 1203 | CCCTCCGG GGCTAGCTACAACGA CGGGCTCC | 3578 |
| 626 | CCGGAGGG G CAGCUUUG | 1204 | CAAAGCTG GGCTAGCTACAACGA CCCTCCGG | 3579 |
| 629 | GAGGGGCA G CUUUGUGG | 1205 | CCACAAAG GGCTAGCTACAACGA TGCCCCTC | 3580 |
| 643 | UGGAGAUG G UGGACAAC | 1206 | GTTGTCCA GGCTAGCTACAACGA CATCTCCA | 3581 |
| 659 | CCUGAGGG G CAAGUCGG | 1207 | CCGACTTG GGCTAGCTACAACGA CCCTCAGG | 3582 |
| 663 | AGGGGCAA G UCGGGGCA | 1208 | TGCCCCGA GGCTAGCTACAACGA TTGCCCCT | 3583 |
| 669 | AAGUCGGG G CAGGGCUA | 1209 | TAGCCCTG GGCTAGCTACAACGA CCCGACTT | 3584 |
| 674 | GGGGCAGG G CUACUACG | 1210 | CGTAGTAG GGCTAGCTACAACGA CCTGCCCC | 3585 |
| 682 | GCUACUAC G UGGAGAUG | 1211 | CATCTCCA GGCTAGCTACAACGA GTAGTAGC | 3586 |
| 694 | AGAUGACC G UGGGCAGC | 1212 | GCTGCCCA GGCTAGCTACAACGA GGTCATCT | 3587 |
| 698 | GACCGUGG G CAGCCCCC | 1213 | GGGGGCTG GGCTAGCTACAACGA CCACGGTC | 3588 |
| 701 | CGUGGGCA G CCCCCCGC | 1214 | GCGGGGGG GGCTAGCTACAACGA TGCCCACG | 3589 |
| 727 | ACAUCCUG G UGGAUACA | 1215 | TGTATCCA GGCTAGCTACAACGA CAGGATGT | 3590 |
| 737 | GGAUACAG G CAGCAGUA | 1216 | TACTGCTG GGCTAGCTACAACGA CTGTATCC | 3591 |
| 740 | UACAGGCA G CAGUAACU | 1217 | AGTTACTG GGCTAGCTACAACGA TGCCTGTA | 3592 |
| 743 | AGGCAGCA G UAACUUUG | 1218 | CAAAGTTA GGCTAGCTACAACGA TGCTGCCT | 3593 |
| 754 | ACUUUGCA G UGGGUGCU | 1219 | AGCACCCA GGCTAGCTACAACGA TGCAAAGT | 3594 |
| 758 | UGCAGUGG G UGCUGCCC | 1220 | GGGCAGCA GGCTAGCTACAACGA CCACTGCA | 3595 |
| 798 | UACCAGAG G CAGCUGUC | 1221 | GACAGCTG GGCTAGCTACAACGA CTCTGGTA | 3596 |
| 801 | CAGAGGCA G CUGUCCAG | 1222 | CTGGACAG GGCTAGCTACAACGA TGCCTCTG | 3597 |
| 809 | GCUGUCCA G CACAUACC | 1223 | GGTATGTG GGCTAGCTACAACGA TGGACAGC | 3598 |
| 833 | CCGGAAGG G UGUGUAUG | 1224 | CATACACA GGCTAGCTACAACGA CCTTCCGG | 3599 |
| 857 | CACCCAGG G CAAGUGGG | 1225 | CCCACTTG GGCTAGCTACAACGA CCTGGGTG | 3600 |
| 861 | CAGGGCAA G UGGGAAGG | 1226 | CCTTCCCA GGCTAGCTACAACGA TTGCCCTG | 3601 |
| 871 | GAAGGGGA G CUGGGCAC | 1227 | GTGCCCAG GGCTAGCTACAACGA TCCCCTTC | 3602 |
| 878 | GGAGCUGG G CACCGACC | 1228 | GGTCGGTG GGCTAGCTACAACGA CCAGCTCC | 3603 |
| 889 | CCGACCUG G UAAGCAUC | 1229 | GATGCTTA GGCTAGCTACAACGA CAGGTCGG | 3604 |
| 893 | CCUGGUAA G CAUCCCCC | 1230 | GGGGGATG GGCTAGCTACAACGA TTACCAGG | 3605 |
| 905 | CCCCCAUG G CCCCAACG | 1231 | CGTTGGGG GGCTAGCTACAACGA CATGGGGG | 3606 |
| 913 | GCCCCAAC G UCACUGUG | 1232 | CACAGTGA GGCTAGCTACAACGA GTTGGGGC | 3607 |
| 923 | CACUGUGC G UGCCAACA | 1233 | TGTTGGCA GGCTAGCTACAACGA GCACAGTG | 3608 |
| 957 | UCAGACAA G UUCUUCAU | 1234 | ATGAAGAA GGCTAGCTACAACGA TTGTCTGA | 3609 |
| 971 | CAUCAACG G CUCCAACU | 1235 | AGTTGGAG GGCTAGCTACAACGA CGTTGATG | 3610 |
| 986 | CUGGGAAG G CAUCCUGG | 1236 | CCAGGATG GGCTAGCTACAACGA CTTCCCAG | 3611 |
| 996 | AUCCUGGG G CUGGCCUA | 1237 | TAGGCCAG GGCTAGCTACAACGA CCCAGGAT | 3612 |
| 1000 | UGGGGCUG G CCUAUGCU | 1258 | AGCATAGG GGCTAGCTACAACGA CAGCCCCA | 3613 |
| 1020 | AUUGCCAG G CCUGACGA | 1239 | TCGTCAGG GGCTAGCTACAACGA CTGGCAAT | 3614 |
| 1038 | UCCCUGGA G CCUUUCUU | 1240 | AAGAAAGG GGCTAGCTACAACGA TCCAGGGA | 3615 |
| 1057 | ACUCUCUG G UAAAGCAG | 1241 | CTGCTTTA GGCTAGCTACAACGA CAGAGAGT | 3616 |
| 1062 | CUGGUAAA G CAGACCCA | 1242 | TGGGTCTG GGCTAGCTACAACGA TTTACCAG | 3617 |
| 1072 | AGACCCAC G UUCCCAAC | 1243 | GTTGGGAA GGCTAGCTACAACGA GTGGGTCT | 3618 |
| 1095 | UCCCUGCA G CUUUGUGG | 1244 | CCACAAAG GGCTAGCTACAACGA TGCAGGGA | 3619 |
| 1103 | GCUUUGUG G UGCUGGCU | 1245 | AGCCAGCA GGCTAGCTACAACGA CACAAAGC | 3620 |
| 1109 | UGGUGCUG G CUUCCCCC | 1246 | GGGGGAAG GGCTAGCTACAACGA CAGCACCA | 3621 |
| 1125 | GUCAACCA G UCUGAAGU | 1247 | ACTTCAGA GGCTAGCTACAACGA TGGTTGAG | 3622 |
| 1132 | AGUCUGAA G UGCUGGCC | 1248 | GGCCAGCA GGCTAGCTACAACGA TTCAGACT | 3623 |
| 1138 | AAGUGCUG G CCUCUGUC | 1249 | GACAGAGG GGCTAGCTACAACGA CAGCACTT | 3624 |
| 1154 | CGGAGGGA G CAUGAUCA | 1250 | TGATCATG GGCTAGCTACAACGA TCCCTCCG | 3625 |
| 1169 | CAUUGGAG G UAUCGACC | 1251 | GGTCGATA GGCTAGCTACAACGA CTCCAATG | 3626 |
| 1193 | GUACACAG G CAGUCUCU | 1252 | AGAGACTG GGCTAGCTACAACGA CTGTGTAC | 3627 |
| 1196 | CACAGGCA G UCUCUGGU | 1253 | ACCAGAGA GGCTAGCTACAACGA TGCCTGTG | 3628 |
| 1203 | AGUCUCUG G UAUACACC | 1254 | GGTGTATA GGCTAGCTACAACGA CAGAGACT | 3629 |
| 1218 | CCCAUCCG G CGGGAGUG | 1255 | CACTCCCG GGCTAGCTACAACGA CGGATGGG | 3630 |
| 1224 | CGGCGGGA G UGGUAUUA | 1256 | TAATACCA GGCTAGCTACAACGA TCCCGCCG | 3631 |
| 1227 | CGGGAGUG G UAUUAUGA | 1257 | TCATAATA GGCTAGCTACAACGA CACTCCCG | 3632 |
| 1237 | AUUAUGAG G UGAUCAUU | 1258 | AATGATCA GGCTAGCTACAACGA CTCATAAT | 3633 |
| 1252 | UUGUGCGG G UGGAGAUC | 1259 | GATCTCCA GGCTAGCTACAACGA CCGCACAA | 3634 |
| 1293 | UGCAAGGA G UACAACUA | 1260 | TAGTTGTA GGCTAGCTACAACGA TCCTTGCA | 3635 |
| 1310 | UGACAAGA G CAUUGUGG | 1261 | CCACAATG GGCTAGCTACAACGA TCTTGTCA | 3636 |
| 1322 | UGUGGACA G UGGCACCA | 1262 | TGGTGCCA GGCTAGCTACAACGA TGTCCACA | 3637 |
| 1325 | GGACAGUG G CACCACCA | 1263 | TGGTGGTG GGCTAGCTACAACGA CACTGTCC | 3638 |
| 1340 | CAACCUUC G UUUGCCCA | 1264 | TGGGCAAA GGCTAGCTACAACGA GAAGGTTG | 3639 |
| 1354 | CCAAGAAA G UGUUUGAA | 1265 | TTCAAACA GGCTAGCTACAACGA TTTCTTGG | 3640 |
| 1363 | UGUUUGAA G CUGCAGUC | 1266 | GACTGCAG GGCTAGCTACAACGA TTCAAACA | 3641 |
| 1369 | AAGCUGCA G UCAAAUCC | 1267 | GGATTTGA GGCTAGCTACAACGA TGCAGCTT | 3642 |
| 1384 | CCAUCAAG G CAGCCUCC | 1268 | GGAGGCTG GGCTAGCTACAACGA CTTGATGG | 3643 |
| 1387 | UCAAGGCA G CCUCCUCC | 1269 | GGAGGAGG GGCTAGCTACAACGA TGCCTTGA | 3644 |
| 1404 | ACGGAGAA G UUCCCUGA | 1270 | TCAGGGAA GGCTAGCTACAACGA TTCTCCGT | 3645 |
| 1415 | CCCUGAUG G UUUCUGGC | 1271 | GCCAGAAA GGCTAGCTACAACGA CATCAGGG | 3646 |
| 1422 | GGUUUCUG G CUAGGAGA | 1272 | TCTCCTAG GGCTAGCTACAACGA CAGAAACC | 3647 |
| 1431 | CUAGGAGA G CAGCUGGU | 1273 | ACCAGCTG GGCTAGCTACAACGA TCTCCTAG | 3648 |
| 1434 | GGAGAGCA G CUGGUGUG | 1274 | CACACCAG GGCTAGCTACAACGA TGCTCTCC | 3649 |
| 1438 | AGCAGCUG G UGUGCUGG | 1275 | CCAGCACA GGCTAGCTACAACGA CAGCTGCT | 3650 |
| 1446 | GUGUGCUG G CAAGCAGG | 1276 | CCTGCTTG GGCTAGCTACAACGA CAGCACAC | 3651 |
| 1450 | GCUGGCAA G CAGGCACC | 1277 | GGTGCCTG GGCTAGCTACAACGA TTGCCAGC | 3652 |
| 1454 | GCAAGCAG G CACCACCC | 1278 | GGGTGGTG GGCTAGCTACAACGA CTGCTTGC | 3653 |
| 1480 | UUUUCCCA G UCAUCUCA | 1279 | TGAGATGA GGCTAGCTACAACGA TGGGAAAA | 3654 |
| 1502 | CCUAAUGG G UGAGGUUA | 1280 | TAACCTCA GGCTAGCTACAACGA CCATTAGG | 3655 |
| 1507 | UGGGUGAG G UUACCAAC | 1281 | GTTGGTAA GGCTAGCTACAACGA CTCACCCA | 3656 |
| 1518 | ACCAACCA G UCCUUCCG | 1282 | CGGAAGGA GGCTAGCTACAACGA TGGTTGGT | 3657 |
| 1545 | CUUCCGCA G CAAUACCU | 1283 | AGGTATTG GGCTAGCTACAACGA TGCGGAAG | 3658 |
| 1557 | UACCUGCG G CCAGUGGA | 1284 | TCCACTGG GGCTAGCTACAACGA CGCAGGTA | 3659 |
| 1561 | UGCGGCCA G UGGAAGAU | 1285 | ATCTTCCA GGCTAGCTACAACGA TGGCCGCA | 3660 |
| 1573 | AAGAUGUG G CCACGUCC | 1286 | GGACGTGG GGCTAGCTACAACGA CACATCTT | 3661 |
| 1578 | GUGGCCAC G UCCCAAGA | 1287 | TCTTGGGA GGCTAGCTACAACGA GTGGCCAC | 3662 |
| 1599 | UGUUACAA G UUUGCCAU | 1288 | ATGGCAAA GGCTAGCTACAACGA TTGTAACA | 3663 |
| 1614 | AUCUCACA G UCAUCCAC | 1289 | GTGGATGA GGCTAGCTACAACGA TGTGAGAT | 3664 |
| 1625 | AUCCACGG G CACUGUUA | 1290 | TAACAGTG GGCTAGCTACAACGA CCGTGGAT | 3665 |
| 1639 | UUAUGGGA G CUGUUAUC | 1291 | GATAACAG GGCTAGCTACAACGA TCCCATAA | 3666 |
| 1655 | CAUGGAGG G CUUCUACG | 1292 | CGTAGAAG GGCTAGCTACAACGA CCTCCATG | 3667 |
| 1663 | GCUUCUAC G UUGUCUUU | 1293 | AAAGACAA GGCTAGCTACAACGA GTAGAAGC | 3668 |
| 1678 | UUGAUCGG G CCCGAAAA | 1294 | TTTTCGGG GGCTAGCTACAACGA CCGATCAA | 3669 |
| 1694 | ACGAAUUG G CUUUGCUG | 1295 | CAGCAAAG GGCTAGCTACAACGA CAATTCGT | 3670 |
| 1706 | UGCUGUCA G CGCUUGCC | 1296 | GGCAAGCG GGCTAGCTACAACGA TGACAGCA | 3671 |
| 1728 | CACGAUGA G UUCAGGAC | 1297 | GTCCTGAA GGCTAGCTACAACGA TCATCGTG | 3672 |
| 1738 | UCAGGACG G CAGCGGUG | 1298 | CACCGCTG GGCTAGCTACAACGA CGTCCTGA | 3673 |
| 1741 | GGACGGCA G CGGUGGAA | 1299 | TTCCACCG GGCTAGCTACAACGA TGCCGTCC | 3674 |
| 1744 | CGGCAGCG G UGGAAGGC | 1300 | GCCTTCCA GGCTAGCTACAACGA CGCTGCCG | 3675 |
| 1751 | GGUGGAAG G CCCUUUUG | 1301 | CAAAAGGG GGCTAGCTACAACGA CTTCCACC | 3676 |
| 1784 | AGACUGUG G CUACAACA | 1302 | TGTTGTAG GGCTAGCTACAACGA CACAGTCT | 3677 |
| 1809 | ACAGAUGA G UCAACCCU | 1303 | AGGGTTGA GGCTAGCTACAACGA TCATCTGT | 3678 |
| 1828 | UGACCAUA G CCUAUGUC | 1304 | GACATAGG GGCTAGCTACAACGA TATGGTCA | 3679 |
| 1840 | AUGUCAUG G CUGCCAUC | 1305 | GATGGCAG GGCTAGCTACAACGA CATGACAT | 3680 |
| 1882 | GCCUCAUG G UGUGUCAG | 1306 | CTGACACA GGCTAGCTACAACGA CATGAGGC | 3681 |
| 1890 | GUGUGUCA G UGGCGCUG | 1307 | CAGCGCCA GGCTAGCTACAACGA TGACACAC | 3682 |
| 1893 | UGUCAGUG G CGCUGCCU | 1308 | AGGCAGCG GGCTAGCTACAACGA CACTGACA | 3683 |
| 1917 | CUGCGCCA G CAGCAUGA | 1309 | TCATGCTG GGCTAGCTACAACGA TGGCGCAG | 3684 |
| 1920 | CGCCAGCA G CAUGAUGA | 1310 | TCATCATG GGCTAGCTACAACGA TGCTGGCG | 3685 |
| 1956 | CUGCUGAA G UGAGGAGG | 1311 | CCTCCTCA GGCTAGCTACAACGA TTCAGCAG | 3686 |
| 1964 | GUGAGGAG G CCCAUGGG | 1312 | CCCATGGG GGCTAGCTACAACGA CTCCTCAC | 3687 |
| 1972 | GCCCAUGG G CAGAAGAU | 1313 | ATCTTCTG GGCTAGCTACAACGA CCATGGGC | 3688 |
| 2006 | ACACCUCC G UGGUUCAC | 1314 | GTGAACCA GGCTAGCTACAACGA GGAGGTGT | 3689 |
| 2009 | CCUCCGUG G UUCACUUU | 1315 | AAAGTGAA GGCTAGCTACAACGA CACGGAGG | 3690 |
| 2019 | UCACUUUG G UCACAAGU | 1316 | ACTTGTGA GGCTAGCTACAACGA CAAAGTGA | 3691 |
| 2026 | GGUCACAA G UAGGAGAC | 1317 | GTCTCCTA GGCTAGCTACAACGA TTGTGACC | 3692 |
| 2042 | CACAGAUG G CACCUGUG | 1318 | CACAGGTG GGCTAGCTACAACGA CATCTGTG | 3693 |
| 2051 | CACCUGUG G CCAGAGCA | 1319 | TGCTCTGG GGCTAGCTACAACGA CACAGGTG | 3694 |
| 2057 | UGGCCAGA G CACCUCAG | 1320 | CTGAGGTG GGCTAGCTACAACGA TCTGGCCA | 3695 |
| 2114 | AGGAAAAG G CUGGCAAG | 1321 | CTTGCCAG GGCTAGCTACAACGA CTTTTCCT | 3696 |
| 2118 | AAAGGCUG G CAAGGUGG | 1322 | CCACCTTG GGCTAGCTACAACGA CAGCCTTT | 3697 |
| 2123 | CUGGCAAG G UGGGUUCC | 1323 | GGAACCCA GGCTAGCTACAACGA CTTGCCAG | 3698 |
| 2127 | CAAGGUGG G UUCCAGGG | 1324 | CCCTGGAA GGCTAGCTACAACGA CCACCTTG | 3699 |
| 2172 | AGAAAGAA G CACUCUGC | 1325 | GCAGAGTG GGCTAGCTACAACGA TTCTTTCT | 3700 |
| 2183 | CUCUGCUG G CGGGAAUA | 1326 | TATTCCCG GGCTAGCTACAACGA CAGCAGAG | 3701 |
| 2198 | UACUCUUG G UCACCUCA | 1327 | TGAGGTGA GGCTAGCTACAACGA CAAGAGTA | 3702 |
| 2214 | AAAUUUAA G UCGGGAAA | 1328 | TTTCCCGA GGCTAGCTACAACGA TTAAATTT | 3703 |
| 2243 | AAACUUCA G CCCUGAAC | 1329 | GTTCAGGG GGCTAGCTACAACGA TGAAGTTT | 3704 |
| 2288 | AACCCAAA G UAUUCUUC | 1330 | GAAGAATA GGCTAGCTACAACGA TTTGGGTT | 3705 |
| 2305 | UUUUCUUA G UUUCAGAA | 1331 | TTCTGAAA GGCTAGCTACAACGA TAAGAAAA | 3706 |
| 2314 | UUUCAGAA G UACUGGCA | 1332 | TGCCAGTA GGCTAGCTACAACGA TTCTGAAA | 3707 |
| 2320 | AAGUACUG G CAUCACAC | 1333 | GTGTGATG GGCTAGCTACAACGA CAGTACTT | 3708 |
| 2333 | ACACGCAG G UUACCUUG | 1334 | CAAGGTAA GGCTAGCTACAACGA CTGCGTGT | 3709 |
| 2342 | UUACCUUG G CGUGUGUC | 1335 | GACACACG GGCTAGCTACAACGA CAAGGTAA | 3710 |
| 2344 | ACCUUGGC G UGUGUCCC | 1336 | GGGACACA GGCTAGCTACAACGA GCCAAGGT | 3711 |
| 2357 | UCCCUGUG G UACCCUGG | 1337 | CCAGGGTA GGCTAGCTACAACGA CACAGGGA | 3712 |
| 2365 | GUACCCUG G CAGAGAAG | 1338 | CTTCTCTG GGCTAGCTACAACGA CAGGGTAC | 3713 |
| 2381 | GAGACCAA G CUUGUUUC | 1339 | GAAACAAG GGCTAGCTACAACGA TTGGTCTC | 3714 |
| 2397 | CCCUGCUG G CCAAAGUC | 1340 | GACTTTGG GGCTAGCTACAACGA CAGCAGGG | 3715 |
| 2403 | UGGCCAAA G UCAGUAGG | 1341 | CCTACTGA GGCTAGCTACAACGA TTTGGCCA | 3716 |
| 2407 | CAAAGUCA G UAGGAGAG | 1342 | CTCTCCTA GGCTAGCTACAACGA TGACTTTG | 3717 |
| 2424 | GAUGCACA G UUUGCUAU | 1343 | ATAGCAAA GGCTAGCTACAACGA TGTGCATC | 3718 |
| 2463 | AUAAACAA G CCUAACAU | 1344 | ATGTTAGG GGCTAGCTACAACGA TTGTTTAT | 3719 |
| 2474 | UAACAUUG G UGCAAAGA | 1345 | TCTTTGCA GGCTAGCTACAACGA CAATGTTA | 3720 |
| 45 | CGAGCUGG A UUAUGGUG | 1346 | CACCATAA GGCTAGCTACAACGA CCAGCTCG | 3721 |
| 67 | AGCAGCCA A CGCAGCCG | 1347 | CGGCTGCG GGCTAGCTACAACGA TGGCTGCT | 3722 |
| 125 | CCGGGGGG A CCAGGGAA | 1348 | TTCCCTGG GGCTAGCTACAACGA CCCCCCGG | 3723 |
| 217 | CCGUGCCG A UGUAGCGG | 1349 | CCGCTACA GGCTAGCTACAACGA CGGCACGG | 3724 |
| 233 | GGCUCCGG A UCCCAGCC | 1350 | GGCTGGGA GGCTAGCTACAACGA CCGGAGCC | 3725 |
| 267 | CUCUGCGG A UCUCCCCU | 1351 | AGGGGAGA GGCTAGCTACAACGA CCGCAGAG | 3726 |
| 277 | CUCCCCUG A CCGCUCUC | 1352 | GAGAGCGG GGCTAGCTACAACGA CAGGGGAG | 3727 |
| 296 | CAGCCCGG A CCCGGGGG | 1353 | CCCCCGGG GGCTAGCTACAACGA CCGGGCTG | 3728 |
| 338 | GCGUCCUG A UGCCCCCA | 1354 | TGGGGGCA GGCTAGCTACAACGA CAGGACGC | 3729 |
| 383 | CCACCCAG A CUUGGGGG | 1355 | CCCCCAAG GGCTAGCTACAACGA CTGGGTGG | 3730 |
| 404 | CGCCAGGG A CGGACGUG | 1356 | CACGTCCG GGCTAGCTACAACGA CCCTGGCG | 3731 |
| 408 | AGGGACGG A CGUGGGCC | 1357 | GGCCCACG GGCTAGCTACAACGA CCGTCCCT | 3732 |
| 487 | UGCUGUGG A UGGGCGCG | 1358 | CGCGCCCA GGCTAGCTACAACGA CCACAGCA | 3733 |
| 592 | CCCGGGAG A CCGACGAA | 1359 | TTCGTCGG GGCTAGCTACAACGA CTCCCGGG | 3734 |
| 596 | GGAGACCG A CGAAGAGC | 1360 | GCTCTTCG GGCTAGCTACAACGA CGGTCTCC | 3735 |
| 640 | UUGUGGAG A UGGUGGAC | 1361 | GTCCACCA GGCTAGCTACAACGA CTCCACAA | 3736 |
| 647 | GAUGGUGG A CAACCUGA | 1362 | TCAGGTTG GGCTAGCTACAACGA CCACCATC | 3737 |
| 650 | GGUGGACA A CCUGAGGG | 1363 | CCCTCAGG GGCTAGCTACAACGA TGTCCACC | 3738 |
| 688 | ACGUGGAG A UGACCGUG | 1364 | CACGGTCA GGCTAGCTACAACGA CTCCACGT | 3739 |
| 691 | UGGAGAUG A CCGUGGGC | 1365 | GCCCACGG GGCTAGCTACAACGA CATCTCCA | 3740 |
| 712 | CCCCGCAG A CGCUCAAC | 1366 | GTTGAGCG GGCTAGCTACAACGA CTGCGGGG | 3741 |
| 719 | GACGCUCA A CAUCCUGG | 1367 | CCAGGATG GGCTAGCTACAACGA TGAGCGTC | 3742 |
| 731 | CCUGGUGG A UACAGGCA | 1368 | TGCCTGTA GGCTAGCTACAACGA CCACCAGG | 3743 |
| 746 | CAGCAGUA A CUUUGCAG | 1369 | CTGCAAAG GGCTAGCTACAACGA TACTGCTG | 3744 |
| 821 | AUACCGGG A CCUCCGGA | 1370 | TCCGGAGG GGCTAGCTACAACGA CCCGGTAT | 3745 |
| 884 | GGGCACCG A CCUGGUAA | 1371 | TTACTAGG GGCTAGCTACAACGA CGGTGCCC | 3746 |
| 911 | UGGCCCCA A CGUCACUG | 1372 | CAGTGACG GGCTAGCTACAACGA TGGGGCCA | 3747 |
| 929 | GCGUGCCA A CAUUGCUG | 1373 | CAGCAATG GGCTAGCTACAACGA TGGCACGC | 3748 |
| 948 | AUCACUGA A UCAGACAA | 1374 | TTGTCTGA GGCTAGCTACAACGA TCAGTGAT | 3749 |
| 953 | UGAAUCAG A CAAGUUCU | 1375 | AGAACTTG GGCTAGCTACAACGA CTGATTCA | 3750 |
| 968 | CUUCAUCA A CGGCUCCA | 1376 | TGGAGCCG GGCTAGCTACAACGA TGATGAAG | 3751 |
| 977 | CGGCUCCA A CUGGGAAG | 1377 | CTTCCCAG GGCTAGCTACAACGA TGGAGCCG | 3752 |
| 1012 | AUGCUGAG A UUGCCAGG | 1378 | CCTGGCAA GGCTAGCTACAACGA CTCAGCAT | 3753 |
| 1025 | CAGGCCUG A CGACUCCC | 1379 | GGGAGTCG GGCTAGCTACAACGA CAGGCCTG | 3754 |
| 1028 | GCCUGACG A CUCCCUGG | 1380 | CCAGGGAG GGCTAGCTACAACGA CGTCAGGC | 3755 |
| 1049 | UUUCUUUG A CUCUCUGG | 1381 | CCAGAGAG GGCTAGCTACAACGA CAAAGAAA | 3756 |
| 1066 | UAAAGCAG A CCCACGUU | 1382 | AACGTGGG GGCTAGCTACAACGA CTGCTTTA | 3757 |
| 1079 | CGUUCCCA A CCUCUUCU | 1383 | AGAAGAGG GGCTAGCTACAACGA TGGGAACG | 3758 |
| 1121 | CCCCCUCA A CCAGUCUG | 1384 | CAGACTGG GGCTAGCTACAACGA TGAGGGGG | 3759 |
| 1159 | GGAGCAUG A UCAUUGGA | 1385 | TCCAATGA GGCTAGCTACAACGA CATGCTCC | 3760 |
| 1175 | AGGUAUCG A CCACUCGC | 1386 | GCGAGTGG GGCTAGCTACAACGA CGATACCT | 3761 |
| 1240 | AUGAGGUG A UCAUUGUG | 1387 | CACAATGA GGCTAGCTACAACGA CACCTCAT | 3762 |
| 1258 | GGGUGGAG A UCAAUGGA | 1388 | TCCATTGA GGCTAGCTACAACGA CTCCACCC | 3763 |
| 1262 | GGAGAUCA A UGGACAGG | 1389 | CCTGTCCA GGCTAGCTACAACGA TGATCTCC | 3764 |
| 1266 | AUCAAUGG A CAGGAUCU | 1390 | AGATCCTG GGCTAGCTACAACGA CCATTGAT | 3765 |
| 1271 | UGGACAGG A UCUGAAAA | 1391 | TTTTCAGA GGCTAGCTACAACGA CCTGTCCA | 3766 |
| 1279 | AUCUGAAA A UGGACUGC | 1392 | GCAGTCCA GGCTAGCTACAACGA TTTCAGAT | 3767 |
| 1283 | GAAAAUGG A CUGCAAGG | 1393 | CCTTGCAG GGCTAGCTACAACGA CCATTTTC | 3768 |
| 1298 | GGAGUACA A CUAUGACA | 1394 | TGTCATAG GGCTAGCTACAACGA TGTACTCC | 3769 |
| 1304 | CAACUAUG A CAAGAGCA | 1395 | TGCTCTTG GGCTAGCTACAACGA CATAGTTG | 3770 |
| 1319 | CAUUGUGG A CAGUGGCA | 1396 | TGCCACTG GGCTAGCTACAACGA CCACAATG | 3771 |
| 1334 | CACCACCA A CCUUCGUU | 1397 | AACGAAGG GGCTAGCTACAACGA TGGTGGTG | 3772 |
| 1374 | GCAGUCAA A UCCAUCAA | 1398 | TTGATGGA GGCTAGCTACAACGA TTGACTGC | 3773 |
| 1412 | GUUCCCUG A UGGUUUCU | 1399 | AGAAACCA GGCTAGCTACAACGA CAGGGAAC | 3774 |
| 1469 | CCCUUGGA A CAUUUUCC | 1400 | GGAAAATG GGCTAGCTACAACGA TCCAAGGG | 3775 |
| 1498 | UCUACCUA A UGGGUGAG | 1401 | CTCACCCA GGCTAGCTACAACGA TAGGTAGA | 3776 |
| 1514 | GGUUACCA A CCAGUCCU | 1402 | AGGACTGG GGCTAGCTACAACGA TGGTAACC | 3777 |
| 1548 | CCGCAGCA A UACCUGCG | 1403 | CGCAGGTA GGCTAGCTACAACGA TGCTGCGG | 3778 |
| 1568 | AGUGGAAG A UGUGGCCA | 1404 | TGGCCACA GGCTAGCTACAACGA CTTCCACT | 3779 |
| 1586 | GUCCCAAG A CGACUGUU | 1405 | AACAGTCG GGCTAGCTACAACGA CTTGGGAC | 3780 |
| 1589 | CCAAGACG A CUGUUACA | 1406 | TGTAACAG GGCTAGCTACAACGA CGTCTTGG | 3781 |
| 1673 | UGUCUUUG A UCGGGCCC | 1407 | GGGCCCGA GGCTAGCTACAACGA CAAAGACA | 3782 |
| 1686 | GCCCGAAA A CGAAUUGG | 1408 | CCAATTCG GGCTAGCTACAACGA TTTCGGGC | 3783 |
| 1690 | GAAAACGA A UUGGCUUU | 1409 | AAAGCCAA GGCTAGCTACAACGA TCGTTTTC | 3784 |
| 1724 | UGUGCACG A UGAGUUCA | 1410 | TGAACTCA GGCTAGCTACAACGA CGTGCACA | 3785 |
| 1735 | AGUUCAGG A CGGCAGCG | 1411 | CGCTGCCG GGCTAGCTACAACGA CCTGAACT | 3786 |
| 1769 | CACCUUGG A CAUGGAAG | 1412 | CTTCCATG GGCTAGCTACAACGA CCAAGGTG | 3787 |
| 1778 | CAUGGAAG A CUGUGGCU | 1413 | AGCCACAG GGCTAGCTACAACGA CTTCCATG | 3788 |
| 1790 | UGGCUACA A CAUUCCAC | 1414 | GTGGAATG GGCTAGCTACAACGA TGTAGCCA | 3789 |
| 1801 | UUCCACAG A CAGAUGAG | 1415 | CTCATCTG GGCTAGCTACAACGA CTGTGGAA | 3790 |
| 1805 | ACAGACAG A UGAGUCAA | 1416 | TTGACTCA GGCTAGCTACAACGA CTGTCTGT | 3791 |
| 1813 | AUGAGUCA A CCCUCAUG | 1417 | CATGAGGG GGCTAGCTACAACGA TGACTCAT | 3792 |
| 1822 | CCCUCAUG A CCAUAGCC | 1418 | GGCTATGG GGCTAGCTACAACGA CATGAGGG | 3793 |
| 1925 | GCAGCAUG A UGACUUUG | 1419 | CAAAGTCA GGCTAGCTACAACGA CATGCTGC | 3794 |
| 1928 | GCAUGAUG A CUUUGCUG | 1420 | CAGCAAAG GGCTAGCTACAACGA CATCATGC | 3795 |
| 1937 | CUUUGCUG A UGACAUCU | 1421 | AGATGTCA GGCTAGCTACAACGA CAGCAAAG | 3796 |
| 1940 | UGCUGAUG A CAUCUCCC | 1422 | GGGAGATG GGCTAGCTACAACGA CATCAGCA | 3797 |
| 1979 | GGCAGAAG A UAGAGAUU | 1423 | AATCTCTA GGCTAGCTACAACGA CTTCTGCC | 3798 |
| 1985 | AGAUAGAG A UUCCCCUG | 1424 | CAGGGGAA GGCTAGCTACAACGA CTCTATCT | 3799 |
| 1995 | UCCCCUGG A CCACACCU | 1425 | AGGTGTGG GGCTAGCTACAACGA CCAGGGGA | 3800 |
| 2033 | AGUAGGAG A CACAGAUG | 1426 | CATCTGTG GGCTAGCTACAACGA CTCCTACT | 3801 |
| 2039 | AGACACAG A UGGCACCU | 1427 | AGGTGCCA GGCTAGCTACAACGA CTGTGTCT | 3802 |
| 2067 | ACCUCAGG A CCCUCCCC | 1428 | GGGGAGGG GGCTAGCTACAACGA CCTGAGGT | 3803 |
| 2085 | CCCACCAA A UGCCUCUG | 1429 | CAGAGGCA GGCTAGCTACAACGA TTGGTGGG | 3804 |
| 2099 | CUGCCUUG A UGGAGAAG | 1430 | CTTCTCCA GGCTAGCTACAACGA CAAGGCAG | 3805 |
| 2136 | UUCCAGGG A CUGUACCU | 1431 | AGGTACAG GGCTAGCTACAACGA CCCTGGAA | 3806 |
| 2152 | UGUAGGAA A CAGAAAAG | 1432 | CTTTTCTG GGCTAGCTACAACGA TTCCTACA | 3807 |
| 2189 | UGGCGGGA A UACUCUUG | 1433 | CAAGAGTA GGCTAGCTACAACGA TCCCGCCA | 3808 |
| 2208 | CACCUCAA A UUUAAGUC | 1434 | GACTTAAA GGCTAGCTACAACGA TTGAGGTG | 3809 |
| 2222 | GUCGGGAA A UUCUGCUG | 1435 | CAGCAGAA GGCTAGCTACAACGA TTCCCGAC | 3810 |
| 2237 | UGCUUGAA A CUUCAGCC | 1436 | GGCTGAAG GGCTAGCTACAACGA TTCAAGCA | 3811 |
| 2250 | AGCCCUGA A CCUUUGUC | 1437 | GACAAAGG GGCTAGCTACAACGA TCAGGGCT | 3812 |
| 2273 | UCCUUUAA A UUCUCCAA | 1438 | TTGGAGAA GGCTAGCTACAACGA TTAAAGGA | 3813 |
| 2281 | AUUCUCCA A CCCAAAGU | 1439 | ACTTTGGG GGCTAGCTACAACGA TGGAGAAT | 3814 |
| 2376 | GAGAAGAG A CCAAGCUU | 1440 | AAGCTTGG GGCTAGCTACAACGA CTCTTCTC | 3815 |
| 2417 | AGGAGAGG A UGCACAGU | 1441 | ACTGTGCA GGCTAGCTACAACGA CCTCTCCT | 3816 |
| 2444 | CUUUAGAG A CAGGGACU | 1442 | AGTCCCTG GGCTAGCTACAACGA CTCTAAAG | 3817 |
| 2450 | AGACAGGG A CUGUAUAA | 1443 | TTATACAG GGCTAGCTACAACGA CCCTGTCT | 3818 |
| 2459 | CUGUAUAA A CAAGCCUA | 1444 | TAGGCTTG GGCTAGCTACAACGA TTATACAG | 3819 |
| 2468 | CAAGCCUA A CAUUGGUG | 1445 | CACCAATG GGCTAGCTACAACGA TAGGCTTG | 3820 |
| 2482 | GUGCAAAG A UUGCCUCU | 1446 | AGAGGCAA GGCTAGCTACAACGA CTTTGCAC | 3821 |
| 2494 | CCUCUUGA A UUAAAAAA | 1447 | TTTTTTAA GGCTAGCTACAACGA TCAAGAGG | 3822 |
| 2507 | AAAAAAAA A CUAGAAAA | 1448 | TTTTCTAG GGCTAGCTACAACGA TTTTTTTT | 3823 |

Input Sequence = AF190725.
Cut Site = G/.
Stem Length = 8.
Core Sequence = GGCTAGCTACAACGA
AF190725 (Homo sapiens beta-site APP cleaving enzyme (BACE) mRNA; 2526 bp)

TABLE VIII

Human BACE Amberzyme Ribozyme and Target Sequence

| | | | | |
|---|---|---|---|---|
| Pos | Substrate | Seq ID | Ribozyme | Rz Seq ID |

| | | | | |
|---|---|---|---|---|
| 11 | ACGCGUCC G CAGCCCGC | 960 | GCGGGCUG GGAGGAAACUCC CU UCAAGGACAUCGUCCGGG GGACGCGU | 3260 |
| 18 | CGCAGCCC G CCCGGGAG | 961 | CUCCCGGG GGAGGAAACUCC CU UCAAGGACAUCGUCCGGG GGGCUGCG | 3261 |
| 29 | CGGGAGCU G CGAGCCGC | 962 | GCGGCUCG GGAGGAAACUCC CU UCAAGGACAUCGUCCGGG AGCUCCCG | 3262 |
| 31 | GGAGCUGC G AGCCGCGA | 963 | UCGCGGCU GGAGGAAACUCC CU UCAAGGACAUCGUCCGGG GCAGCUCC | 3263 |
| 36 | UGCGAGCC G CGAGCUGG | 964 | CCAGCUCG GGAGGAAACUCC CU UCAAGGACAUCGUCCGGG GGCUCGCA | 3264 |
| 38 | CGAGCCGC G AGCUGGAU | 965 | AUCCAGCU GGAGGAAACUCC CU UCAAGGACAUCGUCCGGG GCGGCUCG | 3265 |
| 58 | GGUGGCCU G AGCAGCCA | 966 | UGGCUGCU GGAGGAAACUCC CU UCAAGGACAUCGUCCGGG AGGCCACC | 3266 |
| 69 | CAGCCAAC G CAGCCGCA | 967 | UGCGGCUG GGAGGAAACUCC CU UCAAGGACAUCGUCCGGG GUUGGCUG | 3267 |
| 75 | ACGCAGCC G CAGGAGCC | 968 | GGCUCCUG GGAGGAAACUCC CU UCAAGGACAUCGUCCGGG GGCUGCGU | 3268 |
| 94 | GAGCCCUU G CCCCUGCC | 969 | GGCAGGGG GGAGGAAACUCC CU UCAAGGACAUCGUCCGGG AAGGGCUC | 3269 |
| 100 | UUGCCCCU G CCCGCGCC | 970 | GGCGCGGG GGAGGAAACUCC CU UCAAGGACAUCGUCCGGG AGGGGCAA | 3270 |
| 104 | CCCUGCCC G CGCCGCCG | 971 | CGGCGGCG GGAGGAAACUCC CU UCAAGGACAUCGUCCGGG GGGCAGGG | 3271 |
| 106 | CUGCCCGC G CCGCCGCC | 972 | GGCGGCGG GGAGGAAACUCC CU UCAAGGACAUCGUCCGGG GCGGGCAG | 3272 |
| 109 | CCCGCGCC G CCGCCCGC | 973 | GCGGGCGG GGAGGAAACUCC CU UCAAGGACAUCGUCCGGG GGCGCGGG | 3273 |
| 112 | GCGCCGCC G CCCGCCGG | 974 | CCGGCGGG GGAGGAAACUCC CU UCAAGGACAUCGUCCGGG GGCGGCGC | 3274 |
| 116 | CGCCGCCC G CCGGGGGG | 975 | CCCCCCGG GGAGGAAACUCC CU UCAAGGACAUCGUCCGGG GGGCGGCG | 3275 |
| 137 | GGGAAGCC G CCACCGGC | 976 | GCCGGUGG GGAGGAAACUCC CU UCAAGGACAUCGUCCGGG GGCUUCCC | 3276 |
| 148 | ACCGGCCC G CCAUGCCC | 977 | GGGCAUGG GGAGGAAACUCC CU UCAAGGACAUCGUCCGGG GGGCCGGU | 3277 |
| 153 | CCCGCCAU G CCCGCCCC | 978 | GGGGCGGG GGAGGAAACUCC CU UCAAGGACAUCGUCCGGG AUGGCGGG | 3278 |
| 157 | CCAUGCCC G CCCCUCCC | 979 | GGGAGGGG GGAGGAAACUCC CU UCAAGGACAUCGUCCGGG GGGCAUGG | 3279 |
| 172 | CCAGCCCC G CCGGGAGC | 980 | GCUCCCGG GGAGGAAACUCC CU UCAAGGACAUCGUCCGGG GGGGCUGG | 3280 |
| 183 | GGGAGCCC G CGCCCGCU | 981 | AGCGGGCG GGAGGAAACUCC CU UCAAGGACAUCGUCCGGG GGGCUCCC | 3281 |
| 185 | GAGCCCGC G CCCGCUGC | 982 | GCAGCGGG GGAGGAAACUCC CU UCAAGGACAUCGUCCGGG GCGGGCUC | 3282 |
| 189 | CCGCGCCC G CUGCCCAG | 983 | CUGGGCAG GGAGGAAACUCC CU UCAAGGACAUCGUCCGGG GGGCGCGG | 3283 |
| 192 | CGCCCGCU G CCCAGGCU | 984 | AGCCUGGG GGAGGAAACUCC CU UCAAGGACAUCGUCCGGG AGCGGGCG | 3284 |
| 205 | GGCUGGCC G CCGCCGUG | 985 | CACGGCGG GGAGGAAACUCC CU UCAAGGACAUCGUCCGGG GGCCAGCC | 3285 |
| 208 | UGGCCGCC G CCGUGCCG | 986 | CGGCACGG GGAGGAAACUCC CU UCAAGGACAUCGUCCGGG GGCGGCCA | 3286 |
| 213 | GCCGCCGU G CCGAUGUA | 987 | UACAUCGG GGAGGAAACUCC CU UCAAGGACAUCGUCCGGG ACGGCGGC | 3287 |
| 216 | GCCGUGCC G AUGUAGCG | 988 | CGCUACAU GGAGGAAACUCC CU UCAAGGACAUCGUCCGGG GGCACGGC | 3288 |
| 250 | UCUCCCCU G CUCCCGUG | 989 | CACGGGAG GGAGGAAACUCC CU UCAAGGACAUCGUCCGGG AGGGGAGA | 3289 |
| 258 | GCUCCCGU G CUCUGCGG | 990 | CCGCAGAG GGAGGAAACUCC CU UCAAGGACAUCGUCCGGG ACGGGAGC | 3290 |
| 263 | CGUGCUCU G CGGAUCUC | 991 | GAGAUCCG GGAGGAAACUCC CU UCAAGGACAUCGUCCGGG AGAGCACG | 3291 |
| 276 | UCUCCCCU G ACCGCUCU | 992 | AGAGCGGU GGAGGAAACUCC CU UCAAGGACAUCGUCCGGG AGGGGAGA | 3292 |
| 280 | CCCUGACC G CUCUCCAC | 993 | GUGGAGAG GGAGGAAACUCC CU UCAAGGACAUCGUCCGGG GGUCAGGG | 3293 |
| 320 | AGGGCCCU G CAGGCCCU | 994 | AGGGCCUG GGAGGAAACUCC CU UCAAGGACAUCGUCCGGG AGGGCCCU | 3294 |
| 337 | GGCGUCCU G AUGCCCCC | 995 | GGGGGCAU GGAGGAAACUCC CU UCAAGGACAUCGUCCGGG AGGACGCC | 3295 |
| 340 | GUCCUGAU G CCCCCAAG | 996 | CUUGGGGG GGAGGAAACUCC CU UCAAGGACAUCGUCCGGG AUCAGGAC | 3296 |
| 360 | CCUCUCCU G AGAAGCCA | 997 | UGGCUUCU GGAGGAAACUCC CU UCAAGGACAUCGUCCGGG AGGAGAGG | 3297 |
| 397 | GGGCAGGC G CCAGGGAC | 998 | GUCCCUGG GGAGGAAACUCC CU UCAAGGACAUCGUCCGGG GCCUGCCC | 3298 |
| 420 | GGGCCAGU G CGAGCCCA | 999 | UGGGCUCG GGAGGAAACUCC CU UCAAGGACAUCGUCCGGG ACUGGCCC | 3299 |
| 422 | GCCAGUGC G AGCCCAGA | 1000 | UCUGGGCU GGAGGAAACUCC CU UCAAGGACAUCGUCCGGG GCACUGGC | 3300 |
| 437 | GAGGGCCC G AAGGCCGG | 1001 | CCGGCCUU GGAGGAAACUCC CU UCAAGGACAUCGUCCGGG GGGCCCUC | 3301 |
| 468 | CAAGCCCU G CCCUGGCU | 1002 | AGCCAGGG GGAGGAAACUCC CU UCAAGGACAUCGUCCGGG AGGGCUUG | 3302 |
| 480 | UGGCUCCU G CUGUGGAU | 1003 | AUCCACAG GGAGGAAACUCC CU UCAAGGACAUCGUCCGGG AGGAGCCA | 3303 |
| 493 | GGAUGGGC G CGGGAGUG | 1004 | CACUCCCG GGAGGAAACUCC CU UCAAGGACAUCGUCCGGG GCCCAUCC | 3304 |
| 501 | GCGGGAGU G CUGCCUGC | 1005 | GCAGGCAG GGAGGAAACUCC CU UCAAGGACAUCGUCCGGG ACUCCCGC | 3305 |
| 504 | GGAGUGCU G CCUGCCCA | 1006 | UGGGCAGG GGAGGAAACUCC CU UCAAGGACAUCGUCCGGG AGCACUCC | 3306 |
| 508 | UGCUGCCU G CCCACGGC | 1007 | GCCGUGGG GGAGGAAACUCC CU UCAAGGACAUCGUCCGGG AGGCAGCA | 3307 |
| 537 | AUCCGGCU G CCCCUGCG | 1008 | CGCAGGGG GGAGGAAACUCC CU UCAAGGACAUCGUCCGGG AGCCGGAU | 3308 |
| 543 | CUGCCCCU G CGCAGCGG | 1009 | CCGCUGCG GGAGGAAACUCC CU UCAAGGACAUCGUCCGGG AGGGGCAG | 3309 |
| 545 | GCCCCUGC G CAGCGGCC | 1010 | GGCCGCUG GGAGGAAACUCC CU UCAAGGACAUCGUCCGGG GCAGGGGC | 3310 |
| 562 | UGGGGGGC G CCCCCCUG | 1011 | CAGGGGGG GGAGGAAACUCC CU UCAAGGACAUCGUCCGGG GCCCCCCA | 3311 |
| 576 | CUGGGGCU G CGGCUGCC | 1012 | GGCAGCCG GGAGGAAACUCC CU UCAAGGACAUCGUCCGGG AGCCCCAG | 3312 |
| 582 | CUGCGGCU G CCCCGGGA | 1013 | UCCCGGGG GGAGGAAACUCC CU UCAAGGACAUCGUCCGGG AGCCGCAG | 3313 |
| 595 | GGGAGACC G ACGAAGAG | 1014 | CUCUUCGU GGAGGAAACUCC CU UCAAGGACAUCGUCCGGG GGUCUCCC | 3314 |
| 598 | AGACCGAC G AAGAGCCC | 1015 | GGGCUCUU GGAGGAAACUCC CU UCAAGGACAUCGUCCGGG GUCGGUCU | 3315 |
| 607 | AAGAGCCC G AGGAGCCC | 1016 | GGGCUCCU GGAGGAAACUCC CU UCAAGGACAUCGUCCGGG GGGCUCUU | 3316 |
| 654 | GACAACCU G AGGGGCAA | 1017 | UUGCCCCU GGAGGAAACUCC CU UCAAGGACAUCGUCCGGG AGGUUGUC | 3317 |
| 690 | GUGGAGAU G ACCGUGGG | 1018 | CCCACGGU GGAGGAAACUCC CU UCAAGGACAUCGUCCGGG AUCUCCAC | 3318 |
| 708 | AGCCCCCC G CAGACGCU | 1019 | AGCGUCUG GGAGGAAACUCC CU UCAAGGACAUCGUCCGGG GGGGGGCU | 3319 |
| 714 | CCGCAGAC G CUCAACAU | 1020 | AUGUUGAG GGAGGAAACUCC CU UCAAGGACAUCGUCCGGG GUCUGCGG | 3320 |
| 751 | GUAACUUU G CAGUGGGU | 1021 | ACCCACUG GGAGGAAACUCC CU UCAAGGACAUCGUCCGGG AAAGUUAC | 3321 |
| 760 | CAGUGGGU G CUGCCCCC | 1022 | GGGGGCAG GGAGGAAACUCC CU UCAAGGACAUCGUCCGGG ACCCACUG | 3322 |
| 763 | UGGGUGCU G CCCCCCAC | 1023 | GUGGGGGG GGAGGAAACUCC CU UCAAGGACAUCGUCCGGG AGCACCCA | 3323 |
| 780 | CCCUUCCU G CAUCGCUA | 1024 | UAGCGAUG GGAGGAAACUCC CU UCAAGGACAUCGUCCGGG AGGAAGGG | 3324 |
| 785 | CCUGCAUC G CUACUACC | 1025 | GGUAGUAG GGAGGAAACUCC CU UCAAGGACAUCGUCCGGG GAUGCAGG | 3325 |
| 843 | GUGUAUGU G CCCUACAC | 1026 | GUGUAGGG GGAGGAAACUCC CU UCAAGGACAUCGUCCGGG ACAUACAC | 3326 |
| 883 | UGGGCACC G ACCUGGUA | 1027 | UACCAGGU GGAGGAAACUCC CU UCAAGGACAUCGUCCGGG GGUGCCCA | 3327 |
| 921 | GUCACUGU G CGUGCCAA | 1028 | UUGGCACG GGAGGAAACUCC CU UCAAGGACAUCGUCCGGG ACAGUGAC | 3328 |
| 925 | CUGUGCGU G CCAACAUU | 1029 | AAUGUUGG GGAGGAAACUCC CU UCAAGGACAUCGUCCGGG ACGCACAG | 3329 |
| 934 | CCAACAUU G CUGCCAUC | 1030 | GAUGGCAG GGAGGAAACUCC CU UCAAGGACAUCGUCCGGG AAUGUUGG | 3330 |
| 937 | ACAUUGCU G CCAUCACU | 1031 | AGUGAUGG GGAGGAAACUCC CU UCAAGGACAUCGUCCGGG AGCAAUGU | 3331 |
| 946 | CCAUCACU G AAUCAGAC | 1032 | GUCUGAUU GGAGGAAACUCC CU UCAAGGACAUCGUCCGGG AGUGAUGG | 3332 |
| 1006 | UGGCCUAU G CUGAGAUU | 1033 | AAUCUCAG GGAGGAAACUCC CU UCAAGGACAUCGUCCGGG AUAGGCCA | 3333 |
| 1009 | CCUAUGCU G AGAUUGCC | 1034 | GGCAAUCU GGAGGAAACUCC CU UCAAGGACAUCGUCCGGG AGCAUAGG | 3334 |
| 1015 | CUGAGAUU G CCAGGCCU | 1035 | AGGCCUGG GGAGGAAACUCC CU UCAAGGACAUCGUCCGGG AAUCUCAG | 3335 |
| 1024 | CCAGGCCU G ACGACUCC | 1036 | GGAGUCGU GGAGGAAACUCC CU UCAAGGACAUCGUCCGGG AGGCCUGG | 3336 |
| 1027 | GGCCUGAC G ACUCCCUG | 1037 | CAGGGAGU GGAGGAAACUCC CU UCAAGGACAUCGUCCGGG GUCAGGCC | 3337 |
| 1048 | CUUUCUUU G ACUCUCUG | 1038 | CAGAGAGU GGAGGAAACUCC CU UCAAGGACAUCGUCCGGG AAAGAAAG | 3338 |
| 1092 | UUCUCCCU G CAGCUUUG | 1039 | CAAAGCUG GGAGGAAACUCC CU UCAAGGACAUCGUCCGGG AGGGAGAA | 3339 |
| 1105 | UUUGUGGU G CUGGCUUC | 1040 | GAAGCCAG GGAGGAAACUCC CU UCAAGGACAUCGUCCGGG ACCACAAA | 3340 |
| 1129 | ACCAGUCU G AAGUGCUG | 1041 | CAGCACUU GGAGGAAACUCC CU UCAAGGACAUCGUCCGGG AGACUGGU | 3341 |
| 1134 | UCUGAAGU G CUGGCCUC | 1042 | GAGGCCAG GGAGGAAACUCC CU UCAAGGACAUCGUCCGGG ACUUCAGA | 3342 |
| 1158 | GGGAGCAU G AUCAUUGG | 1043 | CCAAUGAU GGAGGAAACUCC CU UCAAGGACAUCGUCCGGG AUGCUCCC | 3343 |
| 1174 | GAGGUAUC G ACCACUCG | 1044 | CGAGUGGU GGAGGAAACUCC CU UCAAGGACAUCGUCCGGG GAUACCUC | 3344 |
| 1182 | GACCACUC G CUGUACAC | 1045 | GUGUACAG GGAGGAAACUCC CU UCAAGGACAUCGUCCGGG GAGUGGUC | 3345 |
| 1234 | GGUAUUAU G AGGUGAUC | 1046 | GAUCACCU GGAGGAAACUCC CU UCAAGGACAUCGUCCGGG AUAAUACC | 3346 |
| 1239 | UAUGAGGU G AUCAUUGU | 1047 | ACAAUGAU GGAGGAAACUCC CU UCAAGGACAUCGUCCGGG ACCUCAUA | 3347 |
| 1248 | AUCAUUGU G CGGGUGGA | 1048 | UCCACCCG GGAGGAAACUCC CU UCAAGGACAUCGUCCGGG ACAAUGAU | 3348 |
| 1275 | CAGGAUCU G AAAAUGGA | 1049 | UCCAUUUU GGAGGAAACUCC CU UCAAGGACAUCGUCCGGG AGAUCCUG | 3349 |
| 1286 | AAUGGACU G CAAGGAGU | 1050 | ACUCCUUG GGAGGAAACUCC CU UCAAGGACAUCGUCCGGG AGUCCAUU | 3350 |
| 1303 | ACAACUAU G ACAAGAGC | 1051 | GCUCUUGU GGAGGAAACUCC CU UCAAGGACAUCGUCCGGG AUAGUUGU | 3351 |
| 1344 | CUUCGUUU G CCCAAGAA | 1052 | UUCUUGGG GGAGGAAACUCC CU UCAAGGACAUCGUCCGGG AAACGAAG | 3352 |
| 1360 | AAGUGUUU G AAGCUGCA | 1053 | UGCAGCUU GGAGGAAACUCC CU UCAAGGACAUCGUCCGGG AAACACUU | 3353 |
| 1366 | UUGAAGCU G CAGUCAAA | 1054 | UUUGACUG GGAGGAAACUCC CU UCAAGGACAUCGUCCGGG AGCUUCAA | 3354 |
| 1411 | AGUUCCCU G AUGGUUUC | 1055 | GAAACCAU GGAGGAAACUCC CU UCAAGGACAUCGUCCGGG AGGGAACU | 3355 |
| 1442 | GCUGGUGU G CUGGCAAG | 1056 | CUUGCCAG GGAGGAAACUCC CU UCAAGGACAUCGUCCGGG ACACCAGC | 3356 |
| 1504 | UAAUGGGU G AGGUUACC | 1057 | GGUAACCU GGAGGAAACUCC CU UCAAGGACAUCGUCCGGG ACCCAUUA | 3357 |
| 1526 | GUCCUUCC G CAUCACCA | 1058 | UGGUGAUG GGAGGAAACUCC CU UCAAGGACAUCGUCCGGG GGAAGGAC | 3358 |
| 1542 | AUCCUUCC G CAGCAAUA | 1059 | UAUUGCUG GGAGGAAACUCC CU UCAAGGACAUCGUCCGGG GGAAGGAU | 3359 |
| 1554 | CAAUACCU G CGGCCAGU | 1060 | ACUGGCCG GGAGGAAACUCC CU UCAAGGACAUCGUCCGGG AGGUAUUG | 3360 |
| 1588 | CCCAAGAC G ACUGUUAC | 1061 | GUAACAGU GGAGGAAACUCC CU UCAAGGACAUCGUCCGGG GUCUUGGG | 3361 |
| 1603 | ACAAGUUU G CCAUCUCA | 1062 | UGAGAUGG GGAGGAAACUCC CU UCAAGGACAUCGUCCGGG AAACUUGU | 3362 |
| 1672 | UUGUCUUU G AUCGGGCC | 1063 | GGCCCGAU GGAGGAAACUCC CU UCAAGGACAUCGUCCGGG AAAGACAA | 3363 |
| 1682 | UCGGGCCC G AAAACGAA | 1064 | UUCGUUUU GGAGGAAACUCC CU UCAAGGACAUCGUCCGGG GGGCCCGA | 3364 |
| 1688 | CCGAAAAC G AAUUGGCU | 1065 | AGCCAAUU GGAGGAAACUCC CU UCAAGGACAUCGUCCGGG GUUUUCGG | 3365 |
| 1699 | UUGGCUUU G CUGUCAGC | 1066 | GCUGACAG GGAGGAAACUCC CU UCAAGGACAUCGUCCGGG AAAGCCAA | 3366 |
| 1708 | CUGUCAGC G CUUGCCAU | 1067 | AUGGCAAG GGAGGAAACUCC CU UCAAGGACAUCGUCCGGG GCUGACAG | 3367 |
| 1712 | CAGCGCUU G CCAUGUGC | 1068 | GCACAUGG GGAGGAAACUCC CU UCAAGGACAUCGUCCGGG AAGCGCUG | 3368 |
| 1719 | UGCCAUGU G CAGGAUGA | 1069 | UCAUCGUG GGAGGAAACUCC CU UCAAGGACAUCGUCCGGG ACAUGGCA | 3369 |
| 1723 | AUGUGCAC G AUGAGUUC | 1070 | GAACUCAU GGAGGAAACUCC CU UCAAGGACAUCGUCCGGG GUGCACAU | 3370 |
| 1726 | UGCACGAU G AGUUCAGG | 1071 | CCUGAACU GGAGGAAACUCC CU UCAAGGACAUCGUCCGGG AUCGUGCA | 3371 |
| 1807 | AGACAGAU G AGUCAACC | 1072 | GGUUGACU GGAGGAAACUCC CU UCAAGGACAUCGUCCGGG AUCUGUCU | 3372 |
| 1821 | ACCCUCAU G ACCAUAGC | 1073 | GCUAUGGU GGAGGAAACUCC CU UCAAGGACAUCGUCCGGG AUGAGGGU | 3373 |
| 1843 | UCAUGGCU G CCAUCUGC | 1074 | GCAGAUGG GGAGGAAACUCC CU UCAAGGACAUCGUCCGGG AGCCAUGA | 3374 |
| 1850 | UGCCAUCU G CGCCCUCU | 1075 | AGAGGGCG GGAGGAAACUCC CU UCAAGGACAUCGUCCGGG AGAUGGCA | 3375 |
| 1852 | CCAUCUGC G CCCUCUUC | 1076 | GAAGAGGG GGAGGAAACUCC CU UCAAGGACAUCGUCCGGG GCAGAUGG | 3376 |
| 1863 | CUCUUCAU G CUGCCACU | 1077 | AGUGGCAG GGAGGAAACUCC CU UCAAGGACAUCGUCCGGG AUGAAGAG | 3377 |
| 1866 | UUCAUGCU G CCACUCUG | 1078 | CAGAGUGG GGAGGAAACUCC CU UCAAGGACAUCGUCCGGG AGCAUGAA | 3378 |
| 1874 | GCCACUCU G CCUCAUGG | 1079 | CCAUGAGG GGAGGAAACUCC CU UCAAGGACAUCGUCCGGG AGAGUGGC | 3379 |
| 1895 | UCAGUGGC G CUGCCUCC | 1080 | GGAGGCAG GGAGGAAACUCC CU UCAAGGACAUCGUCCGGG GCCACUGA | 3380 |
| 1898 | GUGGCGCU G CCUCCGCU | 1081 | AGCGGAGG GGAGGAAACUCC CU UCAAGGACAUCGUCCGGG AGCGCCAC | 3381 |
| 1904 | CUGCCUCC G CUGCCUGC | 1082 | GCAGGCAG GGAGGAAACUCC CU UCAAGGACAUCGUCCGGG GGAGGCAG | 3382 |
| 1907 | CCUCCGCU G CCUGCGCC | 1083 | GGCGCAGG GGAGGAAACUCC CU UCAAGGACAUCGUCCGGG AGCGGAGG | 3383 |
| 1911 | CGCUGCCU G CGCCAGCA | 1084 | UGCUGGCG GGAGGAAACUCC CU UCAAGGACAUCGUCCGGG AGGCAGCG | 3384 |
| 1913 | CUGCCUGC G CCAGCAGC | 1085 | GCUGCUGG GGAGGAAACUCC CU UCAAGGACAUCGUCCGGG GCAGGCAG | 3385 |
| 1924 | AGCAGCAU G AUGACUUU | 1086 | AAAGUCAU GGAGGAAACUCC CU UCAAGGACAUCGUCCGGG AUGCUGCU | 3386 |
| 1927 | AGCAUGAU G ACUUUGCU | 1087 | AGCAAAGU GGAGGAAACUCC CU UCAAGGACAUCGUCCGGG AUCAUGCU | 3387 |
| 1933 | AUGACUUU G CUGAUGAC | 1088 | GUCAUCAG GGAGGAAACUCC CU UCAAGGACAUCGUCCGGG AAAGUCAU | 3388 |
| 1936 | ACUUUGCU G AUGACAUC | 1089 | GAUGUCAU GGAGGAAACUCC CU UCAAGGACAUCGUCCGGG AGCAAAGU | 3389 |
| 1939 | UUGCUGAU G ACAUCUCC | 1090 | GGAGAUGU GGAGGAAACUCC CU UCAAGGACAUCGUCCGGG AUCAGCAA | 3390 |
| 1950 | AUCUCCCU G CUGAAGUG | 1091 | CACUUCAG GGAGGAAACUCC CU UCAAGGACAUCGUCCGGG AGGGAGAU | 3391 |
| 1953 | UCCCUGCU G AAGUGAGG | 1092 | CCUCACUU GGAGGAAACUCC CU UCAAGGACAUCGUCCGGG AGCAGGGA | 3392 |
| 1958 | GCUGAAGU G AGGAGGCC | 1093 | GGCCUCCU GGAGGAAACUCC CU UCAAGGACAUCGUCCGGG ACUUCAGC | 3393 |
| 2087 | CACCAAAU G CCUCUGCC | 1094 | GGCAGAGG GGAGGAAACUCC CU UCAAGGACAUCGUCCGGG AUUUGGUG | 3394 |
| 2093 | AUGCCUCU G CCUUGAUG | 1095 | CAUCAAGG GGAGGAAACUCC CU UCAAGGACAUCGUCCGGG AGAGGCAU | 3395 |
| 2098 | UCUGCCUU G AUGGAGAA | 1096 | UUCUCCAU GGAGGAAACUCC CU UCAAGGACAUCGUCCGGG AAGGCAGA | 3396 |
| 2179 | AGCACUCU G CUGGCGGG | 1097 | CCCGCCAG GGAGGAAACUCC CU UCAAGGACAUCGUCCGGG AGAGUGCU | 3397 |
| 2227 | GAAAUUCU G CUGCUUGA | 1098 | UCAAGCAG GGAGGAAACUCC CU UCAAGGACAUCGUCCGGG AGAAUUUC | 3398 |
| 2230 | AUUCUGCU G CUUGAAAC | 1099 | GUUUCAAG GGAGGAAACUCC CU UCAAGGACAUCGUCCGGG AGCAGAAU | 3399 |
| 2234 | UGCUGCUU G AAACUUCA | 1100 | UGAAGUUU GGAGGAAACUCC CU UCAAGGACAUCGUCCGGG AAGCAGCA | 3400 |
| 2248 | UCAGCCCU G AACCUUUG | 1101 | CAAAGGUU GGAGGAAACUCC CU UCAAGGACAUCGUCCGGG AGGGCUGA | 3401 |
| 2329 | CAUCACAC G CAGGUUAC | 1102 | GUAACCUG GGAGGAAACUCC CU UCAAGGACAUCGUCCGGG GUGUGAUG | 3402 |
| 2393 | GUUUCCCU G CUGGCCAA | 1103 | UUGGCCAG GGAGGAAACUCC CU UCAAGGACAUCGUCCGGG AGGGAAAC | 3403 |
| 2419 | GAGAGGAU G CACAGUUU | 1104 | AAACUGUG GGAGGAAACUCC CU UCAAGGACAUCGUCCGGG AUCCUCUC | 3404 |
| 2428 | CACAGUUU G CUAUUUGC | 1105 | GCAAAUAG GGAGGAAACUCC CU UCAAGGACAUCGUCCGGG AAACUGUG | 3405 |
| 2435 | UGCUAUUU G CUUUAGAG | 1106 | CUCUAAAG GGAGGAAACUCC CU UCAAGGACAUCGUCCGGG AAAUAGCA | 3406 |
| 2476 | ACAUUGGU G CAAAGAUU | 1107 | AAUCUUUG GGAGGAAACUCC CU UCAAGGACAUCGUCCGGG ACCAAUGU | 3407 |
| 2485 | CAAAGAUU G CCUCUUGA | 1108 | UCAAGAGG GGAGGAAACUCC CU UCAAGGACAUCGUCCGGG AAUCUUUG | 3408 |
| 2492 | UGCCUCUU G AAUUAAAA | 1109 | UUUUAAUU GGAGGAAACUCC CU UCAAGGACAUCGUCCGGG AAGAGGCA | 3409 |
| 219 | GUGCCGAU G UAGCGGGC | 1110 | GCCCGCUA GGAGGAAACUCC CU UCAAGGACAUCGUCCGGG AUCGGCAC | 3410 |
| 483 | CUCCUGCU G UGGAUGGG | 1111 | CCCAUCCA GGAGGAAACUCC CU UCAAGGACAUCGUCCGGG AGCAGGAG | 3411 |
| 634 | GCAGCUUU G UGGAGAUG | 1112 | CAUCUCCA GGAGGAAACUCC CU UCAAGGACAUCGUCCGGG AAAGCUGC | 3412 |
| 804 | AGGCAGCU G UCCAGCAC | 1113 | GUGCUGGA GGAGGAAACUCC CU UCAAGGACAUCGUCCGGG AGCUGCCU | 3413 |
| 835 | GGAAGGGU G UGUAUGUG | 1114 | CACAUACA GGAGGAAACUCC CU UCAAGGACAUCGUCCGGG ACCCUUCC | 3414 |
| 837 | AAGGGUGU G UAUGUGCC | 1115 | GGCACAUA GGAGGAAACUCC CU UCAAGGACAUCGUCCGGG ACACCCUU | 3415 |
| 841 | GUGUGUAU G UGCCCUAC | 1116 | GUAGGGCA GGAGGAAACUCC CU UCAAGGACAUCGUCCGGG AUACACAC | 3416 |
| 919 | ACGUCACU G UGCGUGCC | 1117 | GGCACGCA GGAGGAAACUCC CU UCAAGGACAUCGUCCGGG AGUGACGU | 3417 |
| 1100 | GCAGCUUU G UGGUGCUG | 1118 | CAGCACCA GGAGGAAACUCC CU UCAAGGACAUCGUCCGGG AAAGCUGC | 3418 |
| 1144 | UGGCCUCU G UCGGAGGG | 1119 | CCCUCCGA GGAGGAAACUCC CU UCAAGGACAUCGUCCGGG AGAGGCCA | 3419 |
| 1185 | CACUCGCU G UACACAGG | 1120 | CCUGUGUA GGAGGAAACUCC CU UCAAGGACAUCGUCCGGG AGCGAGUG | 3420 |
| 1246 | UGAUCAUU G UGCGGGUG | 1121 | CACCCGCA GGAGGAAACUCC CU UCAAGGACAUCGUCCGGG AAUGAUCA | 3421 |
| 1315 | AGAGCAUU G UGGACAGU | 1122 | ACUGUCCA GGAGGAAACUCC CU UCAAGGACAUCGUCCGGG AAUGCUCU | 3422 |
| 1356 | AAGAAAGU G UUUGAAGC | 1123 | GCUUCAAA GGAGGAAACUCC CU UCAAGGACAUCGUCCGGG ACUUUCUU | 3423 |
| 1440 | CAGCUGGU G UGCUGGCA | 1124 | UGCCAGCA GGAGGAAACUCC CU UCAAGGACAUCGUCCGGG ACCAGCUG | 3424 |
| 1570 | UGGAAGAU G UGGCCACG | 1125 | CGUGGCCA GGAGGAAACUCC CU UCAAGGACAUCGUCCGGG AUCUUCCA | 3425 |
| 1592 | AGACGACU G UUACAAGU | 1126 | ACUUGUAA GGAGGAAACUCC CU UCAAGGACAUCGUCCGGG AGUCGUCU | 3426 |
| 1630 | CGGGCACU G UUAUGGGA | 1127 | UCCCAUAA GGAGGAAACUCC CU UCAAGGACAUCGUCCGGG AGUGCCCG | 3427 |
| 1642 | UGGGAGCU G UUAUCAUG | 1128 | CAUGAUAA GGAGGAAACUCC CU UCAAGGACAUCGUCCGGG AGCUCCCA | 3428 |
| 1666 | UCUACGUU G UCUUUGAU | 1129 | AUCAAAGA GGAGGAAACUCC CU UCAAGGACAUCGUCCGGG AACGUAGA | 3429 |
| 1702 | GCUUUGCU G UCAGCGCU | 1130 | AGCGCUGA GGAGGAAACUCC CU UCAAGGACAUCGUCCGGG AGCAAAGC | 3430 |
| 1717 | CUUGCCAU G UGCACGAU | 1131 | AUCGUGCA GGAGGAAACUCC CU UCAAGGACAUCGUCCGGG AUGGCAAG | 3431 |
| 1759 | GCCCUUUU G UCACCUUG | 1132 | CAAGGUGA GGAGGAAACUCC CU UCAAGGACAUCGUCCGGG AAAAGGGC | 3432 |
| 1781 | GGAAGACU G UGGCUACA | 1133 | UGUAGCCA GGAGGAAACUCC CU UCAAGGACAUCGUCCGGG AGUCUUCC | 3433 |
| 1834 | UAGCCUAU G UCAUGGCU | 1134 | AGCCAUGA GGAGGAAACUCC CU UCAAGGACAUCGUCCGGG AUAGGCUA | 3434 |
| 1884 | CUCAUGGU G UGUCAGUG | 1135 | CACUGACA GGAGGAAACUCC CU UCAAGGACAUCGUCCGGG ACCAUGAG | 3435 |
| 1886 | CAUGGUGU G UCAGUGGC | 1136 | GCCACUGA GGAGGAAACUCC CU UCAAGGACAUCGUCCGGG ACACCAUG | 3436 |
| 2048 | UGGCACCU G UGGCCAGA | 1137 | UCUGGCCA GGAGGAAACUCC CU UCAAGGACAUCGUCCGGG AGGUGCCA | 3437 |
| 2139 | CAGGGACU G UACCUGUA | 1138 | UACAGGUA GGAGGAAACUCC CU UCAAGGACAUCGUCCGGG AGUCCCUG | 3438 |
| 2145 | CUGUACCU G UAGGAAAC | 1139 | GUUUCCUA GGAGGAAACUCC CU UCAAGGACAUCGUCCGGG AGGUACAG | 3439 |
| 2256 | GAACCUUU G UCCACCAU | 1140 | AUGGUGGA GGAGGAAACUCC CU UCAAGGACAUCGUCCGGG AAAGGUUC | 3440 |
| 2346 | CUUGGCGU G UGUCCCUG | 1141 | CAGGGACA GGAGGAAACUCC CU UCAAGGACAUCGUCCGGG ACGCCAAG | 3441 |
| 2348 | UGGCGUGU G UCCCUGUG | 1142 | CACAGGGA GGAGGAAACUCC CU UCAAGGACAUCGUCCGGG ACACGCCA | 3442 |
| 2354 | GUGUCCCU G UGGUACCC | 1143 | GGGUACCA GGAGGAAACUCC CU UCAAGGACAUCGUCCGGG AGGGACAC | 3443 |
| 2385 | CCAAGCUU G UUUCCCUG | 1144 | CAGGGAAA GGAGGAAACUCC CU UCAAGGACAUCGUCCGGG AAGCUUGG | 3444 |
| 2453 | CAGGGACU G UAUAAACA | 1145 | UGUUUAUA GGAGGAAACUCC CU UCAAGGACAUCGUCCGGG AGUCCCUG | 3445 |
| 14 | CGUCCGCA G CCCGCCCG | 1146 | CGGGCGGG GGAGGAAACUCC CU UCAAGGACAUCGUCCGGG UGCGGACG | 3446 |
| 26 | GCCCGGGA G CUGCGAGC | 1147 | GCUCGCAG GGAGGAAACUCC CU UCAAGGACAUCGUCCGGG UCCCGGGC | 3447 |
| 33 | AGCUGCGA G CCGCGAGC | 1148 | GCUCGCGG GGAGGAAACUCC CU UCAAGGACAUCGUCCGGG UCGCAGCU | 3448 |
| 40 | AGCCGCGA G CUGGAUUA | 1149 | UAAUCCAG GGAGGAAACUCC CU UCAAGGACAUCGUCCGGG UCGCGGCU | 3449 |
| 51 | GGAUUAUG G UGGCCUGA | 1150 | UCAGGCCA GGAGGAAACUCC CU UCAAGGACAUCGUCCGGG CAUAAUCC | 3450 |
| 54 | UUAUGGUG G CCUGAGCA | 1151 | UGCUCAGG GGAGGAAACUCC CU UCAAGGACAUCGUCCGGG CACCAUAA | 3451 |
| 60 | UGGCCUGA G CAGCCAAC | 1152 | GUUGGCUG GGAGGAAACUCC CU UCAAGGACAUCGUCCGGG UCAGGCCA | 3452 |
| 63 | CCUGAGCA G CCAACGCA | 1153 | UGCGUUGG GGAGGAAACUCC CU UCAAGGACAUCGUCCGGG UGCUCAGG | 3453 |
| 72 | CCAACGCA G CCGCAGGA | 1154 | UCCUGCGG GGAGGAAACUCC CU UCAAGGACAUCGUCCGGG UGCGUUGG | 3454 |
| 81 | CCGCAGGA G CCCGGAGC | 1155 | GCUCCGGG GGAGGAAACUCC CU UCAAGGACAUCGUCCGGG UCCUGCGG | 3455 |
| 88 | AGCCCGGA G CCCUUGCC | 1156 | GGCAAGGG GGAGGAAACUCC CU UCAAGGACAUCGUCCGGG UCCGGGCU | 3456 |
| 134 | CCAGGGAA G CCGCCACC | 1157 | GGUGGCGG GGAGGAAACUCC CU UCAAGGACAUCGUCCGGG UUCCCUGG | 3457 |
| 144 | CGCCACCG G CCCGCCAU | 1158 | AUGGCGGG GGAGGAAACUCC CU UCAAGGACAUCGUCCGGG CGGUGGCG | 3458 |
| 167 | CCCUCCCA G CCCCGCCG | 1159 | CGGCGGGG GGAGGAAACUCC CU UCAAGGACAUCGUCCGGG UGGGAGGG | 3459 |
| 179 | CGCCGGGA G CCCGCGCC | 1160 | GGCGCGGG GGAGGAAACUCC CU UCAAGGACAUCGUCCGGG UCCCGGCG | 3460 |
| 198 | CUGCCCAG G CUGGCCGC | 1161 | GCGGCCAG GGAGGAAACUCC CU UCAAGGACAUCGUCCGGG CUGGGCAG | 3461 |
| 202 | CCAGGCUG G CCGCCGCC | 1162 | GGCGGCGG GGAGGAAACUCC CU UCAAGGACAUCGUCCGGG CAGCCUGG | 3462 |
| 211 | CCGCCGCC G UGCCGAUG | 1163 | CAUCGGCA GGAGGAAACUCC CU UCAAGGACAUCGUCCGGG GGCGGCGG | 3463 |
| 222 | CCGAUGUA G CGGGCUCC | 1164 | GGAGCCCG GGAGGAAACUCC CU UCAAGGACAUCGUCCGGG UACAUCGG | 3464 |
| 226 | UGUAGCGG G CUCCGGAU | 1165 | AUCCGGAG GGAGGAAACUCC CU UCAAGGACAUCGUCCGGG CCGCUACA | 3465 |
| 239 | GGAUCCCA G CCUCUCCC | 1166 | GGGAGAGG GGAGGAAACUCC CU UCAAGGACAUCGUCCGGG UGGGAUCC | 3466 |
| 256 | CUGCUCCC G UGCUCUGC | 1167 | GCAGAGCA GGAGGAAACUCC CU UCAAGGACAUCGUCCGGG GGGAGCAG | 3467 |
| 290 | UCUCCACA G CCCGGACC | 1168 | GGUCCGGG GGAGGAAACUCC CU UCAAGGACAUCGUCCGGG UGUGGAGA | 3468 |
| 304 | ACCCGGGG G CUGGCCCA | 1169 | UGGGCCAG GGAGGAAACUCC CU UCAAGGACAUCGUCCGGG CCCCGGGU | 3469 |
| 308 | GGGGGCUG G CCCAGGGC | 1170 | GCCCUGGG GGAGGAAACUCC CU UCAAGGACAUCGUCCGGG CAGCCCCC | 3470 |
| 315 | GGCCCAGG G CCCUGCAG | 1171 | CUGCAGGG GGAGGAAACUCC CU UCAAGGACAUCGUCCGGG CCUGGGCC | 3471 |
| 324 | CCCUGCAG G CCCUGGCG | 1172 | CGCCAGGG GGAGGAAACUCC CU UCAAGGACAUCGUCCGGG CUGCAGGG | 3472 |
| 330 | AGGCCCUG G CGUCCUGA | 1173 | UCAGGACG GGAGGAAACUCC CU UCAAGGACAUCGUCCGGG CAGGGCCU | 3473 |
| 332 | GCCCUGGC G UCCUGAUG | 1174 | CAUCAGGA GGAGGAAACUCC CU UCAAGGACAUCGUCCGGG GCCAGGGC | 3474 |
| 348 | GCCCCCAA G CUCCCUCU | 1175 | AGAGGGAG GGAGGAAACUCC CU UCAAGGACAUCGUCCGGG UUGGGGGC | 3475 |
| 365 | CCUGAGAA G CCACCAGC | 1176 | GCUGGUGG GGAGGAAACUCC CU UCAAGGACAUCGUCCGGG UUCUCAGG | 3476 |
| 372 | AGCCACCA G CACCACCC | 1177 | GGGUGGUG GGAGGAAACUCC CU UCAAGGACAUCGUCCGGG UGGUGGCU | 3477 |
| 391 | ACUUGGGG G CAGGCGCC | 1178 | GGCGCCUG GGAGGAAACUCC CU UCAAGGACAUCGUCCGGG CCCCAAGU | 3478 |
| 395 | GGGGGCAG G CGCCAGGG | 1179 | CCCUGGCG GGAGGAAACUCC CU UCAAGGACAUCGUCCGGG CUGCCCCC | 3479 |
| 410 | GGACGGAC G UGGGCCAG | 1180 | CUGGCCCA GGAGGAAACUCC CU UCAAGGACAUCGUCCGGG GUCCGUCC | 3480 |
| 414 | GGACGUGG G CCAGUGCG | 1181 | CGCACUGG GGAGGAAACUCC CU UCAAGGACAUCGUCCGGG CCACGUCC | 3481 |
| 418 | GUGGGCCA G UGCGAGCC | 1182 | GGCUCGCA GGAGGAAACUCC CU UCAAGGACAUCGUCCGGG UGGCCCAC | 3482 |
| 424 | CAGUGCGA G CCCAGAGG | 1183 | CCUCUGGG GGAGGAAACUCC CU UCAAGGACAUCGUCCGGG UCGCACUG | 3483 |
| 433 | CCCAGAGG G CCCGAAGG | 1184 | CCUUCGGG GGAGGAAACUCC CU UCAAGGACAUCGUCCGGG CCUCUGGG | 3484 |
| 441 | GCCCGAAG G CCGGGGCC | 1185 | GGCCCCGG GGAGGAAACUCC CU UCAAGGACAUCGUCCGGG CUUCGGGC | 3485 |
| 447 | AGGCCGGG G CCCACCAU | 1186 | AUGGUGGG GGAGGAAACUCC CU UCAAGGACAUCGUCCGGG CCCGGCCU | 3486 |
| 457 | CCACCAUG G CCCAAGCC | 1187 | GGCUUGGG GGAGGAAACUCC CU UCAAGGACAUCGUCCGGG CAUGGUGG | 3487 |
| 463 | UGGCCCAA G CCCUGCCC | 1188 | GGGCAGGG GGAGGAAACUCC CU UCAAGGACAUCGUCCGGG UUGGGCCA | 3488 |
| 474 | CUGCCCUG G CUCCUGCU | 1189 | AGCAGGAG GGAGGAAACUCC CU UCAAGGACAUCGUCCGGG CAGGGCAG | 3489 |
| 491 | GUGGAUGG G CGCGGGAG | 1190 | CUCCCGCG GGAGGAAACUCC CU UCAAGGACAUCGUCCGGG CCAUCCAC | 3490 |
| 499 | GCGCGGGA G UGCUGCCU | 1191 | AGGCAGCA GGAGGAAACUCC CU UCAAGGACAUCGUCCGGG UCCCGCGC | 3491 |
| 515 | UGCCCACG G CACCCAGC | 1192 | GCUGGGUG GGAGGAAACUCC CU UCAAGGACAUCGUCCGGG CGUGGGCA | 3492 |
| 522 | GGCACCCA G CACGGCAU | 1193 | AUGCCGUG GGAGGAAACUCC CU UCAAGGACAUCGUCCGGG UGGGUGCC | 3493 |
| 527 | CCAGCACG G CAUCCGGC | 1194 | GCCGGAUG GGAGGAAACUCC CU UCAAGGACAUCGUCCGGG CGUGCUGG | 3494 |
| 534 | GGCAUCCG G CUGCCCCU | 1195 | AGGGGCAG GGAGGAAACUCC CU UCAAGGACAUCGUCCGGG CGGAUGCC | 3495 |
| 548 | CCUGCGCA G CGGCCUGG | 1196 | CCAGGCCG GGAGGAAACUCC CU UCAAGGACAUCGUCCGGG UGCGCAGG | 3496 |
| 551 | GCGCAGCG G CCUGGGGG | 1197 | CCCCCAGG GGAGGAAACUCC CU UCAAGGACAUCGUCCGGG CGCUGCGC | 3497 |
| 560 | CCUGGGGG G CGCCCCCC | 1198 | GGGGGGCG GGAGGAAACUCC CU UCAAGGACAUCGUCCGGG CCCCCAGG | 3498 |
| 573 | CCCCUGGG G CUGCGGCU | 1199 | AGCCGCAG GGAGGAAACUCC CU UCAAGGACAUCGUCCGGG CCCAGGGG | 3499 |
| 579 | GGGCUGCG G CUGCCCCG | 1200 | CGGGGCAG GGAGGAAACUCC CU UCAAGGACAUCGUCCGGG CGCAGCCC | 3500 |
| 603 | GACGAAGA G CCCGAGGA | 1201 | UCCUCGGG GGAGGAAACUCC CU UCAAGGACAUCGUCCGGG UCUUCGUC | 3501 |
| 612 | CCCGAGGA G CCCGGCCG | 1202 | CGGCCGGG GGAGGAAACUCC CU UCAAGGACAUCGUCCGGG UCCUCGGG | 3502 |
| 617 | GGAGCCCG G CCGGAGGG | 1203 | CCCUCCGG GGAGGAAACUCC CU UCAAGGACAUCGUCCGGG CGGGCUCC | 3503 |
| 626 | CCGGAGGG G CAGCUUUG | 1204 | CAAAGCUG GGAGGAAACUCC CU UCAAGGACAUCGUCCGGG CCCUCCGG | 3504 |
| 629 | GAGGGGCA G CUUUGUGG | 1205 | CCACAAAG GGAGGAAACUCC CU UCAAGGACAUCGUCCGGG UGCCCCUC | 3505 |
| 643 | UGGAGAUG G UGGACAAC | 1206 | GUUGUCCA GGAGGAAACUCC CU UCAAGGACAUCGUCCGGG CAUCUCCA | 3506 |
| 659 | CCUGAGGG G CAAGUCGG | 1207 | CCGACUUG GGAGGAAACUCC CU UCAAGGACAUCGUCCGGG CCCUCAGG | 3507 |
| 663 | AGGGGCAA G UCGGGGCA | 1208 | UGCCCCGA GGAGGAAACUCC CU UCAAGGACAUCGUCCGGG UUGCCCCU | 3508 |
| 669 | AAGUCGGG G CAGGGCUA | 1209 | UAGCCCUG GGAGGAAACUCC CU UCAAGGACAUCGUCCGGG CCCGACUU | 3509 |
| 674 | GGGGCAGG G CUACUACG | 1210 | CGUAGUAG GGAGGAAACUCC CU UCAAGGACAUCGUCCGGG CCUGCCCC | 3510 |
| 682 | GCUACUAC G UGGAGAUG | 1211 | CAUCUCCA GGAGGAAACUCC CU UCAAGGACAUCGUCCGGG GUAGUAGC | 3511 |
| 694 | AGAUGACC G UGGGCAGC | 1212 | GCUGCCCA GGAGGAAACUCC CU UCAAGGACAUCGUCCGGG GGUCAUCU | 3512 |
| 698 | GACCGUGG G CAGCCCCC | 1213 | GGGGGCUG GGAGGAAACUCC CU UCAAGGACAUCGUCCGGG CCACGGUC | 3513 |
| 701 | CGUGGGCA G CCCCCCGC | 1214 | GCGGGGGG GGAGGAAACUCC CU UCAAGGACAUCGUCCGGG UGCCCACG | 3514 |
| 727 | ACAUCCUG G UGGAUACA | 1215 | UGUAUCCA GGAGGAAACUCC CU UCAAGGACAUCGUCCGGG CAGGAUGU | 3515 |
| 737 | GGAUACAG G CAGCAGUA | 1216 | UACUGCUG GGAGGAAACUCC CU UCAAGGACAUCGUCCGGG CUGUAUCC | 3516 |
| 740 | UACAGGCA G CAGUAACU | 1217 | AGUUACUG GGAGGAAACUCC CU UCAAGGACAUCGUCCGGG UGCCUGUA | 3517 |
| 743 | AGGCAGCA G UAACUUUG | 1218 | CAAAGUUA GGAGGAAACUCC CU UCAAGGACAUCGUCCGGG UGCUGCCU | 3518 |
| 754 | ACUUUGCA G UGGGUGCU | 1219 | AGCACCCA GGAGGAAACUCC CU UCAAGGACAUCGUCCGGG UGCAAAGU | 3519 |
| 758 | UGCAGUGG G UGCUGCCC | 1220 | GGGCAGCA GGAGGAAACUCC CU UCAAGGACAUCGUCCGGG CCACUGCA | 3520 |
| 798 | UACCAGAG G CAGCUGUC | 1221 | GACAGCUG GGAGGAAACUCC CU UCAAGGACAUCGUCCGGG CUCUGGUA | 3521 |
| 801 | CAGAGGCA G CUGUCCAG | 1222 | CUGGACAG GGAGGAAACUCC CU UCAAGGACAUCGUCCGGG UGCCUCUG | 3522 |
| 809 | GCUGUCCA G CACAUACC | 1223 | GGUAUGUG GGAGGAAACUCC CU UCAAGGACAUCGUCCGGG UGGACAGC | 3523 |
| 833 | CCGGAAGG G UGUGUAUG | 1224 | CAUACACA GGAGGAAACUCC CU UCAAGGACAUCGUCCGGG CCUUCCGG | 3524 |
| 857 | CACCCAGG G CAAGUGGG | 1225 | CCCACUUG GGAGGAAACUCC CU UCAAGGACAUCGUCCGGG CCUGGGUG | 3525 |
| 861 | CAGGGCAA G UGGGAAGG | 1226 | CCUUCCCA GGAGGAAACUCC CU UCAAGGACAUCGUCCGGG UUGCCCUG | 3526 |
| 873 | GAAGGGGA G CUGGGCAC | 1227 | GUGCCCAG GGAGGAAACUCC CU UCAAGGACAUCGUCCGGG UCCCCUUC | 3527 |
| 878 | GGAGCUGG G CACCGACC | 1228 | GGUCGGUG GGAGGAAACUCC CU UCAAGGACAUCGUCCGGG CCAGCUCC | 3528 |
| 889 | CCGACCUG G UAAGCAUC | 1229 | GAUGCUUA GGAGGAAACUCC CU UCAAGGACAUCGUCCGGG CAGGUCGG | 3529 |
| 893 | CCUGGUAA G CAUCCCCC | 1230 | GGGGGAUG GGAGGAAACUCC CU UCAAGGACAUCGUCCGGG UUACCAGG | 3530 |
| 905 | CCCCCAUG G CCCCAACG | 1231 | CGUUGGGG GGAGGAAACUCC CU UCAAGGACAUCGUCCGGG CAUGGGGG | 3531 |
| 913 | GCCCCAAC G UCACUGUG | 1232 | CACAGUGA GGAGGAAACUCC CU UCAAGGACAUCGUCCGGG GUUGGGGC | 3532 |
| 923 | CACUGUGC G UGCCAACA | 1233 | UGUUGGCA GGAGGAAACUCC CU UCAAGGACAUCGUCCGGG GCACAGUG | 3533 |
| 957 | UCAGACAA G UUCUUCAU | 1234 | AUGAAGAA GGAGGAAACUCC CU UCAAGGACAUCGUCCGGG UUGUCUGA | 3534 |
| 971 | CAUCAACG G CUCCAACU | 1235 | AGUUGGAG GGAGGAAACUCC CU UCAAGGACAUCGUCCGGG CGUUGAUG | 3535 |
| 986 | CUGGGAAG G CAUCCUGG | 1236 | CCAGGAUG GGAGGAAACUCC CU UCAAGGACAUCGUCCGGG CUUCCCAG | 3536 |
| 996 | AUCCUGGG G CUGGCCUA | 1237 | UAGGCCAG GGAGGAAACUCC CU UCAAGGACAUCGUCCGGG CCCAGGAU | 3537 |
| 1000 | UGGGGCUG G CCUAUGCU | 1238 | AGCAUAGG GGAGGAAACUCC CU UCAAGGACAUCGUCCGGG CAGCCCCA | 3538 |
| 1020 | AUUGCCAG G CCUGACGA | 1239 | UCGUCAGG GGAGGAAACUCC CU UCAAGGACAUCGUCCGGG CUGGCAAU | 3539 |
| 1038 | UCCCUGGA G CCUUUCUU | 1240 | AAGAAAGG GGAGGAAACUCC CU UCAAGGACAUCGUCCGGG UCCAGGGA | 3540 |
| 1057 | ACUCUCUG G UAAAGCAG | 1241 | CUGCUUUA GGAGGAAACUCC CU UCAAGGACAUCGUCCGGG CAGAGAGU | 3541 |
| 1062 | CUGGUAAA G CAGACCCA | 1242 | UGGGUCUG GGAGGAAACUCC CU UCAAGGACAUCGUCCGGG UUUACCAG | 3542 |
| 1072 | AGACCCAC G UUCCCAAC | 1243 | GUUGGGAA GGAGGAAACUCC CU UCAAGGACAUCGUCCGGG GUGGGUCU | 3543 |
| 1095 | UCCCUGCA G CUUUGUGG | 1244 | CCACAAAG GGAGGAAACUCC CU UCAAGGACAUCGUCCGGG UGCAGGGA | 3544 |
| 1103 | GCUUUGUG G UGCUGGCU | 1245 | AGCCAGCA GGAGGAAACUCC CU UCAAGGACAUCGUCCGGG CACAAAGC | 3545 |
| 1109 | UGGUGCUG G CUUCCCCC | 1246 | GGGGGAAG GGAGGAAACUCC CU UCAAGGACAUCGUCCGGG CAGCACCA | 3546 |
| 1125 | CUCAACCA G UCUGAAGU | 1247 | ACUUCAGA GGAGGAAACUCC CU UCAAGGACAUCGUCCGGG UGGUUGAG | 3547 |
| 1132 | AGUCUGAA G UGCUGGCC | 1248 | GGCCAGCA GGAGGAAACUCC CU UCAAGGACAUCGUCCGGG UUCAGACU | 3548 |
| 1138 | AAGUGCUG G CCUCUGUC | 1249 | GACAGAGG GGAGGAAACUCC CU UCAAGGACAUCGUCCGGG CAGCACUU | 3549 |
| 1154 | CGGAGGGA G CAUGAUCA | 1250 | UGAUCAUG GGAGGAAACUCC CU UCAAGGACAUCGUCCGGG UCCCUCCG | 3550 |
| 1169 | CAUUGGAG G UAUCGACC | 1251 | GGUCGAUA GGAGGAAACUCC CU UCAAGGACAUCGUCCGGG CUCCAAUG | 3551 |
| 1193 | GUACACAG G CAGUCUCU | 1252 | AGAGACUG GGAGGAAACUCC CU UCAAGGACAUCGUCCGGG CUGUGUAC | 3552 |
| 1196 | CACAGGCA G UCUCUGGU | 1253 | ACCAGAGA GGAGGAAACUCC CU UCAAGGACAUCGUCCGGG UGCCUGUG | 3553 |
| 1203 | AGUCUCUG G UAUACACC | 1254 | GGUGUAUA GGAGGAAACUCC CU UCAAGGACAUCGUCCGGG CAGAGACU | 3554 |
| 1218 | CCCAUCCG G CGGGAGUG | 1255 | CACUCCCG GGAGGAAACUCC CU UCAAGGACAUCGUCCGGG CGGAUGGG | 3555 |
| 1224 | CGGCGGGA G UGGUAUUA | 1256 | UAAUACCA GGAGGAAACUCC CU UCAAGGACAUCGUCCGGG UCCCGCCG | 3556 |
| 1227 | CGGGAGUG G UAUUAUGA | 1257 | UCAUAAUA GGAGGAAACUCC CU UCAAGGACAUCGUCCGGG CACUCCCG | 3557 |
| 1237 | AUUAUGAG G UGAUCAUU | 1258 | AAUGAUCA GGAGGAAACUCC CU UCAAGGACAUCGUCCGGG CUCAUAAU | 3558 |
| 1252 | UUGUGCGG G UGGAGAUC | 1259 | GAUCUCCA GGAGGAAACUCC CU UCAAGGACAUCGUCCGGG CCGCACAA | 3559 |
| 1293 | UGCAAGGA G UACAACUA | 1260 | UAGUUGUA GGAGGAAACUCC CU UCAAGGACAUCGUCCGGG UCCUUGCA | 3560 |
| 1310 | UGACAAGA G CAUUGUGG | 1261 | CCACAAUG GGAGGAAACUCC CU UCAAGGACAUCGUCCGGG UCUUGUCA | 3561 |
| 1322 | UGUGGACA G UGGCACCA | 1262 | UGGUGCCA GGAGGAAACUCC CU UCAAGGACAUCGUCCGGG UGUCCACA | 3562 |
| 1325 | GGACAGUG G CACCACCA | 1263 | UGGUGGUG GGAGGAAACUCC CU UCAAGGACAUCGUCCGGG CACUGUCC | 3563 |
| 1340 | CAACCUUC G UUUGCCCA | 1264 | UGGGCAAA GGAGGAAACUCC CU UCAAGGACAUCGUCCGGG GAAGGUUG | 3564 |
| 1354 | CCAAGAAA G UGUUUGAA | 1265 | UUCAAACA GGAGGAAACUCC CU UCAAGGACAUCGUCCGGG UUUCUUGG | 3565 |
| 1363 | UGUUUGAA G CUGCAGUC | 1266 | GACUGCAG GGAGGAAACUCC CU UCAAGGACAUCGUCCGGG UUCAAACA | 3566 |
| 1369 | AAGCUGCA G UCAAAUCC | 1267 | GGAUUUGA GGAGGAAACUCC CU UCAAGGACAUCGUCCGGG UGCAGCUU | 3567 |
| 1384 | CCAUCAAG G CAGCCUCC | 1268 | GGAGGCUG GGAGGAAACUCC CU UCAAGGACAUCGUCCGGG CUUGAUGG | 3568 |
| 1387 | UCAAGGCA G CCUCCUCC | 1269 | GGAGGAGG GGAGGAAACUCC CU UCAAGGACAUCGUCCGGG UGCCUUGA | 3569 |
| 1404 | ACGGAGAA G UUCCCUGA | 1270 | UCAGGGAA GGAGGAAACUCC CU UCAAGGACAUCGUCCGGG UUCUCCGU | 3570 |
| 1415 | CCCUGAUG G UUUCUGGC | 1271 | GCCAGAAA GGAGGAAACUCC CU UCAAGGACAUCGUCCGGG CAUCAGGG | 3571 |
| 1422 | GGUUUCUG G CUAGGAGA | 1272 | UCUCCUAG GGAGGAAACUCC CU UCAAGGACAUCGUCCGGG CAGAAACC | 3572 |
| 1431 | CUAGGAGA G CAGCUGGU | 1273 | ACCAGCUG GGAGGAAACUCC CU UCAAGGACAUCGUCCGGG UCUCCUAG | 3573 |
| 1434 | GGAGAGCA G CUGGUGUG | 1274 | CACACCAG GGAGGAAACUCC CU UCAAGGACAUCGUCCGGG UGCUCUCC | 3574 |
| 1438 | AGCAGCUG G UGUGCUGG | 1275 | CCAGCACA GGAGGAAACUCC CU UCAAGGACAUCGUCCGGG CAGCUGCU | 3575 |
| 1446 | GUGUGCUG G CAAGCAGG | 1276 | CCUGCUUG GGAGGAAACUCC CU UCAAGGACAUCGUCCGGG CAGCACAC | 3576 |
| 1450 | GCUGGCAA G CAGGCACC | 1277 | GGUGCCUG GGAGGAAACUCC CU UCAAGGACAUCGUCCGGG UUGCCAGC | 3577 |
| 1454 | GCAAGCAG G CACCACCC | 1278 | GGGUGGUG GGAGGAAACUCC CU UCAAGGACAUCGUCCGGG CUGCUUGC | 3578 |
| 1480 | UUUUCCCA G UCAUCUCA | 1279 | UGAGAUGA GGAGGAAACUCC CU UCAAGGACAUCGUCCGGG UGGGAAAA | 3579 |
| 1502 | CCUAAUGG G UGAGGUUA | 1280 | UAACCUCA GGAGGAAACUCC CU UCAAGGACAUCGUCCGGG CCAUUAGG | 3580 |
| 1507 | UGGGUGAG G UUACCAAC | 1281 | GUUGGUAA GGAGGAAACUCC CU UCAAGGACAUCGUCCGGG CUCACCCA | 3581 |
| 1518 | ACCAACCA G UCCUUCCG | 1282 | CGGAAGGA GGAGGAAACUCC CU UCAAGGACAUCGUCCGGG UGGUUGGU | 3582 |
| 1545 | CUUCCGCA G CAAUACCU | 1283 | AGGUAUGG GGAGGAAACUCC CU UCAAGGACAUCGUCCGGG UGCGGAAG | 3583 |
| 1557 | UACCUGCG G CCAGUGGA | 1284 | UCCACUGG GGAGGAAACUCC CU UCAAGGACAUCGUCCGGG CGCAGGUA | 3584 |
| 1561 | UGCGGCCA G UGGAAGAU | 1285 | AUCUUCCA GGAGGAAACUCC CU UCAAGGACAUCGUCCGGG UGGCCGCA | 3585 |
| 1573 | AAGAUGUG G CCACGUCC | 1286 | GGACGUGG GGAGGAAACUCC CU UCAAGGACAUCGUCCGGG CACAUCUU | 3586 |
| 1578 | GUGGCCAC G UCCCAAGA | 1287 | UCUUGGGA GGAGGAAACUCC CU UCAAGGACAUCGUCCGGG GUGGCCAC | 3587 |
| 1599 | UGUUACAA G UUUGCCAU | 1288 | AUGGCAAA GGAGGAAACUCC CU UCAAGGACAUCGUCCGGG UGGUAACA | 3588 |
| 1614 | AUCUCACA G UCAUCCAC | 1289 | GUGGAUGA GGAGGAAACUCC CU UCAAGGACAUCGUCCGGG UGUGAGAU | 3589 |
| 1625 | AUCCACGG G CACUGUUA | 1290 | UAACAGUG GGAGGAAACUCC CU UCAAGGACAUCGUCCGGG CCGUGGAU | 3590 |
| 1639 | UUAUGGGA G CUGUUAUC | 1291 | GAUAACAG GGAGGAAACUCC CU UCAAGGACAUCGUCCGGG UCCCAUAA | 3591 |
| 1655 | CAUGGAGG G CUUCUACG | 1292 | CGUAGAAG GGAGGAAACUCC CU UCAAGGACAUCGUCCGGG CCUCCAUG | 3592 |
| 1663 | GCUUCUAC G UUGUCUUU | 1293 | AAAGACAA GGAGGAAACUCC CU UCAAGGACAUCGUCCGGG GUAGAAGC | 3593 |
| 1678 | UUGAUCGG G CCCGAAAA | 1294 | UUUUCGGG GGAGGAAACUCC CU UCAAGGACAUCGUCCGGG CCGAUCAA | 3594 |
| 1694 | ACGAAUUG G CUUUGCUG | 1295 | CAGCAAAG GGAGGAAACUCC CU UCAAGGACAUCGUCCGGG CAAUUCGU | 3595 |
| 1706 | UGCUGUCA G CGCUUGCC | 1296 | GGCAAGCG GGAGGAAACUCC CU UCAAGGACAUCGUCCGGG UGACAGCA | 3596 |
| 1728 | CACGAUGA G UUCAGGAC | 1297 | GUCCUGAA GGAGGAAACUCC CU UCAAGGACAUCGUCCGGG UCAUCGUG | 3597 |
| 1738 | UCAGGACG G CAGCGGUG | 1298 | CACCGCUG GGAGGAAACUCC CU UCAAGGACAUCGUCCGGG CGUCCUGA | 3598 |
| 1741 | GGACGGCA G CGGUGGAA | 1299 | UUCCACCG GGAGGAAACUCC CU UCAAGGACAUCGUCCGGG UGCCGUCC | 3599 |
| 1744 | CGGCAGCG G UGGAAGGC | 1300 | GCCUUCCA GGAGGAAACUCC CU UCAAGGACAUCGUCCGGG CGCUGCCG | 3600 |
| 1751 | GGUGGAAG G CCCUUUUG | 1301 | CAAAAGGG GGAGGAAACUCC CU UCAAGGACAUCGUCCGGG CUUCCACC | 3601 |
| 1784 | AGACUGUG G CUACAACA | 1302 | UGUUGUAG GGAGGAAACUCC CU UCAAGGACAUCGUCCGGG CACAGUCU | 3602 |
| 1809 | ACAGAUGA G UCAACCCU | 1303 | AGGGUUGA GGAGGAAACUCC CU UCAAGGACAUCGUCCGGG UCAUCUGU | 3603 |
| 1828 | UGACCAUA G CCUAUGUC | 1304 | GACAUAGG GGAGGAAACUCC CU UCAAGGACAUCGUCCGGG UAUGGUCA | 3604 |
| 1840 | AUGUCAUG G CUGCCAUC | 1305 | GAUGGCAG GGAGGAAACUCC CU UCAAGGACAUCGUCCGGG CAUGACAU | 3605 |
| 1882 | GCCUCAUG G UGUGUCAG | 1306 | CUGACACA GGAGGAAACUCC CU UCAAGGACAUCGUCCGGG CAUGAGGC | 3606 |
| 1890 | GUGUGUCA G UGGCGCUG | 1307 | CAGCGCCA GGAGGAAACUCC CU UCAAGGACAUCGUCCGGG UGACACAC | 3607 |
| 1893 | UGUCAGUG G CGCUGCCU | 1308 | AGGCAGCG GGAGGAAACUCC CU UCAAGGACAUCGUCCGGG CACUGACA | 3608 |
| 1917 | CUGCGCCA G CAGCAUGA | 1309 | UCAUGCUG GGAGGAAACUCC CU UCAAGGACAUCGUCCGGG UGGCGCAG | 3609 |
| 1920 | CGCCAGCA G CAUGAUGA | 1310 | UCAUCAUG GGAGGAAACUCC CU UCAAGGACAUCGUCCGGG UGCUGGCG | 3610 |
| 1956 | CUGCUGAA G UGAGGAGG | 1311 | CCUCCUCA GGAGGAAACUCC CU UCAAGGACAUCGUCCGGG UUCAGCAG | 3611 |
| 1964 | GUGAGGAG G CCCAUGGG | 1312 | CCCAUGGG GGAGGAAACUCC CU UCAAGGACAUCGUCCGGG CUCCUCAC | 3612 |
| 1972 | GCCCAUGG G CAGAAGAU | 1313 | AUCUUCUG GGAGGAAACUCC CU UCAAGGACAUCGUCCGGG CCAUGGGC | 3613 |
| 2006 | ACACCUCC G UGGUUCAC | 1314 | GUGAACCA GGAGGAAACUCC CU UCAAGGACAUCGUCCGGG GGAGGUGU | 3614 |
| 2009 | CCUCCGUG G UUCACUUU | 1315 | AAAGUGAA GGAGGAAACUCC CU UCAAGGACAUCGUCCGGG CACGGAGG | 3615 |
| 2019 | UCACUUUG G UCACAAGU | 1316 | ACUUGUGA GGAGGAAACUCC CU UCAAGGACAUCGUCCGGG CAAAGUGA | 3616 |
| 2026 | GGUCACAA G UAGGAGAC | 1317 | GUCUCCUA GGAGGAAACUCC CU UCAAGGACAUCGUCCGGG UUGUGACC | 3617 |
| 2042 | CACAGAUG G CACCUGUG | 1318 | CACAGGUG GGAGGAAACUCC CU UCAAGGACAUCGUCCGGG CAUCUGUG | 3618 |
| 2051 | CACCUGUG G CCAGAGCA | 1319 | UGCUCUGG GGAGGAAACUCC CU UCAAGGACAUCGUCCGGG CACAGGUG | 3619 |
| 2057 | UGGCCAGA G CACCUCAG | 1320 | CUGAGGUG GGAGGAAACUCC CU UCAAGGACAUCGUCCGGG UCUGGCCA | 3620 |
| 2114 | AGGAAAAG G CUGGCAAG | 1321 | CUUGCCAG GGAGGAAACUCC CU UCAAGGACAUCGUCCGGG CUUUUCCU | 3621 |
| 2118 | AAAGGCUG G CAAGGUGG | 1322 | CCACCUUG GGAGGAAACUCC CU UCAAGGACAUCGUCCGGG CAGCCUUU | 3622 |
| 2123 | CUGGCAAG G UGGGUUCC | 1323 | GGAACCCA GGAGGAAACUCC CU UCAAGGACAUCGUCCGGG CUUGCCAG | 3623 |
| 2127 | CAAGGUGG G UUCCAGGG | 1324 | CCCUGGAA GGAGGAAACUCC CU UCAAGGACAUCGUCCGGG CCACCUUG | 3624 |
| 2172 | AGAAAGAA G CACUCUGC | 1325 | GCAGAGUG GGAGGAAACUCC CU UCAAGGACAUCGUCCGGG UUCUUUCU | 3625 |
| 2183 | CUCUGCUG G CGGGAAUA | 1326 | UAUUCCCG GGAGGAAACUCC CU UCAAGGACAUCGUCCGGG CAGCAGAG | 3626 |
| 2198 | UACUCUUG G UCACCUCA | 1327 | UGAGGUGA GGAGGAAACUCC CU UCAAGGACAUCGUCCGGG CAAGAGUA | 3627 |
| 2214 | AAAUUUAA G UCGGGAAA | 1328 | UUUCCCGA GGAGGAAACUCC CU UCAAGGACAUCGUCCGGG UUAAAUUU | 3628 |
| 2243 | AAACUUCA G CCCUGAAC | 1329 | GUUCAGGG GGAGGAAACUCC CU UCAAGGACAUCGUCCGGG UGAAGUUU | 3629 |
| 2288 | AACCCAAA G UAUUCUUC | 1330 | GAAGAAUA GGAGGAAACUCC CU UCAAGGACAUCGUCCGGG UUUGGGUU | 3630 |
| 2305 | UUUUCUUA G UUUCAGAA | 1331 | UUCUGAAA GGAGGAAACUCC CU UCAAGGACAUCGUCCGGG UAAGAAAA | 3631 |
| 2314 | UUUCAGAA G UACUGGCA | 1332 | UGCCAGUA GGAGGAAACUCC CU UCAAGGACAUCGUCCGGG UUCUGAAA | 3632 |
| 2320 | AAGUACUG G CAUCACAC | 1333 | GUGUGAUG GGAGGAAACUCC CU UCAAGGACAUCGUCCGGG CAGUACUU | 3633 |
| 2333 | ACACGCAG G UUACCUUG | 1334 | CAAGGUAA GGAGGAAACUCC CU UCAAGGACAUCGUCCGGG CUGCGUGU | 3634 |
| 2343 | UUACCUUG G CGUGUGUC | 1335 | GACACACG GGAGGAAACUCC CU UCAAGGACAUCGUCCGGG CAAGGUAA | 3635 |
| 2344 | ACCUUGGC G UGUGUCCC | 1336 | GGGACACA GGAGGAAACUCC CU UCAAGGACAUCGUCCGGG GCCAAGGU | 3636 |
| 2357 | UCCCUGUG G UACCCUGG | 1337 | CCAGGGUA GGAGGAAACUCC CU UCAAGGACAUCGUCCGGG CACAGGGA | 3637 |
| 2365 | GUACCCUG G CAGAGAAG | 1338 | CUUCUCUG GGAGGAAACUCC CU UCAAGGACAUCGUCCGGG CAGGGUAC | 3638 |
| 2381 | GAGACCAA G CUUGUUUC | 1339 | GAAACAAG GGAGGAAACUCC CU UCAAGGACAUCGUCCGGG UUGGUCUC | 3639 |
| 2397 | CCCUGCUG G CCAAAGUC | 1340 | GACUUUGG GGAGGAAACUCC CU UCAAGGACAUCGUCCGGG CAGCAGGG | 3640 |
| 2403 | UGGCCAAA G UCAGUAGG | 1341 | CCUACUGA GGAGGAAACUCC CU UCAAGGACAUCGUCCGGG UUUGGCCA | 3641 |
| 2407 | CAAAGUCA G UAGGAGAG | 1342 | CUCUCCUA GGAGGAAACUCC CU UCAAGGACAUCGUCCGGG UGACUUUG | 3642 |
| 2424 | GAUGCACA G UUUGCUAU | 1343 | AUAGCAAA GGAGGAAACUCC CU UCAAGGACAUCGUCCGGG UGUGCAUC | 3643 |
| 2463 | AUAAACAA G CCUAACAU | 1344 | AUGUUAGG GGAGGAAACUCC CU UCAAGGACAUCGUCCGGG UUGUUUAU | 3644 |
| 2474 | UAACAUUG G UGCAAAGA | 1345 | UCUUUGCA GGAGGAAACUCC CU UCAAGGACAUCGUCCGGG CAAUGUUA | 3645 |
| 22 | GCCCGCCC G GGAGCUGC | 1449 | GCAGCUCC GGAGGAAACUCC CU UCAAGGACAUCGUCCGGG GGGCGGGC | 3646 |
| 23 | CCCGCCCG G GAGCUGCG | 1450 | CGCAGCUC GGAGGAAACUCC CU UCAAGGACAUCGUCCGGG CGGGCGGG | 3647 |
| 24 | CCGCCCGG G AGCUGCGA | 1451 | UCGCAGCU GGAGGAAACUCC CU UCAAGGACAUCGUCCGGG CCGGGCGG | 3648 |
| 43 | CGCGAGCU G GAUUAUGG | 1452 | CCAUAAUC GGAGGAAACUCC CU UCAAGGACAUCGUCCGGG AGCUCGCG | 3649 |
| 44 | GCGAGCUG G AUUAUGGU | 1453 | ACCAUAAU GGAGGAAACUCC CU UCAAGGACAUCGUCCGGG CAGCUCGC | 3650 |
| 50 | UGGAUUAU G GUGGCCUG | 1454 | CAGGCCAC GGAGGAAACUCC CU UCAAGGACAUCGUCCGGG AUAAUCCA | 3651 |
| 53 | AUUAUGGU G GCCUGAGC | 1455 | GCUCAGGC GGAGGAAACUCC CU UCAAGGACAUCGUCCGGG ACCAUAAU | 3652 |
| 78 | CAGCCGCA G GAGCCCGG | 1456 | CCGGGCUC GGAGGAAACUCC CU UCAAGGACAUCGUCCGGG UGCGGCUG | 3653 |
| 79 | AGCCGCAG G AGCCCGGA | 1457 | UCCGGGCU GGAGGAAACUCC CU UCAAGGACAUCGUCCGGG CUGCGGCU | 3654 |
| 85 | AGGAGCCC G GAGCCCUU | 1458 | AAGGGCUC GGAGGAAACUCC CU UCAAGGACAUCGUCCGGG GGGCUCCU | 3655 |
| 86 | GGAGCCCG G AGCCCUUG | 1459 | CAAGGGCU GGAGGAAACUCC CU UCAAGGACAUCGUCCGGG CGGGCUCC | 3656 |
| 119 | CGCCCGCC G GGGGGACC | 1460 | GGUCCCCC GGAGGAAACUCC CU UCAAGGACAUCGUCCGGG GGCGGGCG | 3657 |
| 120 | GCCCGCCG G GGGGACCA | 1461 | UGGUCCCC GGAGGAAACUCC CU UCAAGGACAUCGUCCGGG CGGCGGGC | 3658 |
| 121 | CCCGCCGG G GGGACCAG | 1462 | CUGGUCCC GGAGGAAACUCC CU UCAAGGACAUCGUCCGGG CCGGCGGG | 3659 |
| 122 | CCGCCGGG G GGACCAGG | 1463 | CCUGGUCC GGAGGAAACUCC CU UCAAGGACAUCGUCCGGG CCCGGCGG | 3660 |
| 123 | CGCCGGGG G GACCAGGG | 1464 | CCCUGGUC GGAGGAAACUCC CU UCAAGGACAUCGUCCGGG CCCCGGCG | 3661 |
| 124 | GCCGGGGG G ACCAGGGA | 1465 | UCCCUGGU GGAGGAAACUCC CU UCAAGGACAUCGUCCGGG CCCCCGGC | 3662 |
| 129 | GGGGACCA G GGAAGCCG | 1466 | CGGCUUCC GGAGGAAACUCC CU UCAAGGACAUCGUCCGGG UGGUCCCC | 3663 |
| 130 | GGGACCAG G GAAGCCGC | 1467 | GCGGCUUC GGAGGAAACUCC CU UCAAGGACAUCGUCCGGG CUGGUCCC | 3664 |
| 131 | GGACCAGG G AAGCCGCC | 1468 | GGCGGCUU GGAGGAAACUCC CU UCAAGGACAUCGUCCGGG CCUGGUCC | 3665 |
| 143 | CCGCCACC G GCCCGCCA | 1469 | UGGCGGGC GGAGGAAACUCC CU UCAAGGACAUCGUCCGGG GGUGGCGG | 3666 |
| 175 | GCCCCGCC G GGAGCCCG | 1470 | CGGGCUCC GGAGGAAACUCC CU UCAAGGACAUCGUCCGGG GGCGGGGC | 3667 |
| 176 | CCCCGCCG G GAGCCCGC | 1471 | GCGGGCUC GGAGGAAACUCC CU UCAAGGACAUCGUCCGGG CGGCGGGG | 3668 |
| 177 | CCCGCCGG G AGCCCGCG | 1472 | CGCGGGCU GGAGGAAACUCC CU UCAAGGACAUCGUCCGGG CCGGCGGG | 3669 |
| 197 | GCUGCCCA G GCUGGCCG | 1473 | CGGCCAGC GGAGGAAACUCC CU UCAAGGACAUCGUCCGGG UGGGCAGC | 3670 |
| 201 | CCCAGGCU G GCCGCCGC | 1474 | GCGGCGGC GGAGGAAACUCC CU UCAAGGACAUCGUCCGGG AGCCUGGG | 3671 |
| 224 | GAUGUAGC G GGCUCCGG | 1475 | CCGGAGCC GGAGGAAACUCC CU UCAAGGACAUCGUCCGGG GCUACAUC | 3672 |
| 225 | AUGUAGCG G GCUCCGGA | 1476 | UCCGGAGC GGAGGAAACUCC CU UCAAGGACAUCGUCCGGG CGCUACAU | 3673 |
| 231 | CGGGCUCC G GAUCCCAG | 1477 | CUGGGAUC GGAGGAAACUCC CU UCAAGGACAUCGUCCGGG GGAGCCCG | 3674 |
| 232 | GGGCUCCG G AUCCCAGC | 1478 | GCUGGGAU GGAGGAAACUCC CU UCAAGGACAUCGUCCGGG CGGAGCCC | 3675 |
| 265 | UGCUCUGC G GAUCUCCC | 1479 | GGGAGAUC GGAGGAAACUCC CU UCAAGGACAUCGUCCGGG GCAGAGCA | 3676 |
| 266 | GCUCUGCG G AUCUCCCC | 1480 | GGGGAGAU GGAGGAAACUCC CU UCAAGGACAUCGUCCGGG CGCAGAGC | 3677 |
| 294 | CACAGCCC G GACCCGGG | 1481 | CCCGGGUC GGAGGAAACUCC CU UCAAGGACAUCGUCCGGG GGGCUGUG | 3678 |
| 295 | ACAGCCCG G ACCCGGGG | 1482 | CCCCGGGU GGAGGAAACUCC CU UCAAGGACAUCGUCCGGG CGGGCUGU | 3679 |
| 300 | CCGGACCC G GGGGCUGG | 1483 | CCAGCCCC GGAGGAAACUCC CU UCAAGGACAUCGUCCGGG GGGUCCGG | 3680 |
| 301 | CGGACCCG G GGGCUGGC | 1484 | GCCAGCCC GGAGGAAACUCC CU UCAAGGACAUCGUCCGGG CGGGUCCG | 3681 |
| 302 | GGACCCGG G GGCUGGCC | 1485 | GGCCAGCC GGAGGAAACUCC CU UCAAGGACAUCGUCCGGG CCGGGUCC | 3682 |
| 303 | GACCCGGG G GCUGGCCC | 1486 | GGGCCAGC GGAGGAAACUCC CU UCAAGGACAUCGUCCGGG CCCGGGUC | 3683 |
| 307 | CGGGGGCU G GCCCAGGG | 1487 | CCCUGGGC GGAGGAAACUCC CU UCAAGGACAUCGUCCGGG AGCCCCCG | 3684 |
| 313 | CUGGCCCA G GGCCCUGC | 1488 | GCAGGGCC GGAGGAAACUCC CU UCAAGGACAUCGUCCGGG UGGGCCAG | 3685 |
| 314 | UGGCCCAG G GCCCUGCA | 1489 | UGCAGGGC GGAGGAAACUCC CU UCAAGGACAUCGUCCGGG CUGGGCCA | 3686 |
| 323 | GCCCUGCA G GCCCUGGC | 1490 | GCCAGGGC GGAGGAAACUCC CU UCAAGGACAUCGUCCGGG UGCAGGGC | 3687 |
| 329 | CAGGCCCU G GCGUCCUG | 1491 | CAGGACGC GGAGGAAACUCC CU UCAAGGACAUCGUCCGGG AGGGCCUG | 3688 |
| 362 | UCUCCUGA G AAGCCACC | 1492 | GGUGGCUU GGAGGAAACUCC CU UCAAGGACAUCGUCCGGG UCAGGAGA | 3689 |
| 382 | ACCACCCA G ACUUGGGG | 1493 | CCCCAAGU GGAGGAAACUCC CU UCAAGGACAUCGUCCGGG UGGGUGGU | 3690 |
| 387 | CCAGACUU G GGGGCAGG | 1494 | CCUGCCCC GGAGGAAACUCC CU UCAAGGACAUCGUCCGGG AAGUCUGG | 3691 |
| 388 | CAGACUUG G GGGCAGGC | 1495 | GCCUGCCC GGAGGAAACUCC CU UCAAGGACAUCGUCCGGG CAAGUCUG | 3692 |
| 389 | AGACUUGG G GGCAGGCG | 1496 | CGCCUGCC GGAGGAAACUCC CU UCAAGGACAUCGUCCGGG CCAAGUCU | 3693 |
| 390 | GACUUGGG G GCAGGCGC | 1497 | GCGCCUGC GGAGGAAACUCC CU UCAAGGACAUCGUCCGGG CCCAAGUC | 3694 |
| 394 | UGGGGGCA G GCGCCAGG | 1498 | CCUGGCGC GGAGGAAACUCC CU UCAAGGACAUCGUCCGGG UGCCCCCA | 3695 |
| 401 | AGGCGCCA G GGACGGAC | 1499 | GUCCGUCC GGAGGAAACUCC CU UCAAGGACAUCGUCCGGG UGGCGCCU | 3696 |
| 402 | GGCGCCAG G GACGGACG | 1500 | CGUCCGUC GGAGGAAACUCC CU UCAAGGACAUCGUCCGGG CUGGCGCC | 3697 |
| 403 | GCGCCAGG G ACGGACGU | 1501 | ACGUCCGU GGAGGAAACUCC CU UCAAGGACAUCGUCCGGG CCUGGCGC | 3698 |
| 406 | CCAGGGAC G GACGUGGG | 1502 | CCCACGUC GGAGGAAACUCC CU UCAAGGACAUCGUCCGGG GUCCCUGG | 3699 |
| 407 | CAGGGACG G ACGUGGGC | 1503 | GCCCACGU GGAGGAAACUCC CU UCAAGGACAUCGUCCGGG CGUCCCUG | 3700 |
| 412 | ACGGACGU G GGCCAGUG | 1504 | CACUGGCC GGAGGAAACUCC CU UCAAGGACAUCGUCCGGG ACGUCCGU | 3701 |
| 413 | CGGACGUG G GCCAGUGC | 1505 | GCACUGGC GGAGGAAACUCC CU UCAAGGACAUCGUCCGGG CACGUCCG | 3702 |
| 429 | CGAGCCCA G AGGGCCCG | 1506 | CGGGCCCU GGAGGAAACUCC CU UCAAGGACAUCGUCCGGG UGGGCUCG | 3703 |
| 431 | AGCCCAGA G GGCCCGAA | 1507 | UUCGGGCC GGAGGAAACUCC CU UCAAGGACAUCGUCCGGG UCUGGGCU | 3704 |
| 432 | GCCCAGAG G GCCCGAAG | 1508 | CUUCGGGC GGAGGAAACUCC CU UCAAGGACAUCGUCCGGG CUCUGGGC | 3705 |
| 440 | GGCCCGAA G GCCGGGGC | 1509 | GCCCCGGC GGAGGAAACUCC CU UCAAGGACAUCGUCCGGG UUCGGGCC | 3706 |
| 444 | CGAAGGCC G GGGCCCAC | 1510 | GUGGGCCC GGAGGAAACUCC CU UCAAGGACAUCGUCCGGG GGCCUUCG | 3707 |
| 445 | GAAGGCCG G GGCCCACC | 1511 | GGUGGGCC GGAGGAAACUCC CU UCAAGGACAUCGUCCGGG CGGCCUUC | 3708 |
| 446 | AAGGCCGG G GCCCACCA | 1512 | UGGUGGGC GGAGGAAACUCC CU UCAAGGACAUCGUCCGGG CCGGCCUU | 3709 |
| 456 | CCCACCAU G GCCCAAGC | 1513 | GCUUGGGC GGAGGAAACUCC CU UCAAGGACAUCGUCCGGG AUGGUGGG | 3710 |
| 473 | CCUGCCCU G GCUCCUGC | 1514 | GCAGGAGC GGAGGAAACUCC CU UCAAGGACAUCGUCCGGG AGGGCAGG | 3711 |
| 485 | CCUGCUGU G GAUGGGCG | 1515 | CGCCCAUC GGAGGAAACUCC CU UCAAGGACAUCGUCCGGG ACAGCAGG | 3712 |
| 486 | CUGCUGUG G AUGGGCGC | 1516 | GCGCCCAU GGAGGAAACUCC CU UCAAGGACAUCGUCCGGG CACAGCAG | 3713 |
| 489 | CUGUGGAU G GGCGCGGG | 1517 | CCCGCGCC GGAGGAAACUCC CU UCAAGGACAUCGUCCGGG AUCCACAG | 3714 |
| 490 | UGUGGAUG G GCGCGGGA | 1518 | UCCCGCGC GGAGGAAACUCC CU UCAAGGACAUCGUCCGGG CAUCCACA | 3715 |
| 495 | AUGGGCGC G GGAGUGCU | 1519 | AGCACUCC GGAGGAAACUCC CU UCAAGGACAUCGUCCGGG GCGCCCAU | 3716 |
| 496 | UGGGCGCG G GAGUGCUG | 1520 | CAGCACUC GGAGGAAACUCC CU UCAAGGACAUCGUCCGGG CGCGCCCA | 3717 |
| 497 | GGGCGCGG G AGUGCUGC | 1521 | GCAGCACU GGAGGAAACUCC CU UCAAGGACAUCGUCCGGG CCGCGCCC | 3718 |
| 514 | CUGCCCAC G GCACCCAG | 1522 | CUGGGUGC GGAGGAAACUCC CU UCAAGGACAUCGUCCGGG GUGGGCAG | 3719 |
| 526 | CCCAGCAC G GCAUCCGG | 1523 | CCGGAUGC GGAGGAAACUCC CU UCAAGGACAUCGUCCGGG GUGCUGGG | 3720 |
| 533 | CGGCAUCC G GCUGCCCC | 1524 | GGGGCAGC GGAGGAAACUCC CU UCAAGGACAUCGUCCGGG GGAUGCCG | 3721 |
| 550 | UGCGCAGC G GCCUGGGG | 1525 | CCCCAGGC GGAGGAAACUCC CU UCAAGGACAUCGUCCGGG GCUGCGCA | 3722 |
| 555 | AGCGGCCU G GGGGGCGC | 1526 | GCGCCCCC GGAGGAAACUCC CU UCAAGGACAUCGUCCGGG AGGCCGCU | 3723 |
| 556 | GCGGCCUG G GGGGCGCC | 1527 | GGCGCCCC GGAGGAAACUCC CU UCAAGGACAUCGUCCGGG CAGGCCGC | 3724 |
| 557 | CGGCCUGG G GGGCGCCC | 1528 | GGGCGCCC GGAGGAAACUCC CU UCAAGGACAUCGUCCGGG CCAGGCCG | 3725 |
| 558 | GGCCUGGG G GGCGCCCC | 1529 | GGGGCGCC GGAGGAAACUCC CU UCAAGGACAUCGUCCGGG CCCAGGCC | 3726 |
| 559 | GCCUGGGG G GCGCCCCC | 1530 | GGGGGCGC GGAGGAAACUCC CU UCAAGGACAUCGUCCGGG CCCCAGGC | 3727 |
| 570 | GCCCCCCU G GGGCUGCG | 1531 | CGCAGCCC GGAGGAAACUCC CU UCAAGGACAUCGUCCGGG AGGGGGGC | 3728 |
| 571 | CCCCCCUG G GGCUGCGG | 1532 | CCGCAGCC GGAGGAAACUCC CU UCAAGGACAUCGUCCGGG CAGGGGGG | 3729 |
| 572 | CCCCCUGG G GCUGCGGC | 1533 | GCCGCAGC GGAGGAAACUCC CU UCAAGGACAUCGUCCGGG CCAGGGGG | 3730 |
| 578 | GGGGCUGC G GCUGCCCC | 1534 | GGGGCAGC GGAGGAAACUCC CU UCAAGGACAUCGUCCGGG GCAGCCCC | 3731 |
| 587 | GCUGCCCC G GGAGACCG | 1535 | CGGUCUCC GGAGGAAACUCC CU UCAAGGACAUCGUCCGGG GGGGCAGC | 3732 |
| 588 | CUGCCCCG G GAGACCGA | 1536 | UCGGUCUC GGAGGAAACUCC CU UCAAGGACAUCGUCCGGG CGGGGCAG | 3733 |
| 589 | UGCCCCGG G AGACCGAC | 1537 | GUCGGUCU GGAGGAAACUCC CU UCAAGGACAUCGUCCGGG CCGGGGCA | 3734 |
| 591 | CCCCGGGA G ACCGACGA | 1538 | UCGUCGGU GGAGGAAACUCC CU UCAAGGACAUCGUCCGGG UCCCGGGG | 3735 |
| 601 | CCGACGAA G AGCCCGAG | 1539 | CUCGGGCU GGAGGAAACUCC CU UCAAGGACAUCGUCCGGG UUCGUCGG | 3736 |
| 609 | GAGCCCGA G GAGCCCGG | 1540 | CCGGGCUC GGAGGAAACUCC CU UCAAGGACAUCGUCCGGG UCGGGCUC | 3737 |
| 610 | AGCCCGAG G AGCCCGGC | 1541 | GCCGGGCU GGAGGAAACUCC CU UCAAGGACAUCGUCCGGG CUCGGGCU | 3738 |
| 616 | AGGAGCCC G GCCGGAGG | 1542 | CCUCCGGC GGAGGAAACUCC CU UCAAGGACAUCGUCCGGG GGGCUCCU | 3739 |
| 620 | GCCCGGCC G GAGGGGCA | 1543 | UGCCCCUC GGAGGAAACUCC CU UCAAGGACAUCGUCCGGG GGCCGGGC | 3740 |
| 621 | CCCGGCCG G AGGGGCAG | 1544 | CUGCCCCU GGAGGAAACUCC CU UCAAGGACAUCGUCCGGG CGGCCGGG | 3741 |
| 623 | CGGCCGGA G GGGCAGCU | 1545 | AGCUGCCC GGAGGAAACUCC CU UCAAGGACAUCGUCCGGG UCCGGCCG | 3742 |
| 624 | GGCCGGAG G GGCAGCUU | 1546 | AAGCUGCC GGAGGAAACUCC CU UCAAGGACAUCGUCCGGG CUCCGGCC | 3743 |
| 625 | GCCGGAGG G GCAGCUUU | 1547 | AAAGCUGC GGAGGAAACUCC CU UCAAGGACAUCGUCCGGG CCUCCGGC | 3744 |
| 636 | AGCUUUGU G GAGAUGGU | 1548 | ACCAUCUC GGAGGAAACUCC CU UCAAGGACAUCGUCCGGG ACAAAGCU | 3745 |
| 637 | GCUUUGUG G AGAUGGUG | 1549 | CACCAUCU GGAGGAAACUCC CU UCAAGGACAUCGUCCGGG CACAAAGC | 3746 |
| 639 | UUUGUGGA G AUGGUGGA | 1550 | UCCACCAU GGAGGAAACUCC CU UCAAGGACAUCGUCCGGG UCCACAAA | 3747 |
| 642 | GUGGAGAU G GUGGACAA | 1551 | UUGUCCAC GGAGGAAACUCC CU UCAAGGACAUCGUCCGGG AUCUCCAC | 3748 |
| 645 | GAGAUGGU G GACAACCU | 1552 | AGGUUGUC GGAGGAAACUCC CU UCAAGGACAUCGUCCGGG ACCAUCUC | 3749 |
| 646 | AGAUGGUG G ACAACCUG | 1553 | CAGGUUGU GGAGGAAACUCC CU UCAAGGACAUCGUCCGGG CACCAUCU | 3750 |
| 656 | CAACCUGA G GGGCAAGU | 1554 | ACUUGCCC GGAGGAAACUCC CU UCAAGGACAUCGUCCGGG UCAGGUUG | 3751 |
| 657 | AACCUGAG G GGCAAGUC | 1555 | GACUUGCC GGAGGAAACUCC CU UCAAGGACAUCGUCCGGG CUCAGGUU | 3752 |
| 658 | ACCUGAGG G GCAAGUCG | 1556 | CGACUUGC GGAGGAAACUCC CU UCAAGGACAUCGUCCGGG CCUCAGGU | 3753 |
| 666 | GGCAAGUC G GGGCAGGG | 1557 | CCCUGCCC GGAGGAAACUCC CU UCAAGGACAUCGUCCGGG GACUUGCC | 3754 |
| 667 | GCAAGUCG G GGCAGGGC | 1558 | GCCCUGCC GGAGGAAACUCC CU UCAAGGACAUCGUCCGGG CGACUUGC | 3755 |
| 668 | CAAGUCGG G GCAGGGCU | 1559 | AGCCCUGC GGAGGAAACUCC CU UCAAGGACAUCGUCCGGG CCGACUUG | 3756 |
| 672 | UCGGGGCA G GGCUACUA | 1560 | UAGUAGCC GGAGGAAACUCC CU UCAAGGACAUCGUCCGGG UGCCCCGA | 3757 |
| 673 | CGGGGCAG G GCUACUAC | 1561 | GUAGUAGC GGAGGAAACUCC CU UCAAGGACAUCGUCCGGG CUGCCCCG | 3758 |
| 664 | UACUACGU G GAGAUGAC | 1562 | GUCAUCUC GGAGGAAACUCC CU UCAAGGACAUCGUCCGGG ACGUAGUA | 3759 |
| 685 | ACUACGUG G AGAUGACC | 1563 | GGUCAUCU GGAGGAAACUCC CU UCAAGGACAUCGUCCGGG CACGUAGU | 3760 |
| 687 | UACGUGGA G AUGACCGU | 1564 | ACGGUCAU GGAGGAAACUCC CU UCAAGGACAUCGUCCGGG UCCACGUA | 3761 |
| 696 | AUGACCGU G GGCAGCCC | 1565 | GGGCUGCC GGAGGAAACUCC CU UCAAGGACAUCGUCCGGG ACGGUCAU | 3762 |
| 697 | UGACCGUG G GCAGCCCC | 1566 | GGGGCUGC GGAGGAAACUCC CU UCAAGGACAUCGUCCGGG CACGGUCA | 3763 |
| 711 | CCCCCGCA G ACGCUCAA | 1567 | UUGAGCGU GGAGGAAACUCC CU UCAAGGACAUCGUCCGGG UGCGGGGG | 3764 |
| 726 | AACAUCCU G GUGGAUAC | 1568 | GUAUCCAC GGAGGAAACUCC CU UCAAGGACAUCGUCCGGG AGGAUGUU | 3765 |
| 729 | AUCCUGGU G GAUACAGG | 1569 | CCUGUAUC GGAGGAAACUCC CU UCAAGGACAUCGUCCGGG ACCAGGAU | 3766 |
| 730 | UCCUGGUG G AUACAGGC | 1570 | GCCUGUAU GGAGGAAACUCC CU UCAAGGACAUCGUCCGGG CACCAGGA | 3767 |
| 736 | UGGAUACA G GCAGCAGU | 1571 | ACUGCUGC GGAGGAAACUCC CU UCAAGGACAUCGUCCGGG UGUAUCCA | 3768 |
| 756 | UUUGCAGU G GGUGCUGC | 1572 | GCAGCACC GGAGGAAACUCC CU UCAAGGACAUCGUCCGGG ACUGCAAA | 3769 |
| 757 | UUGCAGUG G GUGCUGCC | 1513 | GGCAGCAC GGAGGAAACUCC CU UCAAGGACAUCGUCCGGG CACUGCAA | 3770 |
| 795 | UACUACCA G AGGCAGCU | 1574 | AGCUGCCU GGAGGAAACUCC CU UCAAGGACAUCGUCCGGG UGGUAGUA | 3771 |
| 797 | CUACCAGA G GCAGCUGU | 1575 | ACAGCUGC GGAGGAAACUCC CU UCAAGGACAUCGUCCGGG UCUGGUAG | 3772 |
| 818 | CACAUACC G GGACCUCC | 1576 | GGAGGUCC GGAGGAAACUCC CU UCAAGGACAUCGUCCGGG GGUAUGUG | 3773 |
| 819 | ACAUACCG G GACCUCCG | 1577 | CGGAGGUC GGAGGAAACUCC CU UCAAGGACAUCGUCCGGG CGGUAUGU | 3774 |
| 820 | CAUACCGG G ACCUCCGG | 1578 | CCGGAGGU GGAGGAAACUCC CU UCAAGGACAUCGUCCGGG CCGGUAUG | 3775 |
| 827 | GGACCUCC G GAAGGGUG | 1579 | CACCCUUC GGAGGAAACUCC CU UCAAGGACAUCGUCCGGG GGAGGUCC | 3776 |
| 828 | GACCUCCG G AAGGGUGU | 1580 | ACACCCUU GGAGGAAACUCC CU UCAAGGACAUCGUCCGGG CGGAGGUC | 3777 |
| 831 | CUCCGGAA G GGUGUGUA | 1581 | UACACACC GGAGGAAACUCC CU UCAAGGACAUCGUCCGGG UUCCGGAG | 3778 |
| 832 | UCCGGAAG G GUGUGUAU | 1582 | AUACACAC GGAGGAAACUCC CU UCAAGGACAUCGUCCGGG CUUCCGGA | 3779 |
| 855 | UACACCCA G GGCAAGUG | 1583 | CACUUGCC GGAGGAAACUCC CU UCAAGGACAUCGUCCGGG UGGGUGUA | 3780 |
| 856 | ACACCCAG G GCAAGUGG | 1584 | CCACUUGC GGAGGAAACUCC CU UCAAGGACAUCGUCCGGG CUGGGUGU | 3781 |
| 863 | GGGCAAGU G GGAAGGGG | 1585 | CCCCUUCC GGAGGAAACUCC CU UCAAGGACAUCGUCCGGG ACUUGCCC | 3782 |
| 864 | GGCAAGUG G GAAGGGGA | 1586 | UCCCCUUC GGAGGAAACUCC CU UCAAGGACAUCGUCCGGG CACUUGCC | 3783 |
| 865 | GCAAGUGG G AAGGGGAG | 1587 | CUCCCCUU GGAGGAAACUCC CU UCAAGGACAUCGUCCGGG CCACUUGC | 3784 |
| 868 | AGUGGGAA G GGGAGCUG | 1588 | CAGCUCCC GGAGGAAACUCC CU UCAAGGACAUCGUCCGGG UUCCCACU | 3785 |
| 869 | GUGGGAAG G GGAGCUGG | 1589 | CCAGCUCC GGAGGAAACUCC CU UCAAGGACAUCGUCCGGG CUUCCCAC | 3786 |
| 870 | UGGGAAGG G GAGCUGGG | 1590 | CCCAGCUC GGAGGAAACUCC CU UCAAGGACAUCGUCCGGG CCUUCCCA | 3787 |
| 871 | GGGAAGGG G AGCUGGGC | 1591 | GCCCAGCU GGAGGAAACUCC CU UCAAGGACAUCGUCCGGG CCCUUCCC | 3788 |
| 876 | GGGGAGCU G GGCACCGA | 1592 | UCGGUGCC GGAGGAAACUCC CU UCAAGGACAUCGUCCGGG AGCUCCCC | 3789 |
| 877 | GGGAGCUG G GCACCGAC | 1593 | GUCGGUGC GGAGGAAACUCC CU UCAAGGACAUCGUCCGGG CAGCUCCC | 3790 |
| 888 | ACCGACCU G GUAAGCAU | 1594 | AUGCUUAC GGAGGAAACUCC CU UCAAGGACAUCGUCCGGG AGGUCGGU | 3791 |
| 904 | UCCCCCAU G GCCCCAAC | 1595 | GUUGGGGC GGAGGAAACUCC CU UCAAGGACAUCGUCCGGG AUGGGGGA | 3792 |
| 952 | CUGAAUCA G ACAAGUUC | 1596 | GAACUUGU GGAGGAAACUCC CU UCAAGGACAUCGUCCGGG UGAUUCAG | 3793 |
| 970 | UCAUCAAC G GCUCCAAC | 1597 | GUUGGAGC GGAGGAAACUCC CU UCAAGGACAUCGUCCGGG GUUGAUGA | 3794 |
| 980 | CUCCAACU G GGAAGGCA | 1598 | UGCCUUCC GGAGGAAACUCC CU UCAAGGACAUCGUCCGGG AGUUGGAG | 3795 |
| 981 | UCCAACUG G GAAGGCAU | 1599 | AUGCCUUC GGAGGAAACUCC CU UCAAGGACAUCGUCCGGG CAGUUGGA | 3796 |
| 982 | CCAACUGG G AAGGCAUC | 1600 | GAUGCCUU GGAGGAAACUCC CU UCAAGGACAUCGUCCGGG CCAGUUGG | 3797 |
| 985 | ACUGGGAA G GCAUCCUG | 1601 | CAGGAUGC GGAGGAAACUCC CU UCAAGGACAUCGUCCGGG UUCCCAGU | 3798 |
| 993 | GGCAUCCU G GGGCUGGC | 1602 | GCCAGCCC GGAGGAAACUCC CU UCAAGGACAUCGUCCGGG AGGAUGCC | 3799 |
| 994 | GCAUCCUG G GGCUGGCC | 1603 | GGCCAGCC GGAGGAAACUCC CU UCAAGGACAUCGUCCGGG CAGGAUGC | 3800 |
| 995 | CAUCCUGG G GCUGGCCU | 1604 | AGGCCAGC GGAGGAAACUCC CU UCAAGGACAUCGUCCGGG CCAGGAUG | 3801 |
| 999 | CUGGGGCU G GCCUAUGC | 1605 | GCAUAGGC GGAGGAAACUCC CU UCAAGGACAUCGUCCGGG AGCCCCAG | 3802 |
| 1022 | UAUGCUGA G AUUGCCAG | 1606 | CUGGCAAU GGAGGAAACUCC CU UCAAGGACAUCGUCCGGG UCAGCAUA | 3803 |
| 1019 | GAUUGCCA G GCCUGACG | 1607 | CGUCAGGC GGAGGAAACUCC CU UCAAGGACAUCGUCCGGG UGGCAAUC | 3804 |
| 1035 | GACUCCCU G GAGCCUUU | 1608 | AAAGGCUC GGAGGAAACUCC CU UCAAGGACAUCGUCCGGG AGGGAGUC | 3805 |
| 1036 | ACUCCCUG G AGCCUUUC | 1609 | GAAAGGCU GGAGGAAACUCC CU UCAAGGACAUCGUCCGGG CAGGGAGU | 3806 |
| 1056 | GACUCUCU G GUAAAGCA | 1610 | UGCUUUAC GGAGGAAACUCC CU UCAAGGACAUCGUCCGGG AGAGAGUC | 3807 |
| 1065 | GUAAAGCA G ACCCACGU | 1611 | ACGUGGGU GGAGGAAACUCC CU UCAAGGACAUCGUCCGGG UGCUUUAC | 3808 |
| 1102 | AGCUUUGU G GUGCUGGC | 1612 | GCCAGCAC GGAGGAAACUCC CU UCAAGGACAUCGUCCGGG ACAAAGCU | 3809 |
| 1108 | GUGGUGCU G GCUUCCCC | 1613 | GGGGAAGC GGAGGAAACUCC CU UCAAGGACAUCGUCCGGG AGCACCAC | 3810 |
| 1137 | GAAGUGCU G GCCUCUGU | 1614 | ACAGAGGC GGAGGAAACUCC CU UCAAGGACAUCGUCCGGG AGCACUUC | 3811 |
| 1147 | CCUCUGUC G GAGGGAGC | 1615 | GCUCCCUC GGAGGAAACUCC CU UCAAGGACAUCGUCCGGG GACAGAGG | 3812 |
| 1148 | CUCUGUCG G AGGGAGCA | 1616 | UGCUCCCU GGAGGAAACUCC CU UCAAGGACAUCGUCCGGG CGACAGAG | 3813 |
| 1150 | CUGUCGGA G GGAGCAUG | 1617 | CAUGCUCC GGAGGAAACUCC CU UCAAGGACAUCGUCCGGG UCCGACAG | 3814 |
| 1151 | UGUCGGAG G GAGCAUGA | 1618 | UCAUGCUC GGAGGAAACUCC CU UCAAGGACAUCGUCCGGG CUCCGACA | 3815 |
| 1152 | GUCGGAGG G AGCAUGAU | 1619 | AUCAUGCU GGAGGAAACUCC CU UCAAGGACAUCGUCCGGG CCUCCGAC | 3816 |
| 1165 | UGAUCAUU G GAGGUAUC | 1620 | GAUACCUC GGAGGAAACUCC CU UCAAGGACAUCGUCCGGG AAUGAUCA | 3817 |
| 1166 | GAUCAUUG G AGGUAUCG | 1621 | CGAUACCU GGAGGAAACUCC CU UCAAGGACAUCGUCCGGG CAAUGAUC | 3818 |
| 1168 | UCAUUGGA G GUAUCGAC | 1622 | GUCGAUAC GGAGGAAACUCC CU UCAAGGACAUCGUCCGGG UCCAAUGA | 3819 |
| 1192 | UGUACACA G GCAGUCUC | 1623 | GAGACUGC GGAGGAAACUCC CU UCAAGGACAUCGUCCGGG UGUGUACA | 3820 |
| 1202 | CAGUCUCU G GUAUACAC | 1624 | GUGUAUAC GGAGGAAACUCC CU UCAAGGACAUCGUCCGGG AGAGACUG | 3821 |
| 1217 | ACCCAUCC G GCGGGAGU | 1625 | ACUCCCGC GGAGGAAACUCC CU UCAAGGACAUCGUCCGGG GGAUGGGU | 3822 |
| 1220 | CAUCCGGC G GGAGUGGU | 1626 | ACCACUCC GGAGGAAACUCC CU UCAAGGACAUCGUCCGGG GCCGGAUG | 3823 |
| 1221 | AUCCGGCG G GAGUGGUA | 1627 | UACCACUC GGAGGAAACUCC CU UCAAGGACAUCGUCCGGG CGCCGGAU | 3824 |
| 1222 | UCCGGCGG G AGUGGUAU | 1628 | AUACCACU GGAGGAAACUCC CU UCAAGGACAUCGUCCGGG CCGCCGGA | 3825 |
| 1226 | GCGGGAGU G GUAUUAUG | 1629 | CAUAAUAC GGAGGAAACUCC CU UCAAGGACAUCGUCCGGG ACUCCCGC | 3826 |
| 1236 | UAUUAUGA G GUGAUCAU | 1630 | AUGAUCAC GGAGGAAACUCC CU UCAAGGACAUCGUCCGGG UCAUAAUA | 3827 |
| 1250 | CAUUGUGC G GGUGGAGA | 1631 | UCUCCACC GGAGGAAACUCC CU UCAAGGACAUCGUCCGGG GCACAAUG | 3828 |
| 1251 | AUUGUGCG G GUGGAGAU | 1632 | AUCUCCAC GGAGGAAACUCC CU UCAAGGACAUCGUCCGGG CGCACAAU | 3829 |
| 1254 | GUGCGGGU G GAGAUCAA | 1633 | UUGAUCUC GGAGGAAACUCC CU UCAAGGACAUCGUCCGGG ACCCGCAC | 3830 |
| 1255 | UGCGGGUG G AGAUCAAU | 1634 | AUUGAUCU GGAGGAAACUCC CU UCAAGGACAUCGUCCGGG CACCCGCA | 3831 |
| 1257 | CGGGUGGA G AUCAAUGG | 1635 | CCAUUGAU GGAGGAAACUCC CU UCAAGGACAUCGUCCGGG UCCACCCG | 3832 |
| 1264 | AGAUCAAU G GACAGGAU | 1636 | AUCCUGUC GGAGGAAACUCC CU UCAAGGACAUCGUCCGGG AUUGAUCU | 3833 |
| 1265 | GAUCAAUG G ACAGGAUC | 1637 | GAUCCUGU GGAGGAAACUCC CU UCAAGGACAUCGUCCGGG CAUUGAUC | 3834 |
| 1269 | AAUGGACA G GAUCUGAA | 1638 | UUCAGAUC GGAGGAAACUCC CU UCAAGGACAUCGUCCGGG UGUCCAUU | 3835 |
| 1270 | AUGGACAG G AUCUGAAA | 1639 | UUUCAGAU GGAGGAAACUCC CU UCAAGGACAUCGUCCGGG CUGUCCAU | 3836 |
| 1281 | CUGAAAAU G GACUGCAA | 1640 | UUGCAGUC GGAGGAAACUCC CU UCAAGGACAUCGUCCGGG AUUUUCAG | 3837 |
| 1282 | UGAAAAUG G ACUGCAAG | 1641 | CUUGCAGU GGAGGAAACUCC CU UCAAGGACAUCGUCCGGG CAUUUUCA | 3838 |
| 1290 | GACUGCAA G GAGUACAA | 1642 | UUGUACUC GGAGGAAACUCC CU UCAAGGACAUCGUCCGGG UUGCAGUC | 3839 |
| 1291 | ACUGCAAG G AGUACAAC | 1643 | GUUGUACU GGAGGAAACUCC CU UCAAGGACAUCGUCCGGG CUUGCAGU | 3840 |
| 1308 | UAUGACAA G AGCAUUGU | 1644 | ACAAUGCU GGAGGAAACUCC CU UCAAGGACAUCGUCCGGG UUGUCAUA | 3841 |
| 1317 | AGCAUUGU G GACAGUGG | 1645 | CCACUGUC GGAGGAAACUCC CU UCAAGGACAUCGUCCGGG ACAAUGCU | 3842 |
| 1318 | GCAUUGUG G ACAGUGGC | 1646 | GCCACUGU GGAGGAAACUCC CU UCAAGGACAUCGUCCGGG CACAAUGC | 3843 |
| 1324 | UGGACAGU G GCACCACC | 1647 | GGUGGUGC GGAGGAAACUCC CU UCAAGGACAUCGUCCGGG ACUGUCCA | 3844 |
| 1350 | UUGCCCAA G AAAGUGUU | 1648 | AACACUUU GGAGGAAACUCC CU UCAAGGACAUCGUCCGGG UUGGGCAA | 3845 |
| 1383 | UCCAUCAA G GCAGCCUC | 1649 | GAGGCUGC GGAGGAAACUCC CU UCAAGGACAUCGUCCGGG UUGAUGGA | 3846 |
| 1398 | UCCUCCAC G GAGAAGUU | 1650 | AACUUCUC GGAGGAAACUCC CU UCAAGGACAUCGUCCGGG GUGGAGGA | 3847 |
| 1399 | CCUCCACG G AGAAGUUC | 1651 | GAACUUCU GGAGGAAACUCC CU UCAAGGACAUCGUCCGGG CGUGGAGG | 3848 |
| 1401 | UCCACGGA G AAGUUCCC | 1652 | GGGAACUU GGAGGAAACUCC CU UCAAGGACAUCGUCCGGG UCCGUGGA | 3849 |
| 1414 | UCCCUGAU G GUUUCUGG | 1653 | CCAGAAAC GGAGGAAACUCC CU UCAAGGACAUCGUCCGGG AUCAGGGA | 3850 |
| 1421 | UGGUUUCU G GCUAGGAC | 1654 | CUCCUAGC GGAGGAAACUCC CU UCAAGGACAUCGUCCGGG AGAAACCA | 3851 |
| 1426 | UCUGGCUA G GAGAGCAG | 1655 | CUGCUCUC GGAGGAAACUCC CU UCAAGGACAUCGUCCGGG UAGCCAGA | 3852 |
| 1427 | CUGGCUAG G AGAGCAGC | 1656 | GCUGCUCU GGAGGAAACUCC CU UCAAGGACAUCGUCCGGG CUAGCCAG | 3853 |
| 1429 | GGCUAGGA G AGCAGCUG | 1657 | CAGCUGCU GGAGGAAACUCC CU UCAAGGACAUCGUCCGGG UCCUAGCC | 3854 |
| 1437 | GAGCAGCU G GUGUGCUG | 1655 | CAGCACAC GGAGGAAACUCC CU UCAAGGACAUCGUCCGGG AGCUGCUC | 3855 |
| 1445 | GGUGUGCU G GCAAGCAG | 1659 | CUGCUUGC GGAGGAAACUCC CU UCAAGGACAUCGUCCGGG AGCACACC | 3856 |
| 1453 | GGCAAGCA G GCACCACC | 1663 | GGUGGUGC GGAGGAAACUCC CU UCAAGGACAUCGUCCGGG UGCUUGCC | 3857 |
| 1466 | CACCCCUU G GAACAUUU | 1661 | AAAUGUUC GGAGGAAACUCC CU UCAAGGACAUCGUCCGGG AAGGGGUG | 3858 |
| 1467 | ACCCCUUG G AACAUUUU | 1662 | AAAAUGUU GGAGGAAACUCC CU UCAAGGACAUCGUCCGGG CAAGGGGU | 3859 |
| 1500 | UACCUAAU G GGUGAGGU | 1663 | ACCUCACC GGAGGAAACUCC CU UCAAGGACAUCGUCCGGG AUUAGGUA | 3860 |
| 1501 | ACCUAAUG G GUGAGGUU | 1664 | AACCUCAC GGAGGAAACUCC CU UCAAGGACAUCGUCCGGG CAUUAGGU | 3861 |
| 1506 | AUGGGUGA G GUUACCAA | 1665 | UUGGUAAC GGAGGAAACUCC CU UCAAGGACAUCGUCCGGG UCACCCAU | 3862 |
| 1556 | AUACCUGC G GCCAGUGG | 1666 | CCACUGGC GGAGGAAACUCC CU UCAAGGACAUCGUCCGGG GCAGGUAU | 3863 |
| 1563 | CGGCCAGU G GAAGAUGU | 1667 | ACAUCUUC GGAGGAAACUCC CU UCAAGGACAUCGUCCGGG ACUGGCCG | 3864 |
| 1564 | GGCCAGUG G AAGAUGUG | 1668 | CACAUCUU GGAGGAAACUCC CU UCAAGGACAUCGUCCGGG CACUGGCC | 3865 |
| 1567 | CAGUGGAA G AUGUGGCC | 1669 | GGCCACAU GGAGGAAACUCC CU UCAAGGACAUCGUCCGGG UUCCACUG | 3866 |
| 1572 | GAAGAUGU G GCCACGUC | 1670 | GACGUGGC GGAGGAAACUCC CU UCAAGGACAUCGUCCGGG ACAUCUUC | 3867 |
| 1585 | CGUCCCAA G ACGACUGU | 1671 | ACAGUCGU GGAGGAAACUCC CU UCAAGGACAUCGUCCGGG UUGGGACG | 3868 |
| 1623 | UCAUCCAC G GGCACUGU | 1672 | ACAGUGCC GGAGGAAACUCC CU UCAAGGACAUCGUCCGGG GUGGAUGA | 3869 |
| 1624 | CAUCCACG G GCACUGUU | 1673 | AACAGUGC GGAGGAAACUCC CU UCAAGGACAUCGUCCGGG CGUGGAUG | 3870 |
| 1635 | ACUGUUAU G GGAGCUGU | 1674 | ACAGCUCC GGAGGAAACUCC CU UCAAGGACAUCGUCCGGG AUAACAGU | 3871 |
| 1636 | CUGUUAUG G GAGCUGUU | 1675 | AACAGCUC GGAGGAAACUCC CU UCAAGGACAUCGUCCGGG CAUAACAG | 3872 |
| 1637 | UGUUAUGG G AGCUGUUA | 1676 | UAACAGCU GGAGGAAACUCC CU UCAAGGACAUCGUCCGGG CCAUAACA | 3873 |
| 1650 | GUUAUCAU G GAGGGCUU | 1677 | AAGCCCUC GGAGGAAACUCC CU UCAAGGACAUCGUCCGGG AUGAUAAC | 3874 |
| 1651 | UUAUCAUG G AGGGCUUC | 1678 | GAAGCCCU GGAGGAAACUCC CU UCAAGGACAUCGUCCGGG CAUGAUAA | 3875 |
| 1653 | AUCAUGGA G GGCUUCUA | 1679 | UAGAAGCC GGAGGAAACUCC CU UCAAGGACAUCGUCCGGG UCCAUGAU | 3876 |
| 1654 | UCAUGGAG G GCUUCUAC | 1680 | GUAGAAGC GGAGGAAACUCC CU UCAAGGACAUCGUCCGGG CUCCAUGA | 3877 |
| 1676 | CUUUGAUC G GGCCCGAA | 1681 | UUCGGGCC GGAGGAAACUCC CU UCAAGGACAUCGUCCGGG GAUCAAAG | 3878 |
| 1677 | UUUGAUCG G GCCCGAAA | 1682 | UUUCGGGC GGAGGAAACUCC CU UCAAGGACAUCGUCCGGG CGAUCAAA | 3879 |
| 1693 | AACGAAUU G GCUUUGCU | 1683 | AGCAAAGC GGAGGAAACUCC CU UCAAGGACAUCGUCCGGG AAUUCGUU | 3880 |
| 1733 | UGAGUUCA G GACGGCAG | 1684 | CUGCCGUC GGAGGAAACUCC CU UCAAGGACAUCGUCCGGG UGAACUCA | 3881 |
| 1734 | GAGUUCAG G ACGGCAGC | 1685 | GCUGCCGU GGAGGAAACUCC CU UCAAGGACAUCGUCCGGG CUGAACUC | 3882 |
| 1737 | UUCAGGAC G GCAGCGGU | 1686 | ACCGCUGC GGAGGAAACUCC CU UCAAGGACAUCGUCCGGG GUCCUGAA | 3883 |
| 1743 | ACGGCAGC G GUGGAAGG | 1687 | CCUUCCAC GGAGGAAACUCC CU UCAAGGACAUCGUCCGGG GCUGCCGU | 3884 |
| 1746 | GCAGCCGU G GAAGGCCC | 1688 | GGGCCUUC GGAGGAAACUCC CU UCAAGGACAUCGUCCGGG ACCGCUGC | 3885 |
| 1747 | CAGCGGUG G AAGGCCCU | 1689 | AGGGCCUU GGAGGAAACUCC CU UCAAGGACAUCGUCCGGG CACCGCUG | 3886 |
| 1750 | CGGUGGAA G GCCCUUUU | 1690 | AAAAGGGC GGAGGAAACUCC CU UCAAGGACAUCGUCCGGG UUCCACCG | 3887 |
| 1767 | GUCACCUU G GACAUGGA | 1691 | UCCAUGUC GGAGGAAACUCC CU UCAAGGACAUCGUCCGGG AAGGUGAC | 3888 |
| 1768 | UCACCUUG G ACAUGGAA | 1692 | UUCCAUGU GGAGGAAACUCC CU UCAAGGACAUCGUCCGGG CAAGGUGA | 3889 |
| 1773 | UUGGACAU G GAAGACUG | 1693 | CAGUCUUC GGAGGAAACUCC CU UCAAGGACAUCGUCCGGG AUGUCCAA | 3890 |
| 1774 | UGGACAUG G AAGACUGU | 1694 | ACAGUCUU GGAGGAAACUCC CU UCAAGGACAUCGUCCGGG CAUGUCCA | 3891 |
| 1777 | ACAUGGAA G ACUGUGGC | 1695 | GCCACAGU GGAGGAAACUCC CU UCAAGGACAUCGUCCGGG UUCCAUGU | 3892 |
| 1783 | AAGACUGU G GCUACAAC | 1696 | GUUGUAGC GGAGGAAACUCC CU UCAAGGACAUCGUCCGGG ACAGUCUU | 3893 |
| 1800 | AUUCCACA G ACAGAUGA | 1697 | UCAUCUGU GGAGGAAACUCC CU UCAAGGACAUCGUCCGGG UGUGGAAU | 3894 |
| 1804 | CACAGACA G AUGAGUCA | 1698 | UGACUCAU GGAGGAAACUCC CU UCAAGGACAUCGUCCGGG UGUCUGUG | 3895 |
| 1839 | UAUGUCAU G GCUGCCAU | 1699 | AUGGCAGC GGAGGAAACUCC CU UCAAGGACAUCGUCCGGG AUGACAUA | 3896 |
| 1881 | UGCCUCAU G GUGUGUCA | 1700 | UGACACAC GGAGGAAACUCC CU UCAAGGACAUCGUCCGGG AUGAGGCA | 3897 |
| 1892 | GUGUCAGU G GCGCUGCC | 1701 | GGCAGCGC GGAGGAAACUCC CU UCAAGGACAUCGUCCGGG ACUGACAC | 3898 |
| 1960 | UGAAGUGA G GAGGCCCA | 1702 | UGGGCCUC GGAGGAAACUCC CU UCAAGGACAUCGUCCGGG UCACUUCA | 3899 |
| 1961 | GAAGUGAG G AGGCCCAU | 1703 | AUGGGCCU GGAGGAAACUCC CU UCAAGGACAUCGUCCGGG CUCACUUC | 3900 |
| 1963 | AGUGAGGA G GCCCAUGG | 1704 | CCAUGGGC GGAGGAAACUCC CU UCAAGGACAUCGUCCGGG UCCUCACU | 3901 |
| 1970 | AGGCCCAU G GGCAGAAG | 1705 | CUUCUGCC GGAGGAAACUCC CU UCAAGGACAUCGUCCGGG AUGGGCCU | 3902 |
| 1971 | GGCCCAUG G GCAGAAGA | 1706 | UCUUCUGC GGAGGAAACUCC CU UCAAGGACAUCGUCCGGG CAUGGGCC | 3903 |
| 1975 | CAUGGGCA G AAGAUAGA | 1707 | UCUAUCUU GGAGGAAACUCC CU UCAAGGACAUCGUCCGGG UGCCCAUG | 3904 |
| 1978 | GGGCAGAA G AUAGAGAU | 1708 | AUCUCUAU GGAGGAAACUCC CU UCAAGGACAUCGUCCGGG UUCUGCCC | 3905 |
| 1982 | AGAAGAUA G AGAUUCCC | 1709 | GGGAAUCU GGAGGAAACUCC CU UCAAGGACAUCGUCCGGG UAUCUUCU | 3906 |
| 1984 | AAGAUAGA G AUUCCCCU | 1710 | AGGGGAAU GGAGGAAACUCC CU UCAAGGACAUCGUCCGGG UCUAUCUU | 3907 |
| 1993 | AUUCCCCU G GACCACAC | 1711 | GUGUGGUC GGAGGAAACUCC CU UCAAGGACAUCGUCCGGG AGGGGAAU | 3908 |
| 1994 | UUCCCCUG G ACCACACC | 1712 | GGUGUGGU GGAGGAAACUCC CU UCAAGGACAUCGUCCGGG CAGGGGAA | 3909 |
| 2008 | ACCUCCGU G GUUCACUU | 1713 | AAGUGAAC GGAGGAAACUCC CU UCAAGGACAUCGUCCGGG ACGGAGGU | 3910 |
| 2018 | UUCACUUU G GUCACAAG | 1714 | CUUGUGAC GGAGGAAACUCC CU UCAAGGACAUCGUCCGGG AAAGUGAA | 3911 |
| 2029 | CACAAGUA G GAGACACA | 1715 | UGUGUCUC GGAGGAAACUCC CU UCAAGGACAUCGUCCGGG UACUUGUG | 3912 |
| 2030 | ACAAGUAG G AGACACAG | 1716 | CUGUGUCU GGAGGAAACUCC CU UCAAGGACAUCGUCCGGG CUACUUGU | 3913 |
| 2032 | AAGUAGGA G ACACAGAU | 1717 | AUCUGUGU GGAGGAAACUCC CU UCAAGGACAUCGUCCGGG UCCUACUU | 3914 |
| 2038 | GAGACACA G AUGGCACC | 1718 | GGUGCCAU GGAGGAAACUCC CU UCAAGGACAUCGUCCGGG UGUGUCUC | 3915 |
| 2041 | ACACAGAU G GCACCUGU | 1719 | ACAGGUGC GGAGGAAACUCC CU UCAAGGACAUCGUCCGGG AUCUGUGU | 3916 |
| 2050 | GCACCUGU G GCCAGAGC | 1720 | GCUCUGGC GGAGGAAACUCC CU UCAAGGACAUCGUCCGGG ACAGGUGC | 3917 |
| 2055 | UGUGGCCA G AGCACCUC | 1721 | GAGGUGCU GGAGGAAACUCC CU UCAAGGACAUCGUCCGGG UGGCCACA | 3918 |
| 2065 | GCACCUCA G GACCCUCC | 1722 | GGAGGGUC GGAGGAAACUCC CU UCAAGGACAUCGUCCGGG UGAGGUGC | 3919 |
| 2066 | CACCUCAG G ACCCUCCC | 1723 | GGGAGGGU GGAGGAAACUCC CU UCAAGGACAUCGUCCGGG CUGAGGUG | 3920 |
| 2101 | GCCUUGAU G GAGAAGGA | 1724 | UCCUUCUC GGAGGAAACUCC CU UCAAGGACAUCGUCCGGG AUCAAGGC | 3921 |
| 2102 | CCUUGAUG G AGAAGGAA | 1725 | UUCCUUCU GGAGGAAACUCC CU UCAAGGACAUCGUCCGGG CAUCAAGG | 3922 |
| 2104 | UUGAUGGA G AAGGAAAA | 1726 | UUUUCCUU GGAGGAAACUCC CU UCAAGGACAUCGUCCGGG UCCAUCAA | 3923 |
| 2107 | AUGGAGAA G GAAAAGGC | 1727 | GCCUUUUC GGAGGAAACUCC CU UCAAGGACAUCGUCCGGG UUCUCCAU | 3924 |
| 2108 | UGGAGAAG G AAAAGGCU | 1728 | AGCCUUUU GGAGGAAACUCC CU UCAAGGACAUCGUCCGGG CUUCUCCA | 3925 |
| 2113 | AAGGAAAA G GCUGGCAA | 1729 | UUGCCAGC GGAGGAAACUCC CU UCAAGGACAUCGUCCGGG UUUUCCUU | 3926 |
| 2117 | AAAAGGCU G GCAAGGUG | 1730 | CACCUUGC GGAGGAAACUCC CU UCAAGGACAUCGUCCGGG AGCCUUUU | 3927 |
| 2122 | GCUGGCAA G GUGGGUUC | 1731 | GAACCCAC GGAGGAAACUCC CU UCAAGGACAUCGUCCGGG UUGCCAGC | 3928 |
| 2125 | GGCAAGGU G GGUUCCAG | 1732 | CUGGAACC GGAGGAAACUCC CU UCAAGGACAUCGUCCGGG ACCUUGCC | 3929 |
| 2126 | GCAAGGUG G GUUCCAGG | 1733 | CCUGGAAC GGAGGAAACUCC CU UCAAGGACAUCGUCCGGG CACCUUGC | 3930 |
| 2133 | GGGUUCCA G GGACUGUA | 1734 | UACAGUCC GGAGGAAACUCC CU UCAAGGACAUCGUCCGGG UGGAACCC | 3931 |
| 2134 | GGUUCCAG G GACUGUAC | 1735 | GUACAGUC GGAGGAAACUCC CU UCAAGGACAUCGUCCGGG CUGGAACC | 3932 |
| 2135 | GUUCCAGG G ACUGUACC | 1736 | GGUACAGU GGAGGAAACUCC CU UCAAGGACAUCGUCCGGG CCUGGAAC | 3933 |
| 2148 | UACCUGUA G GAAACAGA | 1737 | UCUGUUUC GGAGGAAACUCC CU UCAAGGACAUCGUCCGGG UACAGGUA | 3934 |
| 2149 | ACCUGUAG G AAACAGAA | 1738 | UUCUGUUU GGAGGAAACUCC CU UCAAGGACAUCGUCCGGG CUACAGGU | 3935 |
| 2155 | AGGAAACA G AAAAGAGA | 1739 | UCUCUUUU GGAGGAAACUCC CU UCAAGGACAUCGUCCGGG UGUUUCCU | 3936 |
| 2160 | ACAGAAAA G AGAAGAAA | 1740 | UUUCUUCU GGAGGAAACUCC CU UCAAGGACAUCGUCCGGG UUUUCUGU | 3937 |
| 2162 | AGAAAAGA G AAGAAAGA | 1741 | UCUUUCUU GGAGGAAACUCC CU UCAAGGACAUCGUCCGGG UCUUUUCU | 3938 |
| 2165 | AAAGAGAA G AAAGAAGC | 1742 | GCUUCUUU GGAGGAAACUCC CU UCAAGGACAUCGUCCGGG UUCUCUUU | 3939 |
| 2169 | AGAAGAAA G AAGCACUC | 1743 | GAGUGCUU GGAGGAAACUCC CU UCAAGGACAUCGUCCGGG UUUCUUCU | 3940 |
| 2182 | ACUCUGCU G GCGGGAAU | 1744 | AUUCCCGC GGAGGAAACUCC CU UCAAGGACAUCGUCCGGG AGCAGAGU | 3941 |
| 2185 | CUGCUGGC G GGAAUACU | 1745 | AGUAUUCC GGAGGAAACUCC CU UCAAGGACAUCGUCCGGG GCCAGCAG | 3942 |
| 2186 | UGCUGGCG G GAAUACUC | 1746 | GAGUAUUC GGAGGAAACUCC CU UCAAGGACAUCGUCCGGG CGCCAGCA | 3943 |
| 2187 | GCUGGCGG G AAUACUCU | 1747 | AGAGUAUU GGAGGAAACUCC CU UCAAGGACAUCGUCCGGG CCGCCAGC | 3944 |
| 2197 | AUACUCUU G GUCACCUC | 1748 | GAGGUGAC GGAGGAAACUCC CU UCAAGGACAUCGUCCGGG AAGAGUAU | 3945 |
| 2217 | UUUAAGUC G GGAAAUUC | 1749 | GAAUUUCC GGAGGAAACUCC CU UCAAGGACAUCGUCCGGG GACUUAAA | 3946 |
| 2218 | UUAAGUCG G GAAAUUCU | 1750 | AGAAUUUC GGAGGAAACUCC CU UCAAGGACAUCGUCCGGG CGACUUAA | 3947 |
| 2219 | UAAGUCGG G AAAUUCUG | 1751 | CAGAAUUU GGAGGAAACUCC CU UCAAGGACAUCGUCCGGG CCGACUUA | 3948 |
| 2311 | UAGUUUCA G AAGUACUG | 1752 | CAGUACUU GGAGGAAACUCC CU UCAAGGACAUCGUCCGGG UGAAACUA | 3949 |
| 2319 | GAAGUACU G GCAUCACA | 1753 | UGUGAUGC GGAGGAAACUCC CU UCAAGGACAUCGUCCGGG AGUACUUC | 3950 |
| 2332 | CACACGCA G GUUACCUU | 1754 | AAGGUAAC GGAGGAAACUCC CU UCAAGGACAUCGUCCGGG UGCGUGUG | 3951 |
| 2341 | GUUACCUU G GCGUGUGU | 1755 | ACACACGC GGAGGAAACUCC CU UCAAGGACAUCGUCCGGG AAGGUAAC | 3952 |
| 2356 | GUCCCUGU G GUACCCUG | 1756 | CAGGGUAC GGAGGAAACUCC CU UCAAGGACAUCGUCCGGG ACAGGGAC | 3953 |
| 2364 | GGUACCCU G GCAGAGAA | 1757 | UUCUCUGC GGAGGAAACUCC CU UCAAGGACAUCGUCCGGG AGGGUACC | 3954 |
| 2368 | CCCUGGCA G AGAAGAGA | 1758 | UCUCUUCU GGAGGAAACUCC CU UCAAGGACAUCGUCCGGG UGCCAGGG | 3955 |
| 2370 | CUGGCAGA G AAGAGACC | 1759 | GGUCUCUU GGAGGAAACUCC CU UCAAGGACAUCGUCCGGG UCUGCCAG | 3956 |
| 2373 | GCAGAGAA G AGACCAAG | 1760 | CUUGGUCU GGAGGAAACUCC CU UCAAGGACAUCGUCCGGG UUCUCUGC | 3957 |
| 2375 | AGAGAAGA G ACCAAGCU | 1761 | AGCUUGGU GGAGGAAACUCC CU UCAAGGACAUCGUCCGGG UCUUCUCU | 3958 |
| 2396 | UCCCUGCU G GCCAAAGU | 1762 | ACUUUGGC GGAGGAAACUCC CU UCAAGGACAUCGUCCGGG AGCAGGGA | 3959 |
| 2410 | AGUCAGUA G GAGAGGAU | 1763 | AUCCUCUC GGAGGAAACUCC CU UCAAGGACAUCGUCCGGG UACUGACU | 3960 |
| 2411 | GUCAGUAG G AGAGGAUG | 1764 | CAUCCUCU GGAGGAAACUCC CU UCAAGGACAUCGUCCGGG CUACUGAC | 3961 |
| 2413 | CAGUAGGA G AGGAUGCA | 1765 | UGCAUCCU GGAGGAAACUCC CU UCAAGGACAUCGUCCGGG UCCUACUG | 3962 |
| 2415 | GUAGGAGA G GAUGCACA | 1766 | UGUGCAUC GGAGGAAACUCC CU UCAAGGACAUCGUCCGGG UCUCCUAC | 3963 |
| 2416 | UAGGAGAG G AUGCACAG | 1767 | CUGUGCAU GGAGGAAACUCC CU UCAAGGACAUCGUCCGGG CUCUCCUA | 3964 |
| 2441 | UUGCUUUA G AGACAGGG | 1768 | CCCUGUCU GGAGGAAACUCC CU UCAAGGACAUCGUCCGGG UAAAGCAA | 3965 |
| 2443 | GCUUUAGA G ACAGGGAC | 1769 | GUCCCUGU GGAGGAAACUCC CU UCAAGGACAUCGUCCGGG UCUAAAGC | 3966 |
| 2447 | UAGAGACA G GGACUGUA | 1770 | UACAGUCC GGAGGAAACUCC CU UCAAGGACAUCGUCCGGG UGUCUCUA | 3967 |
| 2448 | AGAGACAG G GACUGUAU | 1771 | AUACAGUC GGAGGAAACUCC CU UCAAGGACAUCGUCCGGG CUGUCUCU | 3968 |
| 2449 | GAGACAGG G ACUGUAUA | 1772 | UAUACAGU GGAGGAAACUCC CU UCAAGGACAUCGUCCGGG CCUGUCUC | 3969 |
| 2473 | CUAACAUU G GUGCAAAG | 1773 | CUUUGCAC GGAGGAAACUCC CU UCAAGGACAUCGUCCGGG AAUGUUAG | 3970 |
| 2481 | GGUGCAAA G AUUGCCUC | 1774 | GAGGCAAU GGAGGAAACUCC CU UCAAGGACAUCGUCCGGG UUUGCACC | 3971 |
| 2511 | AAAAACUA G AAAAAAAA | 1775 | UUUUUUUU GGAGGAAACUCC CU UCAAGGACAUCGUCCGGG UAGUUUUU | 3972 |

Input Sequence = AF190725.
Cut Site = G/
Stem Length = 5.
Core Sequence = GGAGGAAACUCC CU UCAAGGACAUCGUCCGGG
AF190725 (Homo sapiens beta-site APP cleaving enzyme (BACE) mRNA, 2526 bp)

What we claim is:

1. An enzymatic nucleic acid molecule which down regulates expression of a beta site APP-cleaving enzyme (BACE) gene.

2. A nucleic acid molecule which down regulates expression of a presenilin (ps-2) gene.

3. The enzymatic nucleic acid of claim 1, wherein said enzymatic nucleic acid molecule is adapted for use to treat Alzheimer's disease.

4. The nucleic acid molecule of claim 2, wherein said nucleic acid molecule is an enzymatic nucleic acid molecule.

5. The nucleic acid molecule of claim 4, wherein said enzymatic nucleic acid molecule has an endonuclease activity to cleave RNA encoded by said ps-2 gene.

6. The nucleic acid of claim 1, wherein a binding arm of said enzymatic nucleic acid molecule comprise sequences complementary to any of sequences defined as sequence ID Nos. 1-1775.

7. The nucleic acid molecule of claim 1, wherein said enzymatic nucleic acid molecule comprises any of sequences defined as sequence ID Nos. 1776-3972.

8. The nucleic acid molecule of claim 2, wherein said nucleic acid molecule is an antisense nucleic acid molecule.

9. An antisense nucleic acid molecule comprising sequence complementary to any of the sequences defined as sequence ID Nos. 1-1775.

10. The enzymatic nucleic acid molecule of any of claims 1 and 4, wherein said enzymatic nucleic acid molecule is in a hammerhead (HH) motif.

11. The enzymatic nucleic acid molecule of any of claims 1 and 4, wherein said enzymatic nucleic acid molecule is in a hairpin, hepatitis Delta virus, group I intron, VS nucleic acid, amberzyme, zinzyme or RNAse P nucleic acid motif.

12. The enzymatic nucleic acid molecule of claim 11, wherein said zinzyme motif comprises sequences complementary to any of the substrate sequences shown in Table VI.

13. The enzymatic nucleic acid molecule of claim 11, wherein said amberzyme motif comprises sequences complementary to any of the substrate sequences shown in Table VII.

14. The enzymatic nucleic acid molecule of any of claims 1 and 4, wherein said enzymatic nucleic acid molecule is in a NCH motif.

15. The enzymatic nucleic acid molecule of any of claims 1 and 4, wherein said enzymatic nucleic acid molecule is in a G-cleaver motif.

16. The enzymatic nucleic acid molecule of any of claims 1 and 4, wherein said enzymatic nucleic acid molecule is a DNAzyme.

17. The enzymatic nucleic acid molecule of claims 1, wherein said enzymatic nucleic acid molecule comprises between 12 and 100 bases complementary to the RNA of BACE gene.

18. The enzymatic nucleic acid of claim 1. wherein said enzymatic nucleic acid molecule comprises between 14 and 24 bases complementary to the RNA of BACE gene.

19. The enzymatic nucleic acid molecule of claim 1, wherein said enzymatic nucleic acid is chemically synthesized.

20. The enzymatic nucleic acid molecule of claim 1, wherein said enzymatic nucleic acid comprises at least one 2′-sugar modification.

21. The enzymatic nucleic acid molecule of claim 1, wherein said enzymatic nucleic acid comprises at least one nucleic acid base modification.

22. The enzymatic nucleic acid molecule of claim 1, wherein said enzymatic nucleic acid comprises at least one phosphate backbone modification.

23. A mammalian cell including the enzymatic nucleic acid molecule of claim 1, wherein said mammalian cell is not a living human.

24. The mammalian cell of claim 23, wherein said mammalian cell is a human cell.

25. A method of reducing BACE activity in a cell, comprising the step of contacting said cell with the enzymatic nucleic acid molecule of claim 1, under conditions suitable for said inhibition.

26. A method of treatment of a patient having a condition associated with the level of BACE, comprising contacting cells of said patient with the enzymatic nucleic acid molecule of claim 1, under conditions suitable for said treatment.

27. The method of claim 26 further comprising the use of one or more drug therapies under conditions suitable for said treatment.

28. A method of cleaving RNA of BACE gene, comprising, contacting the enzymatic nucleic acid molecule of claim 1, with said RNA under conditions suitable for the cleavage of said RNA.

29. The method of claim 28, wherein said cleavage is carried out in the presence of a divalent cation.

30. The method of claim 29, wherein said divalent cation is Mg²⁺.

31. The enzymatic nucleic acid molecule of claim 1, wherein said enzymatic nucleic acid comprises a cap structure, wherein the cap structure is at the 5′-end or 3′-end or both the 5′-end and the 3′-end.

32. The enzymatic nucleic acid molecule of claim 10, wherein said hammerhead motif comprises sequences complementary to any of sequences shown as Seq ID Nos 1-284.

33. The enzymatic nucleic acid molecule of claim 14, wherein said NCH motif comprises sequences complementary to any of sequences shown as Seq ID Nos 285-959.

34. The enzymatic nucleic acid molecule of claim 15, wherein said G-cleaver motif comprises sequences complementary to any of sequences shown as Seq ID Nos 960-1145.

35. The enzymatic nucleic acid molecule of claim 16, wherein said DNAzyme comprises sequences complementary to any of substrate sequences shown in Table VII.

36. The method of any of claim 25 or 27, wherein said enzymatic nucleic acid molecule is in a hammerhead motif.

37. The method of any of claim 25 or 27, wherein said nucleic acid molecule is a DNAzyme.

38. An expression vector comprising nucleic acid sequence encoding at least one enzymatic nucleic acid molecule of claim 1, in a manner which allows expression of that enzymatic nucleic acid molecule.

39. A mammalian cell including an expression vector of claim 38, wherein said mammalian cell is not a living human.

40. The mammalian cell of claim 39, wherein said mammalian cell is a human cell.

41. The expression vector of claim 38, wherein said enzymatic nucleic acid molecule is in a hammerhead motif.

42. The expression vector of claim 38, wherein said expression vector further comprises a sequence for an antisense nucleic acid molecule complementary to the RNA of BACE gene.

43. The expression vector of claim 38, wherein said expression vector comprises sequence encoding at least two said enzymatic nucleic acid molecules, which may be same or different.

44. The expression vector of claim 43, wherein one said expression vector further comprises sequence encoding antisense nucleic acid molecule complementary to the RNA of BACE gene.

45. A method for treatment of Alzheimer's disease comprising the step of administering to a patient the enzymatic nucleic acid molecule of claim 1 under conditions suitable for said treatment.

46. The method of claim 45, wherein said treatment of Alzheimer's disease is treatment of dementia.

47. A method for treatment of Alzheimer's disease comprising the step of administering to a patient the antisense nucleic acid molecule of claim 9 under conditions suitable for said treatment.

48. The method of claim 45, wherein said enzymatic nucleic acid molecule is in a hammerhead motif.

49. The method of claim 45, wherein said method further comprises administering to said patient the enzymatic nucleic acid molecule in conjunction with one or more of other therapies.

50. The enzymatic nucleic acid molecule of anv of claim 1, wherein said enzymatic nucleic acid molecule comprises at least fine ribose residues; at least ten 2′-O-methyl modifications, and a 3′- end modification.

51. The enzymatic nucleic acid molecule of claim 50, wherein said enzymatic nucleic acid molecule further comprises phosphorothioate linkages on at least three of the 5′ terminal nucleotides.

52. The enzymatic nucleic acid molecule of claim 50, wherein said 3′- end modification is 3′-3′ inverted abasic moiety.

53. The enzymatic nucleic acid molecule of claim 16, wherein said DNAzyme comprises at least ten 2′-O-methyl modifications and a 3′-end modification.

54. The enzymatic nucleic acid molecule of claim 53, wherein said DNAzyme further comprises phosphorothioate linkages on at least three of the 5′ terminal nucleotides.

55. The enzymatic nucleic acid molecule of claim 53, wherein said 3′-end modification is 3′-3′ inverted abasic moiety.
